SEQUENCE LISTING (60 sequences)

SEQ ID NO: 1 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaacttttggatgacctgggtccgccaggctccagggaaggggctggagtgggtggccaacatgaaccaagatggaagtgagaaatactatgtggactctgtgaagggccgattcaccatctccagagacaacgccaagagctcactgtatctgcaaatgaacagcctgagagccgaggacacggctgtgtattactgtgcgagagatcgggaatattgtattagtaccagctgctatgatgactttgactactggggccagggaaccctggtcaccgtctcctca

SEQ ID NO: 2 - protein, 125 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 3 - DNA, 24 bp, synthetic construct
  source 1..24: mol_type = other DNA
ggattcacctttagtaacttttgg

SEQ ID NO: 4 - protein, 8 aa, synthetic construct
GFTFSNFW

SEQ ID NO: 5 - DNA, 24 bp, synthetic construct
  source 1..24: mol_type = other DNA
atgaaccaagatggaagtgagaaa

SEQ ID NO: 6 - protein, 8 aa, synthetic construct
MNQDGSEK

SEQ ID NO: 7 - DNA, 54 bp, synthetic construct
  source 1..54: mol_type = other DNA
gcgagagatcgggaatattgtattagtaccagctgctatgatgactttgactac

SEQ ID NO: 8 - protein, 18 aa, synthetic construct
ARDREYCISTSCYDDFDY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcatagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLHSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 18 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 19 - DNA, 18 bp, synthetic construct
  source 1..18: mol_type = other DNA
cagagcattagcagctat

SEQ ID NO: 20 - protein, 6 aa, synthetic construct
QSISSY

SEQ ID NO: 21-22 - skipped (2 entries)

SEQ ID NO: 23 - DNA, 30 bp, synthetic construct; identical to SEQ ID NO: 15
  source 1..30: mol_type = other DNA

SEQ ID NO: 24 - protein, 10 aa, synthetic construct
QQSYSTPPIT

SEQ ID NO: 25 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcaagggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttatcattatggactggacgtgtggggacaagggacaacagtgacagtgagtagc

SEQ ID NO: 26 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 27 - DNA, 24 bp, synthetic construct
  source 1..24: mol_type = other DNA
gggtttacattcgacgattacagc

SEQ ID NO: 28 - protein, 8 aa, synthetic construct
GFTFDDYS

SEQ ID NO: 29 - DNA, 24 bp, synthetic construct
  source 1..24: mol_type = other DNA
atatcatggaactcaggaagcaag

SEQ ID NO: 30 - protein, 8 aa, synthetic construct
ISWNSGSK

SEQ ID NO: 31 - DNA, 51 bp, synthetic construct
  source 1..51: mol_type = other DNA
gcaaaatacggcagtggttatggcaagttttatcattatggactggacgtg

SEQ ID NO: 32 - protein, 17 aa, synthetic construct
AKYGSGYGKFYHYGLDV

SEQ ID NO: 33 - DNA, 372 bp, synthetic construct
  source 1..372: mol_type = other DNA
gaagtacagcttgtagaatccggcggaggactggtacaacctggaagaagtcttagactgagttgcgcagctagtgggtttacattcgacgattacagcatgcattgggtgaggcaagctcctggtaaaggattggaatgggttagcgggatatcatggaactcaggaagcatcggatacgccgacagcgtgaaaggccgatttacaatatctagggacaacgcaaaaaactctctctaccttcaaatgaactctcttagggcagaagacacagcattgtattattgcgcaaaatacggcagtggttatggcaagttttattattatggaatggacgtgtggggacaagggacaacagtgacagtgagtagc

SEQ ID NO: 34 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 35 - DNA, 24 bp, synthetic construct
  source 1..24: mol_type = other DNA
gggtttacattcgacgattacagc

SEQ ID NO: 36 - protein, 8 aa, synthetic construct
GFTFDDYS

SEQ ID NO: 37 - DNA, 24 bp, synthetic construct
  source 1..24: mol_type = other DNA
atatcatggaactcaggaagcatc

SEQ ID NO: 38 - protein, 8 aa, synthetic construct
ISWNSGSI

SEQ ID NO: 39 - DNA, 51 bp, synthetic construct
  source 1..51: mol_type = other DNA
gcaaaatacggcagtggttatggcaagttttattattatggaatggacgtg

SEQ ID NO: 40 - protein, 17 aa, synthetic construct
AKYGSGYGKFYYYGMDV

SEQ ID NO: 41 - protein, 451 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 43 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 127 aa, synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 45 - protein, 108 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 46 - protein, 123 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 47 - protein, 8 aa, synthetic construct
GFTFNDYA

SEQ ID NO: 48 - protein, 8 aa, synthetic construct
ISWNSDSI

SEQ ID NO: 49 - protein, 20 aa, synthetic construct
AKDNHYGSGSYYYYQYGMDV

SEQ ID NO: 50 - protein, 6 aa, synthetic construct
QSVSSN

SEQ ID NO: 51 - skipped

SEQ ID NO: 52 - protein, 9 aa, synthetic construct
QHYINWPLT

SEQ ID NO: 53 - protein, 8 aa, synthetic construct
GFTFDDYT

SEQ ID NO: 54 - protein, 8 aa, synthetic construct
ISWNSGSI

SEQ ID NO: 55 - protein, 16 aa, synthetic construct
AKDNSGYGHYYYGMDV

SEQ ID NO: 56 - protein, 6 aa, synthetic construct
HHHHHH

SEQ ID NO: 57 - protein, 8 aa, synthetic construct
HHHHHHHH

SEQ ID NO: 58 - protein, 10 aa, synthetic construct
HHHHHHHHHH

SEQ ID NO: 59 - RNA, 100 nt, synthetic construct
  source 1..100: mol_type = other RNA
  misc_difference 1..100: note = This sequence may encompass 20, 30, 40, 50, 60, 70, 80, 90, or 100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - RNA, 100 nt, synthetic construct; identical to SEQ ID NO: 59
  source 1..100: mol_type = other RNA
  misc_difference 1..100: note = This sequence may encompass 95-100 nucleotides

(19) United States
(12) Patent Application Publication
(10) Pub. No.: US 2025/0242018 A1
(43) Pub. Date: Jul. 31, 2025

(54) COMBINATION IMMUNOSUPPRESSION FOR INHIBITING AN IMMUNE RESPONSE AND ENABLING IMMUNOGEN ADMINISTRATION AND RE-ADMINISTRATION

(71) Applicant: Regeneron Pharmaceuticals, Inc., Tarrytown, NY (US)

(72) Inventors: Christos KYRATSOUS, Irvington, NY (US); Andre Limnander, New York, NY (US); Nicholas Giovannone, Palisades, NY (US); Andrew Baik, Scarsdale, NY (US); Katherine Cygnar, New York, NY (US); Jamie M. Orengo, Cortlandt Manor, NY (US)

(73) Assignee: Regeneron Pharmaceuticals, Inc., Tarrytown, NY (US)

(21) Appl. No.: 19/036,433

(22) Filed: Jan. 24, 2025

Related U.S. Application Data
(60) Provisional application No. 63/625,524, filed on Jan. 26, 2024.

(51) Int. Cl. A61K 39/395; A61K 39/00; C12N 15/86

(52) U.S. Cl. CPC A61K 39/3955; C12N 15/86; A61K 2039/507; C12N 2750/14143

(57) ABSTRACT

The present disclosure provides compositions and methods for inhibiting or preventing an immune response to an immunogen (e.g., an immunogenic delivery vehicle) in a subject in need thereof, comprising administering to the subject an effective amount of a plasma cell depleting agent, e.g., an antigen-binding molecule that binds to B cell maturation antigen (BCMA) and cluster of differentiation 3 (CD3) (e.g., an anti-BCMA×CD3 bispecific antibody, or a functional fragment thereof) or a B cell depleting agent, e.g., an antigen-binding molecule that binds to CD20 and CD3 (e.g., an anti-CD20×CD3 bispecific antibody, or a functional fragment thereof), either alone or in combination with one another, and/or in combination with an immunoglobulin depleting agent such as a neonatal fragment crystallizable (Fc) receptor (FcRn) blocker (e.g., efgartigimod).

### CROSS-REFERENCE TO RELATED APPLICATION

This patent application claims the benefit of U.S. Provisional Application No. 63/625,524, filed Jan. 26, 2024, the disclosure of which is incorporated by reference herein in its entirety for all purposes.

### SEQUENCE LISTING

The instant application contains a Sequence Listing which has been submitted electronically in XML file format and is hereby incorporated by reference in its entirety. Said XML copy, created on Jan. 16, 2025, is named 250298_000776_SL.xml and is 54,578 bytes in size.

### FIELD OF THE INVENTION

The present disclosure provides compositions and methods for inhibiting or preventing an immune response to an immunogen (e.g., an immunogenic delivery vehicle) in a subject in need thereof, comprising administering to the subject an effective amount of a plasma cell depleting agent, e.g., an antigen-binding molecule that binds to B cell maturation antigen (BCMA) and CD3 (e.g., an anti-BCMA×CD3 bispecific antibody, or a functional fragment thereof), either alone or in combination with a B cell depleting agent (e.g., anti-CD20×CD3 bispecific antibody, or a functional fragment thereof), and/or an immunoglobulin depleting agent such as a neonatal fragment crystallizable (Fc) receptor (FcRn) blocker (e.g., efgartigimod). Further disclosed herein are methods and compositions for inhibiting an immune response to an immunogen in a subject in need thereof by using a B cell depleting agent administered alone.

### BACKGROUND

Adeno-associated virus (AAV)-based vectors hold tremendous promise to transform treatment of genetic diseases. Yet, the potential of AAV gene therapy has so far been limited by development of host antibodies (e.g., neutralizing antibodies (nAbs)) that block transduction or affect uptake on subsequent exposures. Clinically, the inability to re-dose AAVs presents challenges because efficacy cannot be restored if transgene expression is subtherapeutic or lost (e.g., due to cell division, silencing, or a cytotoxic immune response). Moreover, due to natural AAV exposure, many patients develop nAbs prior to treatment that render them ineligible for even a single dose. Therefore, strategies that prevent or attenuate anti-AAV nAb responses could vastly expand the utility and accessibility of existing AAV gene therapies, while safeguarding eligibility for future AAV-based advances.

### SUMMARY

As specified in the Background section above, there exists a need in the art to enhance the efficacy of treatments with, e.g., recombinant vectors (e.g., AAV). This can be achieved, e.g., by inhibiting or preventing an immune response against such recombinant vectors and/or their transgene products (e.g., therapeutic polypeptides or polynucleotides encoded by the transgene), thereby improving efficacy and reducing toxicity of gene therapy. Such advancements would allow for stepwise dosing and/or effective re-administration (i.e., re-dosing) of the recombinant vectors (e.g., AAV) to increase or maintain the level of a transgene expression. The present disclosure addresses these and other needs.

In one aspect, provided herein is a method for inhibiting or preventing an immune response to an immunogen in a subject in need thereof, wherein the subject has pre-existing immunity against the immunogen, the method comprising administering to the subject an effective amount of a plasma cell depleting agent.

In another aspect, provided herein is a method for inhibiting or preventing generation of antibodies to an immunogen in a subject in need thereof, wherein the subject has pre-existing immunity against the immunogen, the method comprising administering to the subject an effective amount of a plasma cell depleting agent.

In another aspect, provided herein is a method for increasing effectiveness of re-administration of an immunogen to a subject in need thereof, wherein the subject has pre-existing immunity against the immunogen, the method comprising administering to the subject an effective amount of a plasma cell depleting agent.

In some embodiments, the immunogen re-administration occurs via the same administration route as its prior administration.

In some embodiments, the immunogen re-administration occurs via a different administration route than its prior administration.

In some embodiments, the method comprises determining the presence of neutralizing antibodies to the immunogen in the subject.

In some embodiments, the plasma cell depleting agent is administered before the administration of the immunogen.

In some embodiments, the plasma cell depleting agent is administered simultaneously with the administration of the immunogen.

In some embodiments, the plasma cell depleting agent is administered after the administration of the immunogen.

In some embodiments, the immunogen is administered two or more times and the plasma cell depleting agent is administered before and/or between each of the administrations of the immunogen.

In some embodiments, the immunogen is an immunogenic delivery vehicle, a polypeptide, a polynucleotide, a glycan, or a lipid.

In some embodiments, the immunogen is an immunogenic delivery vehicle or a polypeptide or polynucleotide encoded by a transgene contained within the immunogenic delivery vehicle.

In another aspect, provided herein is a method for increasing or maintaining the level of a transgene expression in a subject in need thereof, the method comprising administering to the subject an effective amount of a plasma cell depleting agent.

In some embodiments, the method comprises determining the presence of neutralizing antibodies to the immunogen in the subject.

In some embodiments, the transgene is delivered to the subject via an immunogenic delivery vehicle.

In some embodiments, the level of transgene expression is increased or maintained by inhibiting an immune response to the immunogenic delivery vehicle and/or by inhibiting an immune response to a polypeptide or polynucleotide encoded by the transgene.

In some embodiments, the level of transgene expression is increased or maintained by inhibiting antibody responses to the polypeptide or polynucleotide encoded by the transgene.

In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell.

In some embodiments, the immunogenic delivery vehicle is a viral vector.

In another aspect, provided herein is a method for increasing effectiveness of administration of a subsequently administered viral vector following administration of an originally administered viral vector in a subject in need thereof, the method comprising administering to the subject an effective amount of a plasma cell depleting agent, wherein the subsequently administered viral vector is of the same or similar viral origin as the originally administered viral vector.

In some embodiments, the method comprises determining the presence of neutralizing antibodies to the immunogen in the subject.

In some embodiments, the subsequently administered viral vector is administered via the same administration route as the originally administered viral vector.

In some embodiments, the subsequently administered viral vector is administered via a different administration route from the originally administered viral vector.

In some embodiments, the plasma cell depleting agent is administered before the administration of the subsequently administered viral vector(s).

In some embodiments, the plasma cell depleting agent is administered simultaneously with the administration of the subsequently administered viral vector(s).

In some embodiments, the subsequently administered viral vectors are administered two or more times and the plasma cell depleting agent is administered before and/or between each of the administrations of the subsequently administered viral vectors.

In some embodiments, the viral vector is derived from an adeno-associated virus (AAV), an adenovirus, a retrovirus, or an oncolytic virus.

In some embodiments, the viral vector is AAV.

In some embodiments, the plasma cell depleting agent is capable of depleting long-lived plasma cells (LLPC).

In some embodiments, the plasma cell depleting agent is a B cell maturation antigen (BCMA) targeting agent.

In some embodiments, the BCMA targeting agent is a chimeric antigen receptor (CAR) against BCMA or an anti-BCMA antibody or a functional fragment thereof.

In some embodiments, the anti-BCMA antibody or functional fragment thereof is conjugated to a cytotoxic agent.

In some embodiments, the anti-BCMA antibody is a multispecific antibody or a functional fragment thereof.

In some embodiments, the multispecific anti-BCMA antibody or functional fragment thereof targets BCMA and CD3.

In some embodiments, the multispecific anti-BCMA antibody or functional fragment thereof is anti-BCMA×CD3 bispecific antibody or functional fragment thereof.

In some embodiments, the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B.

In some embodiments, the anti-BCMA×CD3 bispecific antibody or functional fragment thereof comprises a first antigen-binding domain that specifically binds to BCMA comprising three heavy chain CDRs (HCDR1, HCDR2 and HCDR3) contained within a heavy chain variable region (HCVR) comprising the amino acid sequence of SEQ ID NO: 2, and three light chain CDRs (LCDR1, LCDR2 and LCDR3) contained within a light chain variable region (LCVR) comprising the amino acid sequence of SEQ ID NO: 18.

In some embodiments, the first antigen-binding domain that specifically binds to BCMA comprises a HCDR1 comprising the amino acid sequence of SEQ ID NO: 4, a HCDR2 comprising the amino acid sequence of SEQ ID NO: 6, a HCDR3 comprising the amino acid sequence of SEQ ID NO: 8, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some embodiments, the anti-BCMA×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence selected from the group consisting of SEQ ID NOs: 26 and 34, and three light chain CDRs (LCDR1, LCDR2 and LCDR3) contained within a light chain variable region (LCVR) comprising the amino acid sequence of SEQ ID NO: 18.

In some embodiments, the second antigen-binding domain that specifically binds to CD3 comprises a HCDR1 comprising the amino acid sequence of SEQ ID NO: 28 or 36, a HCDR2 comprising the amino acid sequence of SEQ ID NO: 30 or 38, a HCDR3 comprising the amino acid sequence of SEQ ID NO: 32 or 40, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some embodiments, the anti-BCMA×CD3 bispecific antibody or functional fragment thereof comprises:
a) a first antigen-binding domain that comprises HCDR1, HCDR2, and HCDR3 comprising the amino acid sequences of SEQ ID NOs: 4, 6, and 8, respectively, and LCDR1, LCDR2, and LCDR3 comprising the amino acid sequences of SEQ ID NOs: 20, 22, and 24, respectively; and
b) a second antigen-binding domain that comprises HCDR1, HCDR2, and HCDR3 comprising the amino acid sequences of SEQ ID NOs: 28, 30, and 32, respectively, and LCDR1, LCDR2, and LCDR3 comprising the amino acid sequences of SEQ ID NOs: 20, 22, and 24, respectively.

In some embodiments, the anti-BCMA×CD3 bispecific antibody or functional fragment thereof comprises:
a) a first antigen-binding domain that comprises HCDR1, HCDR2, and HCDR3 comprising the amino acid sequences of SEQ ID NOs: 4, 6, and 8, respectively, and LCDR1, LCDR2, and LCDR3 comprising the amino acid sequences of SEQ ID NOs: 20, 22, and 24, respectively; and
b) a second antigen-binding domain that comprises HCDR1, HCDR2, and HCDR3 comprising the amino acid sequences of SEQ ID NOs: 36, 38, and 40, respectively, and LCDR1, LCDR2, and LCDR3 comprising the amino acid sequences of SEQ ID NOs: 20, 22, and 24, respectively.

In some embodiments, the anti-BCMA×CD3 bispecific antibody or functional fragment thereof comprises a human IgG heavy chain constant region.

In some embodiments, the human IgG heavy chain constant region is isotype IgG4 or IgG1.

In some embodiments, the human IgG heavy chain constant region comprises one or more modifications that increase binding to a neonatal Fc receptor (FcRn).

In some embodiments, the human IgG heavy chain constant region comprises one or more modifications that decrease binding to an Fc-gamma receptor (FcγR).

In some embodiments, the method further comprises administering to the subject an effective amount of a B cell depleting agent and/or an immunoglobulin depleting agent.

In some embodiments, the B cell depleting agent is administered before, at the same time as, or after the plasma cell depleting agent.

In some embodiments, the immunoglobulin depleting agent is administered after the plasma cell depleting agent.

In some embodiments, the B cell depleting agent is capable of depleting B cells and plasma cells that express low levels of BCMA.

In some embodiments, the B cell depleting agent is an agent that binds to a B cell surface molecule.

In some embodiments, the B cell depleting agent is selected from anti-CD19 antibodies, anti-CD20 antibodies, anti-CD22 antibodies, anti-CD79 antibodies, multispecific antibodies combining two or more of any of said antibody specificities, multispecific antibodies combining any of said antibody specificities with anti-CD3 antibodies, functional fragments of any of said antibodies, and any combinations thereof.

In some embodiments, the B cell depleting agent comprises an anti-CD20 antibody or a functional fragment thereof and an anti-CD19 antibody or a functional fragment thereof.

In some embodiments, the B cell depleting agent is an anti-CD20 antibody or a functional fragment thereof.

In some embodiments, the anti-CD20 antibody is a multispecific antibody or a functional fragment thereof.

In some embodiments, the multispecific anti-CD20 antibody or functional fragment thereof targets CD20 and CD3.

In some embodiments, the multispecific anti-CD20 antibody or functional fragment thereof is anti-CD20×CD3 bispecific antibody or functional fragment thereof.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises:
a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and
b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some embodiments, the anti-CD20×CD3 bispecific antibody or functional fragment thereof comprises a human IgG heavy chain constant region.

In some embodiments, the human IgG heavy chain constant region is isotype IgG4 or IgG1.

In some embodiments, the human IgG heavy chain constant region comprises one or more modifications that increase binding to a neonatal Fc receptor (FcRn).

In some embodiments, the human IgG heavy chain constant region comprises one or more modifications that decrease binding to an Fc-gamma receptor (FcγR).

In some embodiments, the B cell depleting agent is an agent targeting a B cell survival factor.

In some embodiments, the B cell depleting agent is a BLyS/BAFF inhibitor, an APRIL inhibitor, a BLyS receptor 3/BAFF receptor inhibitor, or any combination thereof.

In some embodiments, the immunoglobulin depleting agent is capable of accelerating IgG clearance.

In some embodiments, the immunoglobulin depleting agent is a neonatal Fc receptor (FcRn) blocker.

In some embodiments, the FcRn blocker is selected from Efgartigimod (ARGX-113), Rozanolixizumab (UCB7665), Batoclimab (RVT-1401), Nipocalimab (M281), Orilanolimab (SYNT001), IMVT-1402, and any combinations thereof.

In some embodiments, the method further comprises plasmapheresis, therapeutic plasma exchange, or immunoadsorption.

In a further aspect, provided herein is a pharmaceutical composition comprising (i) a plasma cell depleting agent, (ii) a B cell depleting agent and/or an immunoglobulin depleting agent, and (iii) a pharmaceutically acceptable carrier and/or excipient.

In a further aspect, provided herein is a pharmaceutical composition comprising (i) an immunogen, (ii) a plasma cell depleting agent, (iii) optionally, a B cell depleting agent and/or an immunoglobulin depleting agent, and (iv) a pharmaceutically acceptable carrier and/or excipient.

In another aspect, provided herein is a kit comprising (i) a plasma cell depleting agent, (ii) a B cell depleting agent and/or an immunoglobulin depleting agent, and (iii) optionally, instructions for use.

In another aspect, provided herein is a kit comprising (i) an immunogen, (ii) a plasma cell depleting agent, (iii) optionally a B cell depleting agent and/or an immunoglobulin depleting agent, and (iv) optionally, instructions for use.

In a further aspect, provided herein is a method for inhibiting or preventing an immune response to an immunogen in a subject in need thereof, the method comprising administering to the subject an effective amount of an anti-CD20×CD3 bispecific antibody or a functional fragment thereof.

In some embodiments, inhibiting the immune response comprises suppression of numbers and frequencies of immunogen-specific B cells.

In some embodiments, inhibiting the immune response comprises suppression of immunogen-specific IgG and/or IgM responses.

In a further aspect, provided herein is a method for inhibiting or preventing generation of neutralizing antibodies to an immunogen in a subject in need thereof, the method comprising administering to the subject an effective amount of an anti-CD20×CD3 bispecific antibody or functional fragment thereof.

In a further aspect, provided herein is a method for increasing effectiveness of re-administration of an immunogen to a subject in need thereof, the method comprising administering to the subject an effective amount of an anti-CD20×CD3 bispecific antibody or functional fragment thereof.

In some embodiments, the immunogen re-administration occurs via the same administration route as its prior administration.

In some embodiments, the immunogen re-administration occurs via a different administration route than its prior administration.

In some embodiments, the subject does not have a pre-existing immunity against the immunogen.

In some embodiments, the anti-CD20×CD3 bispecific antibody or functional fragment thereof is administered before the administration of the immunogen to the subject.

In some embodiments, the anti-CD20×CD3 bispecific antibody or functional fragment thereof is administered simultaneously with the administration of the immunogen to the subject.

In some embodiments, the anti-CD20×CD3 bispecific antibody or functional fragment thereof is administered after the administration of the immunogen to the subject.

In some embodiments, the immunogen is administered to the subject two or more times and the anti-CD20×CD3 bispecific antibody or functional fragment thereof is administered before and/or between each of the administrations of the immunogen.

In some embodiments, the immunogen is an immunogenic delivery vehicle, a polypeptide, a polynucleotide, a glycan, or a lipid.

In some embodiments, the immunogen is an immunogenic delivery vehicle or a polypeptide or polynucleotide encoded by a transgene contained within the immunogenic delivery vehicle.

In another aspect, provided herein is a method for increasing or maintaining the level of a transgene expression in a subject in need thereof, the method comprising administering to the subject an effective amount of an anti-CD20×CD3 bispecific antibody or a functional fragment thereof.

In some embodiments, the transgene is delivered to the subject via an immunogenic delivery vehicle.

In some embodiments, the level of transgene expression is increased or maintained by inhibiting an immune response to the immunogenic delivery vehicle and/or by inhibiting an immune response to a polypeptide or polynucleotide encoded by the transgene.

In some embodiments, the level of transgene expression is increased or maintained by inhibiting antibody responses to a polypeptide or polynucleotide encoded by the transgene.

In some embodiments, the subject does not have a pre-existing immunity against the immunogenic delivery vehicle and/or a polypeptide or polynucleotide encoded by the transgene.

In some embodiments, the anti-CD20×CD3 bispecific antibody or functional fragment thereof is administered before the administration of the immunogenic delivery vehicle to the subject.

In some embodiments, the anti-CD20×CD3 bispecific antibody or functional fragment thereof is administered simultaneously with the administration of the immunogenic delivery vehicle to the subject.

In some embodiments, the anti-CD20×CD3 bispecific antibody or functional fragment thereof is administered after the administration of the immunogenic delivery vehicle to the subject.

In some embodiments, the immunogenic delivery vehicle is administered to the subject two or more times and the anti-CD20×CD3 bispecific antibody or functional fragment thereof is administered before and/or between each of the administrations of the immunogenic delivery vehicle.

In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector.

In some embodiments, the immunogenic delivery vehicle is a viral vector.

In a further aspect, provided herein is a method for increasing effectiveness of a subsequently administered viral vector following an originally administered viral vector in a subject in need thereof, the method comprising administering to the subject an effective amount of an anti-CD20×CD3 bispecific antibody or a functional fragment thereof, wherein the subsequently administered viral vector is of the same or similar viral origin as the originally administered viral vector.

In some embodiments, the subsequently administered viral vector is administered via the same administration route as the originally administered viral vector.

In some embodiments, the subsequently administered viral vector is administered via a different administration route from the originally administered viral vector.

In some embodiments, the subject does not have a pre-existing immunity against the viral vectors.

In some embodiments, the anti-CD20×CD3 bispecific antibody or functional fragment thereof is administered before the administration of the originally administered viral vector to the subject.

In some embodiments, the anti-CD20×CD3 bispecific antibody or functional fragment thereof is administered simultaneously with the administration of the originally administered viral vector and/or subsequently administered viral vector to the subject.

In some embodiments, the anti-CD20×CD3 bispecific antibody or functional fragment thereof is administered after the administration of the originally administered viral vector but before administering the subsequently administered viral vector to the subject.

In some embodiments, the anti-CD20×CD3 bispecific antibody or functional fragment thereof is administered after the administration of the subsequently administered viral vector to the subject.

In some embodiments, the anti-CD20×CD3 bispecific antibody or functional fragment thereof is administered before and/or between each of the administrations of the viral vectors to the subject.

In some embodiments, the viral vectors are derived from an adeno-associated virus (AAV), an adenovirus, or a retrovirus.

In some embodiments, the viral vectors are derived from AAV.

In some embodiments, the subsequently administered AAV vector has a capsid derived from the same AAV serotype as the originally administered AAV vector.

In some embodiments, the retrovirus is a lentivirus.

In some embodiments, the viral vectors are derived from an oncolytic virus.

In some embodiments, the oncolytic virus is an adenovirus, a rhabdovirus, a herpes virus, a measles virus, a coxsackievirus, a poliovirus, a reovirus, a poxvirus, a parvovirus, Maraba virus, or Newcastle disease virus.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises:
a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and
b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some embodiments, the anti-CD20×CD3 bispecific antibody or functional fragment thereof comprises a human IgG heavy chain constant region.

In some embodiments, the human IgG heavy chain constant region is isotype IgG4 or IgG1.

In some embodiments, the human IgG heavy chain constant region comprises one or more modifications that increase binding to a neonatal Fc receptor (FcRn).

In some embodiments, the human IgG heavy chain constant region comprises one or more modifications that decrease binding to an Fc-gamma receptor (FcγR).

In another aspect, provided herein is a composition comprising an immunogen and an anti-CD20×CD3 bispecific antibody or a functional fragment thereof and optionally further comprising a pharmaceutically acceptable carrier and/or excipient.

In some embodiments, the immunogen is an immunogenic delivery vehicle, a polypeptide, a polynucleotide, a glycan, or a lipid.

In some embodiments, the immunogen is an immunogenic delivery vehicle or a polypeptide or polynucleotide encoded by a transgene contained within the immunogenic delivery vehicle.

In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector.

In some embodiments, the immunogenic delivery vehicle is a viral vector.

In some embodiments, the viral vector is derived from an adeno-associated virus (AAV), an adenovirus, or a retrovirus.

In some embodiments, the viral vector is derived from AAV.

In some embodiments, the retrovirus is a lentivirus.

In some embodiments, the viral vector is derived from an oncolytic virus.

In some embodiments, the oncolytic virus is an adenovirus, a rhabdovirus, a herpes virus, a measles virus, a coxsackievirus, a poliovirus, a reovirus, a poxvirus, a parvovirus, Maraba virus, or Newcastle disease virus.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises:
a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and
b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some embodiments, the anti-CD20×CD3 bispecific antibody or functional fragment thereof comprises a human IgG heavy chain constant region.

In some embodiments, the human IgG heavy chain constant region is isotype IgG4 or IgG1.

In some embodiments, the human IgG heavy chain constant region comprises one or more modifications that increase binding to a neonatal Fc receptor (FcRn).

In some embodiments, the human IgG heavy chain constant region comprises one or more modifications that decrease binding to an Fc-gamma receptor (FcγR).

In another aspect, provided herein is a kit comprising (i) an immunogen, (ii) an anti-CD20×CD3 bispecific antibody or a functional fragment thereof, and (iii) optionally, instructions for use.

In some embodiments, the immunogen is an immunogenic delivery vehicle, a polypeptide, a polynucleotide, a glycan, or a lipid.

In some embodiments, the immunogen is an immunogenic delivery vehicle or a polypeptide or polynucleotide encoded by a transgene contained within the immunogenic delivery vehicle.

In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector.

In some embodiments, the immunogenic delivery vehicle is a viral vector.

In some embodiments, the viral vector is derived from an adeno-associated virus (AAV), an adenovirus, or a retrovirus.

In some embodiments, the viral vector is derived from AAV.

In some embodiments, the retrovirus is a lentivirus.

In some embodiments, the viral vector is derived from an oncolytic virus.

In some embodiments, the oncolytic virus is an adenovirus, a rhabdovirus, a herpes virus, a measles virus, a coxsackievirus, a poliovirus, a reovirus, a poxvirus, a parvovirus, Maraba virus, or Newcastle disease virus.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises:
a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and
b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some embodiments, the anti-CD20×CD3 bispecific antibody or functional fragment thereof comprises a human IgG heavy chain constant region.

In some embodiments, the human IgG heavy chain constant region is isotype IgG4 or IgG1.

In some embodiments, the human IgG heavy chain constant region comprises one or more modifications that increase binding to a neonatal Fc receptor (FcRn).

In some embodiments, the human IgG heavy chain constant region comprises one or more modifications that decrease binding to an Fc-gamma receptor (FcγR).

These and other aspects described herein will be apparent to those of ordinary skill in the art in the following description, claims and drawings.

### BRIEF DESCRIPTION OF THE DRAWINGS

FIG. 1 shows an experimental timeline for the study described in Examples 1, 2, and 3.

FIG. 2 shows the effect of plasma cell depletion with anti-BCMA×CD3 bispecific antibody, FcRn blockade via efgartigimod alfa, B cell depletion with anti-CD19 and anti-CD20 antibodies (anti-CD19/CD20 antibodies), or combination thereof, on anti-AAV8 capsid IgG titers over time in mice previously treated with recombinant AAV8 vector.

FIG. 3 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second AAV8 vector in mice previously treated with recombinant AAV8 vector, as measured by Taqman quantitative real-time polymerase chain reaction (PCR) of green fluorescent protein (GFP) transgene DNA.

FIG. 4 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by Taqman quantitative real-time reverse-transcription PCR of GFP transgene RNA.

FIGS. 5A-5B show the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by GFP immunohistochemical (IHC) staining of formalin-fixed paraffin embedded liver sections. FIG. 5A shows GFP-positive area quantified using HALO software (Indica labs). FIG. 5B shows representative images.

FIGS. 6A-6J show flow cytometry analysis of B cell and plasma cell frequencies and counts in bone marrow and spleen following treatment with anti-BCMA×CD3 bispecific antibody, FcRn blockade, anti-CD19/CD20 antibodies, or combinations thereof. FIG. 6A shows bone marrow plasma cell frequencies. FIG. 6B shows spleen plasma cell frequencies. FIG. 6C shows spleen naïve B cell frequencies. FIG. 6D shows spleen total memory B cell frequencies. FIG. 6E shows spleen AAV-specific memory B cell frequencies. FIG. 6F shows bone marrow plasma cell counts. FIG. 6G shows spleen plasma cell counts. FIG. 6H shows spleen naïve B cell counts. FIG. 6I shows spleen total memory B cell counts. FIG. 6J shows spleen AAV-specific memory B cell counts.

FIG. 7 shows the effect of efgartigimod on serum drug concentration of REGN5458 (BCMA×CD3).

FIG. 8 shows an experimental timeline for the study described in Example 10.

FIGS. 9A-9B show the effect of plasma cell depletion, B cell depletion, neonatal Fc receptor blockade, and combinations thereof, on naturally-occurring anti-AAV antibody titers in cynomolgus macaques. AAV8 neutralizing antibody (NAb) titer levels are presented for each treatment group over the duration of the study (FIG. 9A) and specifically at Study Day 29 (FIG. 9B).

FIG. 10 shows an experimental timeline for the study described in Examples 11 and 12.

FIGS. 11A-11C show a comparison of the effect of CD20×CD3-mediated versus anti-CD20-mediated B cell depletion on the development of anti-AAV IgM antibody titers (FIG. 11A) and anti-AAV IgG antibody titers (FIGS. 11B-11C) in mice.

FIGS. 12A-12C show a comparison of the effect of CD20×CD3-mediated versus anti-CD20-mediated B cell depletion on AAV transduction (FIG. 12A) and transgene expression (FIGS. 12B-12C) following vector re-administration in mice.

FIG. 13 shows an experimental timeline for the study described in Examples 13 and 14.

FIGS. 14A-14F show the effect of prophylactic CD20×CD3-mediated B cell depletion on serum anti-AAV8 IgM (FIG. 14A and FIG. 14D), IgG (FIG. 14B and FIG. 14E), and neutralizing antibody (nAb) (FIG. 14C and FIG. 14F) titers in cynomolgus macaques.

FIGS. 15A-15C show the effect of prophylactic CD20×CD3-mediated B cell depletion on AAV transduction (FIG. 15A) and transgene expression (FIGS. 15B-15C) following AAV vector re-administration in cynomolgus macaques.

### DETAILED DESCRIPTION

The present disclosure provides, among other things, a distinct B cell immunosuppression approach that enables AAV vector re-transduction at levels equal to seronegative animals by depleting pre-existing nAbs (e.g., via combined plasma cell and immunoglobulin depletion). Long-lived plasma cells (LLPC) mediate constitutive antibody production to most antigens and are a likely reservoir of persistent anti-AAV antibody immunity. The present disclosure was made in part based on the discovery that pre-existing anti-AAV nAbs could be directly eliminated in vivo by LLPC depletion with linvoseltamab, a fully-human T cell-bridging bispecific antibody targeting B cell maturation antigen (BCMA) and CD3 (anti-BCMA×CD3 bispecific antibody), either alone or in combination with B cell depletion (to eliminate non-LLPC sources of anti-AAV nAbs) and/or FcRn blockade (to accelerate serum IgG clearance). Further, in AAV-naïve patients, prophylactic transient B cell depletion before and during AAV treatment with a bispecific antibody targeting CD20 and CD3 (anti-CD20×CD3 bispecific antibody) alone could be used to prevent an antibody response (e.g., the generation of anti-AAV nAbs), thereby allowing for effective AAV re-dosing.

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “antigen-binding molecule” includes antibodies and antigen-binding fragments of antibodies, including multispecific antibodies, e.g., bispecific antibodies.

The term “antibody,” as used herein, refers to an antigen-binding molecule or molecular complex comprising a set of complementarity determining regions (CDRs) that specifically bind to or interact with a particular antigen (e.g., BCMA, CD20, CD3). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, but are not limited to, the Kabat definition, the Chothia definition, the AbM definition (enhanced Chothia or Martin), the IMGT definition, and the Honneger definition (AHo). In general terms, the Kabat definition is based on sequence variability, the Chothia definition is based on the location of the structural loop regions, and the AbM definition is a compromise between the Kabat and Chothia approaches. See, e.g., Kabat et al., “Sequences of Proteins of Immunological Interest,” National Institutes of Health, Bethesda, Md. (1991); Chothia et al., J Mol Biol (1987), 4:901-17; Al-Lazikani et al., J. Mol. Biol. 273:927-948 (1997); and Martin et al., Proc. Natl. Acad. Sci. USA 86:9268-9272 (1989); see also, Dondelinger et al., Front. Immunol. (2018), 9:2278, doi: 10.3389/fimmu.2018.02278. Public databases are also available for identifying CDR sequences within an antibody.

The term “antibody,” as used herein, also includes antigen-binding fragments of full antibody molecules. The terms “antigen-binding portion” of an antibody, “antigen-binding fragment” of an antibody, “antigen-binding domain,” and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen

Any multispecific antibody format may be adapted for use in the context of an antibody or antigen-binding fragment of an antibody of the present disclosure using routine techniques available in the art. For example, the present disclosure includes bispecific antibodies wherein one arm of an immunoglobulin is specific for an epitope of BCMA or CD20 and the other arm of the immunoglobulin is specific for an epitope of CD3. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency, and geometry. (See, e.g., Kazane et al., J. Am. Chem. Soc. [Epub: Dec. 4, 2012]).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant antibody,” as used herein, is intended to include all antibodies that are prepared, expressed, created, or isolated by recombinant means. The term includes, but is not limited to, antibodies expressed using a recombinant expression vector transfected into a host cell (e.g., Chinese hamster ovary (CHO) cell) or cellular expression system, antibodies isolated from a recombinant, combinatorial human antibody library, and antibodies isolated from a non-human animal (e.g., a mouse, such as a mouse that is transgenic for human immunoglobulin genes (see e.g., Taylor et al. (1992) Nucl. Acids Res. 20:6287-6295). In some embodiments, the recombinant antibody is a recombinant human antibody. In some embodiments, recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less, e.g., 10⁻⁷ M, 10⁻⁸ M, 10⁻⁹ M, 10⁻¹⁰ M, 10⁻¹¹ M, or 10⁻¹² M (a smaller K_{D} denotes a tighter binding). Methods for determining whether an antibody specifically binds to an antigen are known in the art and include, for example, equilibrium dialysis, surface plasmon resonance (e.g., BIACORE™), bio-layer interferometry assay (e.g., Octet® HTX biosensor), solution-affinity ELISA, and the like. In some embodiments, specific binding is measured in a surface plasmon resonance assay, e.g., at 25° C. or 37° C. An antibody or antigen-binding fragment that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example using the BIACORE™ system (Cytiva, Marlborough, MA).

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or discontinuous (e.g., conformational). A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups and, in some embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. An epitope typically includes at least 3, and more usually, e.g., at least 5 or at least 8-10 amino acids, in a unique spatial conformation.

Methods for determining the epitope of an antigen-binding protein, e.g., an antibody or antigen-binding fragment, include alanine scanning mutational analysis, peptide blot analysis (Reineke, Methods Mol Biol 2004, 248:443-463), peptide cleavage analysis, crystallographic studies, and nuclear magnetic resonance (NMR) analysis. In addition, methods such as epitope exclusion, epitope extraction, and chemical modification of antigens can be employed (Tomer, Prot Sci 2000, 9:487-496). Another method that can be used to identify the amino acids within a polypeptide with which an antigen-binding protein (e.g., an antibody or antigen-binding fragment) interacts is hydrogen/deuterium exchange detected by mass spectrometry (HDX). See, e.g., Ehring, Analytical Biochemistry 1999, 267:252-259; Engen and Smith, Anal Chem 2001, 73:256A-265A.

The term “competes,” as used in reference to competing for binding, refers to an antigen-binding protein (e.g., antibody or antigen-binding fragment) that binds to an antigen and inhibits or blocks the binding of another antigen-binding protein (e.g., antibody or antigen-binding fragment) to the antigen. Unless otherwise stated, the term also includes competition between two antigen-binding proteins (e.g., antibodies) in both orientations, i.e., a first antigen that binds an antigen and blocks binding of the antigen by a second antibody, and vice versa. Thus, in some embodiments, competition occurs in one such orientation. In some embodiments, the first antigen-binding protein (e.g., antibody) and second antigen-binding protein (e.g., antibody) may bind to the same epitope. Alternatively, the first and second antigen-binding proteins (e.g., antibodies) may bind to different epitopes, which may be overlapping or non-overlapping, wherein binding of one antigen-binding protein inhibits or blocks the binding of the second antigen-binding protein, e.g., via steric hindrance. Competition between antigen-binding proteins may be measured by methods known in the art, e.g., by a real-time, label-free bio-layer interferometry assay.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

In the context of the present disclosure, the term “neutralizing antibody” or “nAb” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an immunogen, e.g., antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines, from an actively immunized host to a non-immune host.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The terms “viral element” and “viral component” are used herein to refer to viral genes (e.g., genes encoding polymerase or structural proteins) or other elements of the viral genome (e.g., packaging signals, regulatory elements, LTRs, ITRs, etc.).

The term “capsid protein,” “Cap protein,” and the like, includes a protein that is part of the capsid of the virus.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline (tet)-responsive promoter, a tetracycline operator sequence (tetO), a tet-On promoter, or a tet-Off promoter), steroid-regulated promoters (e.g., a rat glucocorticoid receptor, a promoter of an estrogen receptor, or a promoter of an ecdysone receptor), or metal-regulated promoters (e.g., a metalloprotein promoter). Physically-regulated promoters include, for example, temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters.

Developmentally-regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

The term “fusogen” or “fusogenic molecule” is used herein to refer to any molecule that can trigger membrane fusion when present on the surface of a virus particle. A fusogen can be, for example, a protein (e.g., a viral glycoprotein) or a fragment, mutant or derivative thereof.

The term “oncolytic virus” is used herein to refer to a virus that is capable of infecting and replicating in a tumor cell such that the tumor cell may be killed. The oncolytic virus may be replication competent. As a non-limiting example, the oncolytic virus may comprise a rhabdovirus, i.e., any of a group of viruses comprising the family Rhabdoviridae, e.g., a vesicular stomatitis virus (VSV).

The term “T cell” is used herein in its broadest sense to refer to all types of immune cells expressing CD3, including T-helper cells (CD4+ cells), cytotoxic T-cells (CD8+ cells), T-regulatory cells (Treg), and natural killer (NK)-T cells.

“Retargeting” or “redirecting” may include a scenario in which a wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

The term “wild type” or “wild-type” includes entities having a structure and/or activity as found in a normal (as contrasted with mutant, diseased, altered, or so forth) state or context. Wild type genes and polypeptides often exist in multiple different forms (e.g., alleles).

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

“Specific binding pair,” “binding pair,” “protein:protein binding pair,” and the like, includes two members (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope; a covalent bond between e.g., proteins capable of forming isopeptide bonds; split inteins that recognize each other and, through the process of protein trans-splicing, mediate ligation of the flanking proteins and their own removal). In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002; SpyTag:KTag; isopeptag:pilin C, SnoopTag:SnoopCatcher, etc., and variants thereof, e.g., SpyTag003:SpyCatcher003. Generally, a first member of a protein:protein binding pair refers to member of a protein:protein binding pair, which is generally less than 30 amino acids in length, and which forms a spontaneous covalent isopeptide bond with the second cognate protein, wherein the second cognate protein is generally larger, but may also be less than 30 amino acids in length such as in the SpyTag:KTag system.

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 90%, e.g., at least 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and “substantially identical” mean that two peptide sequences, when optimally aligned, share at least about 90% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the disclosure to a database containing a large number of sequences from different organisms is the computer program BLAST, especially BLASTP or TBLASTN, using default parameters. (See, e.g., Altschul et al., 1990, J. Mol. Biol. 215: 403-410 and 1997 Nucleic Acids Res. 25:3389-3402).

A “variant” of a polypeptide, such an immunoglobulin, VH, VL, heavy chain, light chain, or CDR comprising an amino acid sequence specifically set forth herein, refers to a polypeptide comprising an amino acid sequence that is at least about 70%-99.9% (e.g., at least 70, 75, 80, 85, 90, 91, 92, 93, 94, 95, 96, 97, 98, 99, 99.5, or 99.9%) identical to the reference polypeptide sequence (e.g., as set forth in the sequence listing below), when the comparison is performed by a BLAST algorithm wherein the parameters of the algorithm are selected to give the largest match between the respective sequences over the entire length of the respective reference sequences. In some embodiments, a variant of a polypeptide includes a polypeptide having the amino acid sequence of a reference polypeptide sequence (e.g., as set forth in the sequence listing below) but for one or more (e.g., 1 to 10, or less than 20, or less than 10) missense mutations (e.g., conservative substitutions), nonsense mutations, deletions, or insertions.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable” as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

An “individual” or “subject” or “animal” refers to humans, veterinary animals (e.g., cats, dogs, cows, horses, sheep, pigs, etc.) and experimental animal models of diseases (e.g., mice, rats). In a preferred embodiment, the subject is a human.

### Plasma Cell Depleting Agents

In some embodiments, the methods disclosed herein include administering a therapeutically effective amount of a plasma cell depleting agent to a subject in need thereof. As used herein, a “plasma cell depleting agent” refers to any molecule capable of specifically binding to a surface antigen on plasma cells and killing or depleting said plasma cell.

The plasma cell depleting agents can be administered to a subject in need thereof either alone or in combination with a B cell depleting agent, and/or an immunoglobulin depleting agent. In various aspects, a plasma cell depleting agent may be combined or administered in combination with a B cell depleting agent, an immunoglobulin depleting agent, plasmapheresis, therapeutic plasma exchange, immunoadsorption, and/or an immunogen (e.g., an immunogenic delivery vehicle such as, e.g., AAV) disclosed herein. Suitable combinations comprising a plasma cell depleting agent are described in more detail elsewhere herein. In some embodiments, the plasma cell depleting agent of the present disclosure is capable of depleting plasma cells including, without limitation, long-lived plasma cells (LLPCs). In some embodiments, a plasma cell depleting agent is administered to a subject having a pre-existing immunity against an immunogen (e.g. an immunogenic delivery vehicle such as, e.g., AAV)

In some embodiments, the plasma cell depleting agent can be an antibody, a small molecule compound, a nucleic acid, a polypeptide, or a functional fragment or variant thereof. Non-limiting examples of suitable plasma cell depleting agents include B cell maturation antigen (BCMA) targeting agents (described elsewhere herein), proteasome inhibitors [e.g., bortezomib (Velcade), carfilzomib (Kyprolis), ixazomib (Niniaro)], histone deacetylase inhibitors [e.g., panobinostat (Farydak)], B-cell activating factor (BAFF; also referred to as BLyS, TALL-1, or CD257) inhibitors (e.g., anti-BAFF antibodies such as belimumab, tabalumab, AMG570; or anti-BAFF receptor antibodies such as ianalumab), proliferation-inducing ligand (APRIL; also referred to as TNFSF13 or CD256) inhibitors (e.g., anti-APRIL antibodies such as BION-1301 or VIS624), G protein-coupled receptor, class C, group 5, member D (GPRC5D) inhibitors (e.g., anti-GPRC5D antibodies, anti-GPRC5D×CD3 bispecific antibodies such as talquetamab), Fc receptor homolog 5 (FcRH5; also referred to as FcRL5, IRTA2, or CD307) inhibitors (e.g., anti-FcRH5 antibodies, anti-FcRH5×CD3 bispecific antibodies such as Cevostamab), and cluster of differentiation 38 (CD38; also referred to as CADPR1 or ADPRC1) inhibitors (e.g., anti-CD38 antibodies).

In some embodiments, the plasma cell depleting agents used in the methods disclosed herein are BCMA targeting agents. As used herein, the term “BCMA targeting agent” refers to any molecule capable of binding specifically to BCMA that is expressed on the surface of a cell, e.g, a cell in a subject, thus targeting said cell for destruction. BCMA is expressed exclusively in B-cell lineage cells, particularly in the interfollicular region of the germinal center, as well as on plasmablasts and differentiated plasma cells. BCMA is selectively induced during plasma cell differentiation and is required for optimal survival of long-lived plasma cells (LLPCs) in the bone marrow. Thus, a BCMA targeting agent binds to BCMA expressed on a plasma cell surface and mediates killing or depletion of cells that express BCMA (plasma cell depletion). In some embodiments, a BCMA targeting agent comprises a binding moiety that binds to plasma cell-surface-expressed BCMA (an antigen-binding moiety or antigen-binding fragment thereof) and a moiety that facilitates killing of said plasma cell. In some embodiments, the plasma cell-surface-expressed BCMA-binding moiety is an antibody or antigen-binding fragment thereof that binds specifically to BCMA. Such a BCMA-binding moiety can be linked (e.g., covalently bound) to a moiety that facilitates killing or destruction of the targeted plasma cell. The moiety that facilitates targeted killing of the bound plasma cell may be a molecule that directly kills the targeted cell (e.g., a cytotoxic agent) or may be a protein or fragment thereof that mediates killing of the targeted cell, e.g., by an immune cell, e.g., a T-cell. In the context of the present disclosure, the term “BCMA targeting agent” includes, but is not limited to, anti-BCMA antibodies that are conjugated to a therapeutic agent such as a cytotoxic drug (“BCMA ADC” or “anti-BCMA ADC”, e.g., Belantamab Mafodotin (GSK2857916), MEDI2228, HDP-101), chimeric antigenic receptors (CARs) that bind specifically to BCMA, (“BCMA CAR” or “anti-BCMA CAR”) and anti-BCMA×CD3 bispecific antibodies (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B).

In some embodiments, the BCMA targeting agent used in the context of the disclosed methods is an antibody-drug conjugate (ADC) comprising an anti-BCMA antibody and a cytotoxic drug. In some embodiments, the anti-BCMA antibody or antigen-binding fragment thereof and the cytotoxic agent are covalently attached via a linker. In general terms, the ADCs comprise: A-[L-P]_{y}, in which A is an antigen-binding molecule, e.g., an anti-BCMA antibody, or a fragment thereof, L is a linker, P is the payload or therapeutic moiety (e.g., cytotoxic agent), and y is an integer from 1 to 30. Examples of suitable cytotoxic agents and chemotherapeutic agents for forming ADCs are known in the art. Non-limiting examples of suitable cytotoxic agents that can be conjugated to anti-BCMA antibodies for use in the disclosed methods are auristatin such as monomethyl auristatin E (MMAE) or monomethyl auristatin F (MMAF), a tubulysin such as TUB-OH or TUB-OMOM, a tomaymycin derivative, a dolastatin derivative, or a maytansinoid such as DM1 or DM4. In some exemplary embodiments, an anti-BCMA ADC used in the present methods comprises the HCVR, LCVR and/or CDR amino acid sequences of any of the anti-BCMA antigen-binding molecules disclosed herein.

Other anti-BCMA ADCs that can be used in the context of the methods of the present disclosure include, e.g., the ADCs referred to and known in the art as Belantamab Mafodotin (GSK2857916), AMG224, HDP-101, MEDI2228, and TBL-CLN1, or any of the anti-BCMA ADCs set forth, e.g., in International Patent Publications WO2011/108008, WO2014/089335, WO2017/093942, WO2017/143069, or WO2019/025983. The portions of the publications cited herein that identify anti-BCMA ADCs are hereby incorporated by reference.

In some embodiments, the BCMA targeting agent used in the context of the disclosed methods is a chimeric antigen receptor (CAR) that binds specifically to BCMA (“BCMA CAR”). Generally, a “chimeric antigen receptor” (CAR) exhibits a specific anti-target cellular immune activity and comprises a binding domain against a component present on the target cell, for example an antibody-based specificity for a desired antigen (e.g., BCMA on plasma cell), and a T cell receptor-activating intracellular domain. CARs typically comprise an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity. In certain embodiments, the BCMA CAR or antigen-binding fragment thereof comprises a HCVR, LCVR, and/or CDRs comprising the amino acid sequences of any of the antibodies set forth in US Patent Publication No. US 2020/0023010, which is hereby incorporated by reference in its entirety. In some exemplary embodiments, an anti-BCMA CAR used in the present methods comprises the HCVR, LCVR and/or CDR amino acid sequences of any of the anti-BCMA antigen-binding molecules disclosed herein.

Other anti-BCMA CARs that can be used in the context of the methods of the present disclosure include, e.g., the CARs referred to and known in the art as bb2121, LCAR-B38M, and 4C8A, or any of the anti-BCMA CARs set forth, e.g., in patent publications WO2015/052538, WO2015/052536, WO2016/094304, WO2016/166630, WO2016/151315, WO2016/130598, WO2017/183418, WO2017/173256, WO2017211900, WO2017/130223, WO2018/229492, WO2018/085690, WO2018/151836, WO2018/028647, WO2019/006072. The portions of the publications cited herein that identify anti-BCMA CARs are hereby incorporated by reference.

In some exemplary embodiments, the BCMA targeting agent used in the disclosed methods is a multispecific (e.g., bispecific) antibody, or a functional fragment thereof, that specifically binds B cell maturation antigen (BCMA) and CD3 (e.g., an anti-BCMA×CD3 bispecific antibody). The anti-BCMA×CD3 multispecific (e.g., bispecific) antibodies are useful for specific targeting and T-cell-mediated killing of cells that express BCMA. The terms “antibody,” “antigen-binding fragment,” “human antibody,” “recombinant antibody,” and other related terminology are defined above. In the context of anti-BCMA×CD3 antibodies and antigen-binding fragments thereof, the present disclosure includes the use of bispecific antibodies wherein one arm of an immunoglobulin is specific for BCMA or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

An anti-BCMA×CD3 bispecific antibody, or functional fragment thereof, may comprise any of various anti-BCMA×CD3 bispecific antibodies, or functional fragments thereof, disclosed herein, or any other such anti-BCMA×CD3 bispecific antibodies, or functional fragments thereof, known to persons of ordinary skill in the art (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B).

### CD3 Antigen-Binding Molecules

The term “CD3,” as used herein, refers to an antigen which is expressed on T cells as part of the multimolecular T cell receptor (TCR) and which consists of a homodimer or heterodimer formed from the dimeric association of two of four receptor chains: CD3-epsilon, CD3-delta, CD3-zeta, and CD3-gamma (e.g., gamma/epsilon, delta/epsilon, and zeta/zeta). CD3 is required for T cell activation.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). Antibodies against CD3 have been shown to cluster CD3 on T cells, thereby causing T cell activation in a manner similar to the engagement of the TCR by peptide-loaded major histocompatibility complex (MHC) molecules. Thus, bispecific antigen-binding molecules that are capable of binding both CD3 and another antigen (e.g., CD20 or BCMA) would be useful in settings in which specific targeting and T cell-mediated killing of cells that express the non-CD3 antigen (e.g., CD20 or BCMA) is desired.

The antibodies and antigen-binding fragments of the present invention may bind soluble CD3 and/or cell surface-expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

As used herein, the expression “anti-CD3 antibody” includes both monovalent antibodies with a single specificity, as well as bispecific antibodies comprising one arm that binds CD3 and another arm that binds a different antigen, wherein the anti-CD3 arm comprises any of the HCVR/LCVR or CDR sequences, or functional fragments thereof, as set forth in Table 1 or Table 2 herein. Examples of anti-CD3 bispecific antibodies are described elsewhere herein. Exemplary anti-CD3 antibodies are also described in PCT International Application No. PCT/US2013/060511, which is herein incorporated by reference in its entirety.

The present disclosure includes bispecific antibodies and functional fragments thereof that bind human CD3 with high affinity. The present disclosure also includes bispecific antibodies and functional fragments thereof that bind human CD3 with medium or low affinity, depending on the therapeutic context and particular targeting properties that are desired. For example, in the context of a bispecific antigen-binding molecule, wherein one arm binds CD3 and a second arm binds another antigen (e.g., CD20 or BCMA), it may be desirable for the second arm to bind the non-CD3 (e.g., CD20 or BCMA) antigen with high affinity while the anti-CD3 arm binds CD3 with only moderate or low affinity. In this manner, preferential targeting of the antigen-binding molecule to cells expressing the non-CD3 (e.g., CD20 or BCMA) antigen may be achieved while avoiding general/untargeted CD3 binding and the consequent adverse side effects associated therewith.

In certain embodiments, the anti-CD3 antibodies induce T cell proliferation with an EC₅₀ value of less than about 0.33 pM, as measured by an in vitro T cell proliferation assay (e.g., assessing the proliferation of Jurkat cells or PBMCs in the presence of anti-CD3 antibodies). In certain embodiments, the anti-CD3 antibodies induce T cell proliferation (e.g., Jurkat cell proliferation and/or PBMC proliferation) with an EC₅₀ value of less than about 0.32 pM, less than about 0.31 pM, less than about 0.30 PM, less than about 0.28 pM, less than about 0.26 pM, less than about 0.24 pM, less than about 0.22 pM, or less than about 0.20 pM, as measured by an in vitro T cell proliferation assay.

### BCMA×CD3 Antigen-Binding Molecules

The present disclosure provides antigen-binding molecules including multispecific (e.g., bispecific) antibodies that specifically bind B cell maturation antigen (BCMA) and CD3 (e.g., an anti-BCMA×CD3 bispecific antibody). In some embodiments, the antigen-binding molecule is a multispecific (e.g., bispecific) antibody. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. In some embodiments, the multispecific antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity. In some embodiments, the multispecific antibody contains an antigen-binding domain that is specific for BCMA and an antigen-binding domain that is specific for CD3.

In some embodiments, the anti-BCMA×CD3 bispecific antigen-binding molecule comprises a first antigen-binding domain (D1) that binds an epitope of BCMA (e.g., human BCMA), and a second antigen-binding domain (D2) that binds an epitope of CD3 (e.g., human CD3).

In some exemplary embodiments, the anti-BCMA×CD3 bispecific antibody, or antigen-binding fragment thereof, comprises a heavy chain variable region (HCVR), light chain variable region (LCVR), and/or complementarity determining regions (CDRs) comprising the amino acid sequences of any of the anti-BCMA×CD3 antibodies set forth in U.S. Pat. No. 11,384,153 and US Patent Publication No. 2020/0345843, which are hereby incorporated by reference in their entireties.

In some exemplary embodiments, an anti-BCMA×CD3 bispecific antibody or antigen-binding fragment thereof that can be used in the context of the present disclosure comprising a HCVR, a LCVR, and/or CDRs comprising the amino acid sequences of REGN5458 or REGN5459 as set forth in Table 1 below.

TABLE 1
Amino Acid Sequences of Exemplary Anti-BCMA × CD3 Bispecific Antibodies

| | Anti-BCMA | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN5458 | 2 | 4 | 6 | 8 | 26 | 28 | 30 | 32 | 18 | 20 | 22 | 24 |
| REGN5459 | 2 | 4 | 6 | 8 | 34 | 36 | 38 | 40 | 18 | 20 | 22 | 24 |

In some embodiments, the anti-BCMA×CD3 bispecific antibody or antigen-binding fragment thereof that can be used the present disclosure comprises: (a) a first antigen binding domain that binds specifically to BCMA; and (b) a second antigen-binding domain that binds specifically to CD3. In one embodiment, the anti-BCMA antigen-binding domain comprises the heavy chain complementarity determining regions (HCDRs) of a heavy chain variable region (HCVR) comprising the amino acid sequence of SEQ ID NO: 2 and the light chain complementarity determining regions (LCDRs) of a light chain variable region (LCVR) comprising the amino acid sequence of SEQ ID NO: 18. In one embodiment, the first antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 4; the HCDR2 comprises the amino acid sequence of SEQ ID NO: 6; the HCDR3 comprises the amino acid sequence of SEQ ID NO: 8; the LCDR1 comprises the amino acid sequence of SEQ ID NO: 20; the LCDR2 comprises the amino acid sequence of SEQ ID NO: 22; and the LCDR3 comprises the amino acid sequence of SEQ ID NO: 24.

In one embodiment, the second antigen-binding domain comprises the heavy chain complementarity determining regions (HCDRs) of a heavy chain variable region (HCVR) comprising the amino acid sequence of SEQ ID NO: 26 or SEQ ID NO: 34 and the light chain complementarity determining regions (LCDRs) of a light chain variable region (LCVR) comprising the amino acid sequence of SEQ ID NO: 18. In one embodiment, the second antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 28 or 36; the HCDR2 comprises the amino acid sequence of SEQ ID NO: 30 or 38; the HCDR3 comprises the amino acid sequence of SEQ ID NO: 32 or 40; the LCDR1 comprises the amino acid sequence of SEQ ID NO: 20; the LCDR2 comprises the amino acid sequence of SEQ ID NO: 22; and the LCDR3 comprises the amino acid sequence of SEQ ID NO: 24.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs: 4, 6, and 8, and LCDR1, LCDR2, and LCDR3 domains, respectively, comprising the amino acid sequences of SEQ ID NOs: 20, 22, and 24; and (b) a second antigen binding domain that comprises HCDR1, HCDR2, and HCDR3 domains, respectively, comprising the amino acid sequences of SEQ ID NOs: 28, 30, and 32, and LCDR1, LCDR2, and LCDR3 domains, respectively, comprising the amino acid sequences of SEQ ID NOs: 20, 22, and 24. In one embodiment, the anti-BCMA×CD3 bispecific antibody or antigen-binding fragment thereof comprises: (a) a first antigen-binding domain that comprises a HCVR comprising the amino acid sequence of SEQ ID NO: 2 and a LCVR comprising the amino acid sequence of SEQ ID NO: 18; and (b) a second antigen-binding domain that comprises a HCVR comprising the amino acid sequence of SEQ ID NO: 26 and a LCVR comprising the amino acid sequence of SEQ ID NO: 18.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs: 4, 6, and 8, and LCDR1, LCDR2, and LCDR3 domains, respectively, comprising the amino acid sequences of SEQ ID NOs: 20, 22, and 24; and (b) a second antigen binding domain that comprises HCDR1, HCDR2, and HCDR3 domains, respectively, comprising the amino acid sequences of SEQ ID NOs: 36, 38, and 40, and LCDR1, LCDR2, and LCDR3 domains, respectively, comprising the amino acid sequences of SEQ ID NOs: 20, 22, and 24. In one embodiment, the anti-BCMA/anti-CD3 bispecific antibody or antigen-binding fragment thereof comprises: (a) a first antigen-binding domain that comprises a HCVR comprising the amino acid sequence of SEQ ID NO: 2 and a LCVR comprising the amino acid sequence of SEQ ID NO: 18; and (b) a second antigen-binding domain that comprises a HCVR comprising the amino acid sequence of SEQ ID NO: 34 and a LCVR comprising the amino acid sequence of SEQ ID NO: 18.

Exemplary anti-BCMA×CD3 bispecific antibodies include the fully human bispecific antibodies known as REGN5458 and REGN5459. See, e.g., WO 2020/018820, US 2020/0024356, US 2022/0306758, and U.S. Pat. No. 11,384,153, each of which is herein incorporated by reference. According to certain exemplary embodiments, the methods of the present disclosure comprise the use of REGN5458 or REGN5459, or a bioequivalent thereof. As used herein, the term “bioequivalent” with respect to anti-BCMA×CD3 antibodies refers to antibodies or BCMA×CD3 binding proteins or fragments thereof that are pharmaceutical equivalents or pharmaceutical alternatives having a rate and/or extent of absorption that does not show a significant difference with that of a reference antibody (e.g., REGN5458 or REGN5459) when administered at the same molar dose under similar experimental conditions, either single dose or multiple dose; the term “bioequivalent” also includes antigen-binding proteins that bind to BCMA/CD3 and do not have clinically meaningful differences with the reference antibody (e.g., REGN5458 or REGN5459) with respect to safety, purity, and/or potency.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 2 and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 26 and a LCVR having at least 90%, 91%, 92%, 93%, 94%, 95%, 96%, 97%, 98%, or 99% sequence identity to the amino acid sequence of SEQ ID NO: 18. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOs: 4, 6, and 8, respectively, and a HCVR having at least 90%, 91%, 92%, 93%, 94%, 95%, 96%, 97%, 98%, or 99% sequence identity to the amino acid sequence of SEQ ID NO: 2, and comprises three LCDRs (LCDR1, LCDR2 and LCDR3) comprising the amino acid sequences of SEQ ID NOs: 20, 22, and 24, respectively, and a LCVR having at least 90%, 91%, 92%, 93%, 94%, 95%, 96%, 97%, 98%, or 99% sequence identity to the amino acid sequence of SEQ ID NO:18; and (b) a second antigen-binding domain that comprises three HCDRs (HCDR1, HCDR2 and HCDR3) comprising the amino acid sequences of SEQ ID NOs: 28, 30, and 32, respectively, and a HCVR having at least 90%, 91%, 92%, 93%, 94%, 95%, 96%, 97%, 98%, or 99% sequence identity to the amino acid sequence of SEQ ID NO: 26, and comprises three LCDRs (LCDR1, LCDR2 and LCDR3) comprising the amino acid sequences of SEQ ID NOs: 20, 22, and 24, respectively, and a LCVR having at least 90%, 91%, 92%, 93%, 94%, 95%, 96%, 97%, 98%, or 99% sequence identity to the amino acid sequence of SEQ ID NO:18.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 2 and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 34 and a LCVR having at least 90%, 91%, 92%, 93%, 94%, 95%, 96%, 97%, 98%, or 99% sequence identity to the amino acid sequence of SEQ ID NO: 18. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOs: 4, 6, and 8, respectively, and a HCVR having at least 90%, 91%, 92%, 93%, 94%, 95%, 96%, 97%, 98%, or 99% sequence identity to the amino acid sequence of SEQ ID NO: 2, and comprises three LCDRs (LCDR1, LCDR2 and LCDR3) comprising the amino acid sequences of SEQ ID NOs: 20, 22, and 24, respectively, and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises three HCDRs (HCDR1, HCDR2 and HCDR3) comprising the amino acid sequences of SEQ ID NOs: 36, 38, and 40, respectively, and a HCVR having at least 90%, 91%, 92%, 93%, 94%, 95%, 96%, 97%, 98%, or 99% sequence identity to the amino acid sequence of SEQ ID NO: 34, and comprises three LCDRs (LCDR1, LCDR2 and LCDR3) comprising the amino acid sequences of SEQ ID NOs: 20, 22, and 24, respectively, and a LCVR having at least 90%, 91%, 92%, 93%, 94%, 95%, 96%, 97%, 98%, or 99% sequence identity to the amino acid sequence of SEQ ID NO:18.

The present disclosure also includes variants of the anti-BCMA×CD3 antibodies described herein comprising any of the HCVR, LCVR, and/or CDR amino acid sequences disclosed herein with one or more conservative amino acid substitutions. For example, the present disclosure includes use of anti-BCMA×CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, the disclosure includes use of an anti-BCMA×CD3 antibody having HCVR, LCVR, and/or CDR amino acid sequences with 1, 2, 3, or 4 conservative amino acid substitutions relative to any of the HCVR, LCVR, and/or CDR amino acid sequences disclosed herein.

Other anti-BCMA×CD3 antibodies that can be used in the methods of the present disclosure include, e.g., the antibodies referred to and known in the art as pacanalotamab (AMG420), teclistamab (JNJ-64007957), AMG701, alnuctamab (CC-93269), EM801, EM901, elranatamab (PF-06863135), TNB383B (ABBV-383), and TNB384B, or any of the anti-BCMA×CD3 antibodies set forth, e.g., in International Patent Publications WO2013/072415, WO2014/140248, WO2014/122144, WO2016/166629, WO2016/079177, WO2016/020332, WO2017031104, WO2017/223111, WO2017/134134, WO2018/083204, or WO2018/201051. The portions of the publications cited herein that identify anti-BCMA×CD3 antibodies are hereby incorporated by reference.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

The bispecific antigen-binding molecules disclosed herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In some embodiments, the human IgG heavy chain constant region comprises one or more modifications that reduces binding to an Fc receptor. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In some embodiments, the antibody comprises a first heavy chain containing the HCVR of the first antigen-binding domain and a second heavy chain containing the HCVR of the second antigen-binding domain, wherein the first heavy chain comprises residues 1-450 of the amino acid sequence of SEQ ID NO: 41 and the second heavy chain comprises residues 1-449 of the amino acid sequence of SEQ ID NO: 42.

In some embodiments, the antibody comprises a common light chain containing the LCVR of the first and second antigen-binding domains, wherein the common light chain comprises the amino acid sequence of SEQ ID NO: 43.

In some embodiments, the anti-BCMA×CD3 bispecific antibody comprises a first heavy chain comprising the amino acid sequence of SEQ ID NO: 41, a second heavy chain comprising the amino acid sequence of SEQ ID NO: 42, and a common light chain comprising the amino acid sequence of SEQ ID NO: 43. In some cases, the mature form of the antibody may not include the C-terminal lysine residues of SEQ ID NOs: 41 and 42. Thus, in some cases the anti-BCMA binding arm comprises a heavy chain comprising residues 1-450 of SEQ ID NO: 41, and the anti-CD3 binding arm comprises a heavy chain comprising residues 1-449 of SEQ ID NO: 42.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

In some embodiments, a bispecific antigen-binding molecule of the present disclosure comprises two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In some embodiments, the multimerizing domain is an Fc fragment or an amino acid sequence of from 1 to about 200 amino acids in length, containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

### B Cell Depleting Agents

In some embodiments, the methods disclosed herein include administering a therapeutically effective amount of a B cell depleting agent to a subject in need thereof. As used herein, a “B cell depleting agent” refers to any molecule capable of specifically binding to a surface antigen on B cells and killing or depleting said B cell. Thus, in general, a B cell depleting agent can be any agent that binds to a B cell surface molecule. In some embodiments, the B cell depleting agent is capable of depleting B cells and plasma cells that express low levels of BCMA.

In various aspects, the present disclosure provides B cell depleting agents, which may be administered to a subject in need thereof, e.g., either alone or combined with, or administered in combination with, a plasma cell depleting agent (e.g., an anti-BCMA×CD3 bispecific antibody, or a functional fragment thereof), an immunoglobulin depleting agent (e.g., an FcFn blocker such as, e.g., efgartigimod), and/or an immunogen (e.g., an immunogenic delivery vehicle such as, e.g., AAV). In some embodiments plasmapheresis, therapeutic plasma exchange, and/or immunoadsorption may be further combined with the administering of the B cell depleting agent, the plasma cell depleting agent, the immunoglobulin depleting agent, and/or the immunogen. In some embodiments, the subject does not have a pre-existing immunity against the immunogen.

In some embodiments, a B cell depleting agent may be administered alone (e.g., as a monotherapy, in the absence of the administration of any other additional immunomodulators [e.g., plasma cell depleting agents, immunoglobulin depleting agents], and optionally, combined with, or administered in combination with, an immunogen) to a subject in need thereof, e.g., a subject without a pre-existing immunity against an immunogen (e.g., an immunogen to be administered to the subject e.g., an immunogenic delivery vehicle such as, e.g., AAV). In some embodiments, the B cell depleting agent may be administered alone to a subject who is immunologically naïve to an immunogen to be administered to the subject (e.g., AAV). In some embodiments, the B cell depleting agent may be administered alone to an AAV seronegative subject, and the subject is further administered an immunogen (e.g., AAV). In some embodiments, a B cell depleting agent may be useful as a prophylactic treatment to prevent or suppress an immune response (e.g., an anti-AAV IgG, IgM, and/or nAb response) to an immunogen (e.g., AAV) in a subject in need thereof (e.g., a subject without a pre-existing immunity to the immunogen).

In some embodiments, the suppression or prevention of an immune response (e.g., an anti-AAV IgG, IgM, and/or nAb response) to an immunogen in a subject (e.g., a subject without a pre-existing immunity to the immunogen) can be achieved by administering a B cell depleting agent described herein (e.g., an anti-CD20×CD3 bispecific antibody or a functional fragment thereof). Administration of the B cell depleting agent to the subject can suppress or prevent the immune response in the subject following the initial dosing and/or re-dosing of an immunogen (e.g., post-AAV dosing and/or re-dosing). In some embodiments, an immune response may be suppressed in a subject following AAV dosing and/or re-dosing. The immune response may be suppressed by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more, e.g., relative to an immune response in a subject receiving no immunomodulation treatment or treatment with a conventional anti-CD20 therapeutic alone (e.g., rituximab, or derivatives or equivalents thereof). The immune response may be suppressed by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The immune response may be suppressed by about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or even 100%. In some embodiments, the immune response is prevented. In some embodiments, an immune response may be suppressed or prevented in a subject following AAV dosing and/or re-dosing in the subject to achieve levels equivalent to, or even below, those of an AAV-naïve subject. In some embodiments, a B cell depleting agent is sufficient to enable effective re-dosing of an immunogen to a subject. The B cell depleting agent can be administered to the subject prior to the re-dosing of the immunogen any number of times and can be used to maintain a suppressed immune response to the immunogen in the subject for any period of time thereafter.

In some embodiments, a B cell depleting agent is capable of suppressing an anti-immunogen response (e.g., an anti-AAV response) in a subject, and the anti-immunogen response is mounted by the subject in response to repeated doses of the immunogen (e.g. AAV).

It is contemplated that a B cell depleting agent may be used in the suppression or prevention of an anti-immunogen antibody response (e.g., an anti-AAV antibody response) in a subject, and the suppression or prevention of the anti-immunogen antibody response involves B cell depletion in primary and/or secondary lymphoid tissues. Non-limiting examples of primary lymphoid tissues include bone marrow and thymus. In some embodiments, the compositions and methods of the disclosure encompass B cell depletion in secondary lymphoid tissues, for example, and without limitation, spleen and/or lymph nodes. In some embodiments, the compositions and methods of the disclosure relate to B cell depletion in lymph nodes, which is achieved by a B cell depleting agent described herein.

In some embodiments, the present disclosure provides B cell depleting agents combined with, or administered in combination with, plasma cell depleting agents (e.g., an anti-BCMA×CD3 bispecific antibody, or a functional fragment thereof) described herein to subjects, e.g., subjects with or without a pre-existing immunity against an immunogen (i.e., an immunogen administered to the subject, e.g., an immunogenic delivery vehicle such as, e.g., AAV). In some embodiments, the B cell depleting agent may be administered in combination with a plasma cell depleting agent, an immunoglobulin depleting agent, plasmapheresis, therapeutic plasma exchange, immunoadsorption, and/or an immunogen (e.g., an immunogenic delivery vehicle) disclosed herein. In some embodiments, the B cell depleting agent may be administered to subjects without a pre-existing immunity against an immunogen (i.e., an immunogen to be administered to the subject, e.g., an immunogenic delivery vehicle such as, e.g., AAV) not only alone, but also in combination with a plasma cell depleting agent, an immunoglobulin depleting agent, plasmapheresis, therapeutic plasma exchange, or immunoadsorption, and/or an immunogen (e.g., an immunogenic delivery vehicle such as, e.g., AAV) disclosed herein. Suitable combinations comprising a plasma cell depleting agent are described in more detail elsewhere herein.

In some embodiments, the B cell depleting agent is an agent that directly targets a B cell, e.g., an agent that binds to a B cell surface molecule. In some embodiments, the B cell depleting agent causes a reduction in the number of B cells in a subject (e.g., in a blood sample taken from the subject). In some embodiments, a B cell depleting agent may be useful for, e.g., eliminating non-plasma cell (e.g. non-long-lived plasma cell [LLPC] sources of immunogen (e.g., anti-AAV) nAbs. In some embodiments, a B cell depleting agent may be useful for, e.g., preventing formation of non-plasma cell (e.g. non-long-lived plasma cell [LLPC] sources of immunogen (e.g., anti-AAV) nAbs (e.g., in AAV-naïve patients). In some embodiments, the B cell depleting agent may capture a wider range of AAV-specific B cells and plasma cells that may not express high levels of BCMA (e.g., committed memory B cells and early plasmablasts).

In some embodiments, the B cell depleting agent comprises an anti-CD19 antibody (e.g., MEDI-551, tafasitamab, Inebilizumab, loncastuximab), an anti-CD20 antibody (e.g., rituximab, ocrelizumab, obinutuzumab, ublituximab, or ofatumumab), an anti-CD22 antibody (e.g., epratuzumab), an anti-CD79 antibody (e.g., polatuzumab), a bispecific CD20×CD3 B cell depleting antibody (e.g. odronextamab, glofitamab, mosunetuzumab, epcoritamab), a bispecific CD19×CD3 antibody (e.g., blinatumomab), a bispecific CD22×CD3 antibody (e.g., inotuzumab), or functional fragments thereof, or any combination thereof.

In some embodiments, the B cell depleting agent is an agent that indirectly targets a B cell, e.g., by targeting a B cell survival factor. In some embodiments, the B cell depleting agent is a BLyS/BAFF inhibitor (e.g., belimumab, lanalumab, BR3-Fc, AMG-570, or AMG-623), an APRIL inhibitor (e.g., telitacicept, atacicept), or a BLyS receptor 3/BAFF receptor inhibitor (e.g., anti-BR3), or any combination thereof.

In some embodiments, the B cell depleting agent is selected from anti-CD19 antibodies, anti-CD20 antibodies, anti-CD22 antibodies, anti-CD79 antibodies, multispecific antibodies combining two or more of any of said antibody specificities, multispecific antibodies combining any of said antibody specificities with anti-CD3 antibodies, functional fragments of any of said antibodies, and any combinations thereof. In some embodiments, the B cell depleting agent comprises an anti-CD20 antibody or a functional fragment thereof and an anti-CD19 antibody or a functional fragment thereof. In certain embodiments, the B cell depleting agent is an anti-CD20 antibody or a functional fragment thereof. In some embodiments, a multispecific anti-CD20 antibody or functional fragment thereof of the present disclosure targets CD20 and CD19. In some embodiments the multispecific anti-CD20 antibody or functional fragment thereof is anti-CD19×CD20 bispecific antibody, or functional fragment thereof.

In some embodiments, the B cell depleting agent comprises anti-CD19 and anti-CD20 antibodies (also referred to as “anti-CD19/CD20 antibodies” herein), or functional fragments thereof, disclosed herein.

In a specific embodiment, the B cell depleting agent comprises a bispecific antibody that specifically binds CD3 and CD19. Such antibodies may be referred to herein as, e.g., “anti-CD19/anti-CD3,” or “anti-CD19×CD3” or “CD19×CD3” bispecific antibodies, or other similar terminology.

In a specific embodiment, the B cell depleting agent comprises a bispecific antibody that specifically binds CD3 and CD20. Such antibodies may be referred to herein as, e.g., “anti-CD20/anti-CD3,” or “anti-CD20×CD3” or “CD20×CD3” bispecific antibodies, or other similar terminology.

As used herein, the expression “bispecific antibody” refers to an immunoglobulin protein comprising at least a first antigen-binding domain and a second antigen-binding domain. In some embodiments, the first antigen-binding domain specifically binds a first antigen (e.g., CD20), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain can each be connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present invention, the multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present invention typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antibodies of the present invention, Fc domains may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US 2015/0266966, incorporated herein in its entirety.

The present invention also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2] [IgG1 C_{H}3]. These and other examples of chimeric Fc domains that can be included in any of the antigen-binding molecules of the present invention are described in US Patent Publication No. 2014/0243504, which is herein incorporated in its entirety. Chimeric Fc domains having these general structural arrangements, and variants thereof, can have altered Fc receptor binding, which in turn affects Fc effector function.

### CD20×CD3 Antigen-Binding Molecules

The term “CD20,” as used herein, refers to an antigen which is expressed on B cells and which consists of a non-glycosylated phosphoprotein expressed on the cell membranes of mature B cells. The human CD20 protein can have the amino acid sequence as in NCBI Reference Sequence NP_690605.1. As used herein, the expression “anti-CD20 antibody” includes monovalent antibodies with a single specificity, such as RITUXAN® (rituximab), as described in U.S. Pat. No. 7,879,984. Exemplary anti-CD20 antibodies are also described in U.S. Pat. No. 7,879,984 and PCT International Application No. PCT/US2013/060511, filed on Sep. 19, 2013, each incorporated by reference herein.

In some exemplary embodiments, the CD20 targeting agent used in the disclosed methods is a multispecific (e.g., bispecific) antibody, or a functional fragment thereof, that specifically binds CD20 and CD3 (e.g., an anti-CD20×CD3 bispecific antibody). The anti-CD20×CD3 multispecific (e.g., bispecific) antibodies are useful for specific targeting and T-cell-mediated killing of cells that express CD20. The terms “antibody,” “antigen-binding fragment,” “human antibody,” “recombinant antibody,” and other related terminology are defined above. In the context of anti-CD20×CD3 antibodies and antigen-binding fragments thereof, the present disclosure includes the use of bispecific antibodies wherein one arm of an immunoglobulin is specific for CD20 or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

The anti-CD20×CD3 bispecific antibodies are capable of simultaneously binding to human CD3 and human CD20. According to certain embodiments, the anti-CD20×CD3 bispecific antibodies specifically interact with cells that express CD3 and/or CD20. The extent to which the anti-CD20×CD3 bispecific antibodies binds cells that express CD3 and/or CD20 can be assessed by fluorescence activated cell sorting (FACS). In certain embodiments, the anti-CD20×CD3 bispecific antibodies specifically bind human T-cell lines which express CD3 (e.g., Jurkat), human B-cell lines which express CD20 (e.g., Raji), and primate T-cells (e.g., cynomolgus peripheral blood mononuclear cells [PBMCs]).

In some embodiments, the anti-CD20×CD3 bispecific antigen-binding molecule comprises a first antigen-binding domain (D1) that binds an epitope of CD20 (e.g., human CD20), and a second antigen-binding domain (D2) that binds an epitope of CD3 (e.g., human CD3).

According to certain exemplary embodiments of the present invention, the bispecific anti-CD20×CD3 antibody, or antigen-binding fragment thereof comprises heavy chain variable regions (A-HCVR and B-HCVR), light chain variable region (LCVR), and/or complementarity determining regions (CDRs) comprising any of the amino acid sequences of the bispecific anti-CD20×CD3 antibodies as set forth in US Patent Publication No. 20150266966, incorporated herein by reference in its entirety.

In certain exemplary embodiments, the bispecific anti-CD20×CD3 antibody or antigen-binding fragment thereof that can be used in the context of the methods of the present invention comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 44 and the light chain complementarity determining regions (LCDRs) of a light chain variable region (LCVR) comprising the amino acid sequence of SEQ ID NO: 45; and (b) a second antigen-binding arm comprising the heavy chain CDRs (B-HCDR1, B-HCDR2 and B-HCDR3) of a HCVR (B-HCVR) comprising the amino acid sequence of SEQ ID NO: 46 and the light chain CDRs of a LCVR comprising the amino acid sequence of SEQ ID NO: 45. According to certain embodiments, the A-HCDR1 comprises the amino acid sequence of SEQ ID NO: 47; the A-HCDR2 comprises the amino acid sequence of SEQ ID NO: 48; the A-HCDR3 comprises the amino acid sequence of SEQ ID NO: 49; the LCDR1 comprises the amino acid sequence of SEQ ID NO: 50; the LCDR2 comprises the amino acid sequence of SEQ ID NO: 51; the LCDR3 comprises the amino acid sequence of SEQ ID NO: 52; the B-HCDR1 comprises the amino acid sequence of SEQ ID NO: 53; the B-HCDR2 comprises the amino acid sequence of SEQ ID NO: 54; and the B-HCDR3 comprises the amino acid sequence of SEQ ID NO: 55. In yet other embodiments, the bispecific anti-CD20×CD3 antibody or antigen-binding fragment thereof comprises: (a) a first antigen-binding arm comprising a HCVR (A-HCVR) comprising SEQ ID NO: 44 and a LCVR comprising SEQ ID NO: 45; and (b) a second antigen-binding arm comprising a HCVR (B-HCVR) comprising SEQ ID NO: 46 and a LCVR comprising SEQ ID NO: 45.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises: a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

Other bispecific anti-CD20×CD3 antibodies that can be used in the context of the methods of the present invention include, e.g., any of the antibodies as set forth in US 2014/0088295, US 2015/0166661, and US 2017/0174781, the disclosure of each of which is incorporated by reference in its entirely. An exemplary bispecific anti-CD20×CD3 antibody that can be used in the context of the methods of the present invention is the bispecific anti-CD20×CD3 antibody known as REGN1979 or bsAB1.

In some exemplary embodiments, an anti-CD20×CD3 bispecific antibody or antigen-binding fragment thereof that can be used in the context of the present disclosure comprising a HCVR, a LCVR, and/or CDRs comprising the amino acid sequences of REGN1979 as set forth in Table 2 below.

TABLE 2
Amino Acid Sequences of Exemplary Anti-CD20 × CD3 Bispecific Antibodies.

| | Anti-CD20 | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN1979 | 44 | 47 | 48 | 49 | 46 | 53 | 54 | 55 | 45 | 50 | 51 | 52 |

In some embodiments, the anti-CD20×CD3 bispecific antibody or antigen-binding fragment thereof that can be used in the present disclosure comprises: (a) a first antigen binding domain that binds specifically to CD20; and (b) a second antigen-binding domain that binds specifically to CD3. In one embodiment, the anti-CD20 antigen-binding domain comprises the heavy chain complementarity determining regions (A-HCDRs) of a heavy chain variable region (A-HCVR) comprising the amino acid sequence of SEQ ID NO: 44 and the light chain complementarity determining regions (LCDRs) of a light chain variable region (LCVR) comprising the amino acid sequence of SEQ ID NO: 45. In one embodiment, the first antigen-binding domain comprises three HCDRs (A-HCDR1, A-HCDR2 and A-HCDR3) and three LCDRs (LCDR1, LCDR2 and LCDR3), wherein the A-HCDR1 comprises the amino acid sequence of SEQ ID NO: 47; the A-HCDR2 comprises the amino acid sequence of SEQ ID NO: 48; the A-HCDR3 comprises the amino acid sequence of SEQ ID NO: 49; the LCDR1 comprises the amino acid sequence of SEQ ID NO: 50; the LCDR2 comprises the amino acid sequence of SEQ ID NO: 51; and the LCDR3 comprises the amino acid sequence of SEQ ID NO: 52.

In one embodiment, the second antigen-binding domain comprises the heavy chain complementarity determining regions (B-HCDRs) of a heavy chain variable region (B-HCVR) comprising the amino acid sequence of SEQ ID NO: 46 and the light chain complementarity determining regions (LCDRs) of a light chain variable region (LCVR) comprising the amino acid sequence of SEQ ID NO: 45. In one embodiment, the second antigen-binding domain comprises three HCDRs (B-HCDR1, B-HCDR2 and B-HCDR3) and three LCDRs (LCDR1, LCDR2 and LCDR3), wherein the B-HCDR1 comprises the amino acid sequence of SEQ ID NO: 53; the B-HCDR2 comprises the amino acid sequence of SEQ ID NO: 54; the B-HCDR3 comprises the amino acid sequence of SEQ ID NO: 55; the LCDR1 comprises the amino acid sequence of SEQ ID NO: 50; the LCDR2 comprises the amino acid sequence of SEQ ID NO: 51; and the LCDR3 comprises the amino acid sequence of SEQ ID NO: 52.

In one embodiment, the anti-CD20×CD3 bispecific antibody or antigen-binding fragment thereof comprises: (a) a first antigen-binding domain that comprises A-HCDR1, A-CDR2, and A-HCDR3 domains, respectively, comprising the amino acid sequences of SEQ ID NOs: 47, 48, and 49, and LCDR1, LCDR2, and LCDR3 domains, respectively, comprising the amino acid sequences of SEQ ID NOs: 50, 51, and 52; and (b) a second antigen binding domain that comprises B-HCDR1, B-HCDR2, and B-HCDR3 domains, respectively, comprising the amino acid sequences of SEQ ID NOs: 53, 54, and 55, and LCDR1, LCDR2, and LCDR3 domains, respectively, comprising the amino acid sequences of SEQ ID NOs: 50, 51, and 52. In one embodiment, the anti-CD20×CD3 bispecific antibody or antigen-binding fragment thereof comprises: (a) a first antigen-binding domain that comprises a A-HCVR comprising the amino acid sequence of SEQ ID NO: 44 and a LCVR comprising the amino acid sequence of SEQ ID NO: 45; and (b) a second antigen-binding domain that comprises a B-HCVR comprising the amino acid sequence of SEQ ID NO: 46 and a LCVR comprising the amino acid sequence of SEQ ID NO: 45.

Exemplary anti-CD20×CD3 bispecific antibodies include the fully human bispecific antibody known as REGN1979. See, e.g., US 2014/0088295, US 2015/0166661, and US 2017/0174781, each of which is herein incorporated by reference. According to certain exemplary embodiments, the methods of the present disclosure comprise the use of REGN1979, or a bioequivalent thereof. As used herein, the term “bioequivalent” with respect to anti-CD20×CD3 antibodies refers to antibodies or CD20×CD3 binding proteins or fragments thereof that are pharmaceutical equivalents or pharmaceutical alternatives having a rate and/or extent of absorption that does not show a significant difference with that of a reference antibody (e.g., REGN1979) when administered at the same molar dose under similar experimental conditions, either single dose or multiple dose; the term “bioequivalent” also includes antigen-binding proteins that bind to CD20/CD3 and do not have clinically meaningful differences with the reference antibody (e.g., REGN1979) with respect to safety, purity, and/or potency.

In some embodiments, the anti-CD20×CD3 bispecific antibody or antigen-binding fragment thereof comprises: (a) a first antigen-binding domain that comprises a A-HCVR having at least 90%, 91%, 92%, 93%, 94%, 95%, 96%, 97%, 98%, or 99% sequence identity to the amino acid sequence of SEQ ID NO: 44 and a LCVR having at least 90%, 91%, 92%, 93%, 94%, 95%, 96%, 97%, 98%, or 99% sequence identity to the amino acid sequence of SEQ ID NO: 45; and (b) a second antigen-binding domain that comprises a B-HCVR having at least 90%, 91%, 92%, 93%, 94%, 95%, 96%, 97%, 98%, or 99% sequence identity to the amino acid sequence of SEQ ID NO: 46 and a LCVR having at least 90%, 91%, 92%, 93%, 94%, 95%, 96%, 97%, 98%, or 99% sequence identity to the amino acid sequence of SEQ ID NO: 45. In some embodiments, the anti-CD20×CD3 bispecific antibody or antigen-binding fragment thereof comprises: (a) a first antigen-binding domain that comprises three HCDRs (A-HCDR1, A-HCDR2 and A-HCDR3) comprising the amino acid sequences of SEQ ID NOs: 47, 48, and 49, respectively, and an A-HCVR having at least 90%, 91%, 92%, 93%, 94%, 95%, 96%, 97%, 98%, or 99% sequence identity to the amino acid sequence of SEQ ID NO: 44, and comprises three LCDRs (LCDR1, LCDR2 and LCDR3) comprising the amino acid sequences of SEQ ID NOs: 50, 51, and 52, respectively, and a LCVR having at least 90%, 91%, 92%, 93%, 94%, 95%, 96%, 97%, 98%, or 99% sequence identity to the amino acid sequence of SEQ ID NO: 45; and (b) a second antigen-binding domain that comprises three HCDRs (B-HCDR1, B-HCDR2 and B-HCDR3) comprising the amino acid sequences of SEQ ID NOs: 53, 54, and 55, respectively, and a B-HCVR having at least 90%, 91%, 92%, 93%, 94%, 95%, 96%, 97%, 98%, or 99% sequence identity to the amino acid sequence of SEQ ID NO: 46, and comprises three LCDRs (LCDR1, LCDR2 and LCDR3) comprising the amino acid sequences of SEQ ID NOS: 50, 51, and 52, respectively, and a LCVR having at least 90%, 91%, 92%, 93%, 94%, 95%, 96%, 97%, 98%, or 99% sequence identity to the amino acid sequence of SEQ ID NO:45.

The present disclosure also includes variants of the anti-CD20×CD3 antibodies described herein comprising any of the HCVR, LCVR, and/or CDR amino acid sequences disclosed herein with one or more conservative amino acid substitutions. For example, the present disclosure includes use of anti-CD20×CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, the disclosure includes use of an anti-CD20×CD3 antibody having HCVR, LCVR, and/or CDR amino acid sequences with 1, 2, 3, or 4 conservative amino acid substitutions relative to any of the HCVR, LCVR, and/or CDR amino acid sequences disclosed herein.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

In some embodiments, the anti-CD20×CD3 bispecific antibody or functional fragment thereof comprises a human IgG heavy chain constant region. In some embodiments, the human IgG heavy chain constant region is isotype IgG4 or IgG1. In some embodiments, the human IgG heavy chain constant region comprises one or more modifications that reduces binding to an Fc receptor. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

### Sequence Variants

The antigen-binding molecules of the present disclosure may comprise one or more amino acid substitutions, insertions, and/or deletions in the framework and/or CDR regions of the heavy and/or light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen binding fragments which are derived from any of the exemplary amino acid sequences disclosed herein, wherein one or more amino acids within one or more framework and/or CDR regions are mutated to the corresponding residue(s) of the germline sequence from which the antibody was derived, to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired property such as, improved binding specificity, increased binding affinity, improved, or enhanced antagonistic or agonistic biological properties, reduced immunogenicity, etc. Bispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. In some embodiments, the disclosure includes use of an antibody having HCVR, LCVR and/or CDR amino acid sequences with 1, 2, 3, or 4 conservative amino acid substitutions relative to any of the HCVR, LCVR and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate; and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antigen-binding molecules comprising an antigen binding domain with a HCVR, LCVR, and/or CDR amino acid sequence that is substantially identical to any of the HCVR, LCVR, and/or CDR amino acid sequences disclosed herein. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1, wherein the differences in the amino acid residue(s) relative to the sequence disclosed in Table 1 are conservative substitutions or moderately conservative substitutions.

### Antigen-Binding Proteins Comprising Fc Modifications

In some embodiments, an antigen-binding molecule as disclosed herein (e.g., a BCMA×CD3 bispecific antigen-binding molecule such as an anti-BCMA×CD3 bispecific antibody or a CD20×CD3 bispecific antigen-binding molecule such as an anti-CD20×CD3 bispecific antibody) comprises an Fc domain comprising one or more modifications or mutations that enhance or diminish antibody binding to the FcRn receptor. For example, the present disclosure includes antigen-binding molecules comprising one or more mutations in the CH2 and/or CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). See, e.g., Ko et al., BioDrugs 2021, 35:147-157.

In certain embodiments, a BCMA×CD3 bispecific antigen-binding molecule or a CD20×CD3 bispecific antigen-binding molecule comprises an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F).

In some embodiments, the BCMA×CD3 bispecific antigen-binding molecules or the CD20×CD3 bispecific antigen-binding molecules of the present disclosure comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, a modified Fc domain having reduced binding to an Fc receptor (e.g., Fcγ receptor, e.g., FcγRI, FcγRIIA, FcγRIIB, or FcγRIIIA) is a variant IgG1 Fc or a variant IgG4 Fc comprising one or more substitutions or modifications in the hinge region and/or a CH region (e.g., CH2). For example, a modified Fc domain may comprise a variant IgG1 Fc wherein at least one amino acid of an IgG1 Fc hinge region and/or CH region is replaced with the corresponding amino acid from an IgG2 Fc hinge region and/or CH region. In certain embodiments, the modified Fc domain is a variant IgG1 Fc or a variant IgG4 Fc comprising one or more substitutions or modifications in the hinge region. For example, a modified Fc domain may comprise a variant IgG1 Fc, wherein at least one amino acid of the IgG1 Fc hinge region is replaced with the corresponding amino acid from the IgG2 Fc hinge region. In one example, the variant IgG1 Fc can comprise a human IgG2 lower hinge amino acid sequence or can comprise both a human IgG2 lower hinge amino acid sequence and a human IgG4 CH2 amino acid sequence. For example, in some embodiments, the heavy chain constant region can comprise a variant IgG1 Fc in which positions 233-236 by EU numbering are occupied by PVA. See, e.g., U.S. Pat. No. 10,988,537, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by PVA. Alternatively, a modified Fc domain may comprise a variant IgG4 Fc wherein at least one amino acid of an IgG4 Fc hinge region and/or CH region is replaced with the corresponding amino acid from an IgG2 Fc hinge region and/or CH region. Alternatively, a modified Fc domain may comprise a variant IgG4 Fc, wherein at least one amino acid of the IgG4 Fc hinge region is replaced with the corresponding amino acid from the IgG2 Fc hinge region. In one example, the variant IgG4 Fc can comprise a human IgG2 lower hinge amino acid sequence. For example, in some embodiments, the heavy chain constant region can comprise a variant IgG4 Fc in which positions 233-236 by EU numbering are occupied by PVA. See, e.g., U.S. Pat. No. 10,988,537, the disclosure of which is hereby incorporated by reference in its entirety. In some embodiments, a modified Fc domain comprises a modified hinge region in which each of positions 233-236 by EU numbering is occupied by G or is unoccupied. In some embodiments, a modified Fc domain comprises modifications in which each of positions 233-236 by EU numbering is occupied by G or is unoccupied. For example, in some embodiments, a modified Fc domain can comprise a modified hinge region in which positions 233-236 by EU numbering are occupied by GGG. See, e.g., U.S. Pat. No. 11,518,807, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by GGG. Non-limiting, exemplary modified Fc regions that can be used in the context of the present disclosure are set forth in U.S. Pat. No. 11,518,807, the disclosure of which is hereby incorporated by reference in its entirety, as well as any functionally equivalent variants of the modified Fc regions set forth therein. Other modified Fc domains and Fc modifications that can be used in the context of the present disclosure include any of the modifications as set forth in U.S. Pat. Nos. 8,697,396, 10,988,537, US 2014/0171623, US 2014/0134162, US 2014/0243504, and WO 2014/043361, the disclosures of each of which are incorporated by reference herein.

All possible combinations of the foregoing Fc domain mutations, and other mutations within the antibody variable domains disclosed herein, are contemplated within the scope of the present disclosure.

### Polynucleotides, Vectors, and Host Cells

In another aspect, the present disclosure provides nucleic acid molecules comprising one or more polynucleotide sequences encoding the antigen-binding molecules disclosed herein, as well as vectors (e.g., expression vectors) encoding such polynucleotide sequences and host cells into which such vectors have been introduced.

Polynucleotides, as disclosed herein, may encode all or a portion of an antigen-binding molecule, antibody, or antigen-binding fragment as disclosed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR, respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as disclosed above or herein.

In some embodiments, the nucleic acid molecule comprises one or more polynucleotide sequences encoding an antigen-binding molecule disclosed in Table 1.

In some embodiments, the nucleic acid molecule comprises a polynucleotide sequence that encodes an anti-BCMA HCVR comprising the HCDR1, HCDR2, and HCDR3 of SEQ ID NOs: 4, 6, and 8, respectively. In some embodiments, the nucleic acid molecule comprises a polynucleotide sequence that encodes an anti-BCMA HCVR comprising or consisting of the sequence of SEQ ID NO: 2. In some embodiments, the nucleic acid molecule comprises a polynucleotide sequence of SEQ ID NO: 1, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 1.

In some embodiments, the nucleic acid molecule comprises a polynucleotide sequence that encodes an anti-CD3 HCVR comprising the HCDR1, HCDR2, and HCDR3 of SEQ ID NOs: 28, 30, and 32, respectively; or of SEQ ID NOs: 36, 38, and 40, respectively. In some embodiments, the nucleic acid molecule comprises a polynucleotide sequence that encodes an anti-CD3 HCVR comprising or consisting of the sequence of SEQ ID NO: 26 or 34. In some embodiments, the nucleic acid molecule comprises a polynucleotide sequence of SEQ ID NO: 25 or 33, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 25 or 33.

In some embodiments, the nucleic acid molecule comprises a polynucleotide sequence that encodes an LCVR comprising the LCDR1, LCDR2, and LCDR3 of SEQ ID NOs: 20, 22, and 24, respectively. In some embodiments, the nucleic acid molecule comprises a polynucleotide sequence that encodes an LCVR comprising or consisting of the sequence of SEQ ID NO: 18. In some embodiments, the nucleic acid molecule comprises the polynucleotide sequence of SEQ ID NO: 17, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 17.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds BCMA, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds BCMA, a second nucleic acid molecule comprising a polynucleotide sequence encoding an LCVR of a first antigen-binding domain that binds BCMA, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-BCMA HCVR comprises the HCDR1, HCDR2, and HCDR3 of SEQ ID NOs: 4, 6, and 8, respectively. In some embodiments, an anti-BCMA LCVR comprises LCDR1, LCDR2, and LCDR3 of SEQ ID NOs: 20, 22, and 24, respectively. In some embodiments, an anti-CD3 HCVR comprises the HCDR1, HCDR2, and HCDR3 of SEQ ID NOs: 28, 30, and 32, respectively; or the HCDR1, HCDR2, and HCDR3 of SEQ ID NOs: 36, 38, and 40, respectively. In some embodiments, an anti-CD3 LCVR comprises the LCDR1, LCDR2, and LCDR3 of SEQ ID NOs: 20, 22, and 24, respectively.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 2, a nucleotide sequence encoding the HCVR sequence of the anti-CD3 antigen-binding domain comprising SEQ ID NO: 26, and a nucleotide sequence encoding the LCVR sequence comprising SEQ ID NO: 18.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 2, a nucleotide sequence encoding the HCVR sequence of the anti-CD3 antigen-binding domain comprising SEQ ID NO: 34, and a nucleotide sequence encoding the LCVR sequence comprising SEQ ID NO: 18.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

In some embodiments, the nucleic acid molecule comprises one or more polynucleotide sequences encoding an antigen-binding molecule disclosed in Table 2.

In some embodiments, the nucleic acid molecule comprises a polynucleotide sequence that encodes an anti-CD20 HCVR comprising the HCDR1, HCDR2, and HCDR3 of SEQ ID NOs: 47, 48, and 49, respectively. In some embodiments, the nucleic acid molecule comprises a polynucleotide sequence that encodes an anti-CD20 HCVR comprising or consisting of the sequence of SEQ ID NO: 44.

In some embodiments, the nucleic acid molecule comprises a polynucleotide sequence that encodes an anti-CD3 HCVR comprising the HCDR1, HCDR2, and HCDR3 of SEQ ID NOS: 53, 54, and 55, respectively. In some embodiments, the nucleic acid molecule comprises a polynucleotide sequence that encodes an anti-CD3 HCVR comprising or consisting of the sequence of SEQ ID NO: 46.

In some embodiments, the nucleic acid molecule comprises a polynucleotide sequence that encodes an LCVR comprising the LCDR1, LCDR2, and LCDR3 of SEQ ID NOs: 50, 51, and 52, respectively. In some embodiments, the nucleic acid molecule comprises a polynucleotide sequence that encodes an LCVR comprising or consisting of the sequence of SEQ ID NO: 45.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds CD20, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds CD20, a second nucleic acid molecule comprising a polynucleotide sequence encoding an LCVR of a first antigen-binding domain that binds CD20, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-CD20 HCVR comprises the HCDR1, HCDR2, and HCDR3 of SEQ ID NOs: 47, 48, and 49, respectively. In some embodiments, an anti-CD20 LCVR comprises LCDR1, LCDR2, and LCDR3 of SEQ ID NOs: 50, 51, and 52, respectively. In some embodiments, an anti-CD3 HCVR comprises the HCDR1, HCDR2, and HCDR3 of SEQ ID NOS: 53, 54, and 55, respectively. In some embodiments, an anti-CD3 LCVR comprises the LCDR1, LCDR2, and LCDR3 of SEQ ID NOs: 50, 51, and 52, respectively.

In one embodiment, the present disclosure provides a nucleic acid molecule or nucleic acid molecules that comprise a nucleotide sequence encoding the HCVR sequence of the anti-CD20 antigen-binding domain comprising SEQ ID NO: 44, a nucleotide sequence encoding the HCVR sequence of the anti-CD3 antigen-binding domain comprising SEQ ID NO: 46, and a nucleotide sequence encoding the LCVR sequence comprising SEQ ID NO: 45.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

### Characterization of BCMA×CD3 Bispecific Antigen-Binding Molecules

The present disclosure includes bispecific antigen-binding molecules (e.g., bispecific antibodies) and functional fragments thereof that bind to BCMA and CD3 (e.g., human BCMA and CD3) with high affinity.

In some embodiments, the present disclosure includes bispecific antigen-binding molecules (e.g., bispecific antibodies as disclosed herein) that bind BCMA and CD3 (e.g., at 25° C. or 37° C.) with a K_{D} of less than about 75 nM, e.g., as measured by surface plasmon resonance or a substantially similar assay. In certain embodiments, the antigen-binding molecules of the present disclosure bind human BCMA and CD3 with a K_{D} of less than about 75 nM, less than about 70 nM, less than about 60 nM, less than about 50 nM, less than about 40 nM, less than about 30 nM, less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured by surface plasmon resonance or a substantially similar assay.

In some embodiments, the present disclosure includes bispecific antigen-binding molecules (e.g., bispecific antibodies as disclosed herein) that specifically interact (e.g., bind with) cells that express BCMA and/or CD3. The extent to which an antigen-binding molecule binds cells that express BCMA and/or CD3 can be assessed by flow cytometry. For example, in some embodiments, the present disclosure provides anti-BCMA×CD3 bispecific antibodies that specifically bind cells that express BCMA and/or CD3 on the cell surface (e.g., human plasma cells and/or T cells). In some embodiments, the disclosure provides anti-BCMA×CD3 bispecific antibodies that bind BCMA and/or CD3-expressing cells or cell lines with an EC₅₀ value of about 10 nM or less, e.g., from about 0.5 nM to about 10 nM, e.g., an EC₅₀ value of about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM, e.g., as determined by flow cytometry or a substantially similar assay.

### Characterization of CD20×CD3 Bispecific Antigen-Binding Molecules

The present disclosure includes bispecific antigen-binding molecules (e.g., bispecific antibodies) and functional fragments thereof that bind to CD20 and CD3 (e.g., human CD20 and CD3) with high affinity.

In some embodiments, the present disclosure includes bispecific antigen-binding molecules (e.g., bispecific antibodies as disclosed herein) that specifically interact (e.g., bind with) cells that express CD20 and/or CD3. The extent to which an antigen-binding molecule binds cells that express CD20 and/or CD3 can be assessed by an in vitro binding assay. For example, in some embodiments, the present disclosure provides anti-CD20×CD3 bispecific antibodies that specifically bind cells that express CD20 and/or CD3 on the cell surface (e.g., human B cells and/or T cells). In certain embodiments, the anti-CD20×CD3 bispecific antibodies bind Jurkat cells and Raji cells with an EC₅₀ value of less than about 60 nM, as measured by an in vitro binding assay. In certain embodiments, the anti-CD20×CD3 bispecific antibodies bind CD3 or CD20 on the surface of a Jurkat or Raji cell, respectively, with an EC₅₀ value of less than about 1000 mM, less than about 500 nM, less than about 200 nM, less than about 100 nM, less than about 75 nM, less than about 70 nM, less than about 65 nM, less than about 60 nM, less than about 50 nM, less than about 40 nM, less than about 30 nM, less than about 25 nM, less than about 10 nM, less than about 5 nM, less than about 2 nM, less than about 1 nM, less than about 500 pM, less than about 100 pM, less than about 10 pM, or less than about 1 pM as measured by an in vitro binding assay.

### Epitope Mapping and Related Technologies

In some embodiments, the epitope on BCMA and/or CD20 and/or CD3 to which the antigen-binding molecules of the present disclosure bind (e.g., an epitope of BCMA or CD20 to which a first antigen-binding domain (D1) binds, or an epitope of CD3 to which a second antigen-binding domain (D2) binds) may consist of a single contiguous sequence of 3 or more (e.g., 3, 4, 5, 6, 7, 8, 9, 10, 11, 12, 13, 14, 15, 16, 17, 18, 19, 20 or more) amino acids of a BCMA or CD20 or CD3 protein. Alternatively, the epitope may consist of a plurality of non-contiguous amino acids (or amino acid sequences) of a BCMA or CD20 or CD3 protein. The antibodies of the invention may interact with amino acids contained within a single CD3 chain (e.g., CD3-epsilon, CD3-delta or CD3-gamma), or may interact with amino acids on two or more different CD3 chains. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antibody interacts.

The present disclosure also includes antigen-binding molecules (e.g., antibodies or antigen-binding domains thereof) that bind to the same epitope as, or competes for binding with, a bispecific BCMA×CD3 antigen-binding molecule or a bispecific CD20×CD3 antigen-binding molecule described herein. One skilled in the art can determine whether or not a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on BCMA and/or CD20 and/or CD3 as a reference bispecific antigen-binding molecule of the present disclosure, the reference bispecific molecule is first allowed to bind to a BCMA and/or CD20 and/or CD3 protein. Next, the ability of a test antibody to bind to the BCMA and/or CD20 and/or CD3 molecule is assessed. If the test antibody is able to bind to BCMA and/or CD20 and/or CD3 following saturation binding with the reference bispecific antigen-binding molecule, it can be concluded that the test antibody binds to a different epitope of BCMA and/or CD20 and/or CD3 than the reference bispecific antigen-binding molecule. On the other hand, if the test antibody is not able to bind to the BCMA and/or CD20 and/or CD3 molecule following saturation binding with the reference bispecific antigen-binding molecule, then the test antibody may bind to the same epitope of BCMA and/or CD20 and/or CD3 as the epitope bound by the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, radioimmunoassay (RIA), Biacore, flow cytometry or any other quantitative or qualitative antibody-binding assay available in the art. In accordance with certain embodiments of the present disclosure, two antigen-binding proteins bind to the same (or overlapping) epitope if, e.g., a 1-, 2-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a BCMA and/or CD20 and/or CD3 protein under saturating conditions followed by assessment of binding of the test antibody to the BCMA and/or CD20 and/or CD3 molecule. In a second orientation, the test antibody is allowed to bind to a BCMA and/or CD20 and/or CD3 molecule under saturating conditions followed by assessment of binding of the reference antigen-binding molecule to the BCMA and/or CD20 and/or CD3 molecule. If, in both orientations, only the first (saturating) antigen-binding molecule is capable of binding to the BCMA and/or CD20 and/or CD3 molecule, then it is concluded that the test antibody and the reference antigen-binding molecule compete for binding to BCMA and/or CD20 and/or CD3. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Multispecific Antigen-Binding Molecules

Antigen-binding domains specific for particular antigens can be prepared by any antibody generating technology known in the art. Once obtained, two different antigen-binding domains can be appropriately arranged relative to one another to produce a bispecific antigen-binding molecule of the present disclosure using routine methods. A discussion of exemplary bispecific antibody formats that can be used to construct the bispecific antigen-binding molecules of the present disclosure is provided elsewhere herein. In certain embodiments, one or more of the individual components (e.g., heavy, and light chains) of the multispecific antigen-binding molecules are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., BCMA or CD20 or CD3) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules.

In some embodiments, genetically engineered animals may be used to make human bispecific antigen binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454, the entire contents of which are incorporated herein by reference, for a detailed discussion of such engineered mice and the use thereof to produce bispecific antigen-binding molecules). As used herein, “fully human” refers to an antigen-binding molecule, e.g., an antibody, or antigen-binding fragment or immunoglobulin domain thereof, comprising an amino acid sequence encoded by a DNA derived from a human sequence over the entire length of each polypeptide of the antigen-binding molecule, antibody, antigen-binding fragment, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### Bioequivalents

The present disclosure encompasses antigen-binding molecules having amino acid sequences that vary from those of the described antibodies but that retain the ability to bind BCMA and/or CD20 and/or CD3. Such variant molecules comprise one or more additions, deletions, or substitutions of amino acids when compared to the parent sequence, but exhibit biological activity that is essentially equivalent to that of the described antigen-binding molecules. Likewise, the nucleic acid sequences encoding the antigen-binding molecules of the present disclosure encompass sequences that comprise one or more additions, deletions, or substitutions of nucleotides when compared to the disclosed sequence, but that encode an antigen binding molecule that is essentially bioequivalent to the antigen-binding molecules disclosed herein.

The present disclosure includes antigen-binding molecules that are bioequivalent to any of the exemplary antigen-binding molecules set forth herein. Two antigen-binding proteins, e.g., bispecific antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the first antigen-binding protein (e.g., reference product) and the second antigen-binding protein (e.g., biological product) without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other embodiments, bioequivalent antibodies may include the exemplary bispecific antigen-binding molecules set forth herein comprising amino acid changes which modify the glycosylation characteristics of the antibodies, e.g., mutations which eliminate or remove glycosylation.

### Immunoglobulin Depleting Agents

In various aspects, the present disclosure provides immunoglobulin depleting agents, e.g., which may be combined with or administered in combination with plasma cell depleting agents (e.g., an anti-BCMA×CD3 bispecific antibody, or a functional fragment thereof) or B cell depleting agents (e.g., an anti-CD20×CD3 bispecific antibody, or a functional fragment thereof) described herein. In some embodiments, the immunoglobulin depleting agent may be administered in combination with a plasma cell depleting agent, a B cell depleting agent, plasmapheresis, therapeutic plasma exchange, immunoadsorption, and/or an immunogen (e.g., an immunogenic delivery vehicle such as, e.g., AAV) disclosed herein. Suitable combinations comprising a plasma cell depleting agent are described in more detail elsewhere herein. In some embodiments, an immunoglobulin depleting agent may be useful for, e.g., accelerating IgG clearance. In some embodiments, an immunoglobulin depleting agent is capable of accelerating IgG serum clearance.

In some embodiments, an immunoglobulin depleting agent may comprise a neonatal Fc receptor (FcRn) blocker such as, but not limited to, efgartigimod alfa. The mechanistic concept of FcRn-targeting therapeutics is to accelerate IgG catabolism by blocking the FcRn-mediated intracellular IgG recycling pathway, thereby reducing overall plasma IgG levels. FcRn can participate in the maintenance of IgG levels by salvaging IgG from lysosomal degradation, thereby prolonging the half-life of IgG. In some embodiments, FcRn blockers can compete with IgG for binding to FcRn. Due to their higher affinity for FcRn, FcRn blockers can prevent IgG from binding to FcRn and, instead, IgG is transported to the lysosome and degraded, thereby leading to decreased circulating levels of IgG.

In some embodiments, an FcRn blocker can include Efgartigimod (ARGX-113), Rozanolixizumab (UCB7665), Batoclimab (RVT-1401), Nipocalimab (M281), Orilanolimab (SYNT001), IMVT-1402, or any combination thereof. See, e.g., Zuercher et al. (2019) Autoimmun. Rev. 18(10):102366, which is incorporated herein by reference in its entirety.

In some embodiments, an immunoglobulin depleting agent may comprise an IgG degrading enzyme such as IdeS (imlifidase), IdeZ, or IdeXork. IdeS (imlifidase) is an endopeptidase derived from Streptococcus pyogenes which has specificity for human IgG, and when infused intravenously results in rapid cleavage of IgG. IdeZ (immunoglobulin-degrading enzyme from Streptococcus equi subspecies zooepidemicus) is an engineered recombinant protease overexpressed in Escherichia coli. IdeZ specifically cleaves IgG molecules below the hinge region to yield F(ab′)2 and Fc fragments. IdeXork (Xork) is yet another example of an IgG protease. More particular non-limiting examples of IgG degrading enzymes include Imlifidase/IdeS/Fabricator, IdeZ, IceM, IceMG, CYR-212, CYR-241, S-1117, HNSA-5487, and Xork. In some embodiments, an immunoglobulin depleting agent may facilitate IgG degradation via lysosomal destruction. A non-limiting example of an immunoglobulin depleting agent which may facilitate IgG degradation via lysosomal destruction is BHV-1300.

### Plasmapheresis, Therapeutic Plasma Exchange, and Immunoadsorption

In various aspects, the methods disclosed herein can include plasmapheresis, therapeutic plasma exchange, or immunoadsorption. These can be combined, for example, with treatment with plasma cell depleting agents (e.g., an anti-BCMA×CD3 bispecific antibody, or a functional fragment thereof), B cell depleting agents (e.g., an anti-CD20×CD3 bispecific antibody, or a functional fragment thereof), and/or immunoglobulin depleting agents described herein. In some embodiments, the plasmapheresis, therapeutic plasma exchange, or immunoadsorption may be performed in combination with treatment with a plasma cell depleting agent, a B cell depleting agent, and/or an immunogen (e.g., an immunogenic delivery vehicle) disclosed herein. Suitable combinations comprising a plasma cell depleting agent are described in more detail elsewhere herein. Plasmapheresis, therapeutic plasma exchange, and immunoadsorption may be useful strategies for removal of AAV antibodies from patients' blood plasma.

Plasmapheresis is a process used to selectively remove blood components used to treat a variety of conditions including those caused by the acute overproduction of antibodies (e.g., autoimmunity, transplant rejection), in which removal of pathogenic immunoglobulins results in clinical benefit. Immunoadsorption is a selective therapeutic apheresis technique by which immunoglobulins are selectively removed from patients' plasma. The immunoadsorption can be, for example, total immunoglobulin immunoadsorption. See, e.g., Boedecker-Lips et al. (2023) J. Clin. Apher. 38(5):590-601. Alternatively, the immunoadsorption can be AAV capsid specific immunoadsorption. See, e.g., Bertin et al. (2020) Sci. Rep. 10:864.

### Combinations Comprising a Plasma Cell Depleting Agent

A plasma cell depleting agent (e.g., a BCMA×CD3 antigen-binding molecule) can be administered to a subject in need thereof either alone, or in combination with, a B cell depleting agent (e.g., a CD20×CD3 antigen-binding molecule), an immunoglobulin depleting agent (e.g., an FcRn blocker, such as Efgartigimod), and/or an immunogen. In some embodiments, the administration of the plasma cell depleting, the B cell depleting agent, the immunoglobulin depleting agent, and/or the immunogen can be further combined with plasmapheresis, therapeutic plasma exchange, and/or immunoadsorption. As used herein, the term “in combination with”, e.g., a BCMA×CD3 bispecific antigen-binding molecule (or other immunomodulator or immunogen, etc.) means that additional component(s) may be administered prior to, concurrent with, or after the administration of BCMA×CD3 bispecific antigen-binding molecule (or other immunomodulator or immunogen, etc.) molecule (or other immunomodulator or immunogen, etc.). The different components of the combination can be formulated into a single composition, e.g., for simultaneous delivery, or formulated separately into two or more compositions (e.g., a kit including each component, for example, wherein the further agent is in a separate formulation).

For example, a plasma cell depleting agent (e.g., a BCMA×CD3 antigen-binding molecule) can be administered to a subject in need thereof either alone, or in combination with, a B cell depleting agent (e.g., a CD20×CD3 antigen-binding molecule) and/or an immunoglobulin depleting agent (e.g., an FcRn blocker, such as Efgartigimod). In some embodiments, the B cell depleting agent is administered before, at the same time as, or after the plasma cell depleting agent. In some embodiments, the immunoglobulin depleting agent is administered after the plasma cell depleting agent.

In one example, a plasma cell depleting agent (e.g., a BCMA×CD3 antigen-binding molecule) is administered to a subject in need thereof in combination with a B cell depleting agent (e.g., a CD20×CD3 antigen-binding molecule).

In another example, a plasma cell depleting agent (e.g., a BCMA×CD3 antigen-binding molecule) is administered to a subject in need thereof in combination with an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the combination of the plasma cell depleting agent and the immunoglobulin depleting agent, when administered in further combination with an immunogen (e.g., an immunogenic delivery vehicle such as, e.g., AAV) to a subject in need thereof, decreases a level of an anti-immunogen antibody titer (e.g., an anti-AAV antibody titer) in the subject (e.g., such as can be measured in a serum sample isolated from the subject). In some embodiments, the level of the anti-immunogen antibody titer is decreased by about 1-fold to about 20-fold, about 2-fold to about 15-fold, about 4-fold to about 10-fold, about 3-fold to about 18-fold, about 5-fold to about 12-fold, or about 6-fold to about 8-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold, or more. In some embodiments, the anti-immunogen antibody titer is decreased by about 20-fold.

In another example, a plasma cell depleting agent (e.g., a BCMA×CD3 antigen-binding molecule) is administered to a subject in need thereof in combination with a B cell depleting agent (e.g., a CD20×CD3 antigen-binding molecule) and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the combination of the plasma cell-depleting agent, the B cell depleting agent, and the immunoglobulin-depleting agent, when administered in further combination with an immunogen (e.g., an immunogenic delivery vehicle such as, e.g., AAV) to a subject in need thereof, decreases the level of an anti-immunogen antibody titer (e.g., an anti-AAV antibody titer) in the subject (e.g., such as can be measured in a serum sample isolated from the subject). In some embodiments, the level of the anti-immunogen antibody titer may be decreased by about 1-fold to about 20-fold, about 2-fold to about 15-fold, about 4-fold to about 10-fold, about 3-fold to about 18-fold, about 5-fold to about 12-fold, about 6-fold to about 8-fold, about 10-fold to about 30-fold, about 20-fold to about 50-fold, about 30-fold to about 70-fold, about 40-fold to about 90-fold, or about 50-fold to about 100-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold, or more. In some embodiments, the anti-immunogen antibody titer is decreased by about 100-fold.

In another example, a plasma cell depleting agent (e.g., a BCMA×CD3 antigen-binding molecule) is administered to a subject in need thereof in combination with plasmapheresis, therapeutic plasma exchange, or immunoadsorption.

In another example, a plasma cell depleting agent (e.g., a BCMA×CD3 antigen-binding molecule) is administered to a subject in need thereof in combination with plasmapheresis, therapeutic plasma exchange, or immunoadsorption and a B cell depleting agent (e.g., a CD20×CD3 antigen-binding molecule).

In another example, a plasma cell depleting agent (e.g., a BCMA×CD3 antigen-binding molecule) is administered to a subject in need thereof in combination with plasmapheresis, therapeutic plasma exchange, or immunoadsorption and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In another example, a plasma cell depleting agent (e.g., a BCMA×CD3 antigen-binding molecule) is administered to a subject in need thereof in combination with plasmapheresis, therapeutic plasma exchange, or immunoadsorption, a B cell depleting agent (e.g., a CD20×CD3 antigen-binding molecule), and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the B cell depleting agent comprises two or more B cell depleting agents (e.g., an anti-CD19 antigen-binding molecule and an anti-CD20 antigen-binding molecule). In some embodiments, the immunoglobulin depleting agent comprises two or more immunoglobulin depleting agents (e.g., an FcRn blocker and an IgG degrading enzyme).

In embodiments in which a plasma cell depleting agent (e.g., a BCMA×CD3 antigen-binding molecule) is administered to a subject in combination with, a B cell depleting agent (e.g., a CD20×CD3 antigen-binding molecule) and/or an immunoglobulin depleting agent (e.g., an FcRn blocker, such as Efgartigimod) and/or plasmapheresis, therapeutic plasma exchange, or immunoadsorption, one or more or all treatments can occur together or one or more or all treatments can occur sequentially. For example, in some embodiments in which the plasma cell depleting agent (e.g., a BCMA×CD3 antigen-binding molecule) is administered to a subject in need thereof in combination with an IgG degrading enzyme, the plasma cell depleting agent can be administered to the subject first, followed by the IgG degrading enzyme. In another example, in embodiments where an immunoglobulin depleting agent (e.g., FcRn blocker) is administered together with plasmapheresis, therapeutic plasma exchange, or immunoadsorption, the plasmapheresis, therapeutic plasma exchange, or immunoadsorption can be first followed by administration of the immunoglobulin depleting agent (e.g., FcRn blocker).

### Pharmaceutical Compositions

In another aspect, the present disclosure provides pharmaceutical compositions comprising plasma cell depleting agents (e.g., long-lived plasma cell (LLPC) depleting agents such as anti-BCMA×CD3 bispecific antibodies, or functional fragments thereof), B cell depleting agents (e.g., anti-CD19 and anti-CD20 antibodies, or a CD20×CD3 antigen-binding molecule (e.g., REGN1979), or functional fragments thereof), immunoglobulin depleting agents (e.g., neonatal Fc receptor (FcRn) blockers), and/or immunogens (e.g., immunogenic delivery vehicles) disclosed herein, optionally comprising a pharmaceutically acceptable carrier and/or excipient. In one specific embodiment, a composition described herein comprises an immunogen and an anti-CD20×CD3 bispecific antibody, or a functional fragment thereof, and optionally, further comprises a pharmaceutically acceptable carrier and/or excipient. Suitable combinations comprising a plasma cell depleting agent are described in more detail elsewhere herein.

The pharmaceutical compositions are formulated with one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA.

One exemplary embodiment of the present disclosure comprises a pharmaceutical composition comprising (i) a plasma cell depleting agent, (ii) a B cell depleting agent and/or an immunoglobulin depleting agent, and (iii) a pharmaceutically acceptable carrier and/or excipient. Another exemplary embodiment of the present disclosure comprises a pharmaceutical composition comprising (i) an immunogen, (ii) a plasma cell depleting agent, (iii) optionally, a B cell depleting agent and/or an immunoglobulin depleting agent, and (iv) a pharmaceutically acceptable carrier and/or excipient

In some embodiments, the plasma cell depleting agent comprises an antigen-binding molecule that specifically binds B cell maturation antigen (BCMA) and CD3. In some embodiments, the plasma cell depleting agent comprises an anti-BCMA×CD3 bispecific antibody, or functional fragment thereof, disclosed herein. Non-limiting examples of an anti-BCMA×CD3 bispecific antibody include linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B.

In some embodiments, the anti-BCMA×CD3 bispecific antibody comprises:
(a) a first antigen-binding domain (D1) that binds an epitope of human BCMA; and
(b) a second antigen-binding domain (D2) that binds an epitope of human CD3.

In some embodiments, the B cell depleting agent comprises anti-CD19 and anti-CD20 antibodies, or functional fragments thereof, disclosed herein. In some embodiments, the B cell depleting agent comprises a CD20×CD3 antigen-binding molecule (e.g., REGN1979).

In some embodiments, the immunoglobulin depleting agent comprises a neonatal Fc receptor (FcRn) blocker. A non-limiting example of an FcRn blocker is efgartigimod alfa. In some embodiments, the immunoglobulin depleting agent comprises an IgG degrading enzyme.

In some embodiments, the immunogen is an immunogenic delivery vehicle, a polypeptide, a polynucleotide, a glycan, or a lipid. In some embodiments, the immunogen is an immunogenic delivery vehicle or a polypeptide or polynucleotide encoded by a transgene contained within the immunogenic delivery vehicle. In some embodiments, the immunogen is an immunogenic delivery vehicle and/or transgene product(s).

In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell.

In some embodiments, the immunogenic delivery vehicle is a viral vector.

In some embodiments, the viral vector is derived from an adeno-associated virus (AAV), an adenovirus, a retrovirus, or an oncolytic virus.

In some embodiments, the viral vector is AAV. In some embodiments, the viral vector is derived from AAV.

In some embodiments, the retrovirus is a lentivirus.

In some embodiments, the oncolytic virus is an adenovirus, a rhabdovirus, a herpes virus, a measles virus, a coxsackievirus, a poliovirus, a reovirus, a poxvirus, a parvovirus, Maraba virus, or Newcastle disease virus.

In some embodiments, the carrier is suitable for intravenous, intramuscular, oral, intraperitoneal, intratumoral, intrathecal, transdermal, topical, or subcutaneous administration.

In some embodiments, the pharmaceutical composition comprises an injectable preparation, such as a dosage form for intravenous, subcutaneous, intracutaneous, and intramuscular injections, drip infusions, etc. These injectable preparations may be prepared by known methods. For example, the injectable preparations may be prepared, e.g., by dissolving, suspending, or emulsifying the antibody or its salt described above, in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil), etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of a plasma cell depleting agent, a B cell depleting agent, an immunoglobulin depleting agent, and/or an immunogen (e.g., immunogenic delivery vehicle) administered to a patient according to the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions as disclosed herein may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

In some embodiments, e.g., for methods and compositions of the present disclosure involving administration of a plasma cell depleting agent which is a bispecific BCMA×CD3 antibody (e.g., REGN5458) to a subject, the dose of the bispecific BCMA×CD3 antibody (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, the bispecific BCMA×CD3 antibody (e.g., REGN5458) can be administered to the subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the bispecific BCMA×CD3 antibody (e.g., REGN5458) (or pharmaceutical composition thereof) dose is about 20 mg/kg.

In some embodiments, e.g., for methods and compositions of the present disclosure involving administration of a B cell depleting agent which is a bispecific CD20×CD3 antibody (e.g., REGN1979) to a subject, the dose of the bispecific CD20×CD3 antibody (or pharmaceutical compositions thereof) is from about 0.05 mg/kg to about 3 mg/kg, such as from about 0.05 mg/kg to about 0.1 mg/kg, about 0.1 mg/kg to about 0.5 mg/kg, about 0.5 mg/kg to about 1 mg/kg, about 1 mg/kg to about 1.5 mg/kg, about 1.5 mg/kg to about 2 mg/kg, about 2 mg/kg to about 2.5 mg/kg, or about 2.5 mg/kg to about 3 mg/kg. In one specific embodiment, the bispecific CD20×CD3 antibody (e.g., REGN1979) is administered to the subject at a dose of about 0.1 mg/kg. In another specific embodiment, the bispecific CD20×CD3 antibody (e.g., REGN1979) is administered to the subject at a dose of about 1 mg/kg.

In some embodiments, e.g., for methods and compositions of the present disclosure involving administration of an immunoglobulin depleting agent which is efgartigimod to a subject, the dose of efgartigimod (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, efgartigimod can be administered to the subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the efgartigimod (or pharmaceutical composition thereof) dose is about 20 mg/kg.

In some embodiments, e.g., for methods and compositions of the present disclosure involving administration of an immunogen which is an AAV to a subject, the dose of the AAV (or pharmaceutical compositions thereof) administered to a subject is between about 1×10⁵ plaque forming units (pfu) to about 1×10¹⁵ pfu. In some cases, the AAV can be administered to the subject at a dose from about 1×10⁸ pfu to about 1×10¹⁵ pfu, or from about 1×10¹⁰ pfu to about 1×10¹⁵ pfu, or from about 1×10⁸ pfu to about 1×10¹² pfu.

In some embodiments, the dose of the AAV (or pharmaceutical compositions thereof) administered to the subject is between about 1×10⁵ vg to about 1×10¹⁶ vg. In certain embodiments, the dose of the AAV administered to the subject is between about 1×10⁶ vg to about 1×10⁹ vg, about 1×10⁷ vg to about 1×10¹⁰ vg, about 1×10⁸ vg to about 1×10¹¹ vg, about 1×10⁹ vg to about 1×10¹² vg, about 1×10¹⁰ vg to about 1×10¹³ vg, about 1×10¹¹ vg to about 1×10¹⁴ vg, about 1×10¹² vg to about 1×10¹⁵ vg, about 1×10¹³ vg to about 1×10¹⁶ vg, or about 1×10¹⁴ vg to about 1×10¹⁶ vg. In certain embodiments, the dose of the AAV administered to the subject is between about 1×10¹⁰ vg to about 1×10¹⁶ vg. In certain embodiments, the dose of the AAV administered to the subject is at least about 1×10⁶ vg, at least about 1×10⁷ vg, at least about 1×10⁸ vg, at least about 1×10⁹ vg, at least about 1×10¹⁰ vg, at least about 1×10¹¹ vg, at least about 1×10¹² vg, at least about 1×10¹² vg, at least about 1×10¹³ vg, at least about 1×10¹⁴ vg, or at least about 1×10¹⁵ vg. In certain embodiments, the vg is total vector genome per subject.

In some embodiments, the dose of the AAV (or pharmaceutical compositions thereof) administered to the subject is about 1×10¹², 1×10¹³, 1×10¹⁴, 1×10¹⁵, and 1×10¹⁶ vector genomes (vg)/mL. Further examples of doses of AAV include about 1×10¹², about 1×10¹³, about 1×10¹⁴, about 1×10¹⁵, and about 1×10¹⁶ vector genomes (vg)/mL, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/mL.

Other examples of doses of AAV (or pharmaceutical compositions thereof) include about 1×10¹², about 1×10¹³, about 1×10¹⁴, about 1×10¹⁵, and about 1×10¹⁶ vector genomes (vg)/kg of body weight, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/kg of body weight.

In one example, the AAV dose (or pharmaceutical compositions thereof) is between about 1×10¹³ to about 1×10¹⁴ vg/mL or vg/kg. In another example, the AAV dose is between about 1×10¹² to about 1×10¹³ vg/mL or vg/kg (e.g., between about 1×10¹² to about 1×10¹³ vg/kg). In another example, the AAV dose is between about 1×10¹² to about 1×10¹⁴ vg/mL or vg/kg (e.g., between about 1×10¹² to about 1×10¹⁴ vg/kg).

In one specific embodiment, the AAV dose (or pharmaceutical composition thereof) is about 3×10¹¹ vg/kg. In one specific embodiment, the AAV dose (or pharmaceutical composition thereof) is about 6×10¹¹ vg/kg. In another specific embodiment, the AAV dose (or pharmaceutical composition thereof) is about 9×10¹¹ vg/kg. In another specific embodiment, the AAV dose (or pharmaceutical composition thereof) is about 3×10¹² vg/kg. In one specific embodiment, the AAV dose (or pharmaceutical composition thereof) is about 1×10¹³ vg/kg. In another specific embodiment, the AAV dose (or pharmaceutical composition thereof) is about 6×10¹³ vg/kg.

Various delivery systems are known and can be used to administer the pharmaceutical composition, e.g., encapsulation in liposomes, microparticles, microcapsules, recombinant cells capable of expressing, e.g., recombinant viruses comprising any components of the compositions disclosed herein, and a soluble carrier system that takes advantage of receptor mediated endocytosis (see, e.g., Wu et al., 1987, J. Biol. Chem. 262:4429-4432). Methods of administration include, but are not limited to, intradermal, intramuscular, intraperitoneal, intratumoral, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously. In some embodiments, a pharmaceutical composition as disclosed herein is administered intratumorally.

In some embodiments, a plasma cell depleting agent, a B cell depleting agent, an immunoglobulin depleting agent, and/or an immunogen, or a pharmaceutical composition(s) thereof, is contained within a container. Thus, in another aspect, containers comprising an antigen-binding molecule and/or pharmaceutical composition as disclosed herein are provided. For example, in some embodiments, an antibody and/or pharmaceutical composition is contained within a container selected from the group consisting of a glass vial, a syringe, a pen delivery device, and an autoinjector.

In some embodiments, a plasma cell depleting agent, a B cell depleting agent, an immunoglobulin depleting agent, and/or an immunogen, or a pharmaceutical composition(s) thereof, of the present disclosure is delivered, e.g., subcutaneously or intravenously, such as with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to, AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen delivery devices having applications, e.g., in subcutaneous delivery of a pharmaceutical composition of the present invention include, but are not limited to, the SOLOSTAR™ pen (sanofi-aventis), the FLEXPEN™ (Novo Nordisk), the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.).

In some embodiments, the pharmaceutical compositions of the present disclosure can be delivered using a controlled release system. In one embodiment, a pump may be used (see, e.g., Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions as described herein are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antigen-binding molecule contained in the dosage form is about 5 to about 1000 mg, e.g., from about 5 to about 500 mg, from about 5 to about 100 mg, or from about 10 to about 250 mg.

Plasma cell depleting agents, B cell depleting agents, immunoglobulin depleting agents, and/or immunogens, introduced into the subject or cell can be provided in compositions comprising a carrier, thereby increasing the stability of the introduced molecules, e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo. Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of a molecule (e.g., a nucleic acid or protein) into a cell or subject. Methods for introducing molecules into various cell types are known and include, for example, stable transfection methods, transient transfection methods, and virus-mediated methods.

Transfection protocols, as well as protocols for introducing molecules into cells, may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection can include the use of a gene gun or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm, but also through the nuclear membrane and into the nucleus. In addition, use of nucleofection in the methods disclosed herein typically requires much fewer cells than regular electroporation (e.g., only about 2 million cells as compared with 7 million cells by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids or proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors which can be useful in accomplishing virus-mediated delivery include retroviruses, adenoviruses, vaccinia viruses, poxviruses, and herpes simplex viruses. The viruses can infect dividing cells, non-dividing cells, or both dividing and non-dividing cells. The viruses can integrate into the host genome or, alternatively, do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³ between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg).

In yet another aspect, the present disclosure includes compositions and therapeutic formulations comprising any of the plasma cell depleting agents, B cell depleting agents, immunoglobulin depleting agents, and/or immunogens, described herein in combination with one or more additional therapeutic agents, and methods of treatment comprising administering such combinations to subjects in need thereof. In some embodiments, the additional therapeutic agent(s) is an immunomodulatory agent or anti-inflammatory agent. In some embodiments, the additional therapeutic agent(s) is immunosuppressive therapy. In some embodiments, the additional therapeutic agent(s) is a surgical procedure.

Exemplary additional therapeutic agents that may be combined with or administered in combination with any of the plasma cell depleting agents, B cell depleting agents, immunoglobulin depleting agents, and/or immunogens, of the present disclosure include, e.g., an anti-CD38 antibody (e.g., daratumumab), a proteasome inhibitor, a histone deacetylase inhibitor, a B-cell activating factor (BAFF) inhibitor, an APRIL inhibitor, a steroid (e.g., corticosteroids such as topical, systemic, oral, or inhaled corticosteroids including, but not limited to, betamethasone, clobetasol, dexamethasone, fluocinolone, fluocinonide, halobetasol, hydrocortisone, methylprednisolone, prednisone, prednisolone, or triamcinolone); a non-steroidal topical medication such as, but not limited to, a phosphodiesterase 4 (PDE4) inhibitor or a calcineurin inhibitor; a non-steroidal anti-inflammatory drug (NSAID) such as, but not limited to, celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulfasalazinem, sulindac, or tolmetin; an anti-inflammatory antibody or biologic (e.g., an anti-tumor necrosis factor alpha (TNFα) antibody or biologic such as, but not limited to, adalimumab, certolizumab, etanercept, golimumab, or infliximab; an anti-IL1 antibody or biologic such as, but not limited to, LY2189102, anakinra, canakinumab, gerokizumab, or rilonacept; an anti-interleukin 6 (IL6)/IL-6 receptor (R) antibody or biologic such as, but not limited to, sarilumab, siltuximab, or tocilizumab; an anti-IL17A/IL-17R antibody or biologic such as, but not limited to, bimekizumab, brodalumab, ixekizumab, or secukinumab; or an anti-IL12/IL-23 antibody or biologic such as, but not limited to, AMG139, BI655066, brazikumab, briankizumab, guselkumab, mirikizumab, risankizumab, tildrakizumab, or ustekinumab); a JAK inhibitor such as, but not limited to, abrocitinib, baricitinib, fedratinib, filgotinib, ruxolitinib, tofacitinib, or upadacitinib; an immunosuppressive agent (e.g., a systemic immunosuppressant such as, but not limited to, methotrexate, cyclophosphamide, mizoribine, chlorambucil, cyclosporine, mycophenolate mofetil, or azathioprine); a disease-modifying antirheumatic drug (DMARD) such as, but not limited to, apremilast, azathioprine, baricitinib, cyclophosphamide, cyclosporine, hydroxychloroquine, leflunomide, methotrexate, mycophenolate mofetil, sulfasalazine, or tofacitinib; radiation therapy; chemotherapy; intravenous immunoglobulin therapy; or a surgery or a surgical procedure (such as, but not limited to, splenectomy, lymphadenectomy, thyroidectomy, plasmapheresis, leukapheresis, therapeutic plasma exchange, immunoadsorption, or cell, tissue, or organ transplantation). In some embodiments, the surgery or surgical procedure as described herein is used in combination with the anti-BCMA×CD3 bispecific antibody or the anti-CD20×CD3 bispecific antibody and in place of the FcRn blocker.

In some embodiments, a plasma cell depleting agent, a B cell depleting agent, an immunoglobulin depleting agent, and/or an immunogen described herein may be administered with an additional therapeutic agent comprising, e.g., a broad-spectrum immunosuppression methodology, or combination thereof, including broad spectrum immunosuppression, e.g., calcineurin inhibitors (tacrolimus, cyclosporine), rapamycin, MMF, corticosteroids, methotrexate, proteasome inhibitors, costimulation blockade (CTLA4-Ig/abatacept/belatacept), Src kinase inhibitors (dasatinib), Btk inhibitors (acalabrutinib), B cell depleting agents (rituximab), IgG degrading enzymes (IdeS), IgG half-life reducers (FcRn blockers), or combinations thereof.

The additional therapeutically active component(s) may be administered just prior to, concurrent with, or shortly after the administration of the plasma cell depleting agent, the B cell depleting agent, the immunoglobulin depleting agent, and/or the immunogen, or the pharmaceutical composition(s) thereof, of the present disclosure. Such administration regimens can be considered, for example, the administration of a plasma cell depleting agent, a B cell depleting agent, an immunoglobulin depleting agent, and/or an immunogen, or a pharmaceutical composition(s) thereof, “in combination with” an additional therapeutically active component.

The present disclosure includes pharmaceutical compositions in which a plasma cell depleting agent, a B cell depleting agent, an immunoglobulin depleting agent, and/or an immunogen (e.g., an immunogenic delivery vehicle) of the present invention is co-formulated with one or more of the additional therapeutically active component(s) as described elsewhere herein.

Therapeutic or pharmaceutical compositions comprising the compositions or combinations disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

### Methods of Use

In various aspects, the present disclosure provides methods for inhibiting or preventing an immune response to an immunogen (e.g., an immunogenic delivery vehicle such as, e.g., AAV) in a subject in need thereof, the methods comprising administering to the subject an effective amount of a plasma cell depleting agent, a B cell depleting agent, and/or an immunoglobulin depleting agent disclosed herein. In some embodiments, the present disclosure provides methods for inhibiting or preventing generation of antibodies (e.g., neutralizing antibodies) to an immunogen in a subject in need thereof, the methods comprising administering to the subject an effective amount of a plasma cell depleting agent, a B cell depleting agent, and/or an immunoglobulin depleting agent. In some embodiments of methods of the disclosure comprising administering to the subject an effective amount of a plasma cell depleting agent and an immunogen (e.g., an immunogenic delivery vehicle such as, e.g., AAV), the subject has a pre-existing immunity against the immunogen (e.g., AAV). In another aspect, provided herein is a method for inhibiting generation of neutralizing antibodies to an immunogen in a subject in need thereof (e.g., a subject without a pre-existing immunity against the immunogen), the method comprising administering to the subject an effective amount of an anti-CD20×CD3 bispecific antibody or functional fragment thereof. In some embodiments, the present disclosure provides methods for increasing effectiveness of re-administration of an immunogen to a subject in need thereof, the methods comprising administering to the subject an effective amount of a plasma cell depleting agent, a B cell depleting agent, and/or an immunoglobulin depleting agent. In another aspect, provided herein is a method for increasing effectiveness of re-administration of an immunogen to a subject in need thereof (e.g., a subject without a pre-existing immunity against the immunogen), the method comprising administering to the subject an effective amount of an anti-CD20×CD3 bispecific antibody or functional fragment thereof. The term “re-administering” is used synonymously and interchangeably with the term “re-dosing” herein. In some embodiments, the present disclosure provides methods for increasing or maintaining the level of a transgene expression in a subject in need thereof, and the transgene is delivered to the subject via an immunogenic delivery vehicle (e.g., an AAV), the methods comprising administering to the subject an effective amount of a plasma cell depleting agent, a B cell depleting agent, and/or an immunoglobulin depleting agent.

In some embodiments, a plasma cell depleting agent, a B cell depleting agent, and/or an immunoglobulin depleting agent which can be useful in any of the methods or compositions disclosed herein may be further used in combination with a plasmapheresis, therapeutic plasma exchange, and/or immunoadsorption.

In some embodiments, administration of a plasma cell depleting agent, a B cell depleting agent, an immunoglobulin depleting agent, and/or an immunogen prevents or delays the increase of disease symptoms or the progression of disease in a subject having disease or condition.

The term “immune response” refers to a response of a cell of the immune system (e.g., a B-cell, T-cell, macrophage or polymorphonucleocyte) to a stimulus such as an immunogen, e.g., an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). An active immune response can be contrasted with passive immunity, which can be acquired through the transfer of substances such as, e.g., an antibody, a transfer factor, a thymic graft, and/or a cytokine from an actively immunized host to a non-immune host.

In some embodiments, the immune response is a humoral (antibody producing) immune response and/or a cell-mediated immune response in a subject (e.g., a human).

In some embodiments, a plasma cell depleting agent, a B cell depleting agent, and/or an immunoglobulin depleting agent may inhibit an immune response by a cell (e.g., an immune cell such as by a B cell or a T cell) or by an immune system of a subject (e.g., a human) which can be elicited by an immunogen.

As used herein, the term “immunogen” refers to any molecule that is capable of eliciting an immune response. Non-limiting examples of immunogens include immunogenic delivery vehicles such as viral vectors also termed herein “viral particles” (e.g., viral vectors derived from adeno-associated viruses (AAV), adenoviruses, retroviruses [e.g., lentiviruses], or oncolytic viruses [e.g., an adenovirus, a rhabdovirus, a herpes virus, a measles virus, a coxsackievirus, a poliovirus, a reovirus, a poxvirus, a parvovirus, Maraba virus, or Newcastle disease virus]) or portions thereof (e.g., capsid proteins), virus-like particles (VLPs), non-viral vectors (e.g., bacteriophages [such as lambda (X) bacteriophage, EMBL bacteriophage; bacterial vectors such as pBs, phagescript, PsiX174, pBluescript SK, pBs KS, pNH8a, pNH16a, pNH18a, pNH46a; pTrc99A, pKK223-3, pKK233-3, pDR540, and pRIT5]; eukaryotic vectors [such as pWLneo, pSV2cat, pOG44, PXR1, pSG, pSVK3, pBPV, pMSG and pSVL]; transposons [such as Sleeping Beauty transposon and PiggyBac transposon]; bacterial vectors, fungal vectors, and protozoal vectors), liposomes, lipid nanoparticles (LNPs), non-lipid nanoparticles, mammalian cells (e.g., allogeneic cells), and other carriers. Non-limiting examples of immunogens also include polypeptide molecules (e.g., proteins [e.g., therapeutic proteins or antibodies or fragments thereof], peptides), polynucleotide molecules (e.g., mRNAs, interfering nucleic acid molecules [RNAi, siRNA, shRNA], miRNAs, antisense oligonucleotides, ribozymes, aptamers, mixmers, or multimers), antigen-binding molecules fused to a payload, as well as naturally occurring or modified bacteria, fungi, protozoa, parasites, helminths, ectoparasites, or other microorganisms (including bacteria, fungi and other microorganisms found in microbiota). Glycans and lipids are further encompassed by the term immunogen as used herein.

In some embodiments, the immunogen is an immunogenic delivery vehicle, a polypeptide, a polynucleotide, a glycan, or a lipid. In some embodiments, the immunogen is an immunogenic delivery vehicle or a polypeptide or polynucleotide encoded by a transgene contained within the immunogenic delivery vehicle. In some embodiments, the immunogen is an immunogenic delivery vehicle and/or transgene product(s).

In some embodiments, the immunogenic delivery vehicle is a viral vector. In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell. In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector.

In some embodiments, a plasma cell depleting agent, a B cell depleting agent, and/or an immunoglobulin depleting agent may inhibit an immune response by a cell (e.g., an immune cell such as a B cell or a T cell) or by an immune system of a subject (e.g., a human) which can be elicited by an immunogenic delivery vehicle.

In some embodiments, an immunogenic delivery vehicle, e.g., a viral particle or vector disclosed herein, may comprise, e.g., one or more of a heterologous and/or recombinant nucleotide sequence(s) of interest (e.g., a nucleotide sequence encoding a gene, or portion thereof, desired to be expressed in a cell targeted by the viral particle (e.g., a transgene), which nucleotide sequence of interest may be, e.g., DNA or RNA). In some embodiments, the nucleotide sequence can encode a polypeptide of interest disclosed herein. In various embodiments, the nucleotide sequence can encode a transgene product comprising one or more therapeutic agents (e.g., therapeutic proteins or polypeptides) described herein.

In one aspect, the present disclosure provides a method for increasing or maintaining the level of AAV transduction in a target cell and/or tissue, e.g., a target cell and/or tissue within or derived from a subject in need thereof, the method comprising contacting the target cell and/or tissue with, and/or administering to the subject, an effective amount of a plasma cell depleting agent, a B cell depleting agent, and/or an immunoglobulin depleting agent.

In another aspect, provided herein is a method for increasing or maintaining the level of AAV transduction in a target cell and/or tissue, e.g., a target cell and/or tissue within or derived from a subject in need thereof (e.g., a subject without pre-existing immunity against AAV), the method comprising contacting the target cell and/or tissue with, and/or administering to the subject, an effective amount of an anti-CD20×CD3 bispecific antibody or a functional fragment thereof. In some embodiments, the level of AAV transduction in the target cell and/or tissue is increased or maintained by inhibiting or preventing an immune response to the AAV in the subject. In some embodiments, the level of AAV transduction is increased or maintained in the target cell and/or tissue by inhibiting antibody responses to the AAV in the subject.

In some embodiments, the level of AAV transduction in the target cell and/or tissue is increased or maintained by inhibiting or preventing an immune response to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting or preventing an immune response to the AAV in the subject.

In some embodiments, the level of AAV transduction in the target cell and/or tissue is increased or maintained by inhibiting antibody responses to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting antibody responses to the AAV in the subject.

In one aspect, the present disclosure provides a method for increasing or maintaining the level of a transgene expression in a subject in need thereof, the method comprising administering to the subject an effective amount of a plasma cell depleting agent, a B cell depleting agent, and/or an immunoglobulin depleting agent. In some embodiments, the method comprises determining the presence of neutralizing antibodies to the immunogen in the subject. In some embodiments, the transgene is delivered to the subject via an immunogenic delivery vehicle (e.g., AAV).

In another aspect, provided herein is a method for increasing or maintaining the level of a transgene expression in a subject in need thereof, the method comprising administering to the subject an effective amount of an anti-CD20×CD3 bispecific antibody or a functional fragment thereof. In some embodiments, the transgene is delivered to the subject via an immunogenic delivery vehicle (e.g., AAV). In some embodiments, the subject does not have a pre-existing immunity against the immunogenic delivery vehicle and/or transgene product(s).

In some embodiments, the level of transgene expression is increased or maintained by inhibiting an immune response to the immunogenic delivery vehicle and/or by inhibiting an immune response to a polypeptide or polynucleotide encoded by the transgene (i.e., a transgene product). In some embodiments, the level of transgene expression is increased or maintained by inhibiting antibody responses to the polypeptide or polynucleotide encoded by the transgene.

In some embodiments, the level of transgene expression is increased or maintained by inhibiting an immune response to the immunogenic delivery vehicle and/or by inhibiting an immune response to the transgene product(s). In some embodiments, the level of transgene expression is increased or maintained by inhibiting antibody responses to the transgene product(s).

In some embodiments, the level of transgene expression is increased or maintained by inhibiting an immune response to the immunogenic delivery vehicle and/or by inhibiting an immune response to a polypeptide or polynucleotide encoded by the transgene. As a non-limiting example, the level of transgene expression may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of transgene expression may be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of transgene expression may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the immune response to the immunogenic delivery vehicle and/or the immune response to the polypeptide or polynucleotide encoded by the transgene may be inhibited by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The immune response to the immunogenic delivery vehicle and/or the immune response to the polypeptide or polynucleotide encoded by the transgene may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The immune response to the immunogenic delivery vehicle and/or the immune response to the polypeptide or polynucleotide encoded by the transgene may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In another aspect, provided herein is a method for inhibiting or preventing an immune response to an immunogen in a subject in need thereof (e.g., a subject without a pre-existing immunity against the immunogen), the method comprising administering to the subject an effective amount of an anti-CD20×CD3 bispecific antibody or a functional fragment thereof.

In some embodiments, inhibiting or preventing the immune response comprises suppression of numbers and frequencies of immunogen-specific B cells.

In some embodiments of the methods for inhibiting or preventing an immune response to an immunogen described herein, the inhibiting of the immune response can comprise suppression of numbers and/or frequencies of plasma cells and/or B cells.

In some embodiments, the number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the total number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The total number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The total number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, inhibiting the immune response comprises suppression of immunogen-specific IgG and/or IgM responses.

In some embodiments, the responses of IgG and/or IgM may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The responses of IgG may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The responses of IgG may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In one aspect, the present disclosure provides a method for increasing effectiveness of re-administration of an immunogen to a subject in need thereof, comprising administering to the subject an effective amount of a plasma cell depleting agent, a B cell depleting agent, and/or an immunoglobulin depleting agent. As a non-limiting example, the effectiveness of re-administration of an immunogen may be increased by about 1%, about 2%, about 3%, about 4%, about 5%, about 6%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The effectiveness of re-administration of an immunogen be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The effectiveness of re-administration of an immunogen may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the immunogen re-administration occurs via the same administration route as its prior administration. In some embodiments, the immunogen re-administration occurs via a different administration route than its prior administration. In some embodiments, the plasma cell depleting agent, the B cell depleting agent, and/or the immunoglobulin depleting agent is administered before the administration of the immunogen. In some embodiments, the anti-CD20×CD3 bispecific antibody or functional fragment thereof is administered before the administration of the immunogen to the subject. In some embodiments, the anti-CD20×CD3 bispecific antibody or functional fragment thereof is administered before the administration of the immunogenic delivery vehicle to the subject. In some embodiments, the plasma cell depleting agent, the B cell depleting agent, and/or the immunoglobulin depleting agent is administered simultaneously with the administration of the immunogen. In some embodiments, the anti-CD20×CD3 bispecific antibody or functional fragment thereof is administered simultaneously with the administration of the immunogen to the subject. In some embodiments, the anti-CD20×CD3 bispecific antibody or functional fragment thereof is administered simultaneously with the administration of the immunogenic delivery vehicle to the subject. In some embodiments, the plasma cell depleting agent, the B cell depleting agent, and/or the immunoglobulin depleting agent is administered after the administration of the immunogen. In some embodiments, the anti-CD20×CD3 bispecific antibody or functional fragment thereof is administered after the administration of the immunogen to the subject. In some embodiments, the anti-CD20×CD3 bispecific antibody or functional fragment thereof is administered after the administration of the immunogenic delivery vehicle to the subject. In some embodiments, the immunogen is administered two or more times and the plasma cell depleting agent, the B cell depleting agent, and/or the immunoglobulin depleting agent is administered before and/or between each of the administrations of the immunogen. In some embodiments, the immunogen is administered to the subject two or more times and the anti-CD20×CD3 bispecific antibody or functional fragment thereof is administered before and/or between each of the administrations of the immunogen. In some embodiments, the immunogenic delivery vehicle is administered to the subject two or more times and the anti-CD20×CD3 bispecific antibody or functional fragment thereof is administered before and/or between each of the administrations of the immunogenic delivery vehicle. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen, e.g., 2-4 days afterwards as B cell formation may be limited during the initial lag period. Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

### Viral Particles

In one aspect, the present disclosure provides for methods for inhibiting or preventing an immune response to a viral vector in a subject in need thereof, the method comprising administering to the subject an effective amount of a plasma cell depleting agent, a B cell depleting agent, and/or an immunoglobulin depleting agent. The terms “viral vector” and “viral particle” can be used synonymously and interchangeably herein.

In some embodiments, the plasma cell depleting agent, the B cell depleting agent, and/or the immunoglobulin depleting agent is capable of inhibiting and/or preventing an immune response which can be elicited by a viral particle, or a portion thereof (e.g., a capsid protein). In some embodiments, the viral particle can comprise a viral vector (e.g., an adeno-associated virus (AAV) vector, an adenovirus vector, a retrovirus vector, or an oncolytic virus vector) which can comprise one or more of a heterologous and/or recombinant nucleotide sequence(s) of interest (e.g., a nucleotide sequence encoding a gene, or portion thereof, desired to be expressed in a cell targeted by the viral particle (e.g., a transgene), which nucleotide sequence of interest may be, e.g., DNA or RNA). In some embodiments, the nucleotide sequence can encode a polypeptide of interest disclosed herein. In various embodiments, the nucleotide sequence can encode a transgene product comprising one or more therapeutic agents (e.g., therapeutic proteins or polypeptides) described herein. In some embodiments, the nucleotide sequence encodes a therapeutic protein, a suicide gene, an antibody, or a fragment thereof, an antisense oligonucleotide, a ribozyme, an RNAi molecule, and/or a shRNA molecule. In some embodiments, the nucleotide sequence may encode a growth factor, a neurotrophic factor, a disease modifying muscle protein, and/or a metabolic protein, e.g., for muscle atrophy conditions or metabolic diseases.

In some embodiments, a plasma cell depleting agent, a B cell depleting agent, and/or an immunoglobulin depleting agent described herein is capable of inhibiting and/or preventing an immune response which can be elicited by a vector, e.g., viral vector, or a portion thereof, e.g., an adenovirus-associated virus (AAV) vector.

A vector can comprise additional sequences such as, for example, replication origins, promoters, and genes encoding antibiotic resistance. Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Viral vectors can be derived from naturally occurring virus genomes, which typically are modified to be replication incompetent, e.g. non-replicating. Non-replicating viruses require the provision of proteins in trans for replication. Typically, those proteins are stably or transiently expressed in a viral producer cell line, thereby allowing replication of the virus. The viral vectors are, thus, typically infectious, and non-replicating. Non-limiting examples of viral vectors include adenovirus vectors, adeno-associated virus (AAV) vectors (e.g., AAV type 8), alphavirus vectors (e.g., Venezuelan equine encephalitis virus (VEE), Sindbis virus (SIN), Semliki forest virus (SFV), and VEE-SIN chimeras), herpes virus vectors (e.g., vectors derived from cytomegaloviruses, like rhesus cytomegalovirus (RhCMV)), arena virus vectors (e.g. lymphocytic choriomeningitis virus (LCMV) vectors), measles virus vectors, pox virus vectors (e.g., vaccinia virus, modified vaccinia virus Ankara (MVA), NYVAC (derived from the Copenhagen strain of vaccinia), and avipox vectors (canarypox (ALVAC) and fowlpox (FPV) vectors), vesicular stomatitis virus (VSV) vectors, retrovirus vectors, lentivirus vectors, simian virus 40 (SV40), bovine papilloma viruses, Epstein-Barr viruses, Moloney murine leukemia viruses, Harvey murine sarcoma viruses, murine mammary tumor viruses, Rous sarcoma viruses, poxvirus viral like particles, baculoviral vectors and bacterial spores.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette can be derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide.

The ssDNA AAV genome consists of two open reading frames, Rep and Cap, flanked by two inverted terminal repeats (ITRs) that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

As further examples, adenovirus vectors may be derived from human adenovirus (Ad) but also from adenoviruses that infect other species, such as bovine adenovirus (e.g. bovine adenovirus 3, BAdV3), canine adenovirus (e.g. CAdV2), porcine adenovirus (e.g. PAdV3 or 5), or adenoviruses that infect great apes, such as Chimpanzee (Pan), Gorilla (Gorilla), Orangutan (Pongo), Bonobo (Pan paniscus) and common chimpanzee (Pan troglodytes). Poxvirus (Poxviridae) vectors may be derived from smallpox virus (variola), vaccinia virus, cowpox virus or monkeypox virus. Exemplary vaccinia viruses are the Copenhagen vaccinia virus (W), New York Attenuated Vaccinia Virus (NYVAC), ALVAC, TROVAC and Modified Vaccinia Ankara (MVA).

In some embodiments, a plasma cell depleting agent, a B cell depleting agent, and/or an immunoglobulin depleting agent of the present disclosure may inhibit an immune response which may be elicited by a transgene product, e.g., a transgene product (e.g., a therapeutic polypeptide or polynucleotide of interest or disclosed herein which is encoded by the transgene) comprising one or more therapeutic agents. The one or more therapeutic agents may comprise a therapeutic protein (e.g., a therapeutic polypeptide) and/or a therapeutic nucleic acid. Non-limiting examples of therapeutic agents which may be expressed by a transgene using methods of the present disclosure include, e.g., proteins and polypeptides, antisense RNA, or ribozymes, or any combination thereof. In some embodiments, the heterologous and/or recombinant nucleotide sequence becomes integrated into the cell genome. In some embodiments, the heterologous and/or recombinant nucleotide sequence does not become integrated into the cell genome.

Examples of therapeutic proteins and polypeptides suitable for expression methods of the present disclosure include human hormones such as growth hormone, prolactin, insulin, luteinizing hormone, calcitonin, follicle stimulating hormone, chorionic gonadotropin or thyroid stimulating hormone; a chemokine including, MIP-1β and RANTES Iα; a colony stimulating factor, e.g., G-CSF, GM-M-CSF and CSF; growth factors such as IGF-1 and IGF-2; a cytokine, such as interleukin (IL)-1, IL-2 IL-3, IL-4, IL-5, IL-6, IL-7, IL-8, IL-9, IL-10, IL-11, IL-12, IL-13, IL-14 and IL-15, α-interferons, β-interferons, the γ-interferons, LFA-1, tumor necrosis factor, CD3, ICAM-1 and LFA-3; LDL receptor, ornithine transcarbamylase, phenylalanine hydroxylase, and al-antitrypsin.

Additional examples of sequences expressible using the methods described herein include sequences of Protein S and Gas6, thrombin, acidic fibroblast growth factor (FGF-1), basic FGF (FGF-2), keratinocyte growth factor (KGF), TGF, platelet derived growth factor (PDGF), epidermal growth factor (EGF), hepatocyte growth factor (HGF) and HGF activators, PSA, nerve cell growth factor (NCGF), glial cell derived nerve growth factor (GDNF), vascular endothelial growth factor (VEGF), Arg-vasopressin, thyroid hormones azoxymethane, triiodothyronine, LIF, amphiregulin, soluble thrombomodulin, stem cell factor, osteogenic protein 1, the bone morphogenic proteins, MFG, MGSA, heregulins and melanotropin, human growth hormone, leptin, IL-2, erythropoietin, and thrombopoietin (G-CSF).

In some embodiments, vectors and/or viral particles described herein may comprise, e.g., genes encoding apoptotic factors, genes encoding cytotoxic molecules, genes encoding anti-apoptotic factors, genes encoding immune-stimulatory molecules, a TNF-α gene, a p53 gene, interferon genes, “suicide genes” (i.e., the genes which cause a cell to kill itself through apoptosis; non-limiting examples of suicide genes include, e.g., herpes simplex virus thymidine kinase (HSV-TK), which converts ganciclovir (GCV) into cytotoxic compounds, Escherichia coli cytosine deaminase, which allows the formation of a cytotoxic chemotherapeutic agent from a non-toxic precursor, Varicella-zoster virus thymidine kinase, deoxycytidine kinase, purine nucleoside phosphorylase, nitroreductase, β-galactosidase, hepatic cytochrome P450-2B1, linamarase, horseradish peroxidase, and carboxypeptidase).

In some embodiments, a protein or polypeptide encoded by the genes inserted into the vectors and viral particles of the present disclosure can provide one or more antigens or antigenically active fragments thereof associated with, e.g., one or more infectious agents such as a bacteria, virus, parasite, or fungus, or a combination thereof, which may be used to immunize a subject. An active fragment described herein may comprise a polypeptide which contains less than a full-length sequence but that retains sufficient biological activity to be used in the methods of the disclosure.

As a non-limiting example, antigens can include H. pylori antigens VacA (cytotoxin), heat shock protein, CagA (cai antigen) and urease B and HCV antigens, such as HCV NS3, NS4, EI, E2 and/or E2a. In some embodiments, an antigen can include influenza antigens, rabies antigens and bacterial antigens from Bordetella pertussia, Neisseria meningitides (A, B, C, Y 135), gD, gB and other glycoproteins, HSV (herpes simplex virus), MMR and VZV (Varicella Zoster virus) antigens, CMV (cytomegalovirus) gB or gH glycoproteins, hepatitis D virus (HDV) delta antigen HIV gp 120, p24 and other proteins, hepatitis A virus (HAV) antigens, and EBV (Epstein Barr vims).

In some embodiments, a protein or polypeptide encoded by heterologous or recombinant nucleotide sequence(s) inserted into the vectors and viral particles of the present disclosure can provide one or more antigen-binding molecules or antigen-binding fragments thereof, e.g., antibodies or antigen-binding fragments thereof. Antibodies or antigen-binding fragments thereof encompass derivatives, functional equivalents, and homologues of antibodies, humanized antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. A humanized antibody may be a modified antibody having the variable regions of a non-human, e.g. murine, antibody, and the constant region of a human antibody. Examples of antibodies are the immunoglobulin isotypes (e.g., IgG, IgE, IgM, IgD and IgA) and their isotypic subclasses; or fragments that comprise an antigen binding domain such as Fab, scFv, Fv, dAb, Fd; and diabodies. Antibodies may be polyclonal or monoclonal. A monoclonal antibody may be referred to herein as “mAb”. In some embodiments, the antibody is a multispecific antibody. In some embodiments, the antibody is a bispecific antibody. The bispecific antibody may comprise a second targeting moiety that targets to the desired cell or tissue, e.g., liver, or to another desired antigen associated with the same or similar disease or disorder (e.g., liver cancer antigen).

In some embodiments, an antigen-binding molecule or antigen-binding fragment thereof (e.g., an scFv) may be fused to a payload such as, e.g., an alpha-glucosidase polypeptide (GAA) or an arylsulfatase B (ARSB) polypeptide, or variants thereof, to form an antigen-binding molecule:payload fusion protein. The term “fused” with regard to fused polypeptides refers to polypeptides joined directly or indirectly (e.g., via a linker or other polypeptide).

An antisense sequence that can be expressed by the vectors and viral particles of the present disclosure can be an RNA sequence which is capable of preventing or limiting the expression of defective, over-produced, or otherwise undesirable molecules by being sufficiently complementary to a target sequence. In some embodiments, the target sequence may comprise an mRNA that encodes a protein, and the antisense RNA can bind to the mRNA and prevent its translation. In some embodiments, the target sequence can be a portion of a gene that is requisite for transcription, and the antisense RNA can bind to the gene portion and prevent or limit its transcription.

In certain embodiments, the antisense sequence is an antisense oligonucleotide (ASO). An ASO can down regulate a target by inducing RNase H endonuclease cleavage of a target RNA, by steric hindrance of ribosomal activity, by inhibiting 5′ cap formation, or by altering splicing. An ASO can be, but is not limited to, a gapmer or a morpholino. An antisense oligonucleotide typically comprises a short nucleotide sequence which is substantially complementary to a target nucleotide sequence in a pre-mRNA molecule, heterogeneous nuclear RNA (hnRNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Antisense oligonucleotides are often synthetic and chemically modified.

In some embodiments, the vectors and viral particles of the present disclosure can express a ribozyme (ribonucleic acid enzyme). A ribozyme is a molecule, commonly an RNA molecule, that is capable of performing specific biochemical reactions, akin to the action of protein enzymes. Ribozymes comprise molecules possessing catalytic activities such as, but not limited to, the capacity to cleave at specific phosphodiester linkages in RNA molecules to which they have hybridized, e.g., RNA-containing substrates, lncRNAs, mRNAs, and ribozymes. Exemplary ribozymes include ribozymes to, e.g., hepatitis A, hepatitis B and hepatitis C.

In some embodiments, a plasma cell depleting agent, a B cell depleting agent, and/or an immunoglobulin depleting agent inhibitor of the present disclosure may inhibit an immune response via blocking and/or suppressing induced and/or pre-existing immunity of a host (e.g., a subject) against a viral particle or portion thereof, and/or a gene delivery vector (e.g., a viral vector such as a AAV vector), or a transgene product thereof, described herein. In some embodiments, the induced and/or pre-existing host immunity may comprise, e.g., B cell production of at least one antibody (e.g., neutralizing antibody (nAb)) to a viral particle or portion thereof, e.g., capsid protein, and/or a viral vector described herein. An immune response involving production of nAbs induced by viral particles and/or vectors can be associated with T cell and B cell activation.

An antibody may be capable of both binding and neutralizing a viral particle or a portion thereof (e.g., neutralizing antibody (nAb)). In some embodiments, the antibody may affect pharmacokinetic properties or alter uptake of AAV into different cell types. In some embodiments, neutralizing (or neutralize or neutralization and the like) in the context of the present disclosure may comprise an effect of immunoglobulins, such as antibodies generated in a host immune response, in reducing the efficacy and/or delivery of a viral particle. As an example, neutralization by an at least one nAb described herein may be realized such that the nAb is directed to the viral particle surface (e.g., capsid protein) which may result in aggregation of viral particles and/or may be realized by inhibition of the fusion of viral and a cellular membrane(s) after attachment of the viral particle to a target cell, by inhibition of endocytosis, and/or by inhibition of production of viral progeny. In various embodiments, an antibody generated in a subject's host immune response can play a neutralizing role thereby causing the delivery effectiveness of the viral particle to be reduced or eliminated. In some embodiments, the induced and/or pre-existing host immunity may comprise B and/or T cell immune responses described herein. The blockade and/or suppression of induced and/or pre-existing host immunity against viral particles or portions thereof, can improve viral transduction and allow for effective re-administration (i.e., re-dosing) of the viral particles during gene therapy.

In some embodiments, the methods for inhibiting or preventing an immune response described herein may be useful when a subject has or will develop an immune response to one or more viral particles, or portion thereof (e.g., an immune response comprising generation of nAbs), e.g., one or more AAV vector particles comprising a gene or portion thereof contained within the AAV vector which may encode a transgene product comprising one or more therapeutic agent(s), e.g., as part of a gene therapy regimen. In particular, the present disclosure is useful when a subject in need of a therapeutic agent may require multiple or extended treatments, e.g., over a period of years. In some embodiments, the present disclosure allows for multiple administrations (i.e., re-dosing) to the same subject of the same recombinant AAV particles which may have a gene encoding a therapeutic agent needed by the subject, and provides for expression of the therapeutic agent even when a subject has developed an immunity to the AAV of the vector particle. In some embodiments, the present disclosure can be useful for a subject in need of a therapeutic protein that would require and/or benefit from a therapeutic agent on a continuous or bolus basis.

In another aspect, the present disclosure provides for methods for increasing or maintaining the level of a transgene expression in a subject in need thereof, the method comprising administering to the subject an effective amount of a plasma cell depleting agent, a B cell depleting agent, and/or an immunoglobulin depleting agent. In some embodiments, the method comprises determining the presence of neutralizing antibodies to the immunogen in the subject.

In yet another aspect, the present disclosure provides for methods for increasing effectiveness of re-administration of a viral vector of the same or similar viral origin as the originally administered viral vector in a subject in need thereof, the methods comprising administering to the subject an effective amount of a plasma cell depleting agent, a B cell depleting agent, and/or an immunoglobulin depleting agent.

In some embodiments, the present disclosure contemplates a method for increasing effectiveness of administration of a subsequently administered viral vector following administration of an originally administered viral vector in a subject in need thereof, comprising administering to the subject an effective amount of a plasma cell depleting agent, a B cell depleting agent, and/or an immunoglobulin depleting agent, and the subsequently administered viral vector is of the same or similar viral origin as the originally administered viral vector. In some embodiments, the present disclosure contemplates a method for increasing effectiveness of a subsequently administered viral vector following an originally administered viral vector in a subject in need thereof, comprising administering to the subject an effective amount of a plasma cell depleting agent, a B cell depleting agent, and/or an immunoglobulin depleting agent, and the subsequently administered viral vector is of the same or similar viral origin as the originally administered viral vector. In some embodiments, the method comprises determining the presence of neutralizing antibodies to the immunogen in the subject.

In another aspect, provided herein is a method for increasing effectiveness of a subsequently administered viral vector following an originally administered viral vector in a subject in need thereof the method comprising administering to the subject an effective amount of an anti-CD20×CD3 bispecific antibody or a functional fragment thereof, wherein the subsequently administered viral vector is of the same or similar viral origin as the originally administered viral vector. In some embodiments, the subject does not have a pre-existing immunity against the viral vectors.

The viral particles and/or vectors described herein can be derived from any enveloped or non-enveloped virus. Non-limiting examples of enveloped viruses from which the viral particles and/or viral vectors described herein can be derived include, e.g., retroviruses (e.g., rous sarcoma virus, human and bovine T-cell leukaemia virus (HTLV and BLV)), lentiviruses (e.g., human and simian immunodeficiency viruses (HIV and SIV), Mason-Pfizer monkey virus), foamy viruses (e.g., Human Foamy Virus (HFV)), herpes viruses (herpes simplex virus (HSV), varicella-zoster virus, VZVEBV, HCMV, HHV), hantaviruses, pox viruses (e.g., vertebrate and avian poxviruses, vaccinia viruses), orthomyxoviruses (e.g., influenza A, influenza B, influenza C viruses), paramyxoviruses (e.g., parainfluenza virus, respiratory syncytial virus, Sendai virus, mumps virus, measles and measles-like viruses), rhabdoviruses (e.g., vesicular stomatitis virus, rubella virus, rabies virus), coronaviruses (e.g., SARS, MERS), flaviviruses (e.g., Marburg virus, Reston virus, Ebola virus), alphaviruses (e.g., Sindbis virus), bunyaviruses, arenaviruses (e.g., LCMV, GTOV, JUNV, LASV, LUJV, MACV, SABV, WWAV), iridoviruses, and hepadnaviruses.

Non-limiting examples of non-enveloped viruses from which the viral particles and/or viral vectors described herein can be derived include, viruses from the families Picornaviridae, Reoviridae, Caliciviridae, Adenoviridae and Parvoviridae, such as calicivirus, picornavirus, astrovirus, adenovirus, reovirus, polyomavirus, papillomavirus, parvovirus (e.g., adeno-associated virus (AAV)), and type E Hepatitis virus. In some embodiments, the viral vectors are derived from an adeno-associated virus (AAV), an adenovirus, or a retrovirus. In some embodiments, the viral vectors are derived from AAV.

In some embodiments, the viral particles and/or vectors described herein can be derived from an oncolytic virus, including adenovirus, rhabdovirus, herpes virus, measles virus, coxsackievirus, poliovirus, reovirus, poxvirus, parvovirus, Maraba virus, Newcastle disease virus, or paramyxovirus, or any species or strain within these larger groups. A virus disclosed herein may be unaltered from the parental virus species (i.e., wild-type), or with gene modifications, e.g., gene additions.

In some embodiments, the viral particles and/or vectors described herein may be derived from a rhabdovirus. In some embodiments, the recombinant virus may comprise a rhabdovirus genome. The Rhabdoviridae family is mainly composed of a cage, bullet-shaped or bacilliform virus and has a negative-sense single-stranded RNA genome that infects vertebrates, invertebrates, or plants. Non-limiting examples of rhabdoviruses that can be used in the present disclosure include rabies, cytolabudoviruses, dicholabdoviruses, ephemeraviruses, lyssaviruses, nobilabdoviruses, and vesiculoviruses. In some embodiments, the rhabdovirus is a vesiculovirus including, without limitation, a vesicular stomatitis virus (VSV).

In some embodiments, the viral particles and/or vectors described herein is derived from a virus of the family Retroviridae. In one specific embodiment, the viral particle described herein is a retroviral particle. In some embodiments, the viral particle is derived from a lentivirus. Compared to other gene transfer systems, lentiviral and retroviral vectors offer a wide range of advantages, including their ability to transduce a variety of cell types, to stably integrate transferred genetic material into the genome of the targeted host cell, and to express the transduced gene at significant levels. Vectors derived from the gamma-retroviruses, for example, the murine leukemia virus (MLV), have been used in clinical gene therapy trials (Ross et al., Hum. Gen Ther. 7:1781-1790, 1996).

In some embodiments, a viral particle described herein comprises one or more transfer vectors comprising a nucleotide sequence of interest. In some embodiments, the nucleotide sequence of interest is an RNA molecule transcribed by a transfer vector. In some embodiments a viral particle described herein comprises RNA comprising the nucleotide sequence of interest. In some embodiments, a viral particle as described herein comprises one or more transfer vectors, or one or more RNA molecule encoded by the transfer vector, wherein said transfer vector or RNA molecule comprises the nucleotide sequence of interest, and optionally, further comprises a viral element. In some embodiments, upon infection of a target cell with the viral particle, the nucleotide sequence of interest becomes integrated into the cell genome. In some embodiments, the at least one viral element is a retroviral element. In some embodiments, the at least one viral element is a lentiviral element. In some embodiments, the at least one viral element is a Psi (ψ) packaging signal. In some embodiments, in addition to a Psi (ψ) packaging signal, the viral element further comprises a 5′ Long Terminal Repeat (LTR) and/or a 3′ LTR, or a derivative or mutant thereof. In one specific embodiment, the at least one viral element is selected from the group consisting of a 5′ Long Terminal Repeat (LTR), a Psi (ψ) packaging signal, a Rev Response Element (RRE), a promoter that drives expression of the nucleotide sequence of interest, a Central Polypurine Tract (cPPT), a Woodchuck hepatitis virus post-transcriptional regulatory element (WPRE), a Unique 3′ (U3), a Repeat (R) region, a Unique 5′ (U5), a 3′ LTR, a 3′LTR with the U3 element deleted (e.g., to make the lentivirus non-replicative), a Trans-activating response element (TAR), and any combination thereof.

In some embodiments, a viral particle described herein is designed such that a nucleotide of interest is integrated into a genome of a target cell upon infection of the cell by the viral particle. Such integration may be mediated by viral enzymes carried by the viral particle. In some embodiments, a viral particle as described herein further comprises an enzyme, e.g., an integrase, or a nucleic acid encoding same, such that a nucleotide of interest is integrated into a genome of a target cell upon infection of the target cell by the viral particle.

In some embodiments, a viral particle described herein is designed such that a nucleotide of interest remains episomal to a genome of a target cell upon infection of the target cell by the viral particle. In some embodiments, a viral particle described herein lacks an enzyme that mediates integration of a nucleotide of interest, e.g., an integrase, or a nucleic acid encoding same.

In some embodiments, the viral particle described herein comprises components, e.g., capsomers, glycoproteins, etc., from a virus selected from the group consisting of Human Immunodeficiency Virus (HIV), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), or Murine Leukemia Virus (MLV). In some embodiments, a viral particle described herein comprises an HIV capsomer, a plurality of HIV capsomers, and/or an HIV capsid, e.g., is a HIV viral particle and/or is derived from HIV.

In some embodiments, a viral particle as described herein displays a fusogen. In some embodiments, the fusogen is a protein; e.g., a viral protein (e.g., a vesiculovirus protein [e.g., vesicular stomatitis virus G glycoprotein (VSVG)], an alphavirus protein [e.g., a Sindbis virus glycoprotein], an orthomyxovirus protein [e.g., an influenza HA protein], a paramyxovirus protein [e.g., a Nipah virus F protein or a measles virus F protein]), a retrovirus protein, a lentivirus protein, or a fragment, mutant or derivative thereof. In one specific embodiment, the fusogen is heterologous to the reference wild type virus from which the particle is derived. In some embodiments, the fusogen is a mutated protein which does not bind its natural ligand.

In some embodiments, the viral particles described herein are replication deficient and only contain an incomplete genome of the virus from which they are derived. For example, in some embodiments, the lentiviral and retroviral particles do not comprise the genetic information of the gag, env, or pol genes (which may be involved in the assembly of the viral particle), which is a known minimal requirement for successful replication of a lentivirus or retrovirus. In some embodiments, the retrovirus is a lentivirus. In these cases, the minimal set of viral proteins needed to assemble the vector particle are provided in trans by means of a packaging cell line. In one specific embodiment, for lentiviral particles derived from HIV-1, env, tat, vif, vpu and nef genes are lacking and are not provided in trans or are made inactive by the use of frame shift mutation(s).

In some embodiments, an RNA molecule which can be incorporated into the lentiviral or retroviral particles comprises the psi packaging signal and LTRs. In some embodiments, the RNA molecule incorporated into the lentiviral or retroviral particles comprises a nucleotide sequence of interest. To achieve expression of such nucleotide sequence of interest in the target cell, such sequence can be placed under the control of a suitable promoter, for example, the CMV promoter.

In some embodiments of lentiviral and retroviral particles, RNA molecule together with the gag and pol encoded proteins, provided in trans by the packaging cell line, are then assembled into the vector particles, which then infect their target cells, reverse-transcribe the RNA molecule that may comprise a nucleotide sequence of interest under the control of a promoter, and either integrate said genetic information into the genome of the target cells or remain episomal (if one or more of the components required for integration are disrupted). If the genetic information for the gag and pol encoded proteins is not present on the transduced RNA molecule, the vector particles are replication deficient, i.e., no new generation of said vector particles will thus be generated by the transduced cell, thus ensuring safety in clinical applications.

In some embodiments, the viral particle and/or viral vector is derived from an adeno-associated virus (AAV). “AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions, VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99:11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference.

“AAV” encompasses all subtypes and both naturally occurring and modified forms, except where stated otherwise. AAV includes primate AAV (e.g., AAV type 1 (AAV1), primate AAV type 2 (AAV2), primate AAV type 3 (AAV3), primate AAV3B, primate AAV type 4 (AAV4), primate AAV type 5 (AAV5), primate AAV type 6 (AAV6), primate AAV6.2, primate AAV type 7 (AAV7), primate AAV type 8 (AAV8), primate AAV type 9 (AAV9), AAV10, AAV type hu11 (AAV hu11), AAV11, AAV12, AAV13, AAVDJ, Anc80L65, AAV2G9, AAVLK03, AAV type rh32.33 (AAVrh.32.33), AAV retro (AAV retro), AAV PHP.B, AAV PHP.eB, AAV PHP.S, AAVrh.64R1, AAVhu.37, AAVrh.8, AAV2/8, etc.; non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc., “Primate AAV” refers to AAV generally isolated from primates. Similarly, “non-primate animal AAV” refers to AAV isolated from non-primate animals.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA₂ region, VP1-u region, VP1/VP2 common region, VP3 region), nucleotide sequence (e.g., ITR sequence), e.g., a cap gene or capsid protein of AAV etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional cap gene.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein,” and the like, include a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA₂), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional ˜60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A “D” region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep 52 and/or Rep40 Rep proteins. Alternatively, Rep proteins may be expressed individually from an ORF comprising a nucleotide sequence encoding any one of Rep78, Rep68, Rep 52, or Rep40 by use of separate nucleotide sequences operably linked to one expression control sequence for expression in a packaging cell, each producing only one Rep78, Rep68, Rep 52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid. However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

In some embodiments, a Cap protein, e.g., a VP1 capsid protein as described herein, a VP2 capsid protein as described herein, and/or a VP3 capsid protein as described herein, is modified to comprise e.g., a first member of a protein:protein binding pair, a detectable label, point mutation, etc.

Chimerism is a type of modification as described herein. Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide. A modification may include an insertion of, e.g., a first member of a protein:protein binding pair and a point mutation, e.g., such that the natural tropism of the capsid protein is reduced or abolished and/or such that the capsid protein comprises a detectable label. Modifications can include those that either do not alter or decrease the likelihood that the modified capsid will be recognized by pre-existing antibodies found in the general population, e.g., antibodies produced during the course of previous infection(s) with an AAV, e.g., infection(s) with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof. Other modifications as described herein include modification of a capsid protein such that it comprises a first member of a protein:protein binding pair, a detectable label, etc., which modifications generally result from modifications at the genetic level, e.g., via modification of a cap gene.

In some embodiments, a viral capsid comprising a modified viral capsid protein as described herein is a mosaic capsid, e.g., which comprises at least two sets of VP1, VP2, and/or VP3 proteins, each set of which is encoded by a different cap gene. A mosaic capsid herein generally refers to a mosaic of a first viral capsid protein modified to comprise a first member of a protein:protein binding pair and a second corresponding viral capsid protein lacking the first member of a protein:protein binding pair. In relation to a mosaic capsid, the second viral capsid protein lacking the first member of a protein:protein binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a protein:protein binding pair, except that the reference capsid protein lacks the first member of a protein:protein binding pair. In some mosaic capsid embodiments, a VP1, VP2, and/or VP3 reference capsid protein corresponds to the viral VP1, VP2, and/or VP3 capsid protein modified with a first member of a protein:protein binding pair, except that the reference capsid protein lacks the first member of a protein:protein binding pair. In some embodiments, a VP1 reference capsid protein corresponds to the viral VP1 capsid protein modified with a first member of a protein:protein binding pair, except that the reference capsid protein lacks the first member of a protein:protein binding pair. In some embodiments, a VP2 reference capsid protein corresponds to the viral VP2 capsid protein modified with a first member of a protein:protein binding pair, except that the reference capsid protein lacks the first member of a protein:protein binding pair. In some embodiments, a VP3 reference capsid protein corresponds to the viral VP3 capsid protein modified with a first member of a protein:protein binding pair, except that the reference capsid protein lacks the first member of a protein:protein binding pair. In some mosaic capsid embodiments comprising a chimeric VP1, VP2, and/or VP3 capsid protein further modified to comprise a first member of a protein:protein binding pair, a reference protein may be a corresponding capsid protein from which portions thereof form part of the chimeric capsid protein. As a non-limiting example, in some embodiments, a mosaic capsid comprising a chimeric AAV2/AAAV VP1 capsid protein modified to comprise a first member of a protein:protein binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the protein:protein binding pair and is able to form a capsid with the first capsid protein modified with the first member of a protein:protein binding pair.

Generally mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a protein:protein binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a protein:protein binding pair. In some non-mosaic viral particle embodiments, the protein subunit ratio may be 0:1 wherein each capsid protein of the non-mosaic viral particle is not modified with a first member of a protein:protein binding pair.

In some embodiments, a capsid protein of the invention is modified to comprise a detectable label. Many detectable labels are known in the art. (See, e.g.: Nilsson et al. (1997) “Affinity fusion strategies for detection, purification, and immobilization of modified proteins” Protein Expression and Purification 11:1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6 (SEQ ID NO: 56), His-8 (SEQ ID NO: 57), or His-10 (SEQ ID NO: 58)) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag, such as that which is commercially available from Covalys. In some embodiments, a detectable label disclosed herein comprises a detectable label recognized only by an antibody paratope. In some embodiments, a detectable label disclosed herein comprises a detectable label recognized by an antibody paratope and other specific binding pairs.

In some embodiments, the detectable label forms a binding pair with an immunoglobulin constant domain. In some embodiments, the detectable label and/or detectable label does form a binding pair with a metal ion, e.g., Ni²⁺, Co²⁺, Cu²⁺, Zn²⁺, Fe³⁺, etc. In some embodiments, the detectable label is selected from the group consisting of Streptavidin, Strep II, HA, L14, 4C-RGD, LH, and Protein A.

In some embodiments, the detectable label is selected from the group consisting of FLAG, HA, and c-myc.

In some embodiments, a detectable label is a B cell epitope, e.g., which is between about 1 amino acid and about 35 amino acids in length, and forms a binding pair with an antibody paratope, e.g., an immunoglobulin variable domain. In some embodiments, the detectable label comprises a B1 epitope. In some embodiments, a capsid protein is modified to comprise a B1 epitope in the VP3 region.

In some embodiments, a capsid protein of the invention comprises at least a first member of a peptide:peptide binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc. Use of a detectable label as a first member of a protein:protein binding pair is described in, e.g., WO2019006043, incorporated herein in its entirety by reference.

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag:KTag, Isopeptag:pilinC, and SnoopTag:SnoopCatcher. In some embodiments, the first member is SpyTag (or a biologically active portion thereof) and the protein (second cognate member) is SpyCatcher (or a biologically active portion thereof). In some embodiments, the first member is SpyTag (or a biologically active portion thereof) and the protein (second cognate member) is KTag (or a biologically active portion thereof). In some embodiments, the first member is KTag (or a biologically active portion thereof) and the protein (second cognate member) is SpyTag (or a biologically active portion thereof). In some embodiments, the first member is SnoopTag (or a biologically active portion thereof) and the protein (second cognate member) is SnoopCatcher (or a biologically active portion thereof). In some embodiments, the first member is Isopeptag (or a biologically active portion thereof) and the protein (second cognate member) is Pilin-C (or a biologically active portion thereof). In some embodiments, the first member is SpyTag002 (or a biologically active portion thereof) and the protein (second cognate member) is SpyCatcher002 (or a biologically active portion thereof). In some embodiments, a Cap protein of the invention comprises a SpyTag. Use of a first member of a protein:protein binding pair is described in WO2019006046, incorporated herein in its entirety.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length. In some embodiments, the first and second linkers are identical in length, and are each five amino acids in length. In some embodiments, the first and second linkers are identical in length, and are each six amino acids in length. In some embodiments, the first and second linkers are identical in length, and are each seven amino acids in length. In some embodiments, the first and second linkers are identical in length, and are each eight amino acids in length. In some embodiments, the first and second linkers are identical in length, and are each nine amino acids in length. In some embodiments, the first and second linkers are identical in length, and are each ten amino acids in length. In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids and about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

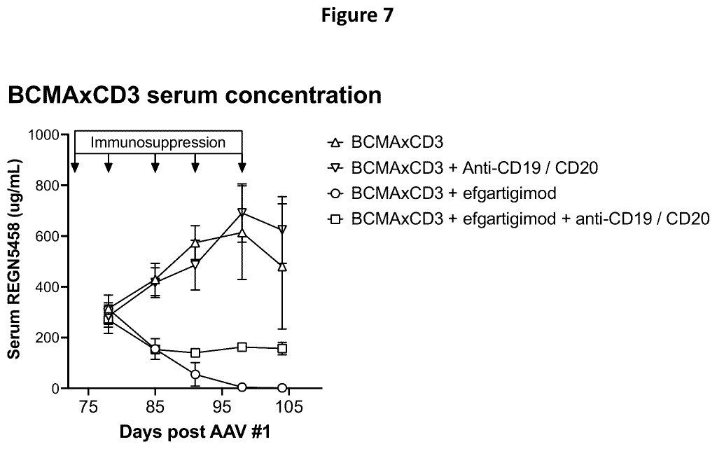

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzsch et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of a first member of a protein:protein binding pair and/or detectable label.

Accordingly, in some embodiments, the first member of a protein:protein binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a protein:protein binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I261, I-266, I-381, I-453, I-447, I-448, I-459, I-471, I-520, I-534, I-570, I-573, I-584, I-587, I-588, I591, I-657, I-664, I-713 and I-716 of the VP1 capsid protein of AAV2 (Wu et al. (2000) J. Virol. 74:8635-8647). In some embodiments, an insertion site of a non-primate animal VP1 capsid protein corresponds to I-453. A modified virus capsid protein as described herein may be a non-primate animal capsid protein comprising a first member of a protein:protein binding pair and/or detectable label inserted into a position corresponding with a position of an AAV2 capsid protein selected from the group consisting of I-1, I-34, I-138, I-139, I-161, I261, I-266, I-381, I-447, I-448, I-453, I-459, I-471, I-520, I-534, I-570, I-573, I-584, I-587, I-588, I591, I-657, I-664, I-713, I-716, and a combination thereof. In some embodiments, an insertion site of a non-primate animal VP1 capsid protein corresponds to I-453. Additional suitable insertion sites of a non-primate animal AAV that include those corresponding to I-587 of AAV1, I-589 of AAV1, I-585 of AAV3, I-585 of AAV4, and I-585 of AAV5. In some embodiments, a modified virus capsid protein as described herein may be a non-primate animal capsid protein comprising a first member of a protein:protein binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a protein:protein binding pair and/or detectable label is inserted in a VP1 capsid protein of a non-primate animal AAV after an amino acid position corresponding with an amino acid position selected from the group consisting of I444 of an avian AAV capsid protein VP1, I580 of an avian AAV capsid protein VP1, I573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and I565 of a sea lion AAV capsid protein VP1.

The nomenclature I-###, I #or the like herein refers to the insertion site (I) with ###naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 1.5-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a protein:protein binding pair (e.g., wherein the second member is operably linked to a targeting ligand, comprises a multispecific binding protein, etc.) is better able to evade neutralization by pre-existing antibodies in serum isolated from a human subject compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a protein:protein binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC₅₀ value that is at least 2-fold that of a control virus particle).

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule comprising (i) an antibody paratope that specifically binds the detectable label and (ii) a second binding domain that specifically binds a receptor targets the viral particle. Such “targeting” or “directing” may include a scenario in which the wildtype viral particle targets several cells within a tissue and/or several organs within an organism, which broad targeting of the tissue or organs is reduced to abolished by insertion of the detectable label, and which retargeting to more specific cells in the tissue or more specific organ in the organism is achieved with the multispecific binding molecule. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule. An antibody paratope as described herein generally comprises at a minimum a complementarity determining region (CDR) that specifically recognizes the detectable label, e.g., a CDR3 region of a heavy and/or light chain variable domain. In some embodiments, a multispecific binding molecule comprises an antibody (or portion thereof) that comprises the antibody paratope that specifically binds the detectable label. For example, a multispecific binding molecule may comprise a single domain heavy chain variable region or a single domain light chain variable region, wherein the single domain heavy chain variable region or single domain light chain variable region comprises an antibody paratope that specifically binds the detectable label. In some embodiments, a multispecific binding molecule may comprise an Fv region, e.g., a multispecific binding molecule may comprise an scFv, that comprises an antibody paratope that specifically binds the detectable label. In some embodiments, a multispecific binding molecule as described herein comprises an antibody paratope that specifically binds c-myc.

One embodiment of the present invention comprises a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a highly preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

A further embodiment of the modified viral capsids described herein is their use for delivering a nucleotide of interest, e.g., a reporter gene and/or a therapeutic gene, to a target cell. Generally, packaging of a nucleotide of interest comprises replacing an AAV genome between AAV ITR sequences with a gene of interest to create a transfer plasmid, which is then encapsulated in an AAV capsid according to well-known methods Thus, a modified viral capsid as described herein may encapsulate a transfer plasmid and/or a nucleotide of interest, which may generally comprise 5′ and 3′ inverted terminal repeat (ITR) sequences flanking a gene of interest, e.g., reporter gene(s) or therapeutic gene(s), or a portion of the gene of interest (which may be under the control of a viral or non-viral promoter). According to well-known methods of packaging AAV viral particles, the modified viral capsids, the 5′ ITR, and the 3′ ITR need not be of the same AAV serotype. In one embodiment, a transfer plasmid and/or nucleotide of interest comprises from 5′ to 3′: a 5′ ITR, a promoter, a gene (e.g., a reporter and/or therapeutic gene) and a 3′ITR.

A consideration for AAV transfer plasmid design is that a wildtype AAV genome is ˜4.7 kb. Thus, included herein are the well-known strategies that provide for packaging nucleotides of interest that exceed the packaging capacity of an individual AAV. Such strategies include, but are not limited to, dual-vector strategies that exploit ITR-mediated recombination to express genes of interest that are larger than a wildtype AAV genome by way of transcript splicing across intermolecularly recombined ITRs from two complementary vector genomes, vector recombination by homology, RNA trans-splicing, and/or protein “trans-splicing” via split intein designs. See, e.g., Nakai, H. et al. (2000) Nat. Biotechnol. 18:527-532; Sun, L. (2000) Nat. Med. 6:599-602 (2000); Ghosh, A., et al. (2008) Mol. Ther. 16:124-130 (2008); Lai, Y (2005) Nat. Biotechnol. 23:1435-1439; Chew, W. L. et al. (2016) Nat. Methods 13:868-874; Li, J. (2008) Hum. Gene Ther. 19:958-964, each of which reference is incorporated herein in its entirety by reference.

Dual AAV vector strategies to transfer of a large gene into target cells have been described, which rely on different mechanisms including, but not limited to, trans-splicing, including overlapping regions in the dual vectors, and a hybrid of the two (see, e.g., Tornabene and Trapani (2020) Human Gene Ther. 31:47 56; see also U.S. Pat. No. 8,236,557, each of which is incorporated herein by reference in its entirety).

A trans-splicing approach takes advantage of the ability of AAV ITR sequences to concatemerized to reconstitute full-length genomes, wherein each of two or more viral capsids respectively encapsulate one of two or more transfer plasmids, each of which transfer plasmid comprises a portion of the gene of interest. For example, in a dual vector approach, the two transfer plasmids may be designed as follows: the 5′-transfer plasmid comprises the promoter, the 5′ portion of the coding sequence of the gene of interest, and a splicing donor (SD) signal; the 3′-transfer plasmid comprises a splicing acceptor (SA) signal, the 3′ portion of the gene of interest, and the polyA signal. Upon tail-to-head ITR-mediated concatemerization of the two AAV genomes, the SD and SA signals will allow splicing of the recombined genome.

A large gene of interest is also split when taking an overlapping region approach. In the overlapping region approach, the 5′ and 3′ portions (and thus the 5′ transfer plasmid and 3′ transfer plasmid) share a recombinogenic sequence, e.g., region of homology, e.g., each portion comprises an overlapping sequence. The gene of interest is made whole in a targeted cell via homologous recombination mediated by the recombinogenic sequence, e.g., homology/overlapping region.

In a hybrid approach, the 5′-transfer plasmid and 3′-transfer plasmid each comprise a highly recombinogenic sequence, wherein the recombinogenic sequence is placed downstream of an SD signal of a 5′ portion of the coding sequence of the gene of interest and upstream of an SA signal of a 3′ portion of the coding sequence of the gene of interest. In this hybrid system, the gene of interest may be made whole either via ITR-mediated concatemerization and splicing and/or by homologous recombination.

Trans-splicing at the RNA or protein levels may also be utilized. In an RNA trans-splicing approach, two transfer plasmids may respectively encode for 5′ and 3′ fragments of the pre-mRNA of a large gene and share an intronic hybridization domain that can favor trans splicing, leading to joining of the two half-transcripts into an intact full-length mRNA.

Protein trans-splicing occurs post-translationally and is catalyzed by intervening proteins called split-inteins. Split-inteins are expressed as two independent polypeptides (N-intein and C-intein) at the extremities of two host proteins. The N-intein and C-intein polypeptides remain catalytically inactive until they encounter each other. Upon encountering each other, each intein precisely excises itself from the host protein while mediating ligation of the N- and C-host polypeptides via a peptide bond. Split-intein use has been used in AAV-based delivery of therapeutic genes of interest in muscle, liver, and retinal diseases. For example, on co-delivery of two halves of the mini-dystrophin cDNA fused to N- and C-intein coding sequences, efficient production of the two polypeptides was shown (see, e.g., Li et al. (2008) Hum Gene Ther 19:958-64).

The above dual vector approaches are well-known in the art. See, e.g., Tornabene and Trapani (2020), supra; U.S. Pat. No. 8,236,557, incorporated by reference in its entirety for all purposes. Thus, in some embodiments, a modified viral capsid described herein encapsulates a nucleotide of interest, wherein the nucleotide of interest comprises a portion of a gene of interest. In some embodiments, a nucleotide of interest comprising a portion of a gene of interest further comprises a splicing donor signal or a splicing acceptor signal and/or a recombinogenic sequence. In some embodiments, a nucleotide of interest comprising a portion of a gene of interest comprises an intronic hybridization domain encoding sequence. In some embodiments, a nucleotide of interest comprising a portion of a gene of interest comprises a N-intein or C-intein encoding sequence.

Design of the transfer plasmid/nucleotide of interest encompasses including one or more regulatory elements, e.g., promoter and/or enhancer elements, that can control expression of the gene of interest. Non-limiting examples of useful promoters include, e.g., cytomegalovirus (CMV)-promoter, the spleen focus forming virus (SFFV)-promoter, the elongation factor 1 alpha (EF1a)-promoter (the 1.2 kb EFIa-promoter or the 0.2 kb EFIa-promoter), the chimeric EF 1 a/IF4-promoter, and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, the CMV enhancer can be used in combination with the chicken β-actin promoter. In some embodiments, tissue specific regulatory elements, e.g., a tissue specific promoter and/or regulatory element may be used to drive the expression of the gene of interest. For example, the use of muscle-specific regulatory elements based on the muscle creatine kinase gene has been employed for muscle gene therapy treatments, such as Duchenne muscular dystrophy (DMD) and limb-girdle muscular dystrophy (LGMD). See, e.g., Salva, M. Z. et al. (2007) Mol. Ther. 15:320-329, incorporated herein in its entirety by reference. In some embodiments, a transfer plasmid and/or nucleotide of interest herein comprises an enhancer and/or promoter of muscle creatine kinase (MCK), wherein the enhancer and/or promoter of MCK drives expression of the gene of interest. In some embodiments, a transfer plasmid and/or nucleotide of interest herein comprises an enhancer and/or promoter element that recruits RNA Polymerase II, wherein the enhancer and/or promoter of MCK drives expression of the gene of interest. In some embodiments, a transfer plasmid and/or nucleotide of interest herein comprises an enhancer and/or promoter element that recruits RNA Polymerase III, wherein the enhancer and/or promoter of MCK drives expression of the gene of interest.

In some embodiments, bidirectional promoters vectors have also been employed for delivery of dual therapeutic gene cassettes. An example of this is the bidirectional chicken β-actin ubiquitous promoter that drives the simultaneous expression of the hexosaminidase α- and β-subunits of the HexA enzyme, the two respective genes involved in Tay-Sachs and Sandhoff diseases. Lahey, et al. (2020) Mol. Ther. 28:2150-2160, incorporated herein in its entirety by reference. In some embodiments, a transfer plasmid and/or nucleotide of interest herein comprises a bidirectional promoter, wherein the bidirectional promoter drives the expression of two different genes of interest

A variety of reporter genes (or detectable moieties) can be encapsidated in a multimeric structure comprising the modified viral capsid proteins described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting particles that employ the use of a reporter gene that encodes green fluorescent protein, however, persons of skill upon reading this disclosure will understand that the viral capsids described herein can be generated in the absence of a reporter gene or with any reporter gene known in the art.

A variety of therapeutic genes can also be encapsidated in a multimeric structure comprising the modified viral capsid proteins described herein, e.g., as part of a transfer particle. Non-limiting examples of a therapeutic gene include those that encode a toxin (e.g., a suicide gene), a therapeutic antibody or fragment thereof, antisense RNA, siRNA, shRNA, etc.

In some embodiments, modifications described herein may pertain to the association (e.g., display, operable linkage, binding) of a targeting ligand to a modified capsid protein and/or capsid comprising a modified capsid protein. In some embodiments, a targeting ligand can bind a surface protein expressed by a mammalian muscle cell, e.g., a protein that is expressed on the surface of a mammalian muscle cell, e.g., a mammalian muscle cell-specific surface protein.

In various embodiments, viral particles as described herein can be particularly suited for the targeted introduction of a nucleotide of interest specifically to a target cell since the viral capsid or viral capsid protein(s) can comprise a targeting ligand that binds a cell-specific surface protein of the target cell. In some embodiments, a viral capsid or viral capsid protein comprises a first member of a binding pair, associated with its cognate second member of the binding pair, and the second member is linked (e.g., fused to) a targeting ligand that binds a cell-specific surface protein. In some embodiments, the targeting ligand is operably linked to the second member, e.g., fused to the second member, optionally via a linker. In some embodiments, a targeting ligand may be a binding moiety, e.g., a natural ligand, antibody, a multispecific binding molecule, etc. In some embodiments, the targeting ligand is an antibody or portion thereof. In some embodiments, the targeting ligand is an antibody comprising a variable domain that binds a cell-specific surface protein on a target cell and a heavy chain constant domain. In some embodiments, the targeting ligand is an antibody comprising a variable domain that binds a cell-specific surface protein on a target cell and an IgG heavy chain constant domain. In some embodiments, the targeting ligand is an antibody comprising a variable domain that binds a cell-specific surface protein on a target cell and an IgG heavy chain constant domain, wherein the IgG heavy chain constant domain is operably linked, e.g., via a linker, to a protein (e.g., second member of a protein:protein binding pair) that forms an isopeptide covalent bond with the first member. In some embodiments, a capsid protein described herein comprises a first member comprising SpyTag operably linked to the viral capsid protein, and covalently linked to the SpyTag, a second member comprising SpyCatcher linked to a targeting ligand comprising an antibody variable domain and an IgG heavy chain domain, wherein SpyCatcher and the IgG heavy chain domain are linked via an amino acid linker.

Non-limiting examples of targeting ligands include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “targeting ligand,” as used herein. In non-limiting embodiments, an targeting ligand useful for retargeting viral capsids as described herein comprise comprises an scFv.

In some embodiments, a targeting ligand that binds a mammalian cell-specific surface protein may be associated with (e.g., displayed by, operably linked to, bound to) a modified AAV capsid protein and resulting AAV capsids according to indirect recombinatorial approaches, wherein the AAV capsid protein is modified to comprise a first member of a binding pair (e.g., a heterologous scaffold), and optionally wherein the first member of the binding pair is linked to (e.g., covalently or non-covalently bound to) a second cognate member of the binding pair (e.g., an adaptor), further optionally wherein the second cognate member of the binding pair is fused to the targeting ligand. Non-limiting and exemplary binding pairs are listed in Buning and Srivastava (2019) Mol. Ther. Methods Clin Dev 12:248-265.

In some embodiments, AAV particles comprising capsid proteins retargeted to a cell-specific surface protein of a target cell may be useful in methods of treating a tissue-related disorder, with the tissue comprising the target cell. Generally, such methods comprise administering to a subject suffering from or at risk for such tissue-related disorder a viral particle or pharmaceutical composition, wherein the viral particle comprises
(i) a viral capsid modified to comprise a first member of a protein:protein binding pair,
(ii) a second member of the protein:protein binding pair, wherein the second member of the protein:protein binding pair comprises a targeting ligand that binds a cell-specific surface protein that is expressed on the surface of a target cell,
   wherein the first member of the protein:protein binding pair and the second member of the protein:protein binding pair are associated to direct the tropism of the viral capsid to the muscle cell in the subject thereof, and
(iii) a nucleotide of interest encapsidated within the viral capsid.

Recombinant AAV particles comprising capsid proteins retargeted to a cell surface protein that allows the viral particles to bind a cell-specific surface protein can be useful for delivering a delivering a nucleotide of interest, e.g., a reporter gene or a therapeutic gene, to a target cell. Non-limiting tissues that may be targeted by a modified viral capsid protein for insertion of a nucleotide of interest, e.g., a reporter gene or a therapeutic gene, include adipose tissue, blood/bone marrow, bone/cartilage/joint, brain/spinal cord/cns/bbb, breast, colon, esophagus, eye, heart, kidney, liver, lung/bronchus, lymph node, ovary, pancreas, pbmc, peripheral nervous system, placenta, prostate, rectum, skeletal muscle, skin, small intestine, spleen, stomach, testis, and uterus. Non-limiting cell types that may be targeted by a modified viral capsid protein for insertion of a nucleotide of interest, e.g., a reporter gene or a therapeutic gene, include endothelial cells, neurons (all types), oligodendrocytes (and precursors), pericytes, meninges/leptomeningeal cells, arachnoid barrier cells, peripheral glia, astrocytes, glia, schwann cells, ependymal cells, microglia, rod photoreceptor cells, muller glia cells, bipolar cells, cone photoreceptor cells, endothelial cells, cornea, sclera, optic nerve, pupillary sphincter, skeletal myocytes, fibroblasts, endothelial cells, macrophages, satellite cells, Adipocytes, fibroblasts, T-cells, Macrophages, B-cells, Dendritic cells, T-cells, B-cells, Macrophages, erythroid cells, plasmid cells, dendritic cells, glandular cells, T-cells, fibroblasts, macrophages, endothelial cells, myoepithelial cells, Adipocytes, Basal respiratory cells, respiratory cilliated cells, club cells, smooth muscle cells, ionocytes, macrophages, alveolar cells (type 1 & 2), T-cells, enothelial cells, distal enterocytes, intestinal goblet cells, undifferentiated cells, T-cells, paneth cells, B-cells, enteroednocrine cells, glandular and luminal cells, endometrial stromal cells, endothelial cells, smooth muscle cells, T-cells, macrophages, fibroblasts, squamous epithelial cells, endothelial cells, smooth muscle cells, macrophages, plasma cells, T-cells, cardiomyocytes, endothelial cells, fibroblasts, macrophages, T-cells, B-cells, Dendritic cells, proximal tubular cells, T-cells, macrophages, collecting duct cells, B-cells, glomeruli, fibroblasts, hepatocytes, B-cells, erythroid cells, B-cells, T-cells, granulosa cells, fibroblasts, smooth muscle cells, macrophages, T-cells, theca cells, fibroblasts, ductal cells, pancreatic endocrine cells, smooth muscle cells, endothelial cells, macrophages, exocrine glandular cells, monocytes, cytotrophoblasts, extravillous trophoblasts, fibroblasts, hofbauer cells, endothelial cells, basal prostatic cells, prostatic glandular cells, urothelial cells, endothelial cells, fibroblasts, smooth muscle cells, macrophages, T-cells, undifferentiated cells, intestinal goblet cells, paneth cells, distal enterocytes, enteroednocrine cells, langerhans cells, fibroblasts, endothelial cells, basal keratinocytes, suprabasal keratinocytes, T-cells, smooth muscle cells, melanocytes, monocytes, T-cells, NK-cells, dendritic cells, proximal enterocytes, undifferentiated cells, intestinal goblet cells, paneth cells, B-cells, T-cells, plasma cells, macrophages, B-cells, T-cells, gastric mucus-secreting cells, plasma cells, fibroblasts, macrophages, leydig cells, late spermatids, spermatogonia, early spermatids, macrophages, spermatocytes, peritubular cells, sertoli cells, endothelial cells, motor neurons, sensory neurons, schwann cells, dorsal root ganglion, chondrocytes, chondroblasts, mesenchymal cells, osteoblasts and osteoclasts.

A variety of therapeutic genes can also be encapsidated in a multimeric structure comprising the capsid proteins retargeted to a cell surface protein that allows the viral particles to bind transferrin receptor 1, e.g., as part of a transfer particle. Non-limiting examples of a therapeutic gene include those that encode a toxin (e.g., a suicide gene), a therapeutic antibody or fragment thereof, antisense RNA, siRNA, shRNA, etc. As a non-limiting example, diseases and the genes which may be a nucleotide of interest and/or for which the reduction of which may be therapeutic that may be suitable for treatment using such viral particles include lysosomal storage diseases with central nervous system manifestations (neuropathic); Fabry disease, Gaucher disease, Pompe disease and many more (Genes encoding the deficient enzyme); Parkinson's/synucleinopathies (Alpha-synuclein SNCA); Parkinson's (Lrrk2); Parkinson's/Gaucher disease (GBA); FTD (Progranulin); Alzheimer's/tauopathies (3R/4R Tau, 4R-specific Tau); Alzheimer's Disease (APP/Abeta, Trem2, ApoE); ALS/FTD/TDP-43 proteinopathies (SOD1, C9orf72, TDP43, SUPT4H1, PUMA, ELOVL1, RPS25, PSMB5); Huntington's disease (HTT); Transthyretin amyloidosis (TTR); Friedreich's Ataxia (FXN); and pain (Nav1.7, Nav1.9, TrpA1, TRPM8, TrkA, FXYD2, AAK1, ASIC3, ASIC1, NGF). In some embodiments, the disease can be Tangier disease, Intellectual developmental disorder with poor growth and with or without seizures or ataxia, Hypermethioninemia due to adenosine kinase deficiency, Aspartylglycosaminuria, Fructose intolerance, hereditary, MEDNIK syndrome, Spastic paraplegia 50, autosomal recessive, Sea-blue histiocyte disease, Adenine phosphoribosyltransferase deficiency, Maroteaux-Lamy syndrome (mucopolysaccharidosis type VI, Mucopolysaccharidosis, type X, Spinocerebellar ataxia, autosomal recessive 31, Cutis laxa, autosomal recessive, type IID, Farber disease, Hermansky-Pudlak syndrome 9, Ceroid lipofuscinosis, neuronal, 6A, Ceroid lipofuscinosis, neuronal, 6B (Kufs type), Ceroid lipofuscinosis, neuronal, 8, Ceroid lipofuscinosis, neuronal, 8, Northern epilepsy variant, Galactosialidosis, cystinosis, Haim-Munk syndrome, Papillon Lefevre Syndrome, Ceroid lipofuscinosis, neuronal, 10, Pycnodysostosis, Imerslund-Grasbeck syndrome 1, Proteinuria, chronic benign], WHIM syndrome 2, Orthostatic hypotension 2, 5-fluorouracil toxicity, Dihydropyrimidine dehydrogenase deficiency, Cone-rod dystrophy 21, Vici syndrome, Arthrogryposis multiplex congenita 2, neurogenic type, Fucosidosis, Cataract 18, autosomal recessive, Pompe disease, Mucopolysaccharidosis IV, Gaucher disease, Fabry disease, Mucolipidosis II alpha/beta, Mucolipidosis III alpha/beta, Mucolipidosis III gamma, Mucopolysaccharidosis Type VII, Tay Sachs Disease, Sandhoff disease, infantile, juvenile, and adult forms, Mucopolysaccharidosis type IIIC (Sanfilippo C), Retinitis pigmentosa 73, Hermansky-Pudlak syndrome 6, Mucopolysaccharidosis I, Mucopolysaccharidosis II, Spastic paraplegia, optic atrophy, and neuropathy, Lysosomal acid lipase deficiency, Danon disease, Immunodeficiency 52, Leydig cell hypoplasia with hypergonadotropic hypogonadism, Leydig cell hypoplasia with pseudohermaphroditism, Luteinizing hormone resistance, female, Immunodeficiency, common variable, 8, with autoimmunity, Keratosis pilaris atrophicans, Chediak-Higashi syndrome, Alpha-Mannosidosis, Spondyloepiphyseal Dysplasia, Kondo-Fu Type, Mucolipidosis IV, Ceroid lipofuscinosis, neuronal, 7, Macular dystrophy with central cone involvement, Megalencephalic leukoencephalopathy with subcortical cysts, Myeloperoxidase deficiency, Deafness, autosomal recessive 2, Usher syndrome, type 1B, Kanzaki disease, Schindler disease, type I, Schindler disease, type III, Niemann-Pick disease, type C1, Niemann-Pick disease, type D, Niemann-pick disease, type C2, Spastic paraplegia 45, autosomal recessive, Sialidosis, Parkinson disease 6, early onset, Osteopetrosis, autosomal recessive 6, Hemophagocytic lymphohistiocytosis, familial, 2, Epilepsy, progressive myoclonic 4, with or without renal failure, Mucopolysaccharidosis type IIIA (Sanfilippo A), Neurodevelopmental disorder with cardiomyopathy, spasticity, and brain abnormalities, Histiocytosis-lymphadenopathy plus syndrome, Niemann-Pick disease, type A/B, acid sphingomyelinase deficiency, Congenital disorder of glycosylation, type IIn, Spinocerebellar ataxia, autosomal recessive 20, Amyotrophic lateral sclerosis 5, juvenile, Charcot-Marie-Tooth disease, axonal, type 2X, Spastic paraplegia 11, autosomal recessive, Warburg micro syndrome 4, Dystonia 32, Leukodystrophy, hypomyelinating, 12, Choreoacanthocytosis, Arthrogryposis, renal dysfunction, and cholestasis 1, Pontocerebellar hypoplasia, type 13, Pontocerebellar hypoplasia, type 2E, Neurodevelopmental disorder with spastic quadriplegia and brain abnormalities with or without seizures, Cerebellar ataxia, mental retardation, and dysequilibrium syndrome 2, Hydrocephalus, congenital, 3, with brain anomalies, Xanthinuria, type I, Spastic paraplegia 15, autosomal recessive, Intellectual disability and myopathy syndrome, Muscular dystrophy, limb-girdle, autosomal recessive 25, Neurodevelopmental disorder with seizures and nonepileptic hyperkinetic movements, Cerebellar atrophy with seizures and variable developmental delay, Ventricular tachycardia, catecholaminergic polymorphic, 2, Lipodystrophy, congenital generalized, type 3, Arrhythmogenic right ventricular dysplasia 11, Arrhythmogenic right ventricular dysplasia 11 with mild palmoplantar keratoderma and woolly hair, Cardiomyopathy, dilated, with woolly hair and keratoderma, Epidermolysis bullosa, lethal acantholytic, Skin fragility-woolly hair syndrome, Congenital heart defects, multiple types, 5, Hemolytic anemia due to glutathione peroxidase deficiency, Naxos disease, Jervell and Lange-Nielsen syndrome 2, Myopathy, myofibrillar, 12, infantile-onset, with cardiomyopathy, Cardiomyopathy, hypertrophic, 8, Nephrotic syndrome, type 22, Developmental and epileptic encephalopathy 52, Dicarboxylic aminoaciduria, Lichtenstein-Knorr syndrome, Hypogonadotropic hypogonadism 11 with or without anosmia, Segawa syndrome, recessive, Intellectual developmental disorder with poor growth and with or without seizures or ataxia, Spondyloepimetaphyseal dysplasia, aggrecan type, Neurodevelopmental disorder with hypotonia, microcephaly, and seizures, Microcephaly 16, primary, autosomal recessive, Spinocerebellar ataxia, autosomal recessive 31, Acromesomelic dysplasia 3, Elsahy-Waters syndrome, Ceroid lipofuscinosis, neuronal, 8, Ceroid lipofuscinosis, neuronal, 8, Northern epilepsy variant, Pitt-Hopkins like syndrome 1, Gaze palsy, familial horizontal, with progressive scoliosis, 2, Short-rib thoracic dysplasia 3 with or without polydactyly, Bleeding disorder, platelet-type, 22, Macrocephaly, dysmorphic facies, and psychomotor retardation, Charcot-Marie-Tooth disease, axonal, type 2S, Neuronopathy, distal hereditary motor, type VI, SESAME syndrome, Goldberg-Shprintzen megacolon syndrome, Obesity, morbid, due to leptin deficiency, Spastic paraplegia 75, autosomal recessive, Hypogonadotropic hypogonadism 27 without anosmia, Seckel syndrome 7, Pitt-Hopkins-like syndrome 2, Oxoglutarate dehydrogenase deficiency, Myopathy, congenital, progressive, with scoliosis, Epilepsy, progressive myoclonic, 10, Lissencephaly 2 (Norman-Roberts type), Thyroid hormone metabolism, abnormal, Thyroid hormone metabolism, abnormal, 1, Neuropathy, hereditary motor and sensory, type VIB, Pontocerebellar hypoplasia, type 1E, Amyotrophic lateral sclerosis 5, juvenile, Charcot-Marie-Tooth disease, axonal, type 2X, Spastic paraplegia 11, autosomal recessive, Netherton syndrome, Joubert syndrome 13, Microphthalmia, syndromic 11, Osteogenesis imperfecta, type XV, Intellectual developmental disorder with poor growth and with or without seizures or ataxia, Microcornea, myopic chorioretinal atrophy, and telecanthus, Microphthalmia, isolated 8, Fructose intolerance, hereditary, Alstrom syndrome, Sea-blue histiocyte disease, Mucopolysaccharidosis, type X, Cutis laxa, autosomal recessive, type IID, Bardet-Biedl syndrome 4, Bardet-Biedl syndrome 7, Acromesomelic dysplasia 3, Cone-rod synaptic disorder, congenital nonprogressive, Joubert syndrome 5, Leber congenital amaurosis 10, Meckel syndrome 4, Senior-Loken syndrome 6, Complement factor D deficiency, Ceroid lipofuscinosis, neuronal, 8, Ceroid lipofuscinosis, neuronal, 8, Northern epilepsy variant, Achromatopsia 2, Focal segmental glomerulosclerosis 9, Ventriculomegaly with cystic kidney disease, Leber congenital amaurosis 7, Cataract 22, Galactosialidosis, Ceroid lipofuscinosis, neuronal, 10, Pycnodysostosis, Imerslund-Grasbeck syndrome 1, Proteinuria, chronic benign], WHIM syndrome 2, Cone-rod dystrophy 21, Vici syndrome, Bleeding disorder, platelet-type, 22, Anterior segment dysgenesis 2, multiple subtypes, Fucosidosis, Cataract 18, autosomal recessive, Ectodermal dysplasia/short stature syndrome, Night blindness, congenital stationary (complete), 1B, autosomal recessive, Growth hormone deficiency with pituitary anomalies, Pituitary hormone deficiency, combined, 5, Septooptic dysplasia, Sandhoff disease, infantile, juvenile, and adult forms, Mucopolysaccharidosis type IIIC (Sanfilippo C), Retinitis pigmentosa 73, Hermansky-Pudlak syndrome 6, Cerebellar atrophy, developmental delay, and seizures, Cornea plana 2, autosomal recessive, Spastic paraplegia, optic atrophy, and neuropathy, Poretti-Boltshauser syndrome, Cortical malformations, occipital, Leydig cell hypoplasia with hypergonadotropic hypogonadism, Leydig cell hypoplasia with pseudohermaphroditism, Luteinizing hormone resistance, female, Immunodeficiency, common variable, 8, with autoimmunity, Microphthalmia/coloboma and skeletal dysplasia syndrome, Charcot-Marie-Tooth disease, axonal, type 2A2B, Neurodevelopmental disorder with progressive microcephaly, spasticity, and brain abnormalities, Ceroid lipofuscinosis, neuronal, 7, Macular dystrophy with central cone involvement, Megalencephalic leukoencephalopathy with subcortical cysts, Myeloperoxidase deficiency, Kanzaki disease, Schindler disease, type I, Schindler disease, type III, Niemann-Pick disease, type C1, Niemann-Pick disease, type D, Niemann-pick disease, type C2, Joubert syndrome 4, Nephronophthisis 1, juvenile, Senior-Loken syndrome-1, Microcephalic osteodysplastic primordial dwarfism, type II, Retinitis pigmentosa 43, Retinitis pigmentosa-40, Cataract 11, multiple types, Cataract 11, syndromic, autosomal recessive, Osteopetrosis, autosomal recessive 6, Hemophagocytic lymphohistiocytosis, familial, 2, Microphthalmia, isolated 6, Anterior segment dysgenesis 7, with sclerocornea, Martsolf syndrome 2, Warburg micro syndrome 1, Retinal dystrophy, iris coloboma, and comedogenic acne syndrome, Leber congenital amaurosis 12, COACH syndrome 3, Joubert syndrome 7, Meckel syndrome 5, Intellectual developmental disorder and retinitis pigmentosa, Epilepsy, progressive myoclonic 4, with or without renal failure, Mucopolysaccharidosis type IIIA (Sanfilippo A), Dicarboxylic aminoaciduria, Histiocytosis-lymphadenopathy plus syndrome, Congenital disorder of glycosylation, type IIn, Heart and brain malformation syndrome, Microphthalmia with limb anomalies, Spinocerebellar ataxia, autosomal recessive 20, Deafness, autosomal recessive 115, Warburg micro syndrome 4, Segawa syndrome, recessive, Night blindness, congenital stationary (complete), 1C, autosomal recessive, Focal facial dermal dysplasia 3, Setleis type, Deafness, autosomal recessive 18A, Usher syndrome, type 1C, Microphthalmia, syndromic 11, Dystonia 32, Leukodystrophy, hypomyelinating, 12, Choreoacanthocytosis, Arthrogryposis, renal dysfunction, and cholestasis 1, Neurodevelopmental disorder with spastic quadriplegia and brain abnormalities with or without seizures, Cerebellar ataxia, mental retardation, and dysequilibrium syndrome 2, Hydrocephalus, congenital, 3, with brain anomalies, Spastic paraplegia 15, autosomal recessive, Deafness, autosomal recessive 44, Microcephaly 5, primary, autosomal recessive, Spinocerebellar ataxia, autosomal recessive 31, Bardet-Biedl syndrome 2, Retinitis pigmentosa 74, Bardet-Biedl syndrome 4, Pitt-Hopkins like syndrome 1, Joubert syndrome 17, Orofaciodigital syndrome VI, Oculocutaneous albinism, type VIII, Hermansky-Pudlak syndrome 7, Short-rib thoracic dysplasia 3 with or without polydactyly, Macrocephaly, dysmorphic facies, and psychomotor retardation, Growth hormone deficiency with pituitary anomalies, Pituitary hormone deficiency, combined, 5, Septooptic dysplasia, Intellectual developmental disorder, autosomal recessive 57, Neurodevelopmental disorder with progressive microcephaly, spasticity, and brain abnormalities, Hypogonadotropic hypogonadism 27 without anosmia, Pitt-Hopkins-like syndrome 2, Oxoglutarate dehydrogenase deficiency, Microcephalic osteodysplastic primordial dwarfism, type II, Intellectual developmental disorder with paroxysmal dyskinesia or seizures, Neurodevelopmental disorder with dysmorphic features, spasticity, and brain abnormalities, Developmental and epileptic encephalopathy 12, Martsolf syndrome 2, Warburg micro syndrome 1, Lissencephaly 2 (Norman-Roberts type), COACH syndrome 3, Joubert syndrome 7, Meckel syndrome 5, Thyroid hormone metabolism, abnormal, Thyroid hormone metabolism, abnormal, 1, Neuropathy, hereditary motor and sensory, type VIB, Pontocerebellar hypoplasia, type 1E, Spinocerebellar ataxia, autosomal recessive 14, Microcephaly-capillary malformation syndrome, Neurodevelopmental disorder, nonprogressive, with spasticity and transient opisthotonus, Intellectual developmental disorder, autosomal recessive 13, Microcephaly 2, primary, autosomal recessive, with or without cortical malformations, Osteogenesis imperfecta, type XV, Diarrhea 9, Neurodevelopmental disorder with hypotonia, microcephaly, and seizures, Ceroid lipofuscinosis, neuronal, 8, Ceroid lipofuscinosis, neuronal, 8, Northern epilepsy variant, Nephrotic syndrome, type 24, Gaze palsy, familial horizontal, with progressive scoliosis, 2, Short-rib thoracic dysplasia 3 with or without polydactyly, Charcot-Marie-Tooth disease, axonal, type 2S, Neuronopathy, distal hereditary motor, type VI, Myopathy, congenital, progressive, with scoliosis, Neu-Laxova syndrome 1, Phosphoglycerate dehydrogenase deficiency, Carpenter syndrome, Lissencephaly 2 (Norman-Roberts type), Joubert syndrome 13, Cerebellar hypoplasia and mental retardation with or without quadrupedal locomotion 1, Osteogenesis imperfecta, type XV, Visceral neuropathy, familial, 2, autosomal recessive, Arthrogryposis multiplex congenita 1, neurogenic, with myelin defect, Multicentric osteolysis, nodulosis, and arthropathy, Charcot-Marie-Tooth disease, type 4D, Hypogonadotropic hypogonadism 27 without anosmia, Charcot-Marie-Tooth disease, type 4C, Neuropathy, hereditary motor and sensory, type VIB, Pontocerebellar hypoplasia, type 1E, Encephalopathy, progressive, with amyotrophy and optic atrophy, Hypoparathyroidism-retardation-dysmorphism syndrome, Kenny-Caffey syndrome, type 1, Brody myopathy, Muscular dystrophy, limb-girdle, autosomal recessive 25, Lipodystrophy, congenital generalized, type 3, Myasthenic syndrome, congenital, 1B, fast-channel, Myasthenic syndrome, congenital, 3B, fast-channel, Myasthenic syndrome, congenital, 3C, associated with acetylcholine receptor deficiency, Ceroid lipofuscinosis, neuronal, 8, Ceroid lipofuscinosis, neuronal, 8, Northern epilepsy variant, Spondylocarpotarsal synostosis syndrome, Hemolytic anemia due to glutathione peroxidase deficiency, Gillespie syndrome, Nemaline myopathy 10, Myopathy, congenital, progressive, with scoliosis, Myasthenic syndrome, congenital, 16, Dystonia, dopa-responsive, due to sepiapterin reductase deficiency, Split-hand/foot malformation 6, Spondyloepimetaphyseal dysplasia, aggrecan type, Bardet-Biedl syndrome 2, Retinitis pigmentosa 74, Acromesomelic dysplasia 3, Osteochondrodysplasia, brachydactyly, and overlapping malformed digits, Temtamy preaxial brachydactyly syndrome, Fibrochondrogenesis 1, Deafness, autosomal recessive 53, Fibrochondrogenesis 2, Otospondylomegaepiphyseal dysplasia, autosomal recessive, Steel syndrome, Pycnodysostosis, Spondyloepimetaphyseal dysplasia, Shohat type, Acromesomelic dysplasia 2A, Acromesomelic dysplasia 2B, Acromesomelic dysplasia 2C, Hunter-Thompson type, Brachydactyly, type A1, C, Leber congenital amaurosis 17, Short-rib thoracic dysplasia 2 with or without polydactyly, Obesity, morbid, due to leptin deficiency, Neurodevelopmental disorder with epilepsy and hypoplasia of the corpus callosum, Myopathy, congenital, progressive, with scoliosis, Rhizomelic limb shortening with dysmorphic features, Short stature, onychodysplasia, facial dysmorphism, and hypotrichosis, Hypoparathyroidism, familial isolated 1, Robinow syndrome, autosomal recessive, Spondyloepimetaphyseal dysplasia, Krakow type, Congenital disorder of glycosylation, type IIn, Waardenburg syndrome, type 2D, Ehlers-Danlos syndrome, cardiac valvular type, Steel syndrome, Factor VII deficiency, Short-rib thoracic dysplasia 2 with or without polydactyly, Keratosis pilaris atrophicans, Short stature, onychodysplasia, facial dysmorphism, and hypotrichosis, Nephrotic syndrome, type 14, Waardenburg syndrome, type 2D, Cone-rod dystrophy 3, Fundus flavimaculatus, Retinal dystrophy, early-onset severe, Retinitis pigmentosa 19, Stargardt disease 1, Lethal congenital contracture syndrome 8, Nephronophthisis 16, Distal renal tubular acidosis 3, with or without sensorineural hearing loss, Distal renal tubular acidosis 2 with progressive sensorineural hearing loss, Bardet-Biedl syndrome 2, Retinitis pigmentosa 74, Deafness, autosomal recessive 93, Cone-rod synaptic disorder, congenital nonprogressive, Joubert syndrome 5, Leber congenital amaurosis 10, Meckel syndrome 4, Senior-Loken syndrome 6, Bartter syndrome, type 3, Deafness, autosomal recessive 103, Ceroid lipofuscinosis, neuronal, 6A, Ceroid lipofuscinosis, neuronal, 6B (Kufs type), Ceroid lipofuscinosis, neuronal, 8, Ceroid lipofuscinosis, neuronal, 8, Northern epilepsy variant, Retinitis pigmentosa 61, Usher syndrome, type 3A, Achromatopsia 2, Fibrochondrogenesis 1, Joubert syndrome 17, Orofaciodigital syndrome VI, Leber congenital amaurosis 7, Cataract 22, Chronic granulomatous disease 4, autosomal recessive, Cone-rod dystrophy 21, Short-rib thoracic dysplasia 3 with or without polydactyly, Leber congenital amaurosis 17, Hyperekplexia 2, Night blindness, congenital stationary (complete), 1B, autosomal recessive, Immunodeficiency-centromeric instability-facial anomalies syndrome 4, Muscular dystrophy, congenital, with cataracts and intellectual disability, Renal hypodysplasia/aplasia 1, SESAME syndrome, Cerebellar atrophy, developmental delay, and seizures, Pseudohypoaldosteronism, type IID, Cortical malformations, occipital, Leber congenital amaurosis 14, Retinal dystrophy, early-onset severe, Retinitis pigmentosa, juvenile, Night blindness, congenital stationary (complete), 1F, autosomal recessive, Metaphyseal anadysplasia 2, Deafness, autosomal recessive 30, Deafness, autosomal recessive 2, Usher syndrome, type 1B, Short-rib thoracic dysplasia 6 with or without polydactyly, Meckel syndrome 7, Nephronophthisis 3, Renal-hepatic-pancreatic dysplasia 1, Boudin-Mortier syndrome, Microcephalic osteodysplastic primordial dwarfism, type II, Retinitis pigmentosa 43, Retinitis pigmentosa-40, Leber congenital amaurosis 12, Bothnia retinal dystrophy, Newfoundland rod-cone dystrophy, COACH syndrome 3, Joubert syndrome 7, Meckel syndrome 5, Nephrotic syndrome, type 14, Leber congenital amaurosis 3, Retinitis pigmentosa 94, variable age at onset, autosomal recessive, Immunodeficiency 31B, mycobacterial and viral infections, autosomal recessive, Corneal dystrophy, gelatinous drop-like, Segawa syndrome, recessive, COACH syndrome 1, Joubert syndrome 6, Meckel syndrome 3, Nephronophthisis 11, RHYNS syndrome, Night blindness, congenital stationary (complete), 1C, autosomal recessive, Diarrhea 9, Nephronophthisis-like nephropathy 1, Combined oxidative phosphorylation deficiency 8, Leukoencephalopathy, progressive, with ovarian failure, 2-methylbutyrylglycinuria, Alpha-methylacetoacetic aciduria, Aicardi-Goutieres syndrome 6, Neurodevelopmental disorder with hypotonia, microcephaly, and seizures, Deafness, autosomal recessive 44, Obesity, susceptibility to, BMIQ19}, Lethal congenital contracture syndrome 8, Hypermethioninemia due to adenosine kinase deficiency, Neurodegeneration, childhood-onset, stress-induced, with variable ataxia and seizures, Alopecia-intellectual disability syndrome 1, Immunodeficiency with hyper-IgM, type 2, Leukodystrophy, hypomyelinating, 3, Autoimmune polyendocrinopathy syndrome, type I, with or without reversible metaphyseal dysplasia, Spermatogenic failure 27, Glycogen storage disease XII, Fructose intolerance, hereditary, Intellectual developmental disorder, autosomal recessive 71, Myopathy due to myoadenylate deaminase deficiency, Pontocerebellar hypoplasia, type 9, Spastic paraplegia 63, Ferguson-Bonni neurodevelopmental syndrome, Scott syndrome, Spastic paraplegia 48, autosomal recessive, Adenine phosphoribosyltransferase deficiency, Ataxia, early-onset, with oculomotor apraxia and hypoalbuminemia, Spinal muscular atrophy with congenital bone fractures 2, Cutis laxa, autosomal recessive, type IID, Distal renal tubular acidosis 2 with progressive sensorineural hearing loss, Muscular dystrophy-dystroglycanopathy (congenital with brain and eye anomalies, type A, 11, Bile acid conjugation defect 1, Agammaglobulinemia 4, Hermansky-Pudlak syndrome 9, Acromesomelic dysplasia 3, Erythrocytosis, familial, 8, Fanconi anemia, complementation group J, Desbuquois dysplasia 1, Epiphyseal dysplasia, multiple, 7, Immunodeficiency 11A, Immunodeficiency, common variable, 3, Lymphoproliferative syndrome 2, Immunodeficiency with hyper-IgM, type 3, Deafness, autosomal recessive 32, with or without immotile sperm, Microcephaly 12, primary, autosomal recessive, Microcephaly 13, primary, autosomal recessive, Nephronophthisis 15, Complement factor B deficiency, Cocoon syndrome, Popliteal pterygium syndrome, Bartsocas-Papas type 2, Cold-induced sweating syndrome 2, Leukodystrophy, hypomyelinating, 20, Pitt-Hopkins like syndrome 1, Neurodegeneration with brain iron accumulation 6, Pontocerebellar hypoplasia, type 12, Carbamoylphosphate synthetase I deficiency, Surfactant metabolism dysfunction, pulmonary, 5, Neutropenia, severe congenital, 7, autosomal recessive, Joubert syndrome 21, Cerebroretinal microangiopathy with calcifications and cysts, Microcephaly, facial dysmorphism, renal agenesis, and ambiguous genitalia syndrome, WHIM syndrome 2, Aromatase deficiency, Bile acid synthesis defect, congenital, 3, Spastic paraplegia 5A, autosomal recessive, Developmental and epileptic encephalopathy 86, Leukoencephalopathy with brain stem and spinal cord involvement and lactate elevation, Oculocutaneous albinism, type VIII, Pentosuria], Aromatic L-amino acid decarboxylase deficiency, Mitochondrial DNA depletion syndrome 3 (hepatocerebral type), Progressive external ophthalmoplegia with mitochondrial DNA deletions, autosomal recessive 4, Miller syndrome, Pyruvate dehydrogenase E2 deficiency, Systemic lupus erythematosus 16, Immunodeficiency-centromeric instability-facial anomalies syndrome 1, Immunodeficiency 40, Congenital disorder of glycosylation, type Ie, 5-fluorouracil toxicity, Dihydropyrimidine dehydrogenase deficiency, Intellectual developmental disorder, autosomal recessive 50, Combined oxidative phosphorylation deficiency 17, Dysautonomia, familial, Spastic paraplegia 64, autosomal recessive, Bleeding disorder, platelet-type, 22, Eosinophil peroxidase deficiency], Visceral neuropathy, familial, 2, autosomal recessive, Fanconi anemia, complementation group Q, XFE progeroid syndrome, Xeroderma pigmentosum, group F, Xeroderma pigmentosum, type F/Cockayne syndrome, Cerebrooculofacioskeletal syndrome 3, Xeroderma pigmentosum, group G, Xeroderma pigmentosum, group G/Cockayne syndrome, Cockayne syndrome, type A, UV-sensitive syndrome 2, Deafness, autosomal recessive 109, Pontocerebellar hypoplasia, type 1F, Dysprothrombinemia, Hypoprothrombinemia, Immunodeficiency 90 with encephalopathy, functional hyposplenia, and hepatic dysfunction, Raine syndrome, Fanconi anemia, complementation group D2, Fanconi anemia, complementation group I, Fanconi anemia, complementation group L, Peroxisomal fatty acyl-CoA reductase 1 disorder, Combined oxidative phosphorylation deficiency 14, Spastic paraplegia 77, autosomal recessive, Rajab interstitial lung disease with brain calcifications 2, Combined oxidative phosphorylation deficiency 44, Parkinson disease 15, autosomal recessive, Leukocyte adhesion deficiency, type III, Siddiqi syndrome, Anterior segment dysgenesis 2, multiple subtypes, T-cell immunodeficiency, congenital alopecia, and nail dystrophy, Combined oxidative phosphorylation deficiency 41, Glutaricaciduria, type I, Diabetes mellitus, permanent neonatal 1, Bleeding disorder, platelet-type, 17, Nonaka myopathy, Hypertriglyceridemia, transient infantile, Chudley-Mccullough syndrome, Jaberi-Elahi syndrome, Combined oxidative phosphorylation deficiency 23, Vertebral, cardiac, renal, and limb defects syndrome 1, T-cell lymphoma, subcutaneous panniculitis-like, Immunodeficiency-centromeric instability-facial anomalies syndrome 4, Hemochromatosis, type 2A, Heme oxygenase-1 deficiency, Dystonia 2, torsion, autosomal recessive, D-bifunctional protein deficiency, Perrault syndrome 1, Premature ovarian failure 19, Immunodeficiency 27A, mycobacteriosis, AR, Charcot-Marie-Tooth disease, axonal, type 2S, Neuronopathy, distal hereditary motor, type VI, Immunodeficiency 15B, Immunodeficiency 29, mycobacteriosis, Immunodeficiency 30, Candidiasis, familial, 9, Immunodeficiency, common variable, 11, Immunodeficiency 56, Immunodeficiency 41 with lymphoproliferation and autoimmunity, Immunodeficiency 63 with lymphoproliferation and autoimmunity, Immunodeficiency 39, Immunodeficiency 32B, monocyte and dendritic cell deficiency, autosomal recessive, Autoimmune disease, multisystem, with facial dysmorphism, Lymphoproliferative syndrome 1, Muscular dystrophy, limb-girdle, autosomal recessive 27, SCID, autosomal recessive, T-negative/B-positive type, Basal ganglia calcification, idiopathic, 8, autosomal recessive, Hemorrhagic destruction of the brain, subependymal calcification, and cataracts, Hydroxykynureninuria, Vertebral, cardiac, renal, and limb defects syndrome 2, Immunodeficiency 52, Immunodeficiency 81, Obesity, morbid, due to leptin deficiency, Obesity, morbid, due to leptin receptor deficiency, Lipodystrophy, familial partial, type 6, Chediak-Higashi syndrome, 3-Methylcrotonyl-CoA carboxylase 2 deficiency, Basel-Vanagait-Smirin-Yosef syndrome, Intellectual developmental disorder, autosomal recessive 72, Mitochondrial DNA depletion syndrome 11, Mismatch repair cancer syndrome 1, Metaphyseal anadysplasia 2, Xanthinuria, type II, Molybdenum cofactor deficiency B, Thrombocytopenia, anemia, and myelofibrosis, Deafness, autosomal recessive 111, Familial adenomatous polyposis 4, Premature ovarian failure 13, Vertebral, cardiac, renal, and limb defects syndrome 3, Encephalopathy, progressive, early-onset, with brain edema and/or leukoencephalopathy, Infantile liver failure syndrome 2, Short stature, optic nerve atrophy, and Pelger-Huet anomaly, Microcephaly 22, primary, autosomal recessive, Chronic granulomatous disease 1, autosomal recessive, Chronic granulomatous disease 2, autosomal recessive, Charcot-Marie-Tooth disease, type 4D, Mitochondrial complex I deficiency, nuclear type 22, Mitochondrial complex I deficiency, nuclear type 37, Mitochondrial complex I deficiency, nuclear type 32, Mitochondrial complex I deficiency, nuclear type 24, Mitochondrial complex I deficiency, nuclear type 5, Dyskeratosis congenita, autosomal recessive 2, Glucocorticoid deficiency 4, with or without mineralocorticoid deficiency, Acromesomelic dysplasia 1, Maroteaux type, Boudin-Mortier syndrome, Spastic paraplegia 45, autosomal recessive, Insensitivity to pain, congenital, with anhidrosis, Striatonigral degeneration, infantile, Nephrotic syndrome, type 17, Oxoglutarate dehydrogenase deficiency, Hyperphenylalaninemia, non-PKU mild], Phenylketonuria, Parkinson disease 7, autosomal recessive early-onset, Myopathy, congenital, progressive, with scoliosis, Intellectual developmental disorder with paroxysmal dyskinesia or seizures, Lacticacidemia due to PDX1 deficiency, Pancreatic agenesis 1, Neuropathy, hereditary motor and sensory, type VIC, with optic atrophy, Glycogen storage disease VII, Immunodeficiency 23, Rhizomelic limb shortening with dysmorphic features, Developmental and epileptic encephalopathy 12, Osteopetrosis, autosomal recessive 6, Short stature, onychodysplasia, facial dysmorphism, and hypotrichosis, Mitochondrial DNA depletion syndrome 16 (hepatic type), Mitochondrial DNA depletion syndrome 16B (neuroophthalmic type), Hemophagocytic lymphohistiocytosis, familial, 2, Immunodeficiency 26, with or without neurologic abnormalities, Dystonia 16, Hypoparathyroidism, familial isolated 1, Hyperphenylalaninemia, BH4-deficient, A, Myopathy, lactic acidosis, and sideroblastic anemia 1, Intellectual developmental disorder with abnormal behavior, microcephaly, and short stature, Leukodystrophy, hypomyelinating, 10, Combined oxidative phosphorylation deficiency 40, Carpenter syndrome, Immunodeficiency 73C with defective neutrophil chemotaxis and hypogammaglobulinemia, Leber congenital amaurosis 12, Deafness, autosomal recessive 24, Aicardi-Goutieres syndrome 3, RIDDLE syndrome, Robinow syndrome, autosomal recessive, Ribose 5-phosphate isomerase deficiency, Mitochondrial complex II deficiency, nuclear type 4, Spinocerebellar ataxia, autosomal recessive, with axonal neuropathy 2, Neurodevelopmental disorder with cardiomyopathy, spasticity, and brain abnormalities, Albinism, oculocutaneous, type VI, Skin/hair/eye pigmentation 4, fair/dark skin], Citrullinemia, adult-onset type II, Citrullinemia, type II, neonatal-onset, Congenital disorder of glycosylation, type IIn, Parkinsonism-dystonia, infantile, 1, Lichtenstein-Knorr syndrome, Heart and brain malformation syndrome, Dentin dysplasia, type I, with microdontia and misshapen teeth, Osteopetrosis, autosomal recessive 8, Ovarian dysgenesis 9, Deafness, autosomal recessive 115, Dystonia, dopa-responsive, due to sepiapterin reductase deficiency, Pyropoikilocytosis, Spherocytosis, type 3, Immunodeficiency 31B, mycobacterial and viral infections, autosomal recessive, Hemophagocytic lymphohistiocytosis, familial, 4, Intellectual developmental disorder, autosomal recessive 40, Hypertryptophanemia], Spinocerebellar ataxia, autosomal recessive, with axonal neuropathy 1, Osteogenesis imperfecta, type XVIII, Catel-Manzke syndrome, Segawa syndrome, recessive, Spinocerebellar ataxia, autosomal recessive 28, Paget disease of bone 5, juvenile-onset, Mosaic variegated aneuploidy syndrome 3, Oocyte maturation defect 9, Intellectual developmental disorder, autosomal recessive 68, Immunodeficiency 35, Beta-ureidopropionase deficiency, Leber congenital amaurosis 19, Combined oxidative phosphorylation deficiency 20, Galloway-Mowat syndrome 6, Microcephaly, growth deficiency, seizures, and brain malformations, Osteogenesis imperfecta, type XV, Split-hand/foot malformation 6, Dyskeratosis congenita, autosomal recessive 3, Xanthinuria, type I, or Spastic paraplegia 15, autosomal recessive. In some embodiments, the genes which may be a nucleotide of interest can be ABCA1, ABCA2, ADK, AGA, ALDOB, AP1S1, AP4M1, APOE, APRT, ARSB, ARSK, ATG7, ATP6V1A, ASAH1, BLOC1S6, CLN6, CLN6, CLN8, CLN8, CTSA, CTNS, CTSC, CTSD, CTSK, CUBN, CUBN, CXCR2, CYB561, DPYD, DPYD, DRAM2, EPG5, ERGIC1, FUCA1, FYCO1, GAA, GALNS, GBA, GLA, GNPTAB, GNPTAB, GNPTG, GUSB, HEXA, HEXB, HGSNAT, HGSNAT, HPS6, IDUA, IDS, KLC2, LAL, LAMP2, LAT, LHCGR, LHCGR, LHCGR, LRBA, LRP1, LYST, MAN2B1, MBTPS1, MCOLN1, MFSD8, MFSD8, MLC1, MPO, MYO7A, MYO7A, NAGA, NAGA, NAGA, NPC1, NPC1, NPC2, NT5C2, NEU1, PINK1, PLEKHM1, PRF1, SCARB2, SGSH, SHMT2, SLC29A3, SMPD1, SLC39A8, SNX14, SPG11, SPG11, SPG11, TBC1D20, VPS11, VPS11, VPS13A, VPS33B, VPS51, VPS53, WDR45B, WDR81, WDR81, XDH, ZFYVE26, ABCC9, BVES, CACNA1B, CACNA2D2, CASQ2, CAV1, DSC2, DSC2, DSP, DSP, DSP, GATA5, GPX1, JUP, KCNE1, MYL2, MYL3, NOS1AP, SCN1B, SLC1A1, SLC9A1, TACR3, TH, ABCA2, ACAN, ADARB1, ANKLE2, ATG7, BMPR1B, CDH11, CLN8, CLN8, CNTNAP2, DCC, DYNC2H1, EPHB2, HERC1, IGHMBP2, IGHMBP2, KCNJ10, KIFBP, LEP, MAG, NHLH2, NIN, NRXN1, OGDH, PAX7, PRDM8, RELN, SECISBP2, SECISBP2, SLC25A46, SLC25A46, SPG11, SPG11, SPG11, SPINK5, TCTN1, VAX1, WNT1, ABCA2, ADAMTS18, ALDH1A3, ALDOB, ALMS1, APOE, ARSK, ATP6V1A, BBS4, BBS7, BMPR1B, CABP4, CEP290, CEP290, CEP290, CEP290, CFD, CLN8, CLN8, CNGA3, CRB2, CRB2, CRX, CRYBB3, CTSA, CTSD, CTSK, CUBN, CUBN, CXCR2, DRAM2, EPG5, EPHB2, FOXE3, FUCA1, FYCO1, GRHL2, GRM6, HESX1, HESX1, HESX1, HEXB, HGSNAT, HGSNAT, HPS6, KCNMA1, KERA, KLC2, LAMA1, LAMC3, LHCGR, LHCGR, LHCGR, LRBA, MAB21L2, MFN2, MFSD2A, MFSD8, MFSD8, MLC1, MPO, NAGA, NAGA, NAGA, NPC1, NPC1, NPC2, NPHP1, NPHP1, NPHP1, PCNT, PDE6A, PDE6B, PITX3, PITX3, PLEKHM1, PRF1, PRSS56, PXDN, RAB3GAP1, RAB3GAP1, RBP4, RD3, RPGRIP1L, RPGRIP1L, RPGRIP1L, SCAPER, SCARB2, SGSH, SLC1A1, SLC29A3, SLC39A8, SMG9, SMOC1, SNX14, SPNS2, TBC1D20, TH, TRPM1, TWIST2, USH1C, USH1C, VAX1, VPS11, VPS11, VPS13A, VPS33B, WDR45B, WDR81, WDR81, ZFYVE26, ADCY1, ASPM, ATG7, BBS2, BBS2, BBS4, CNTNAP2, CPLANE1, CPLANE1, DCT, DTNBP1, DYNC2H1, HERC1, HESX1, HESX1, HESX1, MBOAT7, MFSD2A, NHLH2, NRXN1, OGDH, PCNT, PDE2A, PGAP1, PLCB1, RAB3GAP1, RAB3GAP1, RELN, RPGRIP1L, RPGRIP1L, RPGRIP1L, SECISBP2, SECISBP2, SLC25A46, SLC25A46, SPTBN2, STAMBP, TNR, TRAPPC9, WDR62, WNT1, WNT2B, ADARB1, CLN8, CLN8, DAAM2, DCC, DYNC2H1, IGHMBP2, IGHMBP2, PAX7, PHGDH, PHGDH, RAB23, RELN, TCTN1, VLDLR, WNT1, ERBB2, LGI4, MMP2, NDRG1, NHLH2, SH3TC2, SLC25A46, SLC25A46, TBCE, TBCE, TBCE, ATP2A1, BVES, CAV1, CHRNA1, CHRND, CHRND, CLN8, CLN8, FLNB, GPX1, ITPR1, LMOD3, PAX7, SCN4A, SPR, WNT10B, ACAN, BBS2, BBS2, BMPR1B, CHST11, CHSY1, COL11A1, COL11A2, COL11A2, COL11A2, COL27A1, CTSK, DDRGK1, GDF5, GDF5, GDF5, GDF5, GDF6, IFT80, LEP, LNPK, PAX7, PKDCC, POC1A, PTH, ROR2, SIK3, SLC39A8, SNAI2, COL1A2, COL27A1, F7, IFT80, LRP1, POC1A, SGPL1, SNAI2, ABCA4, ABCA4, ABCA4, ABCA4, ABCA4, ADCY6, ANKS6, ATP6V0A4, ATP6V1B1, BBS2, BBS2, CABP2, CABP4, CEP290, CEP290, CEP290, CEP290, CLCNKB, CLIC5, CLN6, CLN6, CLN8, CLN8, CLRN1, CLRN1, CNGA3, COL11A1, CPLANE1, CPLANE1, CRX, CRYBB3, CYBA, DRAM2, DYNC2H1, GDF6, GLRB, GRM6, HELLS, INPP5K, ITGA8, KCNJ10, KCNMA1, KLHL3, LAMC3, LRAT, LRAT, LRAT, LRIT3, MMP9, MYO3A, MYO7A, MYO7A, NEK1, NPHP3, NPHP3, NPHP3, NPR3, PCNT, PDE6A, PDE6B, RD3, RLBP1, RLBP1, RPGRIP1L, RPGRIP1L, RPGRIP1L, SGPL1, SPATA7, SPATA7, STAT1, TACSTD2, TH, TMEM67, TMEM67, TMEM67, TMEM67, TMEM67, TRPM1, WNT2B, XPNPEP3, AARS2, AARS2, ACADSB, ACAT1, ADAR, ADARB1, ADCY1, ADCY3, ADCY6, ADK, ADPRS, AHSG, AICDA, AIMP1, AIRE, AK7, ALDOA, ALDOB, ALKBH8, AMPD1, AMPD2, AMPD2, ANAPC7, ANO6, AP5Z1, APRT, APTX, ASCC1, ATP6V1A, ATP6V1B1, B3GALNT2, BAAT, BLNK, BLOC1S6, BMPR1B, BPGM, BRIP1, CANT1, CANT1, CARD11, CD19, CD27, CD40, CDC14A, CDK6, CENPE, CEP164, CFB, CHUK, CHUK, CLCF1, CNP, CNTNAP2, COASY, COASY, CPS1, CSF2RB, CSF3R, CSPP1, CTC1, CTU2, CXCR2, CYP19A1, CYP7B1, CYP7B1, DALRD3, DARS2, DCT, DCXR, DDC, DGUOK, DGUOK, DHODH, DLAT, DNASE1L3, DNMT3B, DOCK2, DPM1, DPYD, DPYD, EDC3, ELAC2, ELP1, ENTPD1, EPHB2, EPX, ERBB2, ERCC4, ERCC4, ERCC4, ERCC4, ERCC5, ERCC5, ERCC5, ERCC8, ERCC8, ESRP1, EXOSC1, F2, F2, FADD, FAM20C, FANCD2, FANCI, FANCL, FAR1, FARS2, FARS2, FARSA, FASTKD2, FBXO7, FERMT3, FITM2, FOXE3, FOXN1, GATB, GCDH, GCK, GFI1B, GNE, GPD1, GPSM2, GTPBP2, GTPBP3, HAAO, HAVCR2, HELLS, HJV, HMOX1, HPCA, HSD17B4, HSD17B4, HSF2BP, IFNGR1, IGHMBP2, IGHMBP2, IKBKB, IL12B, IL12RB1, IL17RC, IL21, IL21R, IL2RA, IL2RB, IRF7, IRF8, ITCH, ITK, JAG2, JAK3, JAM2, JAM3, KYNU, KYNU, LAT, LCP2, LEP, LEPR, LIPE, LYST, MCCC2, MED25, METTL5, MGME1, MLH1, MMP9, MOCOS, MOCS2, MPIG6B, MPZL2, MSH3, MSH5, NADSYN1, NAXE, NBAS, NBAS, NCAPD3, NCF1, NCF2, NDRG1, NDUFA10, NDUFA8, NDUFB8, NDUFB9, NDUFS1, NHP2, NNT, NPR2, NPR3, NT5C2, NTRK1, NUP62, NUP85, OGDH, PAH, PAH, PARK7, PAX7, PDE2A, PDHX, PDX1, PDXK, PFKM, PGM3, PKDCC, PLCB1, PLEKHM1, POC1A, POLG2, POLG2, PRF1, PRKDC, PRKRA, PTH, PTS, PUS1, PUS7, PYCR2, QRSL1, RAB23, RAC2, RD3, RDX, RNASEH2C, RNF168, ROR2, RPIA, SDHB, SETX, SHMT2, SLC24A5, SLC24A5, SLC25A13, SLC25A13, SLC39A8, SLC6A3, SLC9A1, SMG9, SMOC2, SNX10, SPIDR, SPNS2, SPR, SPTA1, SPTA1, STAT1, STX11, TAF2, TDO2, TDP1, TENT5A, TGDS, TH, THG1L, TNFRSF11B, TRIP13, TRIP13, TRMT1, TYK2, UPB1, USP45, VARS2, WDR4, WDR4, WNT1, WNT10B, WRAP53, XDH, or ZFYVE26.

CNS disorders and disorders with neurological symptoms amenable to gene therapy include, but are not limited to: Alzheimer's, brain cancer, Behcet's Disease, cerebral Lupus, Creutzfeldt-Jakob Disease, dementia, epilepsy, encephalitis, Friedreich's Ataxia, Guillain-Barre Syndrome, Gaucher Disease, headache, hydrocephalus, Huntington's disease, intracranial hypertension, leukodystrophy, migraine, myasthenia gravis, muscular dystrophy, multiple sclerosis, narcolepsy, neuropathy, Prader-Willi Syndrome, Parkinson's disease, Rett Syndrome, restless leg syndrome, sleep disorders, subarachnoid hemorrhage, stroke, traumatic brain injury, trigeminal neuralgia, transient ischemic attack, and Von Hippel-Lindau Syndrome (angiomatosis).

In some embodiments, a viral capsid as described herein may encapsidate a therapeutic gene in which the expression prevents, alleviates, or otherwise reduces a one or more symptoms of an enzyme-deficiency disease and/or a disease selected from the group consisting of Fabry disease, Gaucher disease, MPS I, MPS II, MPS IIIA, MPS IIIB, MPS IIID, MPS IVB, MPS VI, MPS VII, MPS IX, Pompe disease, Lysosomal acid lipase deficiency, Metachromatic leukodystrophy, Niemann-Pick diseases types A, B, and C2, Alpha mannosidosis, Neuraminidase deficiency, Sialidosis, Aspartylglycosaminuria, Combined saposin deficiency, Atypical Gaucher disease, Farber lipogranulomatosis, Fucosidosis, and Beta mannosidosis.

“Enzyme-deficiency diseases” include non-lysosomal storage disease such as Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, and lysosomal storage diseases. “Lysosomal storage diseases” include any disorder resulting from a defect in lysosome function. Currently, approximately 50 lysosomal storage disorders have been identified, the most well-known of which include Tay-Sachs, Gaucher, and Niemann-Pick disease. The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors, and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect. Thus, as used herein, the term “enzyme” is meant to encompass those other proteins associated with lysosomal storage diseases.

The nature of the molecular lesion affects the severity of the disease in many cases, i.e. complete loss-of-function tends to be associated with pre-natal or neo-natal onset, and involves severe symptoms; partial loss-of-function is associated with milder (relatively) and later-onset disease. Generally, only a small percentage of activity needs to be restored to have to correct metabolic defects in deficient cells. Lysosomal storage diseases are generally described in Desnick and Schuchman, 2012.

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Options for the treatment of lysosomal storage diseases include enzyme replacement therapy (ERT), substrate reduction therapy, pharmacological chaperone-mediated therapy, hematopoietic stem cell transplant therapy, and gene therapy. An example of substrate reduction therapy includes the use of Miglustat or Eliglustat to treat Gaucher Type 1. These drugs act by blocking synthase activity, which reduces subsequent substrate production. Hematopoietic stem cell therapy (HSCT), for example, is used to ameliorate and slow-down the negative central nervous system phenotype in subjects with some forms of MPS. See R. M. Boustany, “Lysosomal storage diseases—the horizon expands,” 9(10) Nat. Rev. Neurol. 583-98, October 2013, which reference is incorporated herein in its entirety by reference.

Two of the most common LSDs are Pompe disease and Fabry disease. Pompe disease, which has an estimated incidence of 1 in 10,000, is caused by defective lysosomal enzyme alpha-glucosidase (GAA), which results in the deficient processing of lysosomal glycogen. Accumulation of lysosomal glycogen occurs predominantly in skeletal, cardiac, and hepatic tissues. Infantile onset Pompe causes cardiomegaly, hypotonia, hepatomegaly, and death due to cardiorespiratory failure, usually before 2 years of age. Adult onset Pompe occurs as late as the second to sixth decade and usually involves only skeletal muscle. Treatments currently available include Genzyme's MYOZYME®/LUMIZYME® (alglucosidase alfa), which is a recombinant human alpha-glucosidase produced in CHO cells and administered by intravenous infusion.

Fabry disease, which has including mild late onset cases an overall estimated incidence of 1 in 3,000, is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments currently available include Genzyme's FABRAZYME® (agalsidase beta), which is a recombinant human alpha-galactosidase A produced in CHO cells and administered by intravenous infusion; Shire's REPLAGAL™ (agalsidase alfa), which is a recombinant human alpha-galactosidase A produced in human fibroblast cells and administered by intravenous infusion; and Amicus's GALAFOLD™ (migalastat or 1-deoxygalactonojirimycin) an orally administered small molecule chaperone that shifts the folding of abnormal alpha-galactosidase A to a functional conformation.

### Nucleic Acid Constructs

In some embodiments, a plasma cell depleting agent, a B cell depleting agent, and/or an immunoglobulin depleting agent may inhibit an immune response elicited by a nucleic acid construct. In some embodiments, a nucleic acid construct comprises a coding sequence for a polypeptide of interest (e.g., an exogenous polypeptide coding sequence). In some embodiments, a nucleic acid construct described herein may comprises a polypeptide of interest coding sequence or a reverse complement of the polypeptide of interest coding sequence (e.g., an exogenous polypeptide coding sequence or a reverse complement of the exogenous polypeptide coding sequence).

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

In some embodiments, a nucleic acid construct described herein may not comprise a promoter that drives the expression of the polypeptide of interest. in other cases the construct may comprise a promoter and/or enhancer, for example a constitutive promoter or an inducible or tissue-specific (e.g., liver- or platelet-specific) promoter that drives expression of the polypeptide of interest in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a polypeptide of interest downstream of and operably linked to a signal sequence encoding a signal peptide

The constructs disclosed herein can be modified to include or exclude any suitable structural feature as needed for any particular use and/or that confers one or more desired function.

The constructs disclosed herein can comprise a polyadenylation sequence or polyadenylation tail sequence (e.g., downstream or 3′ of a polypeptide of interest coding sequence). Methods of designing a suitable polyadenylation tail sequence are well-known. The polyadenylation tail sequence can be encoded, for example, as a “poly-A” stretch downstream of the polypeptide of interest coding sequence. A poly-A tail can comprise, for example, at least 20, 30, 40, 50, 60, 70, 80, 90, or 100 adenines (SEQ ID NO: 59), and optionally up to 300 adenines. In a specific example, the poly-A tail comprises 95, 96, 97, 98, 99, or 100 adenine nucleotides (SEQ ID NO: 60). Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. In another example, the polyadenylation signal is a bovine growth hormone (BGH) polyadenylation signal or a CpG depleted BGH polyadenylation signal.

In some examples, the nucleic acid constructs disclosed herein can be bidirectional constructs. In some examples, the nucleic acid constructs disclosed herein can be unidirectional constructs. Likewise, in some examples, the nucleic acid constructs disclosed herein can be in a vector (e.g., viral vector, such as AAV, or rAAV8) and/or a lipid nanoparticle.

### Polypeptides of Interest

Any polypeptide of interest may be encoded by the nucleic acid constructs disclosed herein. In one example, the polypeptide of interest is a therapeutic polypeptide (e.g., a polypeptide that is lacking or deficient in a subject).

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

In some embodiments, the polypeptide of interest is a multidomain therapeutic protein.

In some embodiments, the polypeptide of interest is a transgene product (e.g., a therapeutic polypeptide of interest or disclosed herein which is encoded by the transgene) disclosed herein.

In another example, the polypeptide of interest is an antigen-binding protein. An “antigen-binding protein” as disclosed herein includes any protein that binds to an antigen. Examples of antigen-binding proteins include an antibody, an antigen-binding fragment of an antibody, a multi-specific antibody (e.g., a bi-specific antibody), an scFv, a bis-scFv, a diabody, a triabody, a tetrabody, a V-NAR, a VHH, a VL, a F (ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus. For example, broadly neutralizing antibodies can focus on conserved functional targets, attacking a vulnerable site on conserved bacterial or viral proteins (e.g., a vulnerable site on the influenza viral protein hemagglutinin). Antibodies developed by the immune system upon infection or vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. Broadly neutralizing antibodies—termed “broadly” because they attack many strains of the bacteria or virus, and “neutralizing” because they attack key functional sites in the bacteria or virus and block infection—can overcome these problems. Unfortunately, however, these antibodies usually come too late and do not provide effective protection from the disease.

The antigen-binding proteins disclosed herein can target any antigen. The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

As one example, the targeted antigen can be a disease-associated antigen. The term “disease-associated antigen” refers to an antigen whose presence is correlated with the occurrence or progression of a particular disease. For example, the antigen can be in a disease-associated protein (i.e., a protein whose expression is correlated with the occurrence or progression of the disease). Optionally, a disease-associated protein can be a protein that is expressed in a particular type of disease but is not normally expressed in healthy adult tissue (i.e., a protein with disease-specific expression or disease-restricted expression). However, a disease-associated protein does not have to have disease-specific or disease-restricted expression.

As one example, a disease-associated antigen can be a cancer-associated antigen. The term “cancer-associated antigen” refers to an antigen whose presence is correlated with the occurrence or progression of one or more types of cancer. For example, the antigen can be in a cancer-associated protein (i.e., a protein whose expression is correlated with the occurrence or progression of one or more types of cancer). For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Optionally, a cancer-associated protein can be a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer.

As another example, a disease-associated antigen can be an infectious-disease-associated antigen. The term “infectious-disease-associated antigen” refers to an antigen whose presence is correlated with the occurrence or progression of a particular infectious disease. For example, the antigen can be in an infectious-disease-associated protein (i.e., a protein whose expression is correlated with the occurrence or progression of the infectious disease). Optionally, an infectious-disease-associated protein can be a protein that is expressed in a particular type of infectious disease but is not normally expressed in healthy adult tissue (i.e., a protein with infectious-disease-specific expression or infectious-disease-restricted expression). However, an infectious-disease-associated protein does not have to have infectious-disease-specific or infectious-disease-restricted expression. For example, the antigen can be a viral antigen or a bacterial antigen. Such antigens include, for example, molecular structures on the surface of viruses or bacteria (e.g., viral proteins or bacterial proteins) that are recognized by the immune system and are capable of triggering an immune response.

Examples of viral antigens include antigens within proteins expressed by the Zika virus or influenza (flu) viruses. Zika is a virus spread to people primarily through the bite of an infected Aedes species mosquito (Aedes aegypti and Aedes albopictus). Zika virus infection during pregnancy can cause microcephaly and other severe brain defects. For example, a Zika antigen can be, but is not limited to, an antigen within a Zika virus envelope (Env) protein. Influenza virus is a virus that causes an infectious disease called influenza (commonly known as “the flu”). Three types of influenza viruses affect people, called Type A, Type B, and Type C. An influenza antigen can be, but is not limited to, an antigen within the hemagglutinin protein. Viral antigens and bacterial antigens also include antigens on other viruses and other bacteria. Examples of antibodies targeting influenza hemagglutinin are provided, e.g., in WO 2016/100807, herein incorporated by reference in its entirety for all purposes.

Examples of bacterial antigens include antigens within proteins expressed by Pseudomonas aeruginosa (e.g., an antigen within PcrV, which is a type III virulence system translocating protein). Pseudomonas aeruginosa is an opportunistic bacterial pathogen that causes fatal acute lung infections in critically ill individuals. Its pathogenesis is associated with bacterial virulence conferred by the type Ill secretion system (TTSS), through which P. aeruginosa causes necrosis of the lung epithelium and disseminates into the circulation, resulting in bacteremia, sepsis, and mortality. TTSS allows P. aeruginosa to directly translocate cytotoxins into eukaryotic cells, inducing cell death. The P. aeruginosa V-antigen PcrV, a homolog of the Yersinia V-antigen LcrV, is an indispensable contributor to TTS toxin translocation.

The antigen-binding protein can be a single-chain antigen-binding protein such as an scFv. Alternatively, the antigen-binding protein is not a single-chain antigen-binding protein. For example, the antigen-binding protein can include separate light and heavy chains. The heavy chain coding sequence can be upstream of the light chain coding sequence, or the light chain coding sequence can be upstream of the heavy chain coding sequence.

Signal sequences (i.e., N-terminal signal sequences) mediate targeting of nascent secretory and membrane proteins to the endoplasmic reticulum (ER) in a signal recognition particle (SRP)-dependent manner. Usually, signal sequences are cleaved off co-translationally so that signal peptides and mature proteins are generated. Examples of exogenous signal sequences or signal peptides that can be used include, for example, the signal sequence/peptide from mouse albumin, human albumin, mouse ROR1, human ROR1, human azurocidin, Cricetulus griseus Ig kappa chain V III region MOPC 63 like, and human Ig kappa chain V III region VG. Any other known signal sequence/peptide can also be used.

One or more of the nucleic acids in the antigen-binding-protein coding sequence (e.g., a heavy chain coding sequence and a light chain coding sequence) can be together in a multicistronic expression construct. For example, a nucleic acid encoding a heavy chain and a light chain can be together in a bicistronic expression construct. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin Biol Ther 5:627-638, herein incorporated by reference in its entirety for all purposes. Examples of 2A peptides that can be used include Thosea asigna virus 2A (T2A); porcine teschovirus-1 2A (P2A); equine rhinitis A virus (ERAV) 2A (E2A); and FMDV 2A (F2A). GSG residues can be added to the 5′ end of any of these peptides to improve cleavage efficiency.

In some nucleic acid constructs, a nucleic acid encoding a furin cleavage site is included between the light chain coding sequence and the heavy chain coding sequence. In some nucleic acid construct, a nucleic acid encoding a linker (e.g., GSG) is included between the light chain coding sequence and the heavy chain coding sequence (e.g., directly upstream of the 2A peptide coding sequence). For example, a furin cleavage site can be included upstream of a 2A peptide, with both the furin cleavage site and the 2A peptide being located between the light chain and the heavy chain (i.e., upstream chain—furin cleavage site—2A peptide-downstream chain). During translation, a first cleavage event will occur at the 2A peptide sequence. However, most of the 2A peptide will remain attached as a remnant to the C-terminus of the upstream chain (e.g., light chain if the light chain is upstream of the heavy chain, or heavy chain if the heavy chain is upstream of the light chain), with one amino acid added to the N-terminus of the downstream chain (or the N-terminus of a signal sequence, if a signal sequence is included upstream of the downstream chain). A second cleavage event, initiated at the furin cleavage site, yields the upstream chain without the 2A remnants in order to obtain a more native heavy chain or light chain by post-translational processing.

### Lipid Nanoparticles

In some embodiments, a plasma cell depleting agent, a B cell depleting agent, and/or an immunoglobulin depleting agent may inhibit an immune response elicited by a lipid nanoparticle. As a non-limiting example, lipid nanoparticles can comprise a nucleic acid construct encoding a polypeptide of interest disclosed herein (e.g., a transgene product including a therapeutic agent, e.g., a therapeutic polypeptide).

Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In some LNPs, the lipid component comprises an amine lipid such as a biodegradable, ionizable lipid. In some instances, the lipid component comprises biodegradable, ionizable lipid, cholesterol, DSPC, and PEG-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy) hexadecanoyl)oxy) propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl) methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (I-[8′-(Cholest-5-en-3 [beta]-oxy) carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes

### Dosage and Administration Regimens

In some embodiments, an amount of a plasma cell depleting agent, e.g., an antigen-binding molecule that binds to B cell maturation antigen (BCMA) and CD3 (e.g., an anti-BCMA×CD3 bispecific antibody), a B cell depleting agent, (e.g., anti-CD19 and anti-CD20 antibodies, or a CD20×CD3 antigen-binding molecule (e.g., REGN1979)), an immunoglobulin depleting agent such as a neonatal Fc receptor (FcRn) blocker (e.g., efgartigimod alfa), and/or an immunogen (e.g., an immunogenic delivery vehicle), or pharmaceutical composition thereof which is administered to a subject according to the methods disclosed herein is a therapeutically effective amount. As used herein, the phrase “therapeutically effective amount” means an amount that produces the desired effect for which it is administered. The subject can be from any suitable species, such as eukaryotic or mammalian subjects (e.g., non-human mammalian subject or human subject). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, humans, rodents, mice, rats, and non-human primates. In a specific example, the subject is a human. The human may be a patient. Likewise, cells can be any suitable type of cell. In a specific example, the cell or cells are a liver cell or liver cells such as a hepatocyte or hepatocytes (e.g., human liver cell(s) or human hepatocyte(s)). In some embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., an immunogenic delivery vehicle), or pharmaceutical composition thereof, is administered to a subject as a weight-based dose. A “weight-based dose” (e.g., a dose in mg/kg) is a dose of the plasma cell depleting agent, the B cell depleting agent, the immunoglobulin depleting agent, and/or the immunogenic delivery vehicle that will change depending on the subject's weight.

In other embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., an immunogenic delivery vehicle), is administered as a fixed dose. A “fixed dose” (e.g., a dose in mg) means that one dose of the plasma cell depleting agent, the B cell depleting agent, the immunoglobulin depleting agent, and/or the immunogen (e.g., an immunogenic delivery vehicle), is used for all subjects regardless of any specific subject-related factors, such as weight. In one particular embodiment, a fixed dose of a plasma cell depleting agent, a B cell depleting agent, an immunoglobulin depleting agent, and/or an immunogen is based on a predetermined weight or age.

Typically, a suitable dose of the plasma cell depleting agent and/or the B cell depleting agent and/or the immunoglobulin depleting agent and/or the immunogen can be in the range of about 0.001 to about 200.0 milligram per kilogram body weight of the recipient, generally in the range of about 1 to 50 mg per kilogram body weight. For example, the plasma cell depleting agent and/or the B cell depleting agent and/or the immunoglobulin depleting agent and/or the immunogen can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure.

In some embodiments, the plasma cell depleting agent (e.g., an anti-BCMA×CD3 bispecific antibody) is administered at a dose of about 25, about 20 to about 30, about 15 to about 35, about 10 to about 40, about 10 to about 25, about 15 to about 25, about 20 to about 25, about 25 to about 30, about 25 to about 35, or about 25 to about 40 mg/kg. In some embodiments, the plasma cell depleting agent (e.g., an anti-BCMA×CD3 bispecific antibody) is administered at a dose of about 20 to about 30 mg/kg. In some embodiments, the plasma cell depleting agent (e.g., an anti-BCMA×CD3 bispecific antibody) is administered at a dose of about 25 mg/kg.

In some embodiments, the immunoglobulin depleting agent (e.g., FcRn blocker, such as efgartigimod alfa) is administered at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg. In some embodiments, the immunoglobulin depleting agent (e.g., FcRn blocker, such as efgartigimod alfa) is administered at a dose of about 10 to about 20 mg/kg. In some embodiments, the immunoglobulin depleting agent (e.g., FcRn blocker, such as efgartigimod alfa) is administered at a dose of about 25 to about 24 mg/kg. In some embodiments, the immunoglobulin depleting agent (e.g., FcRn blocker, such as efgartigimod alfa) is administered at a dose of about 20 mg/kg. In some embodiments, the immunoglobulin depleting agent (e.g., FcRn blocker, such as efgartigimod alfa) is administered at a dose of about 10 mg/kg.

In some embodiments, the immunoglobulin depleting agent (e.g., FcRn blocker, such as efgartigimod alfa) is administered at a dose of about 10 mg/kg weekly for about 1 week, about 2 weeks, about 3 weeks, about 4 weeks, about 5 weeks, about 6 weeks, about 7 weeks, about 8 weeks, about 9 weeks, or about 10 weeks, or more. In some embodiments, the immunoglobulin depleting agent (e.g., FcRn blocker, such as efgartigimod alfa) is administered at a dose of about 10 mg/kg weekly for about 4 weeks. In various embodiments, such as when a dose of an immunoglobulin depleting agent (e.g., FcRn blocker, such as efgartigimod alfa) is administered, e.g., in combination with a plasma cell depleting agent and/or a B cell depleting agent, and optionally, an immunogen (e.g., an immunogenic delivery vehicle such as, e.g., AAV), described herein, the first dose of the immunoglobulin depleting agent may be delayed as compared to the first dose of the plasma cell depleting agent, the first dose of the B cell depleting agent, and/or the first dose of the immunogen. In some embodiments, the first dose of the immunoglobulin depleting agent is delayed by about 5 to about 20 days as compared to first dose of the plasma cell depleting agent, the first dose of the B cell depleting agent, and/or and/or the first dose of the immunogen. In some embodiments, the first dose of the immunoglobulin depleting agent is delayed by about 7 to about 15 days as compared to first dose of the plasma cell depleting agent, the first dose of the B cell depleting agent, and/or the first dose of the immunogen. In some embodiments, the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen. In some embodiments, the first dose of the immunoglobulin depleting agent is delayed by about 12 days as compared to first dose of the plasma cell depleting agent, the first dose of the B cell depleting agent, and/or the first dose of the immunogen. In some embodiments, the first dose of the immunoglobulin depleting agent is delayed by about 13 days as compared to first dose of the plasma cell depleting agent, the first dose of the B cell depleting agent, and/or the first dose of the immunogen. In some embodiments, the first dose of the immunoglobulin depleting agent is delayed by about 14 days as compared to first dose of the plasma cell depleting agent, the first dose of the B cell depleting agent, and/or the first dose of the immunogen.

In one specific embodiment, the immunoglobulin depleting agent (e.g., FcRn blocker, such as efgartigimod alfa) is administered at a dose of about 10 mg/kg weekly for about 4 weeks and the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 9 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen.

In some embodiments, the B cell depleting agent (e.g., anti-CD19 antibody or anti-CD20 antibody) is administered at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg. In some embodiments, the B cell depleting agent (e.g., anti-CD19 antibody or anti-CD20 antibody) is administered at a dose of about 10 to about 20 mg/kg. In some embodiments, the B cell depleting agent (e.g., anti-CD19 antibody or anti-CD20 antibody) is administered at a dose of about 25 to about 24 mg/kg. In some embodiments, the B cell depleting agent (e.g., anti-CD19 antibody or anti-CD20 antibody) is administered at a dose of about 20 mg/kg.

In some embodiments, the B cell depleting agent (e.g., anti-CD20×CD3 bispecific antibody) is administered at a dose of about 0.4 to about 0.6, 0.3 to about 0.7, 0.2, to about 0.8, 0.1 to about 0.9, 0.1 to about 0.5, 0.2 to about 0.5, 0.3 to about 0.5, 0.4 to about 0.5, 0.5 to about 0.6, 0.5 to about 0.7, 0.5 to about 0.8, or 0.5 to about 0.9 mg/kg. In some embodiments, the B cell depleting agent (e.g., anti-CD20×CD3 bispecific antibody) is administered at a dose of about 0.4 to about 0.6 mg/kg. In some embodiments, the B cell depleting agent (e.g., anti-CD20×CD3 bispecific antibody) is administered at a dose of about 0.3 to about 0.7 mg/kg. In some embodiments, the B cell depleting agent (e.g., anti-CD20×CD3 bispecific antibody) is administered at a dose of about 0.5 mg/kg.

In some embodiments, the plasma cell depleting agent and/or the B cell depleting agent and/or the immunoglobulin depleting agent and/or the immunogen is administered as a fixed dose of between about 5 mg to about 2500 mg. In some embodiments, the plasma cell depleting agent and/or the B cell depleting agent and/or the immunoglobulin depleting agent is administered as a fixed dose of about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

In one embodiment, for a plasma cell depleting agent (e.g., an anti-BCMA/anti-CD3 bispecific antibody), a therapeutically effective amount can be from about 0.05 mg to about 500 mg, from about 1 mg to about 500 mg, from about 10 mg to about 450 mg, from about 50 mg to about 400 mg, from about 75 mg to about 350 mg, or from about 100 mg to about 300 mg of the antibody. For example, in various embodiments, the amount of the plasma cell depleting agent is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, of the plasma cell depleting agent.

In some embodiments, the plasma cell depleting agent (e.g., an anti-BCMA×CD3 bispecific antibody) and/or the B cell depleting agent (e.g., anti-CD19/CD20 antibodies, or a CD20×CD3 antigen-binding molecule (e.g., REGN1979)) and/or the immunoglobulin depleting agent (e.g., efgartigimod alfa) and/or the immunogen (e.g., an immunogenic delivery vehicle) is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogenic delivery vehicle can be administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogen (e.g., an immunogenic delivery vehicle) can be administered at a dosing frequency of about four times a year, twice a year, once a week, once every two years, once every three years, once every four years, once every five years, once every six years, once every eight years, once every twelve years, or less frequently so long as a therapeutic response is achieved.

Dose ranges and frequency of administration of an immunogen, e.g., an immunogenic delivery vehicle such as a vector (e.g., a viral vector such as an AAV vector) described herein can vary depending on the nature of, and/or the medical condition, as well as parameters of a specific subject and the route of administration used. As a non-limiting example, vector compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the vector compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

In some embodiments, multiple doses of a plasma cell depleting agent (e.g., an anti-BCMA×CD3 bispecific antibody), a B cell depleting agent (e.g., anti-CD19/CD20 antibodies, or a CD20×CD3 antigen-binding molecule (e.g., REGN1979)), an immunoglobulin depleting agent (e.g., efgartigimod alfa), and/or an immunogen (e.g., an immunogenic delivery vehicle) are administered to a subject over a defined time course. In some embodiments, the methods of the present disclosure comprise sequentially administering to a subject multiple doses of the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., an immunogenic delivery vehicle).

In some embodiments, the immunogen (e.g., an immunogenic delivery vehicle such as a vector e.g. an AAV vector) may be administered in accordance with a repeat dosing regimen wherein the immunogen (e.g., an immunogenic delivery vehicle) may be administered a first time (e.g., in an initial dose) and then re-administered any number of subsequent times thereafter at any amount over the time course of treatment of a subject. For example, the immunogen may be re-administered once, twice, three times, four times, five times, six times, seven times, eight times, nine times, ten times, or more, over the time course of the treatment of a subject which can occur over any number of days, weeks, or years. In some embodiments, when the immunogen (e.g., an immunogenic delivery vehicle) comprises a vector, e.g., a viral vector such as an AAV vector, the vector which is administered first in a repeat dosing regimen may comprise the same vector which is re-administered second in the repeat dosing regimen, or any number of subsequent times thereafter. In some embodiments, when the immunogen (e.g., an immunogenic delivery vehicle) comprises a vector, e.g., a viral vector such as an AAV vector, the vector which is administered first in a repeat dosing regimen may comprise a different vector than is re-administered second in the repeat dosing regimen, or any number of subsequent times thereafter.

In some embodiments, immunogen (e.g., an immunogenic delivery vehicle), e.g., a viral vector such as an AAV vector, may be administered in accordance with a stepwise dosing regimen. Stepwise dosing of an immunogen can refer to breaking up (i.e., dividing) dosing of the same immunogen over multiple administrations. In some embodiments, the dosing of the same immunogen is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of an immunogen, e.g., an immunogenic delivery vehicle, e.g., a viral vector such as an AAV vector, the stepwise dosing regimen may result in a gradual increase in therapeutic transgene levels with each administration of the immunogen. Without wishing to be bound by theory, a stepwise dosing regimen used in the administration of an immunogen comprising an immunogenic delivery vehicle, e.g., a viral vector such as an AAV vector, can result in enhanced control over transgene expression in a cell and/or subject, since for some transgenes too much expression can result in its own pathology.

In some embodiments, an immunogen (e.g., an immunogenic delivery vehicle) is administered to a subject in combination with a plasma cell depleting agent, a B cell depleting agent, and/or an immunoglobulin depleting agent. The plasma cell depleting agent and/or the B cell depleting agent and/or the immunoglobulin depleting agent can be administered prior to, simultaneously with, or after the immunogen. In one example, the plasma cell depleting agent and/or B cell depleting agent and/or immunoglobulin depleting agent is administered prior to the immunogen. In another example, the plasma cell depleting agent and/or B cell depleting agent and/or immunoglobulin depleting agent is administered prior to and after the immunogen. In another example, the plasma cell depleting agent and/or B cell depleting agent and/or immunoglobulin depleting agent is administered simultaneously with the immunogen. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen, e.g., 2-4 days afterwards as B cell formation may be limited during the initial lag period. Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, when the plasma cell depleting agent and/or the B cell depleting agent and/or the immunoglobulin depleting agent is administered prior to, simultaneously with, and/or after the immunogen (e.g., an immunogenic delivery vehicle), the plasma cell depleting agent and/or the B cell depleting agent and/or the immunoglobulin depleting agent is administered once, twice, three times, four times, five times, six times, seven times, eight times, nine times, ten times, eleven times, twelve times, thirteen times, fourteen times, fifteen times, sixteen times, seventeen times, eighteen times, nineteen times, or twenty times or more, prior to, simultaneously with, and/or after the administration of the immunogen (e.g., an immunogenic delivery vehicle). In some embodiments, when an immunogen is administered in accordance with a repeat dosing regimen, a plasma cell depleting agent and/or a B cell depleting agent and/or an immunoglobulin depleting agent may be administered any number of times prior to, simultaneously with, and/or after a first and/or second administration of the immunogen, and/or any number of subsequent administrations of the immunogen thereafter. Without wishing to be bound by theory, when a plasma cell depleting agent and/or a B cell depleting agent and/or an immunoglobulin depleting agent is administered to inhibit an immune response to an immunogen in a subject in need thereof, e.g., an anti-drug antibody response to an immunogenic protein, the plasma cell depleting agent and/or the B cell depleting agent and/or the immunoglobulin depleting agent may be co-administered (e.g., administered prior to, simultaneously with, and/or after the immunogen) to prevent the response of the immune system of the subject on each dose of the immunogen. As an example, an immunogen comprising a bacterial IgG cleaving enzyme IdeS/imlifidase may be administered to a subject for overcoming AAV pre-existing immunity; however, IdeS itself is immunogenic and can only be administered once. Co-administration of a plasma cell depleting agent and/or a B cell depleting agent and/or an immunoglobulin depleting agent described herein with IdeS/imlifidase can prevent the de novo response to IdeS protein.

According to certain embodiments of the present disclosure, a plasma cell depleting agent and/or a B cell depleting agent and/or an immunoglobulin depleting agent may be administered to a subject separately from an immunogen (e.g., an immunogenic delivery vehicle) described herein.

In some embodiments, when a plasma cell depleting agent and/or a B cell depleting agent and/or an immunoglobulin depleting agent and an immunogen (e.g., an immunogenic delivery vehicle) are administered separately, the plasma cell depleting agent and/or the B cell depleting agent and/or the immunoglobulin depleting agent may be administered simultaneously with the administration of the immunogen. In some embodiments, the plasma cell depleting agent and/or the B cell depleting agent and/or the immunoglobulin depleting agent is administered one or more times during the administration of the immunogen. In some embodiments, the immunogen is administered one or more times during the administration of the plasma cell depleting agent and/or the B cell depleting agent and/or the immunoglobulin depleting agent. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen, e.g., 2-4 days afterwards as B cell formation may be limited during the initial lag period. Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, a B cell depleting agent is administered alone (e.g., without a plasma cell depleting agent). In some embodiments, a B cell depleting agent is administered prior to a plasma cell depleting agent. In some embodiments, a B cell depleting agent is administered simultaneously with a plasma cell depleting agent. In some embodiments, a B cell depleting agent is administered subsequent to a plasma cell depleting agent. In some embodiments, a B cell depleting agent is administered prior to and subsequent to a plasma cell depleting agent. In some embodiments, a B cell depleting agent is administered prior to and simultaneously with a plasma cell depleting agent. In some embodiments, a B cell depleting agent is administered simultaneously with and subsequent to a plasma cell depleting agent. In theory, B cell depletion could be conducted before or after plasma cell depletion with the same effect, provided that B cells remain depleted up until the time of dosing with the immunogen.

In some embodiments, an immunoglobulin depleting agent is administered subsequent to a plasma cell depleting agent. In some embodiments, an immunoglobulin depleting agent is administered subsequent to a B cell depleting agent. In some embodiments, an immunoglobulin depleting agent is administered subsequent to a plasma cell depleting agent and a B cell depleting agent. For example, if the plasma cell depleting agent is an anti-BCMA×CD3 bispecific antibody, administering the immunoglobulin depleting agent after the plasma cell depleting agent will prevent more rapid clearance of the plasma cell depleting agent. For example, if the B cell depleting agent is an anti-CD20×CD3 bispecific antibody, administering the immunoglobulin depleting agent after the B cell depleting agent will prevent more rapid clearance of the B cell depleting agent. The timing can be affected by what immunoglobulin depleting agent is used. For example, different treatment regimens would be expected for FcRn blockers vs. IgG degrading enzymes (e.g., IdeS). IdeS is an enzyme and therefore acts much more rapidly than FcRn blockade, clearing IgGs within hours to days. For FcRn blockade, several weeks of treatment may be required to fully clear anti-AAV IgGs from circulation.

In some embodiments, a plasma cell depleting agent and/or a B cell depleting agent and/or an immunoglobulin depleting agent and an immunogen (e.g., an immunogenic delivery vehicle) may be administered separately over a defined time course. In certain embodiments, multiple doses of a plasma cell depleting agent and/or a B cell depleting agent and/or an immunoglobulin depleting agent and/or an immunogen (e.g., an immunogenic delivery vehicle) described herein may be administered to a subject over a defined time course. The methods according to such aspects of the disclosure may comprise sequentially administering to a subject multiple doses of a plasma cell depleting agent and/or a B cell depleting agent and/or an immunoglobulin depleting agent and/or immunogen of the disclosure. In some embodiments, when the plasma cell depleting agent and/or the B cell depleting agent and/or the immunoglobulin depleting agent and immunogen are administered sequentially, the plasma cell depleting agent and/or the B cell depleting agent and/or the immunoglobulin depleting agent can be administered before and/or in between each of the administrations of the immunogen(s). As used herein, “sequentially administering” means that each dose of the plasma cell depleting agent, the B cell depleting agent, the immunoglobulin depleting agent, and/or the immunogenic delivery vehicle, is administered to the subject at a different point in time, e.g., on different days separated by a predetermined interval (e.g., hours, days, weeks, months, or years). In some embodiments, the methods of the disclosure comprise sequentially administering to the subject a single initial dose of the plasma cell depleting agent and/or the B cell depleting agent and/or the immunoglobulin depleting agent and/or the immunogen, followed by one or more secondary doses of the plasma cell depleting agent and/or the B cell depleting agent and/or the immunoglobulin depleting agent and/or the immunogen (e.g., an immunogenic delivery vehicle), and optionally followed by one or more tertiary doses of the plasma cell depleting agent and/or the B cell depleting agent and/or the immunoglobulin depleting agent and/or the immunogen.

The terms “initial dose,” “secondary dose(s),” and “tertiary dose(s)” refer to the temporal sequence of administration of the plasma cell depleting agent, the B cell depleting agent, the immunoglobulin depleting agent, and/or the immunogen (e.g., an immunogenic delivery vehicle). Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. In some embodiments, the initial, secondary, and tertiary doses may all contain the same amount of the plasma cell depleting agent, the B cell depleting agent, the immunoglobulin depleting agent, and/or the immunogen but may differ from one another in terms of frequency of administration. In some embodiments, the amount of the plasma cell depleting agent, B cell depleting agent, immunoglobulin depleting agent, and/or the immunogen contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the plasma cell depleting agent, the B cell depleting agent, the immunoglobulin depleting agent, and/or the immunogen. In other embodiments, the initial dose comprises a first amount of the plasma cell depleting agent, the B cell depleting agent, the immunoglobulin depleting agent, and/or the immunogen (e.g., an immunogenic delivery vehicle) and the one or more secondary doses each comprise a second amount of the plasma cell depleting agent, the B cell depleting agent, the immunoglobulin depleting agent, and/or the immunogen. For example, the first amount of the plasma cell depleting agent, the B cell depleting agent, the immunoglobulin depleting agent, and/or the immunogen (e.g., an immunogenic delivery vehicle) can be 1.5×, 2×, 2.5×, 3×, 3.5×, 4×, 5× or more than the second amount of the plasma cell depleting agent, the B cell depleting agent, the immunoglobulin depleting agent, and/or the immunogen.

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the plasma cell depleting agent, the B cell depleting agent, the immunoglobulin depleting agent, and/or the immunogen (e.g., an immunogenic delivery vehicle) that is administered to a subject prior to the administration of the very next dose in the sequence with no intervening doses.

The methods of the disclosure may comprise administering to a subject any number of secondary and/or tertiary doses of a plasma cell depleting agent, a B cell depleting agent, an immunoglobulin depleting agent, and/or an immunogen (e.g., an immunogenic delivery vehicle). For example, in certain embodiments, only a single secondary dose is administered to the subject. In other embodiments, two or more (e.g., 2, 3, 4, 5, 6, 7, 8, or more) secondary doses are administered to the subject. Likewise, in certain embodiments, only a single tertiary dose is administered to the subject. In other embodiments, two or more (e.g., 2, 3, 4, 5, 6, 7, 8, or more) tertiary doses are administered to the subject.

In some embodiments involving multiple secondary doses, each secondary dose is administered at the same frequency as the other secondary doses. For example, each secondary dose may be administered to the subject 1, 2, 3, or 4 weeks after the immediately preceding dose. Similarly, in some embodiments involving multiple tertiary doses, each tertiary dose is administered at the same frequency as the other tertiary doses. Alternatively, the frequency at which the secondary and/or tertiary doses are administered to a subject can vary over the course of the treatment regimen. The frequency of administration may also be adjusted during the course of treatment by a physician depending on the needs of the individual subject following clinical examination.

In some embodiments, the secondary and/or tertiary doses of an immunogen (e.g., an immunogenic delivery vehicle) comprising a viral particle or vector (e.g., a viral vector such as an AAV vector) administered to the subject is of the same or similar viral origin as the initial dose. In some embodiments, the secondary and/or tertiary doses of an immunogen (e.g., an immunogenic delivery vehicle) comprising a viral particle or vector administered to the subject is of a different viral origin then the initial dose.

In some embodiments, the subsequently administered viral vector is administered via the same administration route as the originally administered viral vector. In some embodiments, the subsequently administered viral vector is administered via a different administration route from the originally administered viral vector.

In some embodiments, when a plasma cell depleting agent and/or a B cell depleting agent and/or an immunoglobulin depleting agent and/or an immunogen (e.g., an immunogenic delivery vehicle) herein are sequentially administered, the immunogen (e.g., an immunogenic delivery vehicle) may be administered as a first component of the dosing regimen and the plasma cell depleting agent, B cell depleting agent, and/or immunoglobulin depleting agent may be administered as a second component of the dosing regimen (i.e., the immunogen may be administered before the plasma cell depleting agent and/or the B cell depleting agent and/or the immunoglobulin depleting agent). In some embodiments, the immunogen may be administered as a second component of the dosing regimen and the plasma cell depleting agent and/or the B cell depleting agent and/or the immunoglobulin depleting agent may be administered as a first component of a dosing regimen (i.e., the immunogen may be administered after the plasma cell depleting agent and/or the B cell depleting agent and/or the immunoglobulin depleting agent). In some embodiments, an immunogen and a plasma cell depleting agent and/or a B cell depleting agent and/or an immunoglobulin depleting agent may be sequentially administered, in either of the above-described orders, with variable time intervals between administration. For example, the time interval between administration of the immunogen (e.g., an immunogenic delivery vehicle) and the plasma cell depleting agent and/or B cell depleting agent and/or immunoglobulin depleting agent may be at least about 30 seconds, at least about 35 seconds, at least about 40 seconds, at least about 45 seconds, at least about 50 seconds, at least about 55 seconds, at least about 1 minute, at least about 2 minutes, at least about 5 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 10 to 12 hours, at least about 12 to 14 hours, at least about 14 to 16 hours, at least about 16 to 18 hours, at least about 18 to 20 hours, at least about 20 to 22 hours, at least about 22 to 24 hours, at least about 1 day, at least about 2 days, at least about 3 days, at least about 4 days, at least about 5 days, at least about 6 days, at least about 7 days, at least about 8 days, at least about 9 days, at least about 10 days, at least about 10 to 12 days, at least about 12 to 14 days, at least about 14 to 16 days, at least about 16 to 18 days, at least about 18 to 20 days, at least about 20 to 22 days, at least about 22 to 24 days, at least about 24 to 26 days, at least about 28 days, at least about 29 days, at least about 30 days, at least about 31 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, or more.

Any of the above methods can further comprise any of various subsequent administration steps described herein. The subsequent administration step can comprise, for example, administering the plasma cell depleting agent and/or the B cell depleting agent and/or the immunoglobulin depleting agent and an immunogen (e.g., an immunogenic delivery vehicle) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can comprise, for example, administering a second immunogen (e.g., an immunogenic delivery vehicle), e.g. an immune delivery vehicle comprising, e.g., a vector comprising a coding sequence for a second polypeptide of interest (e.g., that is different from a first polypeptide of interest encoded by a first vector administered in an initial administration step) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some embodiments, the present disclosure provides a method for increasing effectiveness of a subsequently administered viral vector following an originally administered viral vector in a subject in need thereof, comprising administering to the subject an effective amount of a plasma cell depleting agent, a B cell depleting agent, and/or an immunoglobulin depleting agent, and the subsequently administered viral vector is of the same or similar viral origin as the originally administered viral vector.

In some embodiments, the subsequently administered viral vector is administered via the same administration route as the originally administered viral vector.

In some embodiments, the subsequently administered viral vector is administered via a different administration route from the originally administered viral vector

In some embodiments, the plasma cell depleting agent is administered before the administration of the subsequently administered viral vector(s).

In some embodiments, the plasma cell depleting agent is administered simultaneously with the administration of the subsequently administered viral vector(s).

In some embodiments, the subsequently administered viral vectors are administered two or more times and the plasma cell depleting agent is administered before and/or between each of the administrations of the subsequently administered viral vectors.

In some embodiments, the plasma cell depleting agent, the B cell depleting agent, and/or the immunoglobulin depleting agent is administered before the administration of the viral vector(s). In some embodiments, the anti-CD20×CD3 bispecific antibody or functional fragment thereof is administered before the administration of the originally administered viral vector to the subject.

In some embodiments, the plasma cell depleting agent, the B cell depleting agent, and/or the immunoglobulin depleting agent is administered simultaneously with the administration of the viral vector(s). In some embodiments, the anti-CD20×CD3 bispecific antibody or functional fragment thereof is administered simultaneously with the administration of the originally administered viral vector and/or subsequently administered viral vector to the subject.

In some embodiments, the plasma cell depleting agent, the B cell depleting agent, and/or the immunoglobulin depleting agent is administered after the administration of the viral vector(s). In some embodiments, the anti-CD20×CD3 bispecific antibody or functional fragment thereof is administered after the administration of the originally administered viral vector but before administering the subsequently administered viral vector to the subject. In some embodiments, the anti-CD20×CD3 bispecific antibody or functional fragment thereof is administered after the administration of the subsequently administered viral vector to the subject.

In some embodiments, the viral vectors are administered two or more times and the plasma cell depleting agent, the B cell depleting agent, and/or the immunoglobulin depleting agent is administered before and/or between each of the administrations of the viral vectors. In some embodiments, the anti-CD20×CD3 bispecific antibody or functional fragment thereof is administered before and/or between each of the administrations of the viral vectors to the subject.

In any of the above methods, the plasma cell depleting agent and/or the B cell depleting agent and/or the immunoglobulin depleting agent can be administered simultaneously with the immunogen (e.g., an immunogenic delivery vehicle) or not simultaneously (e.g., sequentially in any combination). For example, in a method comprising administering a composition comprising a plasma cell depleting agent and/or a B cell depleting agent and/or an immunoglobulin depleting agent and an immunogen, they can be administered separately (e.g., the plasma cell depleting agent and/or the B cell depleting agent and/or the immunoglobulin depleting agent separately from the immunogen). For example, the plasma cell depleting agent and/or B cell depleting agent and/or immunoglobulin depleting agent can be administered prior to the immunogen, subsequent to the immunogen, prior to and subsequent to the immunogen, or at the same time as immunogen. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen (e.g., 2-4 days afterwards as plasma cell formation may be limited during the initial lag period). In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen (e.g., 2-4 days afterwards as B cell formation may be limited during the initial lag period). Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, the plasma cell depleting agent and/or the B cell depleting agent and/or the immunoglobulin depleting agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the immunogen.

In one example, the plasma cell depleting agent and/or B cell depleting agent and/or immunoglobulin depleting agent is administered about 4 hours, about 8 hours, about 12 hours, about 18 hours, about 1 day, about 2 days, about 3 days, about 4 days, about 5 days, about 6 days, or about 1 week prior to administering the immunogen (e.g., an immunogenic delivery vehicle). In another example, the plasma cell depleting agent and/or B cell depleting agent and/or immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the immunogen (e.g., an immunogenic delivery vehicle). In another example, the plasma cell depleting agent and/or B cell depleting agent and/or immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and/or subsequent to administering the immunogen (e.g., an immunogenic delivery vehicle).

In one example, the plasma cell depleting agent and/or the B cell depleting agent and/or the immunoglobulin depleting agent is administered about 4 hours, about 8 hours, about 12 hours, about 18 hours, about 1 day, about 2 days, about 3 days, about 4 days, about 5 days, about 6 days, or about 1 week prior to and subsequent to administering the immunogen (e.g., an immunogenic delivery vehicle). In another example, the plasma cell depleting agent and/or the B cell depleting agent and/or the immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering immunogen (e.g., an immunogenic delivery vehicle). In another example, the plasma cell depleting agent and/or the B cell depleting agent and/or the immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the immunogen (e.g., an immunogenic delivery vehicle).

In one example, the plasma cell depleting agent and/or B cell depleting agent and/or immunoglobulin depleting agent is administered about 4 hours, about 8 hours, about 12 hours, about 18 hours, about 1 day, about 2 days, about 3 days, about 4 days, about 5 days, about 6 days, or about 1 week after administering the immunogen (e.g., an immunogenic delivery vehicle). In another example, the plasma cell depleting agent and/or B cell depleting agent and/or immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the immunogen (e.g., an immunogenic delivery vehicle). In another example, the plasma cell depleting agent and/or B cell depleting agent and/or immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the immunogen (e.g., an immunogenic delivery vehicle).

Administration in vivo can be by any suitable route including, for example, parenteral, intravenous, oral, subcutaneous, intra-arterial, intracranial, intrathecal, intratumoral, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjunctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, intratumoral, topical, intranasal, or intramuscular. A specific example is intravenous infusion.

Administration in vivo can be by any suitable route including, for example, systemic routes of administration such as parenteral administration, e.g., intravenous, subcutaneous, intra-arterial, or intramuscular. In a specific example, administration in vivo is intravenous.

The frequency of administration and the number of dosages can depend on a number of factors. The introduction of an immunogen into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the immunogen, e.g., a vector, is sufficient to increase expression of polypeptide of interest to a desirable level. In other methods, more than one administration may be beneficial to maximize therapeutic effect.

The methods disclosed herein can increase polypeptide of interest protein levels and/or polypeptide of interest activity levels in a cell or subject (e.g., circulating, serum, or plasma levels in a subject) and can comprise measuring polypeptide of interest protein levels and/or polypeptide of interest activity levels in a cell or subject (e.g., circulating, serum, or plasma levels in a subject).

In some methods, polypeptide of interest activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, polypeptide of interest activity and/or expression levels in a subject are increased to about or at least about 40%, about or at least about 50%, about or at least about 75%, or at least about 100%, or more, of normal level.

In some methods, circulating polypeptide of interest levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, polypeptide of interest levels are at least about 1 μg/mL or about 1 μg/mL. In some methods, polypeptide of interest levels are at least about 2 μg/mL or about 2 μg/mL. In some methods, polypeptide of interest levels are at least about 5 μg/mL or about 5 μg/mL. In some methods, polypeptide of interest levels are about 1 μg/mL to about 30 g/mL, about 2 μg/mL to about 30 μg/mL, about 3 μg/mL to about 30 μg/mL, about 4 μg/mL to about 30 g/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in polypeptide of interest levels of about 2 μg/mL to about 30 μg/mL or 2 μg/mL to about 20 μg/mL. For example, the method can result in polypeptide of interest levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of polypeptide of interest over the subject's baseline expression and/or activity (i.e., expression and/or activity prior to administration). In some methods, the method increases expression and/or activity of polypeptide of interest over the subject's baseline expression and/or activity (i.e., expression and/or activity prior to administration. In some methods, polypeptide of interest activity and/or polypeptide of interest expression or serum levels in a subject are increased by about or at least about 10%, about or at least about 25%, about or at least about 50%, about or at least about 75%, or about or at least about 100%, or more, as compared to the subject's polypeptide of interest expression or serum levels and/or activity before administration (i.e., the subject's baseline levels). In certain embodiments, the loss of function is nearly complete such that a relative activity cannot be determined. In certain embodiments, the level of expression is sufficient to treat at least one sign or symptom resulting from the loss of function of the polypeptide of interest.

In some methods, the method increases expression and/or activity of the polypeptide of interest over the cell's baseline expression and/or activity (i.e., expression and/or activity prior to administration). In some methods, the method increases expression and/or activity of polypeptide of interest over the cell's baseline expression and/or activity (i.e., expression and/or activity prior to administration. In some methods, polypeptide of interest activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the polypeptide of interest activity and/or expression levels before administration (i.e., the subject's baseline levels). In certain embodiments, the polypeptide of interest loss of function is nearly complete such that a relative activity cannot be determined. In certain embodiments, the level of expression is sufficient to treat at least one sign or symptom resulting from the loss of function of the polypeptide of interest.

In a specific example, the polypeptide of interest activity levels in a subject are increased to no more than about 300%, no more than about 250%, no more than about 200%, or no more than about 150% of normal polypeptide of interest activity levels.

In a specific example, the polypeptide of interest activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels. In a specific example, the polypeptide of interest activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels.

In some methods, the method results in increased expression of the polypeptide of interest in the subject (e.g., neonatal subject) compared to a method comprising administering an episomal expression vector encoding the polypeptide of interest in a control subject. In some methods, the method results in increased serum levels of the polypeptide of interest in the subject (e.g., neonatal subject) compared to a method comprising administering an episomal expression vector encoding the polypeptide of interest to a control subject.

In some methods, the method increases expression or activity of the polypeptide of interest over the subject's baseline expression or activity of the polypeptide of interest (i.e., any percent change in expression that is larger than typical error bars). In some methods, the method results in expression of the polypeptide of interest at a detectable level above zero, e.g., at a statistically significant level, a clinically relevant level.

In some methods, the expression or activity of the polypeptide of interest is at least 50% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at one year after the administering. In some methods, the expression or activity of the polypeptide of interest is at least 60% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at two years after the administering. In some methods, the expression or activity of the polypeptide of interest is at least 60% of the expression or activity of the polypeptide of interest at a peak level of expression measured for the subject at 2 years after the administering. In some methods, the expression or activity of the polypeptide of interest is at least 60% of the expression or activity of the polypeptide of interest at a peak level of expression measured for the subject at 24 weeks after the administering.

In some methods, the method further comprises assessing pre-existing anti-polypeptide of interest immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant polypeptide of interest protein. In some methods, the subject has previously been administered recombinant polypeptide of interest protein.

In some methods, the method further comprises assessing pre-existing anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase). In some embodiments, the subject does not have pre-existing anti-AAV immunity. In some embodiments, the subject does have pre-existing AAV immunity.

### Kits

The present disclosure further comprises a kit which may comprise any of various compositions of the present disclosure, including the plasma cell depleting agents, the B cell depleting agents, the immunoglobulin depleting agents, and/or the immunogens (e.g., immunogenic delivery vehicles), or pharmaceutical compositions thereof, of the disclosure.

One exemplary embodiment of the present disclosure comprises a kit comprising (i) a plasma cell depleting agent, (ii) a B cell depleting agent and/or an immunoglobulin depleting agent, and (iii) optionally, instructions for use. Another exemplary embodiment of the present disclosure comprises a kit comprising (i) an immunogen, (ii) a plasma cell depleting agent, (iii) optionally a B cell depleting agent and/or an immunoglobulin depleting agent, and (iv) optionally, instructions for use. Yet another exemplary embodiment of the present disclosure comprises a kit comprising (i) an immunogen, (ii) an anti-CD20×CD3 bispecific antibody or a functional fragment thereof, and (iii) optionally, instructions for use.

In one aspect, the present disclosure may include a kit comprising, for example: (a) a container that contains a pharmaceutical composition disclosed herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, the kit may further comprise, for example, without limitation, one or more of (i) a buffer, (ii) a diluent, (iii) a filter, (iv) a needle, and/or (v) a syringe. As a non-limiting example, the container may be a bottle, a vial, a syringe, or test tube. In some embodiments, the container may be a multi-use container. In some the pharmaceutical composition may be lyophilized.

Kits of the present disclosure may comprise a lyophilized formulation of the present disclosure in a suitable container and instructions for its reconstitution and/or use. Suitable containers include, for example, bottles, vials (e.g. dual chamber vials), syringes (such as dual chamber syringes) and test tubes. The container may be formed from a variety of materials such as glass or plastic. The kit and/or container may contain instructions on or associated with the container that indicate directions for reconstitution of the lyophilized formulation and/or use of the kit. For example, the label may indicate that the lyophilized formulation is to be reconstituted to an appropriate peptide concentration. The label may indicate that the formulation is useful or intended for any route of administration disclosed herein, e.g., parenteral administration routes disclosed herein.

The container holding the formulation may be a multi-use vial, which may allow for repeat administrations (e.g., from 2-6 administrations) of the reconstituted formulation. The kit may further comprise a second container comprising a suitable diluent (e.g., sodium bicarbonate solution).

Upon mixing of the diluent and the lyophilized formulation, the final peptide concentration in the reconstituted formulation is reached. The kit may further include other materials desirable from a commercial and/or user standpoint, including, for example, without limitation, other buffers, diluents, filters, needles, syringes, and/or package inserts which may comprise, e.g., instructions for use.

Kits of the present disclosure may have a single container that contains the formulation of the pharmaceutical compositions according to the present disclosure with or without other components (e.g., other compounds or pharmaceutical compositions of these other compounds) or may have a distinct container for each component.

In some embodiments, kits of the disclosure may include a formulation of the disclosure packaged for use in combination with the coadministration of a second compound (such as adjuvants (e.g., GM-CSF, a chemotherapeutic agent, a natural product, a hormone or antagonist, an anti-angiogenesis agent or inhibitor, an apoptosis-inducing agent, or a chelator) or a pharmaceutical composition thereof. The components of the kit may be pre-complexed or each component may be in a separate distinct container prior to administration to a patient. The components of the kit may be provided in one or more liquid solutions. A liquid solutions described herein may be an aqueous solution, for example, a sterile aqueous solution. The components of the kit may also be provided as solids, which may be converted into liquids such as by addition of suitable solvents, which may be provided in another distinct container.

The container of a therapeutic kit may be a vial, test tube, flask, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiment, a kit may contain an apparatus (e.g., one or more needles, syringes, eye droppers, pipettes, etc.), which may allow for administration of the agents of the disclosure that are components of the present kit.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Plasma Cell Depletion in Combination with Neonatal Fc Receptor (FcRn) Blockade Elicits Rapid and Deep Suppression of Pre-Existing Anti-AAV Antibody Titers

For adeno-associated virus gene therapy, the generation of neutralizing antibodies after exposure precludes the ability to re-administer AAV vectors of the same or related serotypes, despite therapeutic need. Furthermore, due to natural exposure to wild-type AAVs, roughly 30-60% of individuals harbor pre-existing antibodies to AAV that prevent administration of even a single AAV vector. Therefore, strategies that can attenuate pre-existing anti-AAV antibody responses induced by either recombinant or wild type AAVs have the potential to greatly expand the versatility and accessibility of AAV gene therapies to a broader patient population. In some embodiments, the subsequently administered AAV vector has a capsid derived from the same AAV serotype as the originally administered AAV vector.

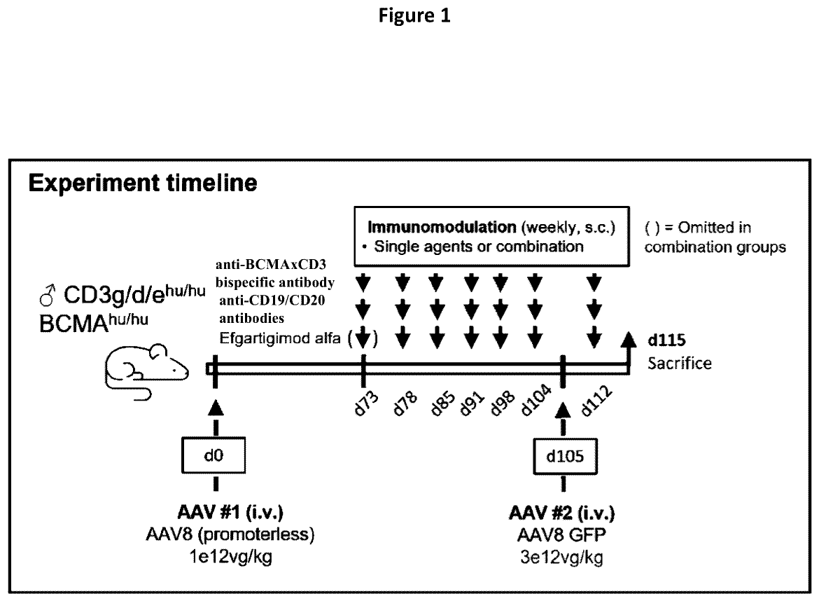

Because plasma cells are the source of long-lived antibody responses, it was reasoned that antibody-mediated plasma cell depletion may suppress pre-existing antibody responses to AAV sufficiently to enable AAV vector transduction or re-transduction in seropositive animals. To test this, B cell maturation antigen (BCMA)- and CD3 gamma-, CD3 delta-, and CD3 epsilon-humanized mice (n=6 per group) were treated with 1e12 vector genomes (vg) per kilogram (kg) recombinant AAV8 (encoding a promoterless transgene) to induce a strong anti-capsid antibody response (e.g., high-titer nAbs). 73 days later, a timepoint deemed sufficient to account for long-lived plasma cell differentiation, mice were injected subcutaneously weekly for five weeks with 25 milligrams (mg) per kg with linvoseltamab, also known as REGN5458, a fully-human T cell-bridging bispecific antibody targeting B cell maturation antigen and CD3 (referred to herein as “anti-BCMA×CD3 bispecific antibody”) to induce plasma cell depletion. Additionally, because the half-life of immunoglobulin G is relatively long (˜6-8 days in mice and ˜21 days in humans) due to the action of neonatal Fc receptor (“FcRn”), it was also evaluated whether additional blockade of FcRn with efgartigimod alfa, administered subcutaneously weekly at 20 mg/kg, could further accelerate and improve titer reductions elicited by plasma cell depletion. Finally, to capture a wider range of AAV-specific B cells that may not express high levels of BCMA, such as committed memory B cells and early plasmablasts, it was also tested whether additional B cell depletion with a cocktail of anti-CD19/CD20 antibodies, administered subcutaneously weekly at 25 mg/kg each, may further improve the therapeutic effect of plasma cell depletion with anti-BCMA×CD3 bispecific antibody. Mice were bled at defined intervals for serum anti-AAV antibody analysis. A schematic of the full experimental design is presented in FIG. 1. To prevent any initial impact of efgartigimod alfa on the therapeutic effect of anti-BCMA×CD3 bispecific antibody or anti-CD19/CD20 antibodies, which are themselves immunoglobulins, efgartigimod alfa was omitted from the first week's treatment cocktails.

To evaluate the impact of plasma cell depletion, FcRn blockade, B cell depletion, or combinations thereof on anti-AAV8 IgG titers, anti-capsid IgG levels were measured in serum of mice over time. Specifically, 96 well flat-bottom plates were coated with 1e9 vg/well recombinant AAV8 vector in DPBS overnight. The next day, plates were washed and blocked with 0.5% bovine serum albumin in DPBS for 1 hr. Serum samples were then diluted 3x, beginning at an initial dilution of 1:300 and ending at a dilution of 53,144, 100. Diluted serum was then transferred to the assay plate and incubated overnight at 4° C. The next day, the assay plates were repeatedly washed prior to incubation with an anti-mouse-IgG Fc-gamma Fragment-HRP-conjugated polyclonal secondary antibody (Jackson Immunoresearch, West Grove, PA). Plates were again repeatedly washed prior to development with TMB substrate solution. After 20 minutes, the reaction was stopped by addition of 2N phosphoric acid. Absorbance at 450 nm (OD450) was measured on a SpectraMax i3 plate reader (Molecular Devices, San Jose, CA). Relative levels of serum anti-AAV8 IgG were determined and plotted as titer values using Prism v.9 software (GraphPad, Boston, MA). Titer was defined as the dilution factor required to achieve an OD450 reading equal to 2-fold higher than background values.

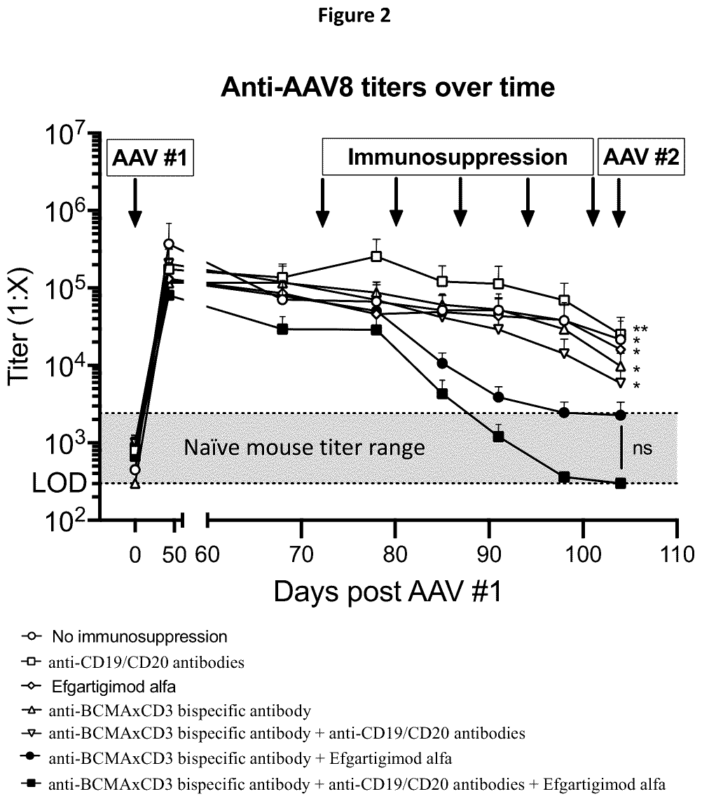

It was found that while anti-AAV8 antibody titers showed minor declines over time in mice administered either anti-BCMA×CD3 bispecific antibody, efgartigimod alfa, or anti-CD19/CD20 antibodies individually, the titer reductions were minor and not statistically different from AAV-treated mice that received no immunomodulation. By contrast, mice receiving a cocktail of anti-BCMA×CD3 bispecific antibody and efgartigimod alfa showed rapid titer declines to naïve or near-naive levels, and mice additionally treated with anti-CD19/CD20 antibodies showed even more rapid and complete titer declines, with all 6/6 mice exhibiting titers below the limit of detection by the cessation of the five-week treatment period (FIG. 2). Thus, these data demonstrate that anti-AAV8 titers can be suppressed by therapeutic plasma cell depletion, with the timeframe required for anti-AAV8 titer depletion reduced by FcRn blockade. Additionally, the depth of titer reduction can be further enhanced with B cell depletion in mice.

### Example 2: Plasma Cell Depletion in Combination with FcRn Blockade Enables AAV Vector Re-Administration

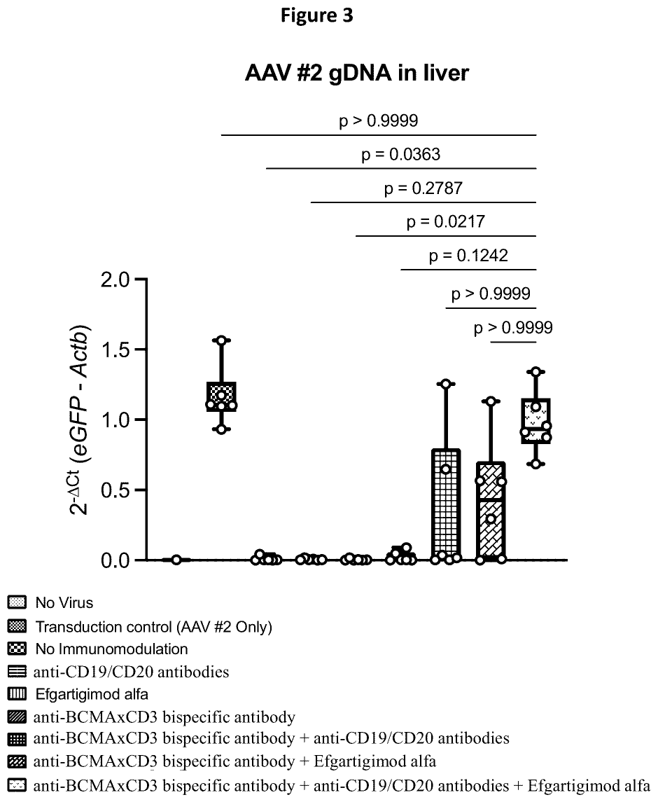

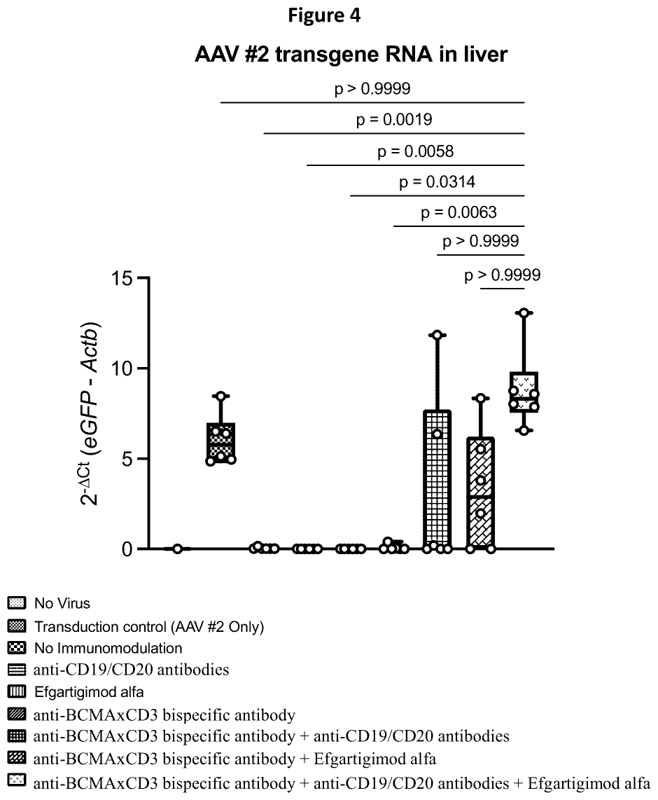

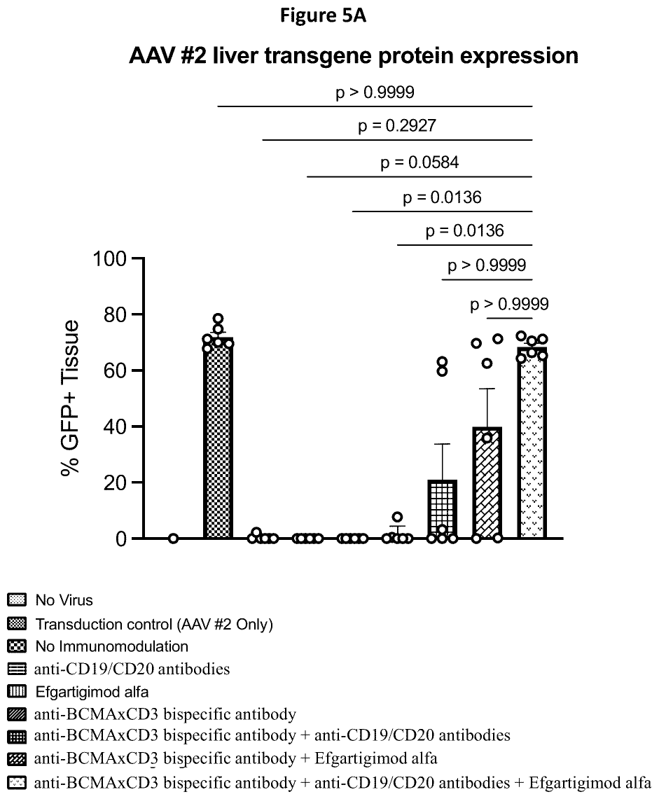

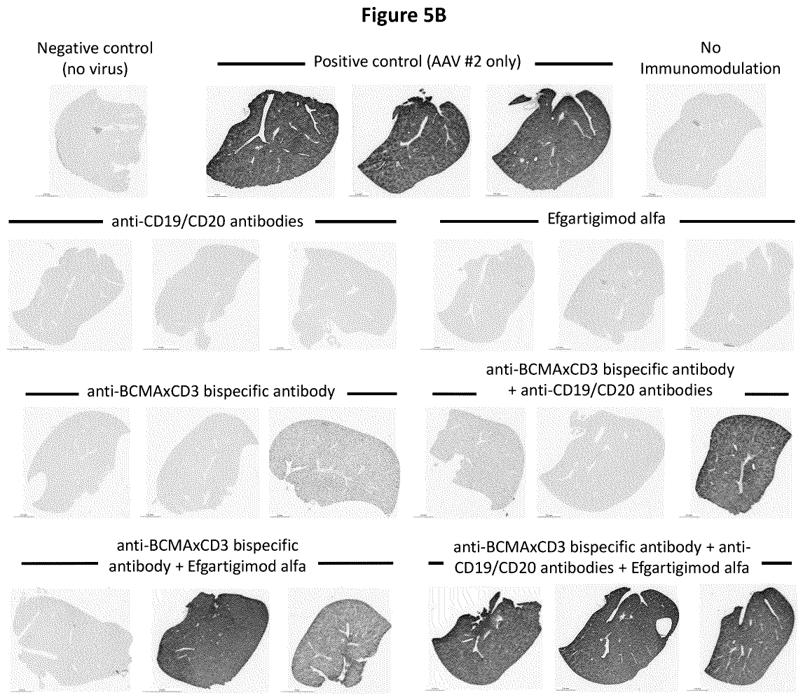

Next was evaluated whether the deep titer reductions observed in mice following combination treatment of anti-BCMA×CD3 bispecific antibody and efgartigimod alfa, and following combination treatment of anti-BCMA×CD3 bispecific antibody, efgartigimod alfa, and anti-CD19/CD20 antibodies, could enable re-transduction with a second AAV vector. To this end, the mice from Example 1 were treated intravenously with 3e12 vg/kg AAV8 GFP, then sacrificed 10 days later to evaluate transgene expression in liver (FIG. 1). While nearly all mice receiving no immunomodulation or single-agent immunomodulation failed to achieve any re-transduction in liver, due to presence of anti-AAV8 antibodies from previous AAV8 exposure, significant levels of GFP transgene DNA (FIG. 3) and RNA (FIG. 4) were observed in mice receiving anti-BCMA×CD3 bispecific antibody+FcRn blocker, and less frequently in mice receiving anti-BCMA×CD3 bispecific antibody+anti-CD19/CD20 antibodies, as measured by quantitative real-time PCR and quantitative real-time reverse transcription PCR, respectively. 3/6 mice receiving anti-BCMA×CD3 bispecific antibody+FcRn blocker achieved re-transduction equivalent to seronegative control mice. The greatest level of re-transduction was observed in the triple combination group, with 6/6 mice achieving transgene levels equivalent to that of previously naive mice. Immunohistochemical staining of formalin-fixed, paraffin-embedded liver sections for GFP transgene protein corroborated these findings (FIGS. 5A-5B). Together, these data indicate that anti-BCMA×CD3 bispecific antibody-mediated plasma cell depletion, particularly in combination with FcRn blockade, can enable AAV re-dosing, or other immunogenic gene therapy vectors, regardless of serostatus, and that the success rate for re-dosing may be further enhanced by B cell depletion in mice.

### Example 3: Analysis of Plasma Cell Frequencies and Counts in Spleen and Bone Marrow Following Anti-BCMA×CD3 Bispecific Antibody Treatment

To confirm on-target activity of anti-BCMA×CD3 bispecific antibody, plasma cell numbers in bone marrow and spleen were evaluated by flow cytometry at the time of sacrifice (FIG. 1). Specifically, single-cell splenocyte suspensions were prepared by mechanical disruption of spleen. For bone marrow extraction, femurs were cut at both ends, placed in a PCR plate with holes punched at the bottom, and spun down for 3 minutes at 500 g. Red blood cells were lysed using ACK lysis buffer. Cells were transferred to a 96 well U-bottom plate, centrifuged at 400 g for 4 minutes and stained with LIVE/DEAD Fixable Blue Dead Cell dye (ThermoFisher) for 15 minutes at room temperature. Cells were washed and incubated in Fc block (Tonbo Biosciences) for 15 to 30 minutes at 4° C. For detection of AAV-specific B cells, splenocytes were incubated with recombinant AAV8 for 1 hr on ice, (multiplicity of infection [MOI] of 10,000) to facilitate interactions between AAV particles and antigen-specific BCRs, followed by washing and labeling with anti-AAV8 biotinylated antibody (clone ADK8, Progen) and surface stain antibody cocktail (Table 3) for 30 minutes at 4° C. in Brilliant Stain buffer (BD Biosciences). Cells were again washed and then stained with Streptavidin-PE conjugate (Biolegend) for an additional 20 minutes at 4° C., followed by washing and fixation with BD Cytofix (BD Biosciences). For intracellular staining, samples were washed and incubated in 1× Perm/Wash buffer (BD Biosciences) for 20 minutes and resuspended in intracellular stain (Table 3) for 30 minutes at 4° C. followed by washing and fixation with BD Cytofix (BD Biosciences). CountBright Absolute counting Beads (ThermoFisher) were used according to the manufacturer's protocol to enumerate absolute cell counts. Acquisition was performed on a BD FACSymphony A5 using FACSDiva software. Analysis was performed using FlowJo or OMIQ software. All B cells were first gated according to light scatter properties, then negatively gated to exclude viability dye positive and non-B cell lineage marker positive cells. Specific B cell populations were then gated as follows: Naïve B cells, CD19+ B220+ CD1d− IgD+ CD38+; Memory B cells, CD19+ B220+ CD1d− IgD− CD38+ AAV+/−; Plasma cells: B220− IgD− CD138+ Light Chain+.

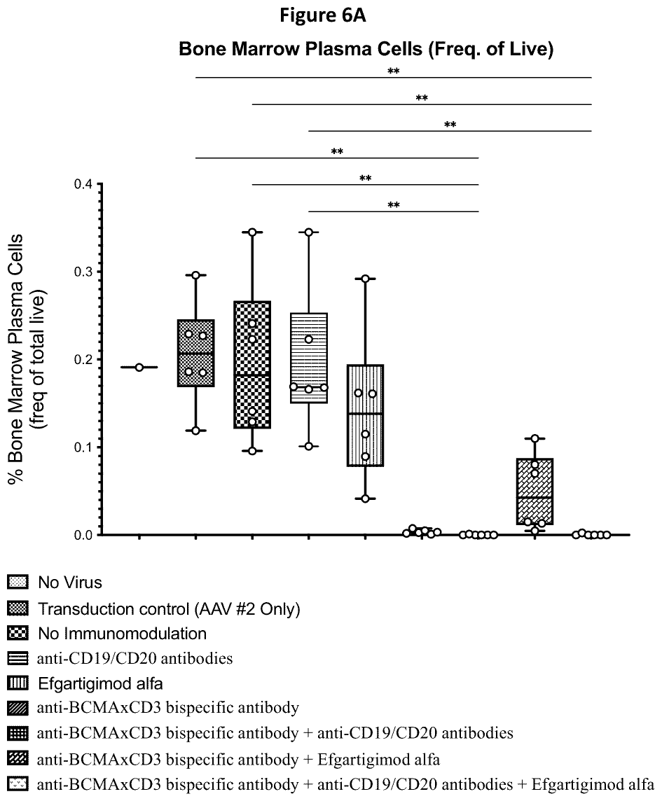

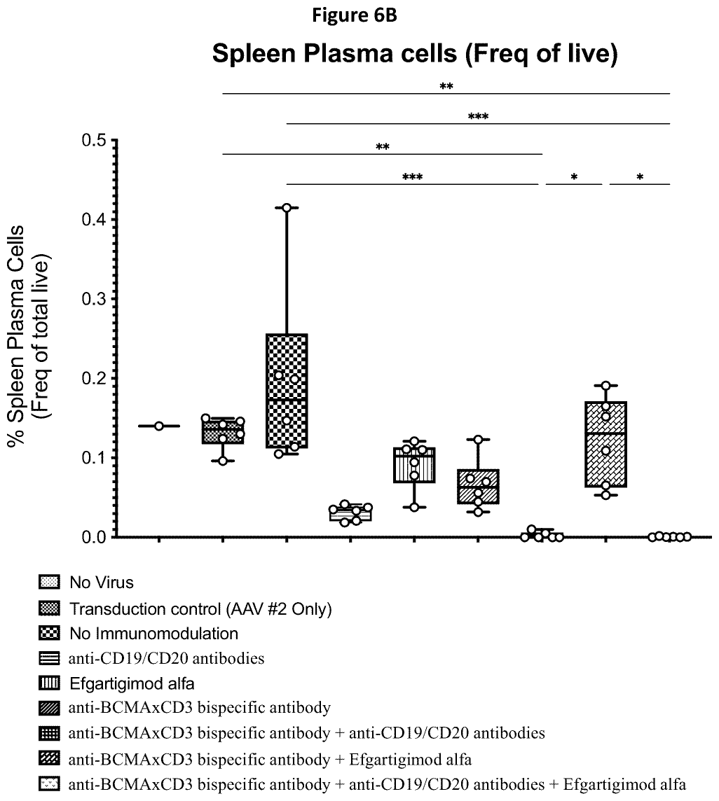

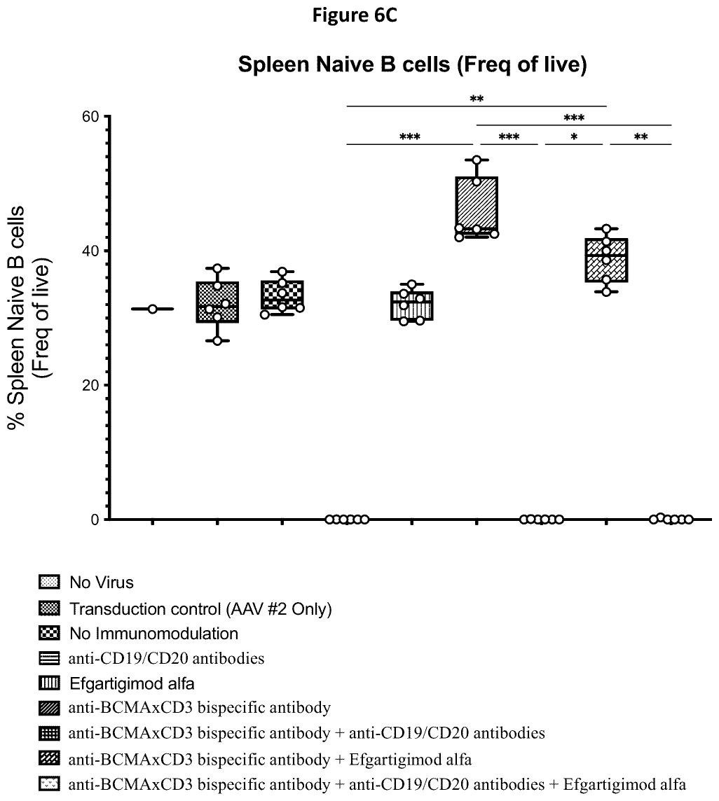

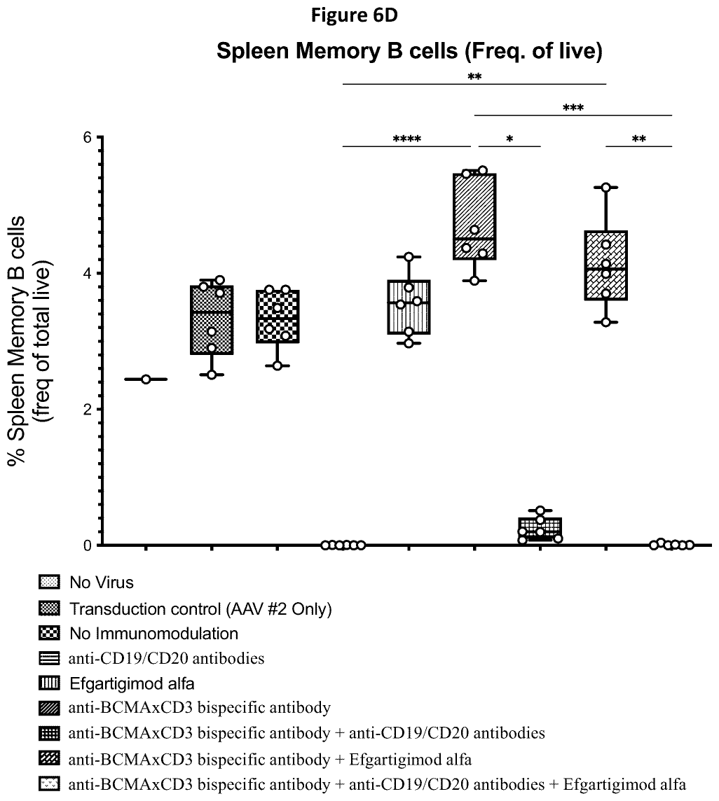

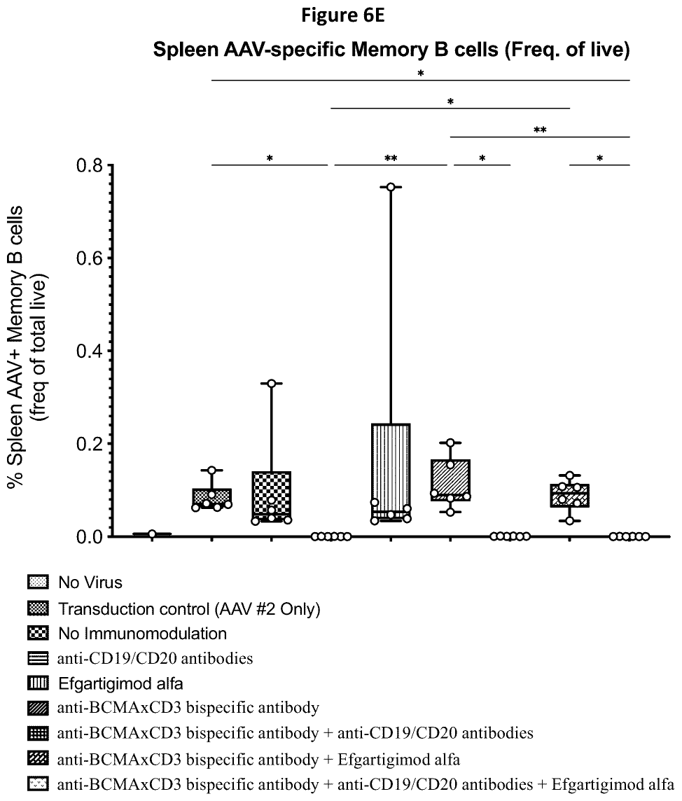

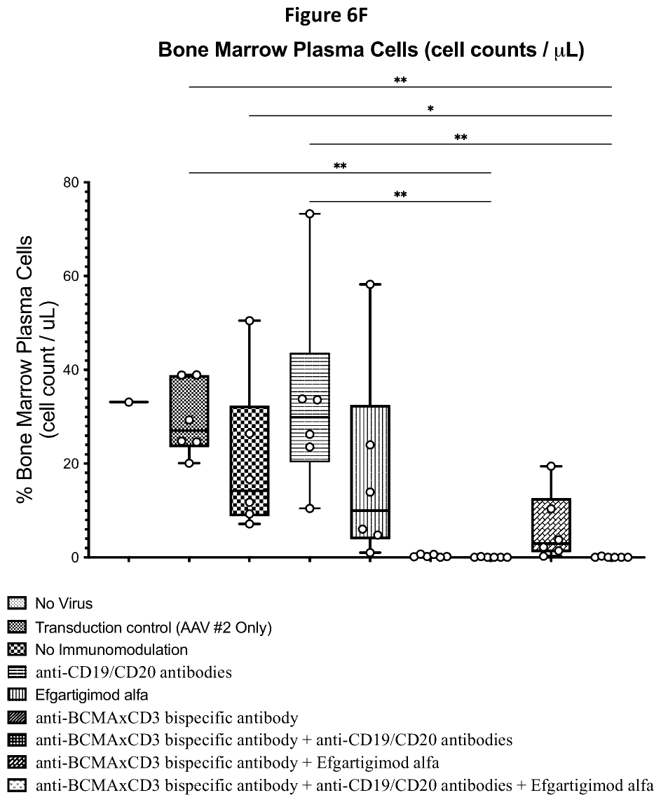

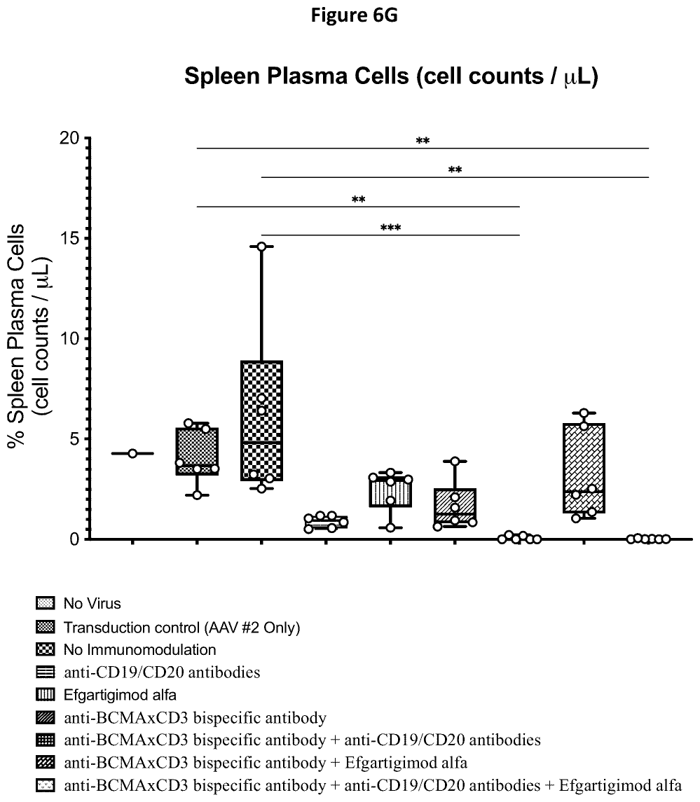

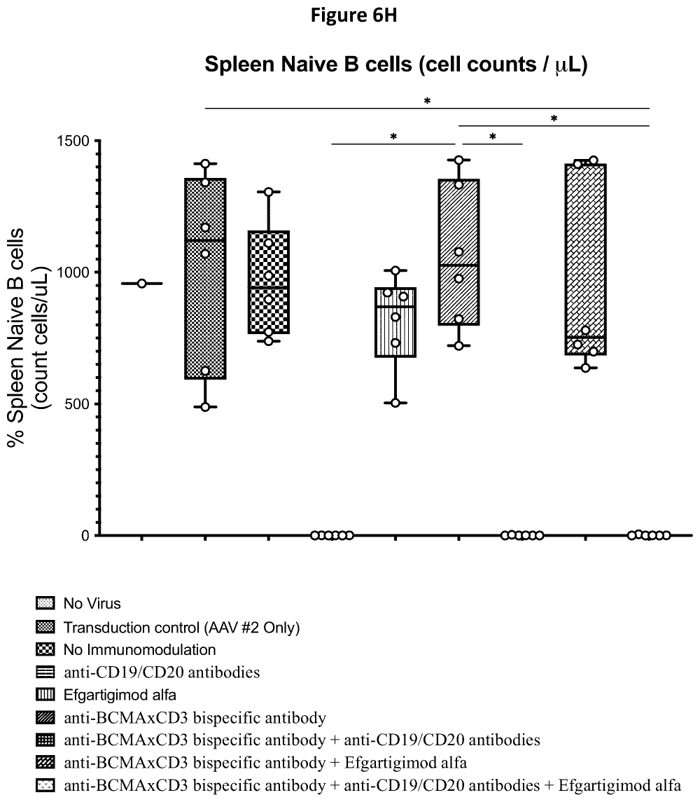

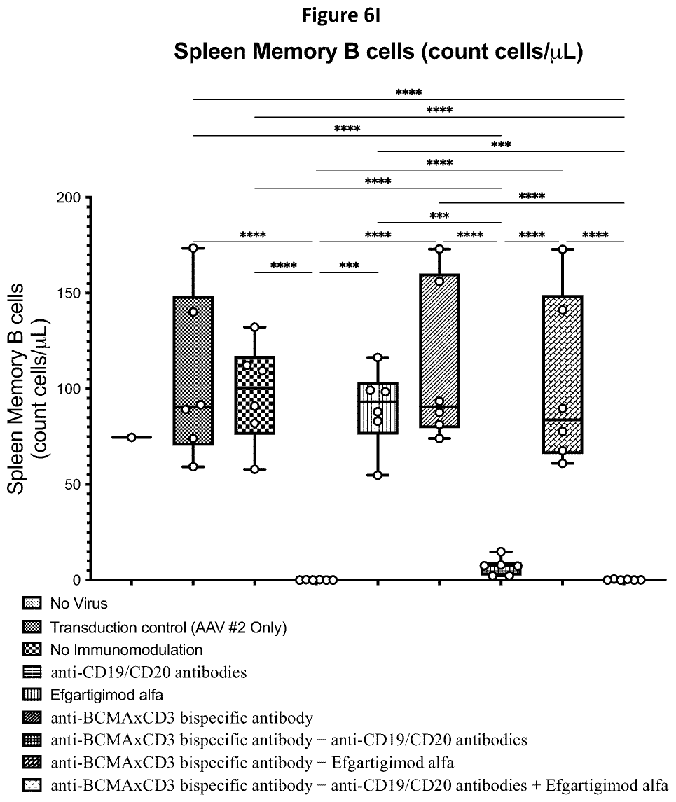

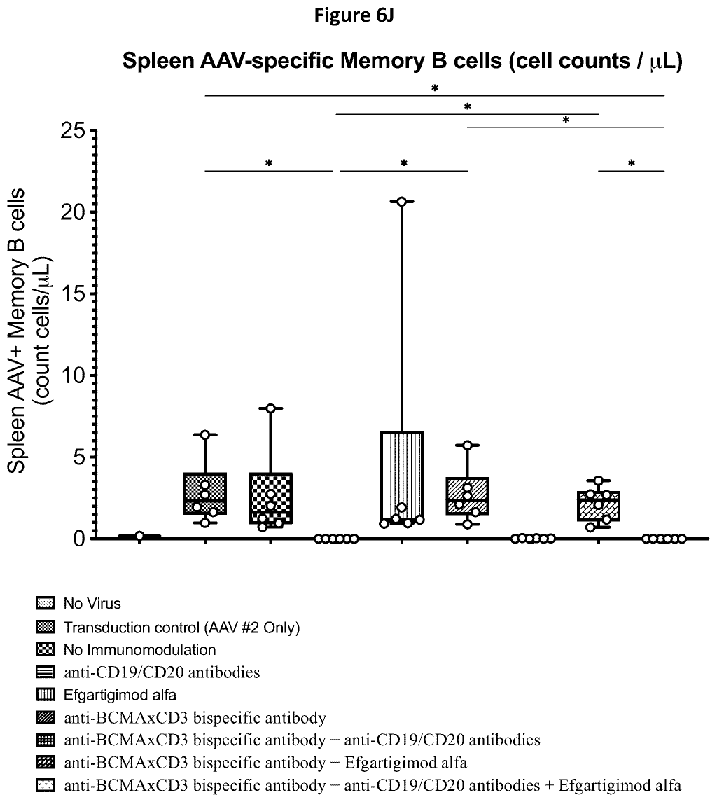

TABLE 3
The flow cytometry antibody staining panel used in FIGS. 6A-6J.
Pre-Stain

| Reagent/Antigen | Conjugate | Reactivity | Host | Clone | Isotype | Supplier |
|---|---|---|---|---|---|---|
| Live/Dead | Blue | N/A | N/A | N/A | N/A | Invitrogen |
| AAV8 | None | N/A | N/A | N/A | N/A | N/A |
| Fc Block (CD16/32) | None | Human | Rat | 2.462 | N/A | TONGO biosciences |

Surface stain

| Antigen | Conjugate | Reactivity | Host | Clone | Isotype | Supplier |
|---|---|---|---|---|---|---|
| CD38 | BUV395 | Mouse/Human | Rat | 90/CD38 | IgG2a, k | BD |
| CD138 | BV711 | Mouse | Rat | 281-2 | IgG2a, κ | BD |
| CD95 | BV421 | Mouse | Hamster | Jo2 | IgG2, λ | BD |
| GL-7 | PerCP CY5.5 | Mouse/Human | Rat | GL7 | IgM, k | Biolegend |
| IgD | BV786 | Mouse | Rat | 11-26c.2a | IgG2a, k | BD |
| IgA | FITC | Mouse | Rat | C10-3 | IgG1, κ | BD |
| IgG1 | BV510 | Mouse | Rat | A85-1 | IgG1, κ | BD |
| CD19 | BUV737 | Mouse | Rat | 1D3 | IgG2a, κ | BD |
| B220 | PE-Cy7 | Mouse | Rat | RA3-682 | IgG2a, κ | BD |
| CD98 | BV605 | Mouse | Hamster | H202-141 | IgG2a, κ | BD |
| CD1d | BUV563 | Mouse | Rat | WTH2 | IgG2a, κ | BD |
| Biotinylated anti-AAV8 IgG | N/A | | Mouse | ADK8 | IgG2a | Progen |
| TCRβ | APC | Mouse | Hamster | H57-597 | IgG2, λ1 | BD |
| CD200R3 | APC | Mouse | Rat | Ba13 | IgG2a, κ | Biolegend |
| Ly6G | APC | Mouse | Rat | 1A8-Ly6g | IgG2a, κ | eBioscience |
| CD49b | APC | Mouse | Rat | DX5 | IgM, κ | Biolegend |
| CD11b | APC | Mouse | Rat | M1/70 | IgG2b, k | Biolegend |

Secondary Stain

| Reagent | Conjugate | Reactivity | Host | Clone | Isotype | Supplier |
|---|---|---|---|---|---|---|
| Streptavidin | PE | N/A | N/A | N/A | N/A | Biolegend |

Intracellullar stain

| Reagent | Conjugate | Reactivity | Host | Clone | Isotype | Supplier |
|---|---|---|---|---|---|---|
| Light Chain k | BV650 | Mouse | Rat | 187.1 | IgG1, k | BD |
| Light Chain l | BV650 | Mouse | Rat | R26-46 | IgG2a, κ | BD |
| IgG1 | BV510 | Mouse | Rat | A85-1 | IgG1, k | BD |
| IgA | FITC | Mouse | Rat | C10-3 | IgG1, κ | BD |

Analysis of B cell and plasma cell frequencies (FIGS. 6A-6E) and cell counts (FIGS. 6F-6J) in the bone marrow and spleen revealed that plasma cells were fully depleted in groups receiving anti-BCMA×CD3 bispecific antibody+anti-CD19/CD20 antibodies and the triple combination of anti-BCMA×CD3 bispecific antibody+efgartigimod alfa+anti-CD19/CD20 antibodies, consistent with reductions in anti-AAV8 titers seen in these groups. However, plasma cells were incompletely depleted in anti-BCMA×CD3 bispecific antibody groups that did not also receive anti-CD19/CD20 antibodies, suggesting either that ongoing plasma cell formation is a significant contributor to the anti-AAV8 IgG antibody pool in this model, or that mice developed anti-drug antibodies that react with anti-BCMA×CD3 bispecific antibody (a human IgG) that may have limited its therapeutic effect in the absence of B cell depletion (which would also deplete anti-human IgG-specific B cells). The effectiveness of anti-CD19/CD20 antibodies-mediated B cell depletion was also confirmed in analyses of naïve, memory, and AAV-specific B cells, with all subsets fully depleted except for a small subset of total memory B cells that were not depleted in the anti-BCMA×CD3 bispecific antibody+anti-CD19/CD20 antibodies combination group. Collectively, these data show that the titer reductions observed in the anti-BCMA×CD3 bispecific antibody+efgartigimod alfa+anti-CD19/CD20 antibodies are consistent with the expected mechanism of action, and that, by comparison, incomplete titer reductions observed in the anti-BCMA×CD3 bispecific antibody+efgartigimod alfa group may be explained by incomplete plasma cell depletion, possibly due to development of anti-drug antibodies reactive with anti-BCMA×CD3 bispecific antibody, efgartigimod, or both.

### Example 4: Use of BCMA×CD3 and FcRn Blockade for Suppressing Pre-Existing Anti-AAV Antibody Titers Arising from Natural AAV Exposure to Enable Recombinant AAV Transduction

In this example, plasma cell depletion with BCMA×CD3 is utilized in combination with FcRn antagonism to suppress pre-existing antibody titers to AAV arising from natural AAV exposure, and consequently enable recombinant AAV transduction. Cynomolgus macaques with pre-existing total AAV antibody titers and AAV nAb titers ranging from low to high titer, are treated with BCMA×CD3 with or without efgartigimod alfa (or alternative FcRn blocking therapeutic), and with or without CD3×CD20 bispecific antibody (or alternative B cell depletion therapeutic) weekly. Anti-AAV8 neutralizing and total antibody titers are evaluated biweekly. Several weeks later, monkeys are dosed intravenously with a recombinant AAV8 vector encoding GFP transgene. Monkeys treated with BCMA×CD3 alone are expected to show gradual decline in pre-existing anti-AAV8 antibody titers, but the effect is expected to be accelerated and magnified in groups additionally receiving FcRn antagonist, or groups additionally receiving FcRn antagonist and B cell depletion, with titers being reduced to sub-neutralizing levels. Monkeys not receiving BCMA×CD3 are not expected to show the same frequency, rate, or magnitude of titer decline. Monkeys receiving BCMA×CD3+FcRn antagonist and BCMA×CD3+FcRn antagonist+B cell depletion combination treatment groups are expected to exhibit successful transduction with a second AAV8 vector at levels higher than achieved by any single agent alone or any combination that does not include BCMA×CD3.

### Example 5: Use of BCMA×CD3 in Combination with IgG-Degrading Enzyme to Suppress Pre-Existing Anti-AAV Titers to Enable Recombinant AAV Transduction

In this example, plasma cell depletion with BCMA×CD3 is utilized in combination with IgG degrading enzyme to suppress pre-existing antibody titers to AAV arising from natural AAV exposure, thereby enabling recombinant AAV transduction. Cynomolgus macaques with pre-existing total AAV antibody titers and AAV nAb titers ranging from low to high titer, are treated with BCMA×CD3 with or without CD20×CD3 bispecific antibody (or alternative B cell depletion therapeutic) weekly for several weeks, followed by one or two doses of IgG-degrading enzyme (IdeS or similar). Anti-AAV8 neutralizing and total antibody titers are evaluated biweekly prior to IdeS administration, and daily following IdeS administration. Antibody titers are expected to decline gradually after BCMA×CD3 and BCMA×CD3+CD20×CD3 treatment, but show rapid and greater magnitude decline with additional IdeS treatment, with titers being reduced to sub-neutralizing levels. Monkeys not receiving BCMA×CD3 are not expected to show the same frequency, rate, or magnitude of titer decline. Monkeys are then treated intravenously with an AAV8 vector encoding transgene of interest. Monkeys receiving BCMA×CD3+IdeS and BCMA×CD3+IdeS+B cell depletion combination treatment groups are expected to exhibit successful transduction with the second AAV8 vector at levels higher than achieved by any single agent alone or any combination that does not include BCMA×CD3.

### Example 6: Use of BCMA×CD3 in Combination with IgG-Degrading Enzyme to Suppress Anti-AAV Titers Following Recombinant AAV Exposure to Enable AAV Vector Re-Administration

In this example, plasma cell depletion with BCMA×CD3 is utilized in combination with IgG degrading enzyme to suppress antibody titers arising from recombinant AAV treatment to enable repeated recombinant AAV transduction. AAV seronegative cynomolgus macaques are first treated with an AAV8 vector, resulting in high titer of neutralizing anti-AAV8 antibodies. Monkeys are then treated with BCMA×CD3, with or without CD20×CD3 bispecific antibody (or alternative B cell depletion therapeutic) weekly for several weeks, followed by one or two doses of IgG-degrading enzyme (IdeS or similar). Anti-AAV8 neutralizing and total antibody titers are evaluated biweekly prior to IdeS administration, and daily following IdeS administration. Antibody titers are expected to decline gradually after BCMA×CD3 and BCMA×CD3+CD20×CD3 treatment, but show rapid and greater magnitude decline with additional IdeS treatment, with titers being reduced to sub-neutralizing levels. Monkeys not receiving BCMA×CD3 are not expected to show the same frequency, rate, or magnitude of titer decline. Monkeys are then treated intravenously with a second AAV8 vector encoding the same or second transgene of interest. Monkeys receiving BCMA×CD3+IdeS and BCMA×CD3+IdeS+B cell depletion combination treatment groups are expected to exhibit successful transduction with the second AAV8 vector at levels higher than achieved by any single agent alone or any combination that does not include BCMA×CD3.

### Example 7: Use of BCMA×CD3 in Combination with Therapeutic Plasma Exchange to Suppress Pre-Existing Anti-AAV Titers to Enable Recombinant AAV Transduction

In this example, plasma cell depletion with BCMA×CD3 is utilized in combination with therapeutic plasma exchange (TPE) to suppress pre-existing antibody titers to AAV arising from natural AAV exposure, thereby enabling recombinant AAV transduction. Cynomolgus macaques with pre-existing total AAV antibody titers and AAV nAb titers ranging from low to high titer, are treated with BCMA×CD3 with or without CD20×CD3 bispecific antibody (or alternative B cell depletion therapeutic) weekly for several weeks. Monkeys are then subjected to one or more rounds of TPE. Anti-AAV8 neutralizing and total antibody titers are evaluated prior to and following each round of TPE. Antibody titers are expected to decline gradually after BCMA×CD3 and BCMA×CD3+CD20×CD3 treatment, but show accelerated and substantial decline following each round of TPE, with titers ultimately being reduced to sub-neutralizing levels. Monkeys not receiving BCMA×CD3 are not expected to show the same frequency, rate, or magnitude of titer decline. Monkeys are then treated intravenously with an AAV8 vector encoding transgene of interest. Monkeys receiving BCMA×CD3+TPE, or BCMA×CD3+B cell depletion+TPE combination treatment are expected to exhibit successful transduction with the second AAV8 vector at levels higher than achieved by any single agent alone or any combination that does not include BCMA×CD3.

### Example 8: Use of BCMA×CD3 in Combination with Therapeutic Plasma Exchange to Suppress Anti-AAV Titers Following Recombinant AAV Exposure to Enable AAV Vector Re-Administration

In this example, plasma cell depletion with BCMA×CD3 is utilized in combination with therapeutic plasma exchange (TPE) to suppress antibody titers arising from recombinant AAV treatment to enable repeated recombinant AAV transduction. AAV seronegative cynomolgus macaques are first treated with an AAV8 vector, resulting in high titer of neutralizing anti-AAV8 antibodies. Monkeys are then treated with BCMA×CD3 with or without CD20×CD3 bispecific antibody (or alternative B cell depletion therapeutic) weekly for several weeks. Monkeys are then subjected to one or more rounds of TPE. Anti-AAV8 neutralizing and total antibody titers are evaluated prior to and following each round of TPE. Antibody titers are expected to decline gradually after BCMA×CD3 and BCMA×CD3+CD20×CD3 treatment, but show accelerated and substantial decline following each round of TPE, with titers ultimately being reduced to sub-neutralizing levels. Monkeys not receiving BCMA×CD3 are not expected to show the same frequency, rate, or magnitude of titer decline. Monkeys are then treated intravenously with a second AAV8 vector encoding the same or second transgene of interest. Monkeys receiving BCMA×CD3+plasmapheresis, and BCMA×CD3+B cell depletion+TPE combination treatment groups are expected to exhibit successful transduction with the second AAV8 vector at levels higher than achieved by any single agent alone or any combination that does not include BCMA×CD3.

### Example 9: Negative Impact of Efgartigimod on BCMA×CD3 Serum Drug Concentration, which is Partially Preventable with B Cell Depletion, is Consistent with Cross-Reactive Anti-Drug Antibody Formation

Robust bone marrow plasma cell depletion was observed in BCMA×CD3, BCMA×CD3+anti-CD19/CD20, and BCMA×CD3+anti-CD19/CD20+efgartigimod treatment groups that was consistent with the expected mechanism of action of BCMA×CD3 observed in previous studies (Limnander et al., 2023). However, mice treated with BCMA×CD3+efgartigimod, but not anti-CD19/CD20, unexpectedly showed incomplete bone marrow plasma cell depletion (FIG. 6A and FIG. 6F). To better understand the impact of efgartigimod on the efficacy of BCMA×CD3, serum BCMA×CD3 concentrations were evaluated during the immunomodulation treatment period. Repeated injections of BCMA×CD3 in BCMA×CD3 and BCMA×CD3+anti-CD19/20 treatment groups resulted in expected increases in serum concentrations of BCMA×CD3 (FIG. 7). Moreover, mice treated with BCMA×CD3+anti-CD19/20+efgartigimod showed significantly lower levels of serum BCMA×CD3, as expected from the mechanism of action of efgartigimod, which blocks FcRn-mediated recycling of IgG, including recycling of therapeutic IgGs such as BCMA×CD3. However, mice receiving BCMA×CD3+efgartigimod without anti-CD19/20 exhibited an even greater rate of BCMA×CD3 antibody clearance that resulted in complete loss of BCMA×CD3 in serum starting 13 days after the initial efgartigimod dose (FIG. 7).

A faster clearance rate, reversible with B cell depletion, suggests that a humoral immune response may be contributing to drug clearance via development of anti-drug antibodies. Efgartigimod is a human IgG1 antibody fragment and is known to be immunogenic in mice. BCMA×CD3 is a human IgG4, which possesses >90% sequence identity to hIgG1. Therefore, it was concluded that efgartigimod, in the absence of additional B cell depletion, induced human IgG1/IgG4 cross-reactive anti-drug antibodies that accelerated BCMA×CD3 clearance. It was further concluded that this anti-drug antibody response, in the absence of additional B cell depletion, negatively impacted BCMA-mediated bone marrow plasma cell depletion and consequently AAV titer reductions. Thus, the contribution of anti-CD19/20 antibodies to the efficacy of BCMA×CD3 in non-human systems, in the presence of efgartigimod, may be model-specific through prevention of a xenogeneic anti-drug antibody response.

### Example 10: Plasma Cell Depletion in Combination with Neonatal Fc Receptor (FcRn) Blockade Potently Reduces Naturally-Occurring AAV Titers in AAV-Seropositive Cynomolgus Macaques

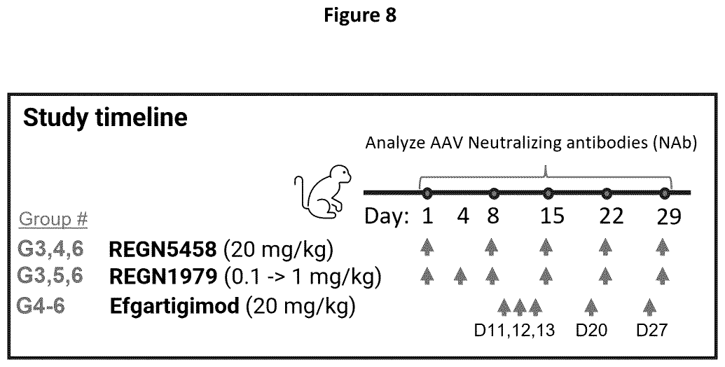

To evaluate whether plasma cell depletion with BCMA×CD3 could similarly suppress naturally-occurring AAV titers arising from exposure to wild-type AAVs, as is common in humans, a non-human primate study was initiated with AAV8-seropositive macaques. Macaques were divided by AAV neutralizing antibody (nAb) titer into five treatment groups (n=3-5 each), each containing animals of high nAb titer (>1:450), as well as one group of seronegative control animals (n=3). Subsequently, animals were treated with various combinations of a plasma cell-depleting bispecific BCMA×CD3 antibody (REGN5458, 20 mg/kg weekly), a B cell-depleting bispecific CD20×CD3 antibody (REGN1979, 0.1 mg/kg on Study Day 1, 1 mg/kg on Study Day 4, 8, and weekly thereafter), and/or an FcRn blocker (efgartigimod, 20 mg/kg on Study Day 11, 12, 13, 20, and 27). Efgartigimod dosing was delayed relative to REGN5458 and REGN1979 dosing to minimize the impact of efgartigimod on REGN5458 and REGN1979 drug half-life due to FcRn blockade and/or cross-reactive anti-drug antibody development. NAb titers were analyzed weekly by cell-based neutralization assay, conducted by VRL Diagnostics (San Antonio, Texas). A schematic of the study design is shown is FIG. 8.

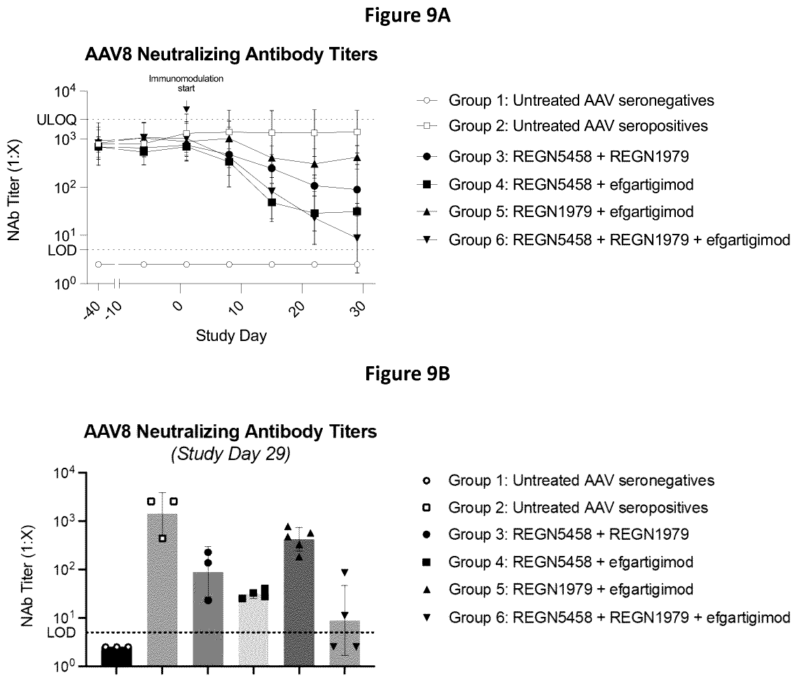

Longitudinal analysis of NAb titers revealed that only groups treated with immunomodulation cocktails containing REGN5458 showed substantive geometric mean titer reductions by ˜4 weeks after the start of immunomodulation. Whereas macaques receiving REGN5458-containing cocktails showed titer reductions of >10-fold, macaques receiving a cocktail containing efgartigimod and REGN1979, but not REGN5458, showed only marginal geometric mean nAb titer reduction of ˜2-fold (FIG. 9A). Similar to findings in mice, cocktails containing both a plasma cell depleter (REGN5458) and an FcRn blocker (efgartigimod), or all three immunomodulators, elicited the greatest titer reductions, with the triple combination of REGN5458, REGN1979, and efgartigimod inducing a >100-fold reduction in nAb titer (FIG. 9A). On Study Day 29, two animals in the triple combination group exhibited nAb titers below the limit of detection of the assay (FIG. 9B), suggesting that these animals could be successfully dosed with an AAV vector. Thus, plasma cell depletion is an effective strategy for suppressing naturally-occurring anti-AAV antibody titers, even of high-titer, to a level that is compatible with AAV dosing.

### Example 11: Prophylactic B Cell Depletion with CD20×CD3 More Effectively Suppresses the Antibody Response to AAV Vectors than Conventional Anti-CD20 Monoclonal Antibodies in Mice

B cell depletion with rituximab (or anti-CD20 mouse equivalents) have been evaluated in both preclinical and clinical settings as a strategy to prevent the antibody response to AAV and enable AAV vector re-administration. However, in both preclinical and clinical settings, prophylactic treatment with anti-CD20 antibodies has been shown to only partially suppress the antibody response to AAV vectors, and thus have failed to enable re-dosing (Salabarria et al 2024, Choi et al 2023, Meliani et al 2018, Unzu et al 2012).

It was reasoned herein that rituximab and other anti-CD20 monoclonal antibodies may sub-optimally suppress anti-AAV antibody responses due to incomplete B cell depletion in secondary lymphoid tissues, the sites where antibody responses are initiated. It was further reasoned that CD20×CD3 bispecific antibodies, which achieve superior depletion of B cells in lymphoid tissues versus rituximab (Smith et al 2015), would consequently be more effective at preventing the antibody response to AAV.

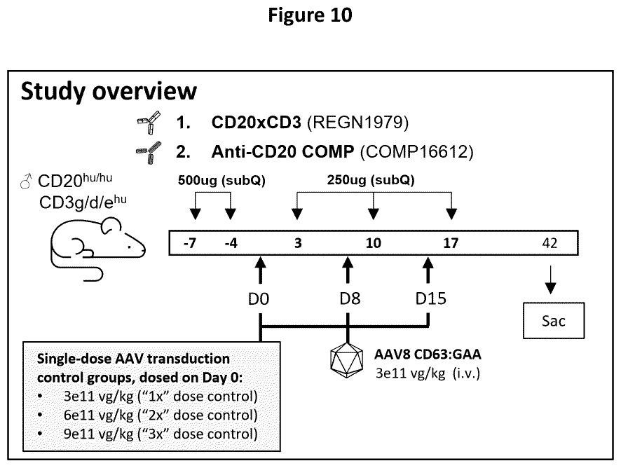

To test this, the effectiveness of a CD20×CD3 bispecific antibody (REGN1979) versus a rituximab comparator molecule (“Anti-CD20 COMP”) at suppressing the anti-AAV antibody response to repeated doses of AAV in mice was compared. A schematic of the study design is shown in FIG. 10. Specifically, mice humanized for CD20 and CD3 (gamma, delta, and epsilon chains) were treated prophylactically with two doses of either REGN1979 or anti-CD20 COMP (each 500 μg per mouse subQ (subcutaneously, s.c.)) on Study Days −7 and −4, then weekly for three weeks (250 μg per mouse per dose) starting on Study Day 3 to maintain B cell depletion. A separate group of mice received no B cell depleting agent (“No immunomodulation”). On Study Day 1, an AAV8 vector encoding acid alpha glucosidase fused to a single chain variable fragment targeting human CD63 (hereinafter, “anti-CD63 scFv:GAA”) was dosed intravenously at 3e11 vector genomes (vg) per kilogram (kg), then subsequently re-administered at the same dose (3e11 vg/kg) on Study Days 8 and 15. As AAV transduction controls, separate groups of mice were administered a single dose of AAV equal to one, two, or three doses (3e11, 6e11, or 9e11 vg/kg) on Study Day 1 only, in the absence of any B cell depletion agent.

Antibody titers were evaluated using anti-AAV8 IgM and IgG ELISA. Briefly, 96-well flat-bottom plates were coated with 1e9 vg/well recombinant AAV8 vector in DPBS overnight. The next day, plates were washed and blocked with a milk-based blocking agent (KPL SeraCare Milk Blocking Solution) for 1 hr. Serum samples were then diluted in the same blocker, beginning at an initial dilution of 1:300 followed by three-fold serial dilutions to a final dilution of 53,144,100. Diluted serum was then transferred to the assay plate and incubated overnight at 4° C. The next day, the assay plates were repeatedly washed prior to 1 hr incubation with a polyclonal secondary antibody targeting either mouse IgM (HRP-conjugated AffiniPure Goat Anti-Mouse IgM, μ chain specific, Jackson ImmunoResearch) or mouse IgG (HRP-conjugated anti-mouse Fcγ Fragment, Jackson ImmunoResearch), each diluted 1:5000 in DPBS+0.5% BSA. Plates were again repeatedly washed prior to development with TMB substrate solution. After 15-20 minutes, the reaction was stopped by addition of 2N phosphoric acid. Absorbance at 450 nm (OD450) was measured on a SpectraMax i3 plate reader (Molecular Devices, San Jose, CA). Anti-AAV8 IgM and IgG titers, defined as the dilution factor required to achieve an OD450 reading equal to 2-fold background, were determined and plotted using Prism software (v.10.1, GraphPad, Boston, MA).

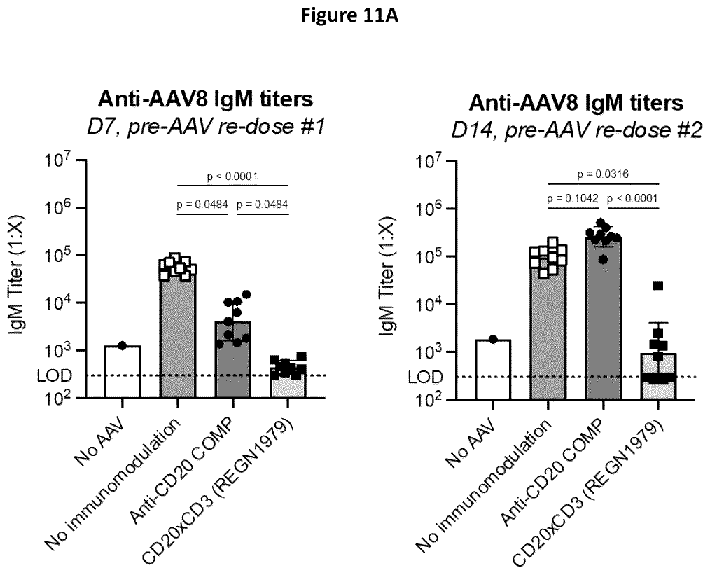

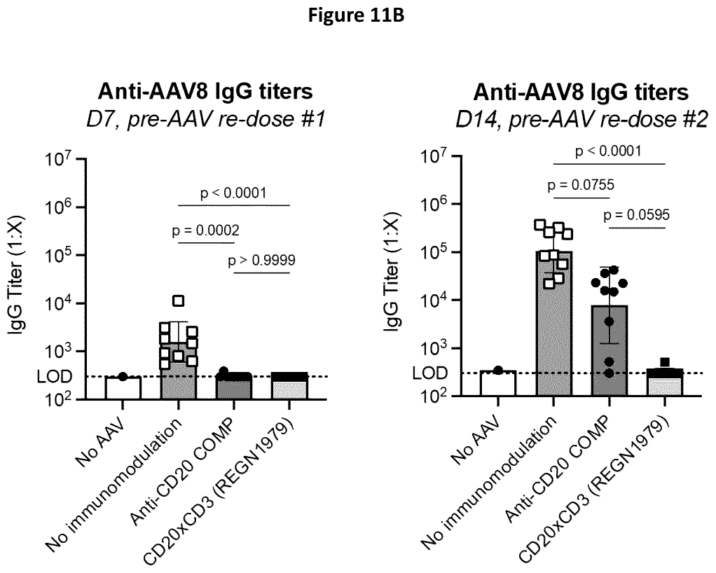

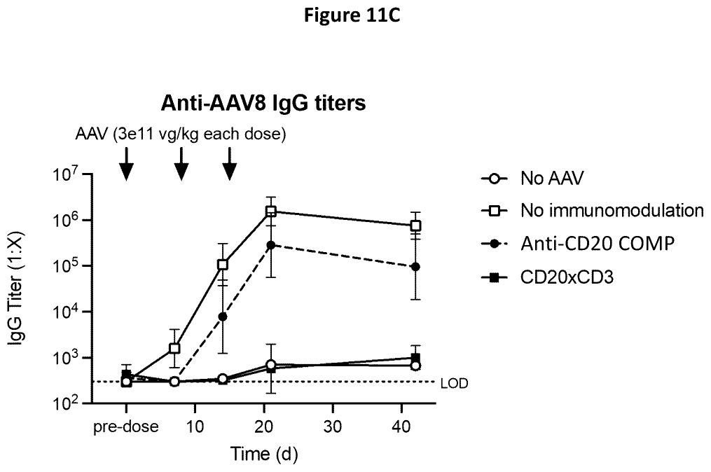

The results showed that AAV IgM titers just prior to AAV re-dose #1 (on Study Day 7) and AAV re-dose #2 (on Study Day 14) were more significantly suppressed in REGN1979-treated mice versus anti-CD20 COMP-treated mice, with REGN1979-treated mice exhibiting geometric mean titers that were essentially at undetectable levels at both timepoints (equivalent to or below that of a control AAV-naïve mouse) (FIG. 11A). By contrast, anti-CD20 COMP-treated mice showed only partial IgM titer suppression at the time of AAV re-dose #1, and no suppression at the time of AAV re-dose #2. Similarly, evaluation of anti-AAV8 IgG titers revealed that REGN1979-treated mice exhibited complete suppression of the anti-AAV8 IgG response at both timepoints, whereas anti-CD20 COMP-treated mice showed clearly detectable IgG titers by Study Day 14 (FIG. 11B). Longitudinal examination of anti-AAV IgG titers showed that REGN1979-treated mice maintained strong IgG titer suppression until study termination (Day 42), whereas anti-CD20 COMP-treated animals eventually mounted a strong IgG response that peaked only slightly below levels of control mice receiving no immunomodulation FIG. 11C. Thus, mice treated with CD20×CD3 bispecific antibody show superior suppression of anti-AAV antibody titers versus mice receiving conventional anti-CD20 monoclonal antibody.

### Example 12: Prophylactic B Cell Depletion with CD20×CD3, but not Conventional Anti-CD20 Monoclonal Antibody, Enables Systemic AAV8 Vector Re-Administration in Mice

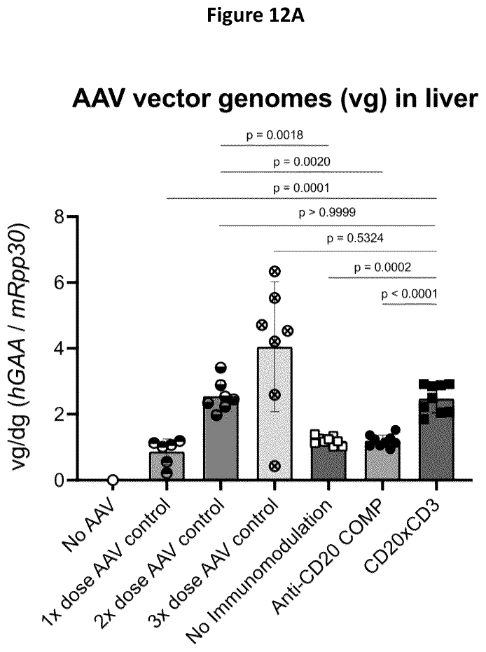

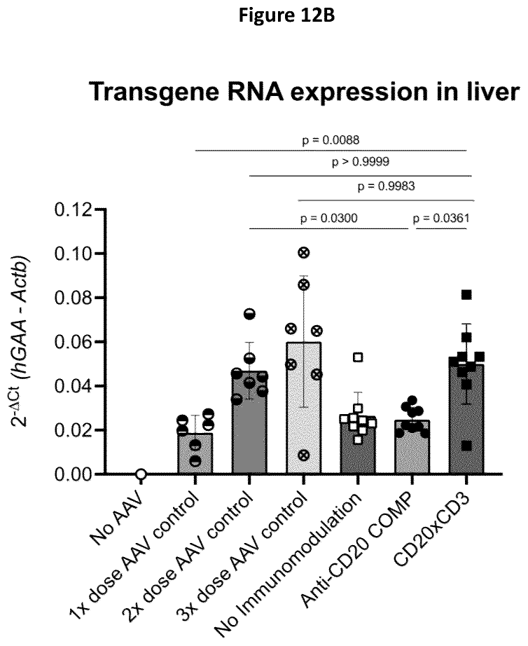

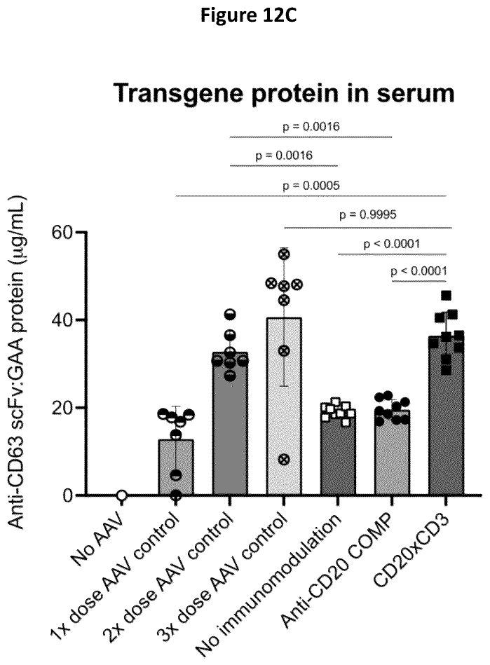

To evaluate whether the levels of anti-AAV titer suppression achieved by REGN1979 were sufficient to enable AAV re-dosing, mice from Example 11 were sacrificed on Study Day 42 for analysis of AAV transduction in liver. Analysis of AAV vector genomes by digital PCR revealed that REGN1979-treated mice exhibited levels of transduction in liver that were significantly higher than anti-CD20-treated mice and No Immunomodulation controls, and comparable to transduction control animals that received the equivalent of two AAV doses as a single dose (total 6e13 vg/kg) (FIG. 12A). By contrast, anti-CD20-treated mice showed no benefit versus no immunomodulation mice, failing to achieve meaningful levels of re-transduction. Transgene RNA expression analysis in liver (by RT-qPCR), and protein expression in serum (by ELISA) showed similar findings, with REGN1979-treated mice again exhibiting significantly higher levels of liver transgene RNA and protein expression versus anti-CD20-treated mice, at levels between that of transduction control mice receiving the equivalent of 2 or 3 AAV doses as a single dose (FIGS. 12B-12C). Taken together, these findings demonstrate that prophylactic B cell depletion with CD20×CD3 bispecific antibody, but not conventional anti-CD20 monoclonal antibody, allows for successful AAV vector re-administration.

### Example 13: Prophylactic B Cell Depletion with CD20×CD3 Fully Suppresses the IgM, IgG, and Neutralizing Antibody (nAb) Response to AAV in Non-Human Primates

Previous reports have shown that B cell depletion with rituximab is insufficient to prevent antibody responses to systemic AAV in non-human primates, and ultimately fails to enable re-dosing (Unzu et al 2012). It was thus investigated whether more potent B cell depletion with CD20×CD3 bispecific antibody could achieve superior suppression of the antibody response post-AAV dosing, to levels that would facilitate re-dosing.

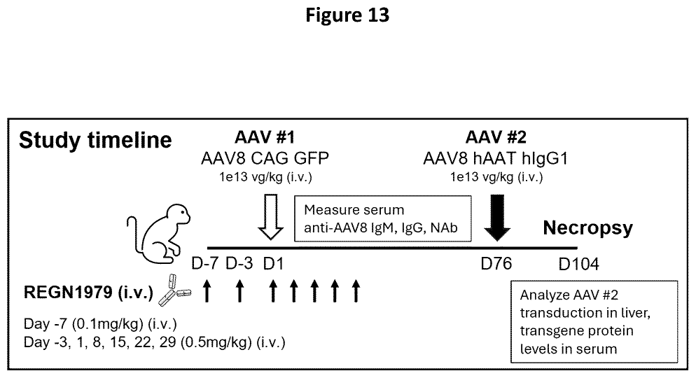

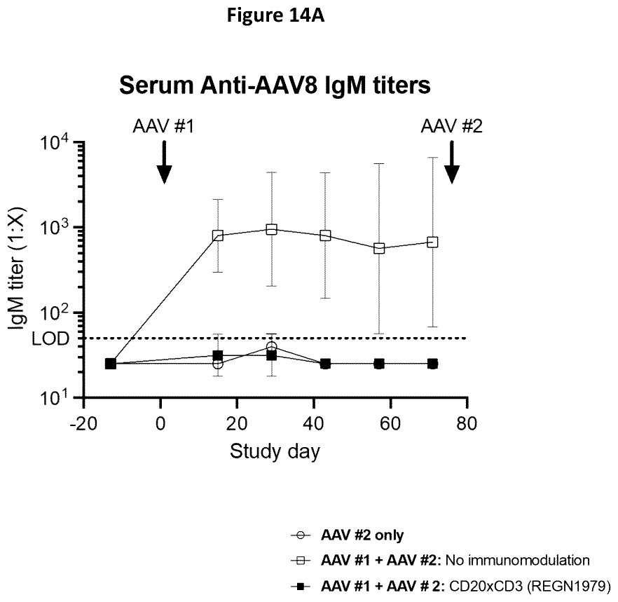

AAV8-seronegative cynomolgus macaques received either no immunomodulation (n=4) or CD20×CD3 bispecific antibody (REGN1979, 0.1 mg/kg initial dose then 0.5 mg/kg weekly for 5 weeks; n=6). After three initial doses of REGN1979, animals in both groups were administered AAV8 CAG eGFP (“AAV #1”) intravenously (i.v.) at 1e13 vector genomes per kilogram (vg/kg). A third group was not administered the 1st AAV (“AAV #2 only”; n=6). Blood was sampled for 10 weeks for longitudinal antibody titer analysis. A schematic of the study design is shown in FIG. 13.

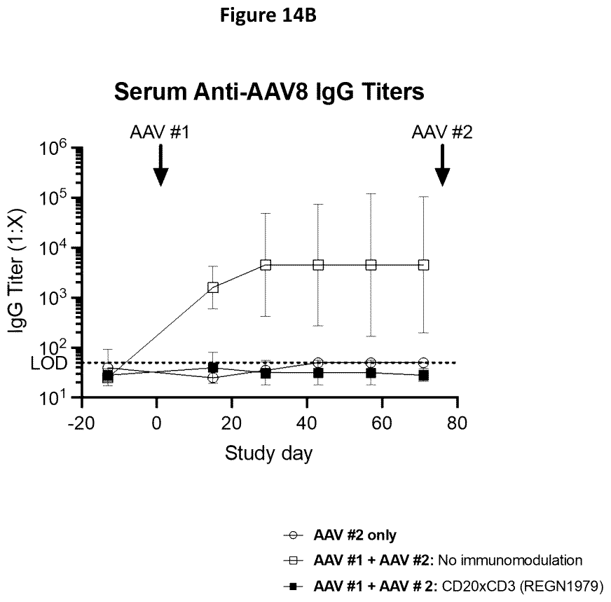

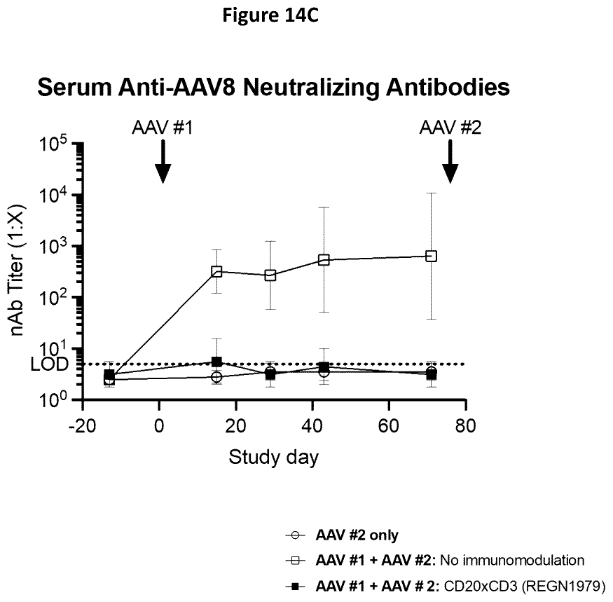

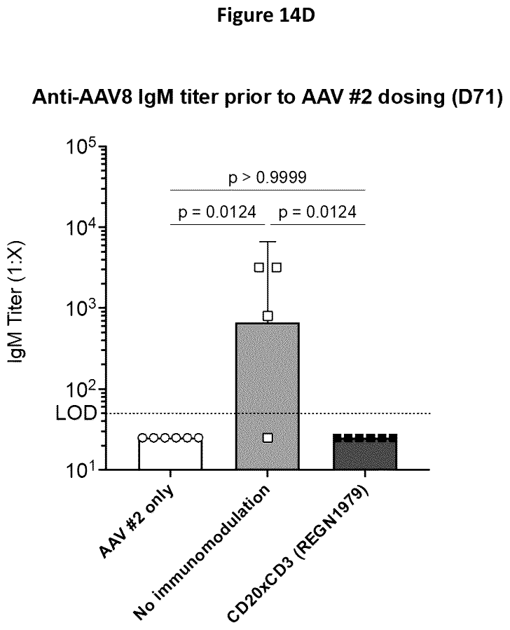

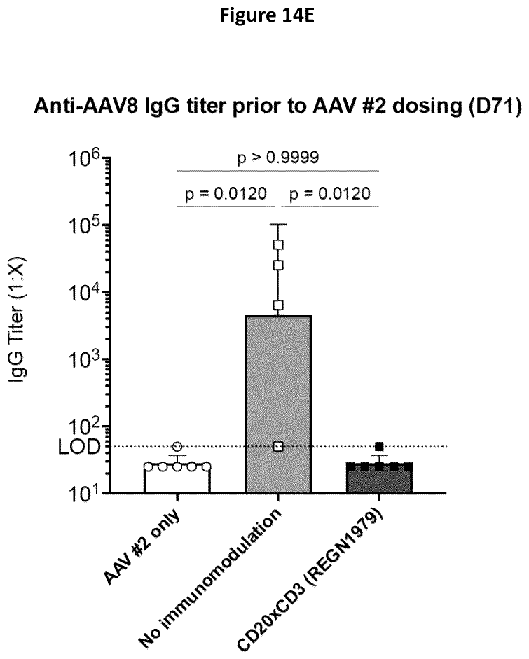

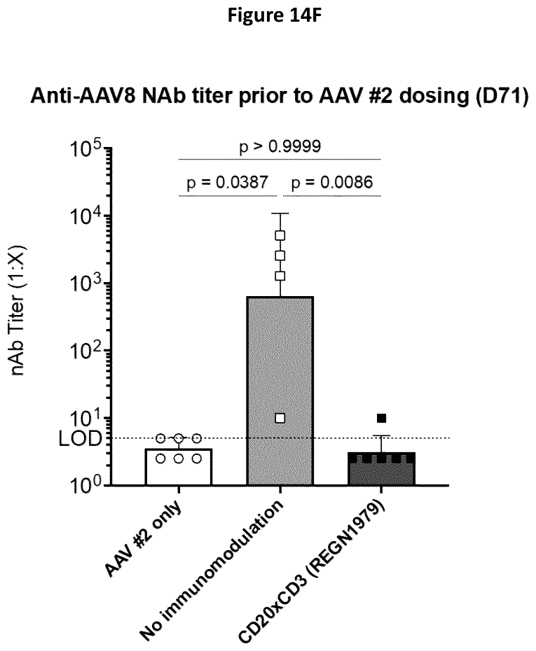

Analysis of anti-AAV IgM, IgG, and neutralizing antibody (nAb) titers was conducted by AAV titer ELISA or cell-based neutralization assay, according to standard methods known in the art. Results indicated that, as expected, macaques receiving no immunomodulation mounted a robust AAV8 IgM, IgG, and nAb response after the first AAV exposure (FIGS. 14A-14C). Strikingly, however, prophylactic B cell depletion with REGN1979 completely prevented an IgM, IgG, and nAb response for the entire 10-week analysis period (FIGS. 14A-14C). Individual animal IgM, IgG, and nAb titer data are shown for Study Day 71, 5 days prior to AAV re-dosing, in FIGS. 14D-14F. Thus, these data indicate that prophylactic B cell depletion with CD20×CD3 bispecific antibody can fully prevent the antibody response to AAV in non-human primates, in contrast to comparable studies evaluating a conventional anti-CD20 therapeutic (rituximab), which failed to prevent the anti-AAV NAb response (Unzu et al 2012).

### Example 14: Prophylactic B Cell Depletion with CD20×CD3 Enables Systemic AAV Re-Dosing in Non-Human Primates

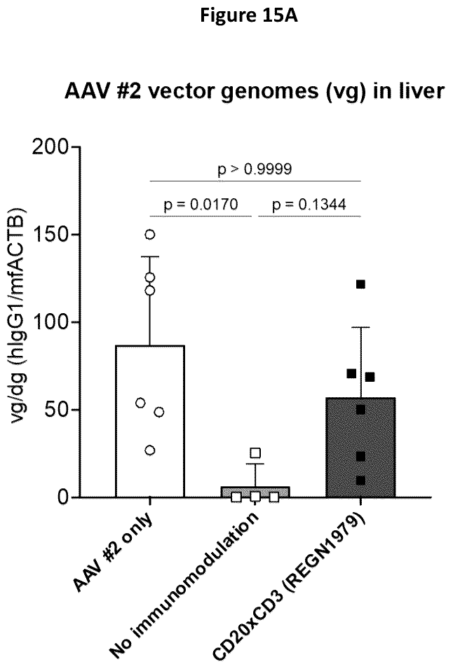

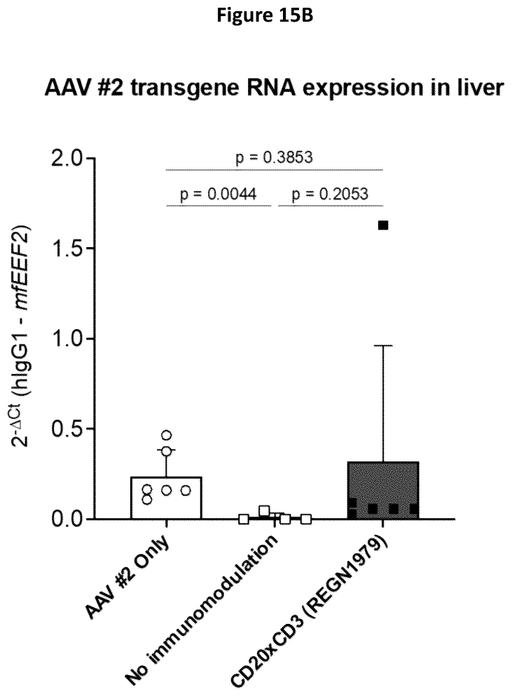

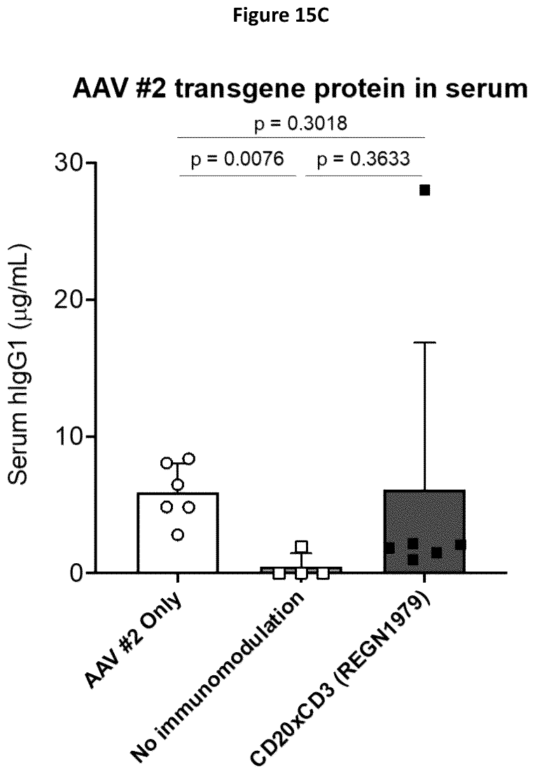

Next was evaluated whether the level of anti-AAV antibody titer suppression achieved in CD20×CD3-treated macaques (described in Example 13) was sufficient to enable systemic AAV re-dosing. All animals (including “AAV #2 only” animals that had not received AAV #1) were intravenously (i.v.) administered a second AAV8 vector (“AAV #2”) encoding a secreted human IgG1 monoclonal antibody expressed from a liver-specific promoter at 1e13 vg/kg. Four weeks later, animals were necropsied, and liver transduction was evaluated by digital PCR. As expected, few detectable vector genomes per diploid genome were observed in control animals that previously received AAV #1 but no immunomodulation (FIG. 15A). By contrast, CD20×CD3-treated macaques achieved robust transduction approaching that of previously-naïve control animals (“AAV #2 only”). Similarly, transgene mRNA (FIG. 15B) and hIgG1 protein (FIG. 15C) were readily detectable in liver and serum by RT-qPCR and ELISA, respectively, from CD20×CD3-treated and AAV #2 single-dosed control animals, but not re-dosed control animals that received no immunomodulation. Taken together, these findings show that systemic AAV vector re-administration is achievable via B cell depletion with bispecific CD20×CD3 antibody, likely due to the deeper level of B depletion achievable in lymph nodes by this approach versus conventional anti-CD20 therapeutics.

### REFERENCES

Choi et al., Successful AAV8 readministration: Suppression of capsid-specific neutralizing antibodies by a combination treatment of bortezomib and CD20 mAb in a mouse model of Pompe disease. J Gene Med. 2023; 25(8):e3509. doi: 10.1002/jgm.3509.
Limnander et al., A therapeutic strategy to target distinct sources of IgE and durably reverse allergy. Sci Transl Med. 2023; 15(726):eadf9561. doi: 10.1126/scitranslmed.adf9561.
Meliani et al., Antigen-selective modulation of AAV immunogenicity with tolerogenic rapamycin nanoparticles enables successful vector re-administration. Nat Commun. 2018; 9(1):4098. doi: 10.1038/s41467-018-06621-3.
Salabarria et al., Thrombotic microangiopathy following systemic AAV administration is dependent on anti-capsid antibodies. J Clin Invest. 2024; 134(1):e173510. doi: 10.1172/JCI173510.
Smith et al., A novel, native-format bispecific antibody triggering T-cell killing of B-cells is robustly active in mouse tumor models and cynomolgus monkeys. Sci Rep. 2015; 5:17943. doi: 10.1038/srep17943.
Unzu et al., Transient and intensive pharmacological immunosuppression fails to improve AAV-based liver gene transfer in non-human primates. J Transl Med. 2012; 10:122. doi: 10.1186/1479-5876-10-122.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. The disclosures of all patents and non-patent literature cited herein are expressly incorporated in their entirety by reference.

List of Sequences

SEQ ID NO: 1 Anti-BCMA HCVR DNA sequence
gaggtgcagc tggtggagtc tgggggaggc ttggtccagc ctggggggtc cctgagactc 60
tcctgtgcag cctctggatt cacctttagt aacttttgga tgacctgggt ccgccaggct 120
ccagggaagg ggctggagtg ggtggccaac atgaaccaag atggaagtga gaaatactat 180
gtggactctg tgaagggccg attcaccatc tccagagaca acgccaagag ctcactgtat 240
ctgcaaatga acagcctgag agccgaggac acggctgtgt attactgtgc gagagatcgg 300
gaatattgta ttagtaccag ctgctatgat gactttgact actggggcca gggaaccctg 360
gtcaccgtct cctca 375
SEQ ID NO: 2-Anti-BCMA HCVR Protein sequence
EVQLVESGGGLVQPGGSLRLSCAASGFTFSNFWMTWVRQAPGKGLEWVANMNQDGSEKYY
VDSVKGRFTISRDNAKSSLYLQMNSLRAEDTAVYYCARDREYCISTSCYDDFDYWGQGTL
VTVSS
SEQ ID NO: 3-Anti-BCMA HCDR1 DNA sequence
ggattcacct ttagtaactt ttgg
SEQ ID NO: 4-Anti-BCMA HCDR1 Protein sequence
GFTFSNFW
SEQ ID NO: 5-Anti-BCMA HCDR2 DNA sequence
atgaaccaag atggaagtga gaaa
SEQ ID NO: 6-Anti-BCMA HCDR2 Protein sequence
MNQDGSEK
SEQ ID NO: 7-Anti-BCMA HCDR3 DNA sequence
gcgagagatc gggaatattg tattagtacc agctgctatg atgactttga ctac
SEQ ID NO: 8-Anti-BCMA HCDR3 Protein sequence
ARDREYCISTSCYDDFDY
SEQ ID NO: 9-Anti-BCMA LCVR DNA sequence
gacatccaga tgacccagtc tccatcctcc ctgtctgcat ctgtaggaga cagagtcacc 60
atcacttgcc gggcaagtca gagcattage agctatttaa attggtatca gcagaaacca 120
gggaaagccc ctaagctcct gatctatgct gcatccagtt tgcatagtgg ggtcccatca 180
aggttcagtg gcagtggatc tgggacagat ttcactctca ccatcagcag tctgcaacct 240
gaagattttg caacttacta ctgtcaacag agttacagta cccctccgat caccttcggc 300
caagggacac gactggagat taaa 324
SEQ ID NO: 10-Anti-BCMA LCVR protein sequence
DIQMTQSPSSLSASVGDRVTITCRASQSISSYLNWYQQKPGKAPKLLIYAASSLHSGVPS
RFSGSGSGTDFTLTISSLQPEDFATYYCQQSYSTPPITFGQGTRLEIK
SEQ ID NO: 11-Anti-BCMA LCDR1 DNA sequence
cagagcatta gcagctat
SEQ ID NO: 12-Anti-BCMA LCDR1 protein sequence
QSISSY
SEQ ID NO: 13-Anti-BCMA LCDR2 DNA sequence
gctgcatcc
SEQ ID NO: 14-Anti-BCMA LCDR2 protein sequence
AAS
SEQ ID NO: 15-Anti-BCMA LCDR3 DNA sequence
caacagagtt acagtacccc tccgatcacc
SEQ ID NO: 16-Anti-BCMA LCDR3 protein sequence
QQSYSTPPIT
SEQ ID NO: 17-Common LCVR DNA sequence
gacatccaga tgacccagtc tccatcctcc ctgtctgcat ctgtaggaga cagagtcacc 60
atcacttgcc gggcaagtca gagcattagc agctatttaa attggtatca gcagaaacca 120
gggaaagccc ctaagctcct gatctatgct gcatccagtt tgcaaagtgg ggtcccgtca 180
aggttcagtg gcagtggatc tgggacagat ttcactctca ccatcagcag tctgcaacct 240
gaagattttg caacttacta ctgtcaacag agttacagta cccctccgat caccttcggc 300
caagggacac gactggagat taaa 324
SEQ ID NO: 18-Common LCVR protein sequence
DIQMTQSPSSLSASVGDRVTITCRASQSISSYLNWYQQKPGKAPKLLIYAASSLQSGVPS
RFSGSGSGTDFTLTISSLQPEDFATYYCQQSYSTPPITFGQGTRLEIK
SEQ ID NO: 19-Common LCDR1 DNA sequence
cagagcatta gcagctat
SEQ ID NO: 20-Common LCDR1 protein sequence
QSISSY
SEQ ID NO: 21-Common LCDR2 DNA sequence
gctgcatcc
SEQ ID NO: 22-Common LCDR2 protein sequence
AAS
SEQ ID NO: 23-Common LCDR3 DNA sequence
caacagagtt acagtacccc tccgatcacc
SEQ ID NO: 24-Common LCDR3 protein sequence
QQSYSTPPIT
SEQ ID NO: 25-Anti-CD3 HCVR DNA sequence
gaagtacagc ttgtagaatc cggcggagga ctggtacaac ctggaagaag tcttagactg 60
agttgcgcag ctagtgggtt tacattcgac gattacagca tgcattgggt gaggcaagct 120
cctggtaaag gattggaatg ggttagcggg atatcatgga actcaggaag caagggatac 180
gccgacagcg tgaaaggccg atttacaata tctagggaca acgcaaaaaa ctctctctac 240
cttcaaatga actctcttag ggcagaagac acagcattgt attattgcgc aaaatacggc 300
agtggttatg gcaagtttta tcattatgga ctggacgtgt ggggacaagg gacaacagtg 360
acagtgagta gc 372
SEQ ID NO: 26-Anti-CD3 HCVR protein sequence
EVQLVESGGGLVQPGRSLRLSCAASGFTFDDYSMHWVRQAPGKGLEWVSGISWNSGSKGYA
DSVKGRFTISRDNAKNSLYLQMNSLRAEDTALYYCAKYGSGYGKFYHYGLDVWGQGTTVTVS
S
SEQ ID NO: 27-Anti-CD3 HCDR1 DNA sequence
gggtttacat tcgacgatta cagc
SEQ ID NO: 28-Anti-CD3 HCDR1 protein sequence
GFTFDDYS
SEQ ID NO: 29-Anti-CD3 HCDR2 DNA sequence
atatcatgga actcaggaag caag
SEQ ID NO: 30-Anti-CD3 HCDR2 protein sequence
ISWNSGSK
SEQ ID NO: 31-Anti-CD3 HCDR3 DNA sequence
gcaaaatacg gcagtggtta tggcaagttt tatcattatg gactggacgt g
SEQ ID NO: 32-Anti-CD3 HCDR3 protein sequence
AKYGSGYGKFYHYGLDV
SEQ ID NO: 33-Anti-CD3 HCVR DNA sequence
gaagtacagc ttgtagaatc cggcggagga ctggtacaac ctggaagaag tcttagactg 60
agttgcgcag ctagtgggtt tacattcgac gattacagca tgcattgggt gaggcaagct 120
cctggtaaag gattggaatg ggttagcggg atatcatgga actcaggaag catcggatac 180
gccgacagcg tgaaaggccg atttacaata totagggaca acgcaaaaaa ctctctctac 240
cttcaaatga actctcttag ggcagaagac acagcattgt attattgcgc aaaatacggc 300
agtggttatg gcaagtttta ttattatgga atggacgtgt ggggacaagg gacaacagtg 360
acagtgagta gc 372
SEQ ID NO: 34-Anti-CD3 HCVR protein sequence
EVQLVESGGGLVQPGRSLRLSCAASGFTFDDYSMHWVRQAPGKGLEWVSGISWNSGSIGY
ADSVKGRFTISRDNAKNSLYLQMNSLRAEDTALYYCAKYGSGYGKFYYYGMDVWGQGTTV
TVSS
SEQ ID NO: 35-Anti-CD3 HCDR1 DNA sequence
gggtttacat tcgacgatta cagc
SEQ ID NO: 36-Anti-CD3 HCDR1 protein sequence
GFTFDDYS
SEQ ID NO: 37-Anti-CD3 HCDR2 DNA sequence
atatcatgga actcaggaag catc
SEQ ID NO: 38-Anti-CD3 HCDR2 protein sequence
ISWNSGSI
SEQ ID NO: 39-Anti-CD3 HCDR3 DNA sequence
gcaaaatacg gcagtggtta tggcaagttt tattattatg gaatggacgt g
SEQ ID NO: 40-Anti-CD3 HCDR3 protein sequence
AKYGSGYGKFYYYGMDV
SEQ ID NO: 41-Anti-BCMA Heavy Chain Protein Sequence
(IgG4 Heavy Chain Constant Region)
EVQLVESGGGLVQPGGSLRLSCAASGFTFSNFWMTWVRQAPGKGLEWVANMNQDGSEKYY
VDSVKGRFTISRDNAKSSLYLQMNSLRAEDTAVYYCARDREYCISTSCYDDFDYWGQGTLVTV
SSASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPPVAGPSVFLFPP
KPKDTLMISRTPEVTCVVVDVSQEDPEVQFNWYVDGVEVHNAKTKPREEQFNSTYRVVSVLTV
LHQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALH
NHYTQKSLSLSLGK
SEQ ID NO: 42-Anti-CD3 Heavy Chain Protein Sequence
(IgG4 Heavy Chain Constant Region with H435R/Y436F)
EVQLVESGGGLVQPGRSLRLSCAASGFTFDDYSMHWVRQAPGKGLEWVSGISWNSGSKGYA
DSVKGRFTISRDNAKNSLYLQMNSLRAEDTALYYCAKYGSGYGKFYHYGLDVWGQGTTVTVS
SASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPPCPAPPVAGPSVFLFPPK
PKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHN
RFTQKSLSLSPGK
SEQ ID NO: 43-Common Anti-BMCA and Anti-CD3 Light Chain Protein Sequence
(Kappa Light Chain Constant Region)
DIQMTQSPSSLSASVGDRVTITCRASQSISSYLNWYQQKPGKAPKLLIYAASSLQSGVPSRFSG
SGSGTDFTLTISSLQPEDFATYYCQQSYSTPPITFGQGTRLEIKRTVAAPSVFIFPPSDEQLKSG
TASVVCLLNNFYPREAKVQWKVDNALQSGNSQESVTEQDSKDSTYSLSSTLTLSKADYEKHKV
YACEVTHQGLSSPVTKSFNRGEC
SEQ ID NO: 44-Anti-CD20 HCVR Protein Sequence
EVQLVESGGGLVQPGRSLRLSCVASGFTFNDYAMHWVRQAPGKGLEWVSVISWNSDSIGYA
DSVKGRFTISRDNAKNSLYLQMHSLRAEDTALYYCAKDNHYGSGSYYYYQYGMDVWGQGTT
VTVSS
SEQ ID NO: 45-Common LCVR Protein Sequence
EIVMTQSPATLSVSPGERATLSCRASQSVSSNLAWYQQKPGQAPRLLIYGASTRATGIPARFS
GSGSGTEFTLTISSLQSEDFAVYYCQHYINWPLTFGGGTKVEIKR
SEQ ID NO: 46-Anti-CD3 HCVR Protein Sequence
EVQLVESGGGLVQPGRSLRLSCAASGFTFDDYTMHWVRQAPGKGLEWVSGISWNSGSIGYA
DSVKGRFTISRDNAKKSLYLQMNSLRAEDTALYYCAKDNSGYGHYYYGMDVWGQGTTVTVAS
SEQ ID NO: 47-Anti-CD20 HCDR1 Protein Sequence
GFTFNDYA
SEQ ID NO: 48-Anti-CD20 HCDR2 Protein Sequence
ISWNSDSI
SEQ ID NO: 49-Anti-CD20 HCDR3 Protein Sequence
AKDNHYGSGSYYYYQYGMDV
SEQ ID NO: 50-Common LCDR1 Protein Sequence
QSVSSN
SEQ ID NO: 51-Common LCDR2 Protein Sequence
GAS
SEQ ID NO: 52-Common LCDR3 Protein Sequence
QHYINWPLT
SEQ ID NO: 53-Anti-CD3 HCDR1 Protein Sequence
GFTFDDYT
SEQ ID NO: 54-Anti-CD3 HCDR2 Protein Sequence
ISWNSGSI
SEQ ID NO: 55-Anti-CD3 HCDR3 Protein Sequence
AKDNSGYGHYYYGMDV
SEQ ID NO: 56-His-6
HHHHHH
SEQ ID NO: 57-His-8
HHHHHHHH
SEQ ID NO: 58-His-10
HHHHHHHHHH
SEQ ID NO: 59-A poly-A tail comprising 20, 30, 40, 50, 60, 70,
80, 90, or 100 adenines
aaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaa
SEQ ID NO: 60-A poly-A tail comprising 95-100 adenines
aaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaa

1. A method for inhibiting or preventing an immune response to an immunogen in a subject in need thereof, wherein the subject has pre-existing immunity against the immunogen, said method comprising administering to the subject an effective amount of a plasma cell depleting agent.

2. The method of claim 1, wherein the method results in inhibiting or preventing generation of antibodies to the immunogen in the subject.

3. A method for increasing effectiveness of re-administration of an immunogen to a subject in need thereof, wherein the subject has pre-existing immunity against the immunogen, said method comprising administering to the subject an effective amount of a plasma cell depleting agent.

4.-5. (canceled)

6. The method of claim 1, comprising determining the presence of neutralizing antibodies to the immunogen in the subject.

7. The method of claim 1, wherein the plasma cell depleting agent is administered before, at the same time as, or after the administration of the immunogen.

8.-9. (canceled)

10. The method of claim 1, wherein the immunogen is administered two or more times and the plasma cell depleting agent is administered before and/or between each of the administrations of the immunogen.

11. The method of claim 1, wherein the immunogen is an immunogenic delivery vehicle, a polypeptide encoded by a transgene contained within an immunogenic delivery vehicle, a polynucleotide encoded by a transgene contained within an immunogenic delivery vehicle, a polypeptide, a polynucleotide, a glycan, or a lipid.

12. (canceled)

13. A method for increasing or maintaining the level of a transgene expression in a subject in need thereof, said method comprising administering to the subject an effective amount of a plasma cell depleting agent.

14.-17. (canceled)

18. The method of claim 11, wherein the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell.

19. (canceled)

20. The method of claim 3, wherein the method is for increasing effectiveness of administration of a subsequently administered viral vector following administration of an originally administered viral vector, and wherein the subsequently administered viral vector is of the same or similar viral origin as the originally administered viral vector.

21.-26. (canceled)

27. The method of claim 20, wherein the viral vector is derived from an adeno-associated virus (AAV), an adenovirus, a retrovirus, or an oncolytic virus.

28. (canceled)

29. The method of claim 1, wherein the plasma cell depleting agent is capable of depleting long-lived plasma cells (LLPC).

30. The method of claim 1, wherein the plasma cell depleting agent is a B cell maturation antigen (BCMA) targeting agent.

31. The method of claim 30, wherein the BCMA targeting agent is a chimeric antigen receptor (CAR) against BCMA or an anti-BCMA antibody or a functional fragment thereof, optionally, conjugated to a cytotoxic agent.

32. (canceled)

33. The method of claim 31, wherein the anti-BCMA antibody is a multispecific antibody or a functional fragment thereof.

34. The method of claim 33, wherein the multispecific anti-BCMA antibody or functional fragment thereof targets BCMA and CD3.

35. The method of claim 34, wherein the multispecific anti-BCMA antibody or functional fragment thereof is anti-BCMA×CD3 bispecific antibody or functional fragment thereof.

36. The method of claim 35, wherein the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B.

37.-46. (canceled)

47. The method of claim 1, further comprising administering to the subject an effective amount of a B cell depleting agent and/or an immunoglobulin depleting agent.

48. The method of claim 47, wherein the B cell depleting agent is administered before, at the same time as, or after the plasma cell depleting agent, and/or the immunoglobulin depleting agent is administered after the plasma cell depleting agent.

49. (canceled)

50. The method of claim 47, wherein the B cell depleting agent is capable of depleting B cells and plasma cells that express low levels of BCMA, and/or is an agent that binds to a B cell surface molecule, and/or is an agent targeting a B cell survival factor.

51. (canceled)

52. The method of claim 47, wherein the B cell depleting agent is selected from anti-CD19 antibodies, anti-CD20 antibodies, anti-CD22 antibodies, anti-CD79 antibodies, multispecific antibodies combining two or more of any of said antibody specificities, multispecific antibodies combining any of said antibody specificities with anti-CD3 antibodies, functional fragments of any of said antibodies, and any combinations thereof.

53.-55. (canceled)

56. The method of claim 52, wherein the B cell depleting agent is a multispecific anti-CD20 antibody or functional fragment thereof which targets CD20 and CD3.

57.-67. (canceled)

68. The method of claim 47, wherein the B cell depleting agent is a BLyS/BAFF inhibitor, an APRIL inhibitor, a BLyS receptor 3/BAFF receptor inhibitor, or any combination thereof.

69. (canceled)

70. The method of claim 47, wherein the immunoglobulin depleting agent is a neonatal Fc receptor (FcRn) blocker.

71. The method of claim 70, wherein the FcRn blocker is selected from Efgartigimod (ARGX-113), Rozanolixizumab (UCB7665), Batoclimab (RVT-1401), Nipocalimab (M281), Orilanolimab (SYNT001), IMVT-1402, and any combinations thereof.

72.-73. (canceled)

74. A pharmaceutical composition comprising (i) optionally, an immunogen, (ii) a plasma cell depleting agent, (iii) optionally, a B cell depleting agent and/or an immunoglobulin depleting agent, and (iv) a pharmaceutically acceptable carrier and/or excipient.

75. (canceled)

76. A kit comprising (i) optionally, an immunogen, (ii) a plasma cell depleting agent, (iii) optionally, a B cell depleting agent and/or an immunoglobulin depleting agent, and (iv) instructions for use.

77.-163. (canceled)
